(19) United States
(12) Patent Application Publication
(10) Pub. No.: US 2012/0165310 A1
(43) Pub. Date: Jun. 28, 2012

(54) ETHER DERIVATIVES OF BICYCLIC HETEROARYLS

(75) Inventors: Bei Chen, San Diego, CA (US); Robin Alec Fairhurst, West Sussex (GB); Andreas Floersheimer, Dornach (CH); Pascal Furet, Thann (FR); Vito Guagnano, Basel (CH); Jiang Songchun, San Diego, CA (US); Wenshuo Lu, San Diego, CA (US); Thomas H. Marsilje, III, San Diego, CA (US); Clive McCarthy, Froidefontaine (FR); Pierre-Yves Michellys, San Marcos, CA (US); Frederic Stauffer, Hesingue (CH); Stefan Stutz, Basel (CH); Andrea Vaupel, Riehen (CH)

(73) Assignee: NOVARTIS AG, Basel (CH)

(21) Appl. No.: 13/394,534

(22) PCT Filed: Sep. 10, 2010
(86) PCT No.: PCT/EP2010/063334

§ 371 Date: Mar. 6, 2012

Related U.S. Application Data
(60) Provisional application No. 61/241,182, filed on Sep. 10, 2009.

(51) Int. Cl. A61K 31/519; A61K 31/541; C07D 493/08; A61K 31/5377; A61K 31/554; A61P 19/00; A61K 31/5386; C07D 495/08; A61P 35/00; A61P 11/00; A61P 1/16; C07D 487/04; C07D 487/08

(52) U.S. Cl. 514/210.21; 544/280; 544/58.2; 514/228.5; 514/252.16; 514/265.1; 544/117; 514/234.2; 540/544; 514/211.15; 544/230; 544/71; 514/230.5

(57) ABSTRACT

The invention relates to compounds of formula I,

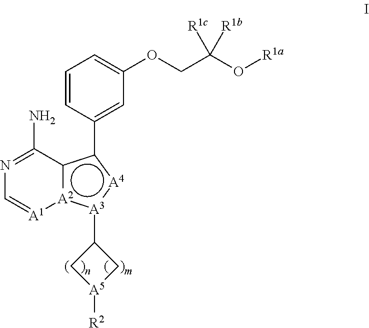

wherein the substituents are as defined in the specification; to processes for the preparation of such compounds; pharmaceutical compositions comprising such compounds; such compounds as a medicament; such compounds for the treatment of a proliferative disease.

### BACKGROUND OF THE INVENTION

The invention relates to new ether derivatives of bicyclic heteroaryls; processes for the preparation of such derivatives; pharmaceutical compositions comprising such derivatives optionally in combination with one or more other pharmaceutically active compounds; such derivatives optionally in combination with one or more other pharmaceutically active compounds as a medicament; such derivatives optionally in combination with one or more other pharmaceutically active compounds for the treatment of a proliferative disease, such as a tumour disease (also including a method for the treatment of such diseases in mammals, especially in humans); and the use of such derivatives for the preparation of a pharmaceutical composition (medicament) for the treatment of a proliferative disease, such as a tumour.

Insulin-like growth factor (IGF-1) signaling is highly implicated in cancer, with the IGF-1 receptor (IGF-1R) as the predominating factor. IGR-1R is important for tumor transformation and survival of malignant cells, but is only partially involved in normal cell growth. Targeting of IGF-1R has been suggested to be a promising option for cancer therapy. (Larsson et al., Br. J. Cancer 92:2097-2101 (2005)). WO 2005/097800 discloses certain 6,6-bicyclic ring substituted heterobicyclic derivatives having therapeutic activity as IGF-1R inhibitors. WO 2005/037836 discloses certain imidazopyrazine derivatives having therapeutic activity as IGF-1R inhibitors. WO 97/028161 discloses certain pyrrolopyrimidine derivatives having therapeutic activity as inhibitors of tyrosine protein kinase. WO 2002/092599 discloses certain pyrrolopyrimidine derivatives having therapeutic activity as IGF-1R inhibitors. Mulvihill et al. (Bioorg. Med. Chem. Lett. 17 (2007) 1091 ff) disclose certain imidazopyrazines as IGF-1R inhibitors.

Because of the emerging disease-related roles of IGF-1R, there is a continuing need for compounds which may be useful for treating and preventing a disease which responds to inhibition of IGF-1R, particularly for compounds with improved efficacy, tolerabilty and/or selectivity.

Surprisingly, it has now been found that the compounds of formula I, described below, are potent inhibitors of the tyrosine kinase activity of the Insulin-like growth factor I receptor (IGF-IR) and inhibit IGF-IR-dependent cell proliferation. The presence of the substituents of the scaffold as defined below is considered important for the efficacy, tolerability and/or the selectivity of the compounds of the present invention as IGF-IR tyrosine kinase inhibitors and their potential to inhibit IGF-IR-dependent cell proliferation. The compounds of formula I therefore permit, for example, a therapeutic approach, especially for diseases in the treatment of which, and also for the prevention of which, an inhibition of the IGF-IR tyrosine kinase and/or of the IGF-IR-dependent cell proliferation shows beneficial effects. Such diseases include proliferative diseases, such as tumours, like for example breast, renal, prostate, colorectal, thyroid, ovarian, pancreas, neuronal, lung, uterine and gastro-intestinal tumours as well as osteosarcomas and melanomas. Compounds of the invention show improved efficacy, tolerability and/or selectivity when compared to known IGF-1R inhibitors. Without being bound to theory, it is believed that several factors contribute to the improvements in efficacy and tolerability, for example, increased metabolic stability and the reduced formation of multiple kinase-active metabolites. Although known compounds have been shown to produce desirable effects in in-vivo models through the inhibition of IGF-1 receptor activity, they have been found to undergo extensive metabolism. This not only limits the pharmacokinetic profile of such derivatives, but also generates metabolites, which show multiple potent kinase activities.

### SUMMARY OF THE INVENTION

The invention relates to compounds of formula I,

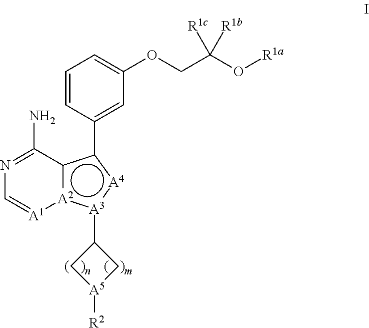

wherein A¹-A⁵, R^{1a}, R^{1b}, R^{1c}, R², m, and n are as defined below; to processes for the preparation of such compounds; pharmaceutical compositions comprising such compounds; such compounds as a medicament; and such compounds for the treatment of a proliferative disease.

### DETAILED DESCRIPTION OF THE INVENTION

The invention relates in a first aspect to compounds of formula (I)

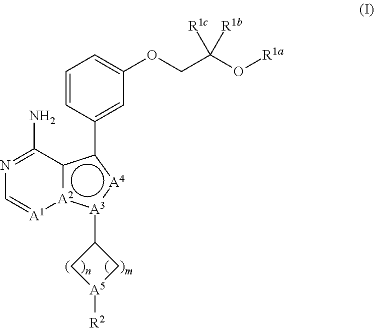

or a salt thereof, wherein
A¹ represents N, A² represents C, A³ represents N, and A⁴ represents CH; or
A¹ represents CH, A² represents N, A³ represents C, and A⁴ represents N;
R^{1a} and R^{1b} together with the atoms to which they are attached form a 3-12 membered monocyclic or bicyclic, saturated or partly saturated, heterocyclyl having 1-3 oxygen atoms, 0-3 nitrogen atoms, and 0-2 sulfur atoms; said heterocyclyl being optionally substituted with one to three substituents each independently selected from the group consisting of C₁₋₇alkyl; C₁₋₇alkoxy; halo; cyano; hydroxy; oxo; nitro; amino; C₁₋₇alkylamino; and di(C₁₋₇alkyl)amino; and
R^{1c} represents hydrogen or C₁₋₇alkyl; or
R^{1a} and R^{1b} and R^{1c} together with the atoms to which they are attached form a 6-12 membered bicyclic, saturated or partly saturated, heterocyclyl, having 1-3 oxygen atoms, 0-3 nitrogen atoms, and 0-2 sulfur atoms; said heterocyclyl being optionally substituted with one to three substituents each independently selected from the group consisting of C₁₋₇alkyl; C₁₋₇alkoxy; halo; cyano; hydroxy; oxo; nitro; amino C₁₋₇alkylamino; and di(C₁₋₇alkyl)amino; or
R^{1a} represents branched C₃₋₈alkyl or C₃₋₁₀cycloalkyl;
R^{1b} represents hydrogen or C₁₋₇alkyl; and
R^{1c} represents hydrogen or C₁₋₇alkyl;
m represents 1 or 2;
n represents 1 or 2;
A⁵-R² represents N—R², NC(H)R²R³, CR²R³, or CR³—CH₂—R²;
R³ represents hydrogen, C₁₋₇alkyl, or hydroxy; and
R² represents a 3-12 membered monocyclic or bicyclic, saturated or partly saturated, heterocyclyl having 1-3 nitrogen atoms, 0-3 oxygen atoms, and 0-3 sulfur atoms; said heterocyclyl being optionally substituted with one to four substituents each independently selected from the group consisting of halo; cyano; oxo; hydroxy; carboxy; amino; nitro; SO₂R⁴; COR⁵; C₁₋₇alkyl; C₁₋₇alkyl halo optionally substituted with one hydroxy; C₁₋₇alkoxy; hydroxy-C₁₋₇alkyl; piperazinyl C₁₋₃alkyl; aminocarbonyl; C₁₋₇alkylaminocarbonyl; and di(C₁₋₇alkyl)aminocarbonyl; or
R² represents OH; SH; C₁₋₇alkoxy; C₁₋₇alkylthio; C₁₋₇alkyl optionally substituted with one SO₂R⁴ or NHR⁴ group; NHR⁵; NHC(O)R⁵; NHC(O)NHR⁵; NHC(O)OR⁵; SO₂R⁴; NHSO₂R⁵; NHNHC(O)R⁴; imidazolyl optionally substituted with one methyl, CH₂OH or C(O)OR⁴; tetrazolyl optionally substituted with one methyl; or oxazoly; or
R² and R³ together with

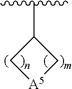

the moiety form 5,7-dioxa-spiro[3.4]octanyl, 5-oxa-7-aza-spiro[3.4]octanyl, or 5-oxa-8-aza-spiro[3.5]nonanyl, each of which is optionally substituted with one to three substituents each independently selected from the group consisting of: halo; cyano; oxo; hydroxy; amino; nitro; C₁₋₇alkyl; C₁₋₇alkoxy; hydroxy-C₁₋₇alkyl; aminocarbonyl; C₁₋₇alkylaminocarbonyl; and di(C₁₋₇alkyl)aminocarbonyl;
R⁴ represents hydrogen or C₁₋₇alkyl; and
R⁵ represents hydrogen; C₁₋₇alkyl; hydroxy-C₁₋₇alkyl; C₁₋₇alkyl halo; C₃₋₇ cycloalkyl optionally substituted with one or two C₁₋₃alkyl groups; piperazinly optionally substituted with one C₇₋₃alkyl; tetrahydropyranyl; pyridinyl optionally substituted with one methyl or cyano.

The invention may be more fully appreciated by reference to the following description, including the following glossary of terms and the concluding examples. As used herein, the terms “including”, “containing” and “comprising” are used herein in their open, non-limiting sense.

Where compounds of formula I are mentioned, this is meant to include also the tautomers, N-oxides, and S-oxides of the compounds of formula I.

Where the plural form is used for compounds, salts, and the like, this is taken to mean also a single compound, salt, or the like.

Any formula given herein is intended to represent compounds having structures depicted by the structural formula as well as certain variations or forms. In particular, compounds of any formula given herein may have asymmetric centers and therefore exist in different enantiomeric forms. If at least one asymmetrical carbon atom is present in a compound of the formula I, such a compound may exist in optically active form or in the form of a mixture of optical isomers, e.g. in the form of a racemic mixture. All optical isomers and their mixtures, including the racemic mixtures, are part of the present invention. Thus, any given formula given herein is intended to represent a racemate, one or more enantiomeric forms, one or more diastereomeric forms, one or more atropisomeric forms, and mixtures thereof. Furthermore, certain structures may exist as geometric isomers (i.e. cis and trans isomers), as tautomers, or as atropisomers.

“Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers or axes and may thus give rise to enantiomers, diastereomers, and other stereoisomeric forms that may be defined, in terms of absolute stereochemistry, as (R)— or (S)—. The present invention is meant to include all such possible isomers, including racemic mixtures, optically pure forms and intermediate mixtures. Optically active (R)— and (S)— isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

Any formula given herein is intended to represent hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F, ³¹P, ³²P, ³⁵S, ³⁶Cl, ¹²⁵I respectively. The invention includes various isotopically labeled compounds as defined herein, for example those into which radioactive isotopes, such as ³H, ¹³C, and ¹⁴C, are present.

Such isotopically labelled compounds are useful in metabolic studies (preferably with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly preferred for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Pharmaceutically acceptable solvates in accordance with the invention include those wherein the solvent of crystallization may be isotopically substituted, e.g. D₂O, d₆-acetone, d₆-DMSO.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula I. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this invention any atom specifically designated as a deuterium (D) is meant to represent deuterium, for example in the ranges given above.

Compounds of the invention, i.e. compounds of formula I that contain groups capable of acting as donors and/or acceptors for hydrogen bonds may be capable of forming co-crystals with suitable co-crystal formers. These co-crystals may be prepared from compounds of formula I by known co-crystal forming procedures. Such procedures include grinding, heating, co-subliming, co-melting, or contacting in solution compounds of formula I with the co-crystal former under crystallization conditions and isolating co-crystals thereby formed. Suitable co-crystal formers include those described in WO 2004/078163. Hence the invention further provides co-crystals comprising a compound of formula I.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula (where one or more up to all more general expressions in embodiments characterized as preferred above or below can be replaced with a more specific definition, thus leading to a more preferred embodiment of the invention, respectively).

The general terms used hereinbefore and hereinafter preferably have within the context of this disclosure the following meanings, unless otherwise indicated:

Carbon containing groups, moieties or molecules contain 1 to 7, preferably 1 to 6, more preferably 1 to 4 or 1 to 3, most preferably 1 or 2, carbon atoms. Any non-cyclic carbon containing group or moiety with more than 1 carbon atom is straight-chain or branched. The prefix “lower” denotes a radical having 1 to 7, preferably 1 to 4 or 1 to 3 carbon atoms, the radicals in question being either unbranched or branched with single or multiple branching.

The term “alkyl” refers to a straight-chain or branched-chain alkyl group, preferably represents a straight-chain or branched-chain C₁₋₁₂alkyl, for example, methyl, ethyl, n- or isopropyl, n-, iso-, sec- or tert-butyl, n-pentyl, n-hexyl, n-heptyl, n-octyl, 2-ethylhexyl, n-nonyl, n-decyl, n-undecyl, n-dodecyl. A “lower alkyl” is, for example, methyl, ethyl, n-propyl, isopropyl, n-butyl, isobutyl, sec-butyl, tert-butyl, n-pentyl, isopentyl, neopentyl, n-hexyl or n-heptyl. Thus, C₁₋₇alkyl are either unbranched or branched (with single or multiple branching) alkyl radicals having from 1 to 7 carbon atoms, respectively, and include methyl, ethyl, n-propyl, 2-propyl, n-butyl, sec-butyl, t-butyl, and the like.

Each alkyl part of other groups like “alkoxy”, “alkoxyalkyl”, “alkoxycarbonyl”, “alkoxycarbonylalkyl”, “alkylsulfonyl”, “alkylsulfoxyl”, “alkylamino”, “haloalkyl” shall have the same meaning as described in the above-mentioned definition of “alkyl”.

The term “alkoxy” refers to alkyl-O—, wherein alkyl is defined herein above. Representative examples of alkoxy include, but are not limited to, methoxy, ethoxy, propoxy, 2-propoxy, butoxy, tert-butoxy, pentyloxy, hexyloxy, cyclopropyloxy-, cyclohexyloxy- and the like. Typically, alkoxy groups have about 1-7, more preferably about 1-4 or 1-3 carbons.

The term “alkyl halo” refers to an alkyl as defined herein that is substituted by one or more halo groups as defined herein. The haloalkyl can be monohaloalkyl, dihaloalkyl or polyhaloalkyl including perhaloalkyl. A monohaloalkyl can have one iodo, bromo, chloro or fluoro within the alkyl group. Dihaloalky and polyhaloalkyl groups can have two or more of the same halo atoms or a combination of different halo groups within the alkyl. Typically the polyhaloalkyl contains up to 12, or 10, or 8, or 6, or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms.

The term “amino” refers to a NH₂ radical.

The term “aminocarbonyl” refers to a C(O)NH₂ radical.

The term “aryl” refers to an aromatic hydrocarbon group having 6-20 carbon atoms in the ring portion. Typically, aryl is monocyclic, bicyclic or tricyclic aryl having 6-20 carbon atoms. Furthermore, the term “aryl” as used herein, refers to an aromatic substituent which can be a single aromatic ring, or multiple aromatic rings that are fused together. Non-limiting examples include phenyl, naphthyl or tetrahydronaphthyl, each of which may optionally be substituted by 1-4 substituents, such as alkyl, trifluoromethyl, cycloalkyl, halogen, hydroxy, alkoxy, acyl, alkyl-C(O)—O—, aryl-O—, heteroaryl-O—, amino, thiol, alkyl-S—, aryl-S—, nitro, cyano, carboxy, alkyl-O—C(O)—, carbamoyl, alkyl-S(O)—, sulfonyl, sulfonamido, phenyl, and heterocyclyl.

The term “carboxy” refers to a COOH radical

The term “cycloalkyl” refers to a saturated or partially saturated, monocyclic, fused polycyclic, or Spiro polycyclic, carbocycle having from 3 to 12, preferably 3 to 10, most preferably 3 to 7 ring atoms per carbocycle. Illustrative examples of cycloalkyl groups include the following moieties: cyclopropyl, cyclobutyl, cyclpentyl, cylclohexyl, and cycloheptyl. The term cycloalkyl excludes “aryl”.

The term “halogen” (or halo) denotes fluorine, bromine, chlorine or iodine, in particular fluorine, chlorine. Halogen-substituted groups and moieties, such as alkyl substituted by halogen (haloalkyl) can be mono-, di-, poly- or per-halogenated.

Hetero atoms are atoms other than Carbon and Hydrogen, preferably nitrogen (N), oxygen (O) or sulfur (S), in particular nitrogen or oxygen.

The term “heterocyclyl” refers to a heterocyclic radical that is saturated or partially saturated and is preferably a monocyclic or a polycyclic ring (in case of a polycyclic ring particularly a bicyclic, tricyclic or spirocyclic ring); and has 3 to 24, more preferably 4 to 16 or 3 to 12, most preferably 5 to 10 and most preferably 4, 5, 6 or 7 ring atoms; wherein one or more, preferably one to four, especially one or two ring atoms are a heteroatom (the remaining ring atoms therefore being carbon). The bonding ring (i.e. the ring connecting to the molecule) preferably has 4 to 12, especially 5 to 7 ring atoms. The term heterocyclyl excludes heteroaryl. The heterocyclic radical (heterocyclyl) may be unsubstituted or substituted by one or more, especially 1 to 3, substituents. A polycyclic heterocyclic moiety may be annellated to a further saturated, partly saturated or unsaturated ring, forming a polycyclic heterocyclic radical.

Such polycyclic heterocyclic radical includes moieties wherein one or two benzene radicals are annellated to a moncyclic heterocyclic radical as defined above. Further, a polycyclic heterocyclic moiety may be bridged by an alkandiyl or alkendiyl as defined herein. Further, a polycyclic heterocyclic moiety may be connected to a further heterocyclyl or cycloalkyl via one connecting atom to form a spirocyclic heterocyclic moiety. Examples of heterocyclyl groups include azetidinyl, oxetanyl, thietanyl, pyrrolidinyl, piperidinyl, piperazinyl, morpholinyl, thiomorpholinyl, thiazolidinyl, tetrahydropyranyl, tetrahydrofuranyl, azepanyl, thiazepanyl, azabicyclo[2.2.1]heptanyl, azabicyclo[3.1.0]hexanyl, diaza-bicyclo[2.2.1]heptanyl, 2-thia-5-aza-bicyclo[2.2.1]heptanyl, 8-aza-bicyclo[3.2.1]octanyl, 2,3-dihydro-1H-isoindolyl, and 7-oxabicyclo[2.2.1]heptyl.

In the context of R², preferred heterocyclic radicals contain at least one nitrogen ring atom whereby the binding of the heterocyclic radical to the radical of the molecule of formula (I) occurs preferably via a nitrogen ring atom. Most preferably a heterocyclic radical is azetidinyl, pyrrolidinyl, piperidinyl, azepanyl, piperazinyl, tetrahydropyranyl, morpholinyl or thiomorpholinyl, wherein said radicals are optionally substituted by one to four, preferably one or two substituents, each independently selected from the group consisting of C₁₋₃alkyl; fluoro; hydroxy; oxo; carboxy; C₁₋₃alkoxy carbonyl; C₁₋₃alkyl halo optionally substituted with one hydroxy; hydroxy-C₁₋₃alkyl; piperazinly C₁₋₃alkyl; aminocarbonyl; C₁₋₃alkylaminocarbonyl; methoxycarbonyl; methylsulfonyl; and methylcarboxy.

In the context of R^{1a} and R^{1b} together with the CH₂O resdiue forming a heterocyclyl, preferred heterocyclic radicals contain no additional hetero atom. Most preferably a heterocyclic radical is selected from the group consisting of tetrahydrofuryl and tetrahydro-2H-pyranyl wherein said radicals are optionally substituted by one or more, preferably one or two substituents each independently selected from the group consisting of C₁₋₇alkyl; C₁₋₇alkoxy; halo; cyano; hydroxy; oxo; nitro; amino; C₁₋₇alkylamino; and di(C₁₋₇alkyl)amino.

In the context of R^{1a}, R^{1b} and R^{1b} together with the CH₂O residue forming a heterocyclyl, preferred heterocyclic radicals contain no additional hetero atom. Most preferably such a heterocyclic radical selected from the group consisting of 2-oxabicyclo[1.1.1]pentanyl; 5-oxabicyclo[2.1.1]hexanyl; 2-oxabicyclo[2.1.1]hexanyl; 6-oxabicyclo[3.1.1]heptanyl; 2-oxabicyclo[2.2.1]heptanyl; 2-oxabicyclo[3.1.1]heptanyl; 7-oxabicyclo[2.2.1]heptanyl; d₉-7-oxabicyclo[2.2.1]heptanyl, 8-oxabicyclo[3.2.1]octanyl; 2-oxabicyclo[2.2.2]octanyl; 6-oxabicyclo[3.2.1]octanyl and 2-oxabicyclo[3.2.1]octanyl, wherein said radicals are optionally substituted by one or more, preferably one or two substituents each independently selected from the group consisting of C₁₋₇alkyl, C₁₋₇alkoxy halo, cyano, hydroxy, oxo, nitro, amino, C₁₋₇alkylamino, and di(C₁₋₇alkyl)amino.

“Treatment” includes prophylactic (preventive) and therapeutic treatment as well as the delay of progression of a disease or disorder.

“Salts” (which, what is meant by “or salts thereof” or “or a salt thereof”), can be present alone or in mixture with free compound of the formula I and are preferably pharmaceutically acceptable salts. Such salts are formed, for example, as acid addition salts, preferably with organic or inorganic acids, from compounds of formula I with a basic nitrogen atom, especially the pharmaceutically acceptable salts. Suitable inorganic acids are, for example, halogen acids, such as hydrochloric acid, sulfuric acid, or phosphoric acid. Suitable organic acids are, e.g., carboxylic acids or sulfonic acids, such as fumaric acid or methansulfonic acid. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient. The salts of compounds of formula I are preferably pharmaceutically acceptable salts; suitable counter-ions forming pharmaceutically acceptable salts are known in the field.

“Combination” refers to either a fixed combination in one dosage unit form, or a kit of parts for the combined administration where a compound of the formula I and a combination partner (e.g. an other drug as explained below, also referred to as “therapeutic agent” or “co-agent”) may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula I and a combination partner, are both administered to a patient simultaneously in the form of a single entity or dosage. The term “non-fixed combination” means that the active ingredients, e.g. a compound of formula I and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

In preferred embodiments, which are preferred independently, collectively or in any combination or sub-combination, the invention relates to a compound of the formula I, in free base form or in acid addition salt form, wherein the substituents are as defined herein.

The invention further relates to pharmaceutically acceptable prodrugs of a compound of formula I. Particularly, the present invention also relates to pro-drugs of a compound of formula I as defined herein that convert in vivo to the compound of formula I as such. Any reference to a compound of formula I is therefore to be understood as referring also to the corresponding pro-drugs of the compound of formula I, as appropriate and expedient.

The invention further relates to pharmaceutically acceptable metabolites of a compound of formula I.

One embodiment of the present invention is a compound according to formula I, or a salt thereof, wherein:
A¹ represents N, A² represents C, A³ represents N, and A⁴ represents CH; or
A¹ represents CH, A² represents N, A³ represents C, and A⁴ represents N;
and
R^{1a} and R^{1b} together with the atoms to which they are attached form a monocyclic or polycyclic, saturated or partly saturated, heterocyclyl,
   said heterocyclyl containing 3-12 ring forming atoms,
   said heterocyclyl containing 1-3 oxygen atoms, 0-3 nitrogen atoms, 0-2 sulfur atoms,
   said heterocyclyl being optionally substituted, the substituents being selected from the group consisting of C₁₋₇alkyl, C₁₋₇alkoxy, halo, cyano hydroxy, oxo, nitro, amino C₁₋₇alkylamino, and di(C₁₋₇alkyl)amino; and
R^{1c} represents hydrogen or C₁₋₇alkyl;
or
R^{1a} and R^{1b} and R^{1c} together with the atoms to which they are attached form a bicyclic or bicyclic fused, saturated or partly saturated, heterocyclyl,
   said heterocyclyl containing 6-12 ring forming atoms,
   said heterocyclyl containing 1-3 oxygen atoms, 0-3 nitrogen atoms, 0-2 sulfur atoms,
   said heterocyclyl being optionally substituted, the substituents being selected from the group consisting of C₁₋₇alkyl, C₁₋₇alkoxy, halo, cyano, hydroxy, oxo, nitro, amino C₁₋₇alkylamino, and di(C₁₋₇alkyl)amino; or
R^{1a} represents branched C₃₋₈ alkyl or C₃₋₁₀ cycloalkyl; and
R^{1b} represents hydrogen or C₁₋₇alkyl; and
R^{1c} represents hydrogen or C₁₋₇alkyl;
and
m represents 1 or 2;
n represents 1 or 2;
A⁵-R² represents N—R², NC(H)R²R³, CR²R³ or CR³—CH₂—R²;
R³ represents hydrogen or C₁₋₇alkyl;
R² represents heterocyclyl, said heterocyclyl
   containing 3-12 ring forming atoms,
   containing 1-3 nitrogen atoms, 0-3 oxygen atoms, 0-3 sulfur atoms,
   being saturated or partly saturated,
   being optionally substituted by one to four substituents, the substituents being independently selected from the group consisting of halo, cyano, oxo, hydroxy, amino, nitro, C₁₋₇alkyl, C₁₋₇alkoxy, hydroxy-C₁₋₇alkyl, aminocarbonyl, C₁₋₇alkylaminocarbonyl, di(C₁₋₇alkyl)aminocarbonyl, or
R² represents OH, SH, C₁₋₇alkoxy, C₁₋₇alkylthio.

In an advantageous embodiment, the present invention provides a compound of formula I, depicted by formula I-1

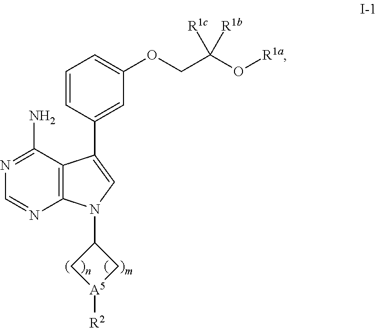

wherein the substituents are as defined herein. Compounds of formula I-1 may be considered as 5,7-disubstituted derivatives of 7H-pyrrolo[2,3-d]pyrimidin-4-amine.

In a further advantageous embodiment, the present invention provides a compound of formula I, depicted by formula I-2

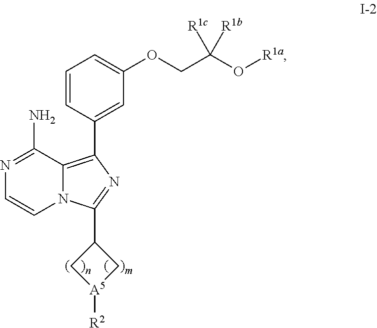

wherein the substituents are as defined herein. Compounds of formula I-2 may be considered as 1,3-disubstituted derivatives of imidazo[1,5-a]pyrazin-8-amine.

In a further advantageous embodiment, the present invention provides a compound of formula I, depicted by formula I-3

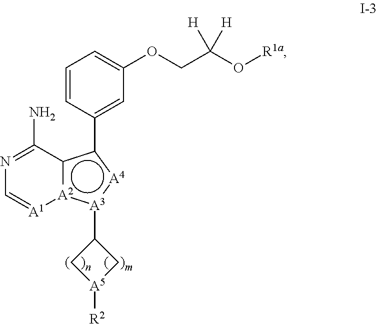

wherein the substituents are as defined herein and R^{1a} represent a residue selected from the group consisting of C₃₋₇ branched alkyl and C₃₋₁₀cycloalkyl. R^{1b} and R^{1c}, as defined in formula I, represent hydrogen and are explicitly included in this formula I-3.

In a further advantageous embodiment, the present invention provides a compound of formula I, depicted by formula I-4

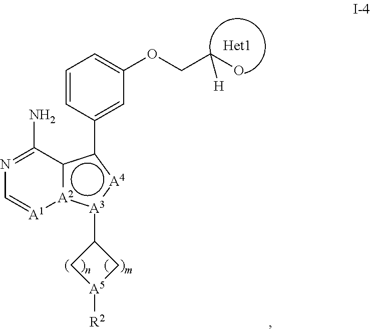

wherein the substituents are as defined herein. In this embodiment, Het1 represents a heterocyclyl as defined in formula I by R^{1a} and R^{1b} together with the —CH—O— residue. R^{1c}, as defined in formula I, represents hydrogen and is explicitly included in this formula I-4.

In a further advantageous embodiment, the present invention provides a compound of formula I, depicted by formula I-5

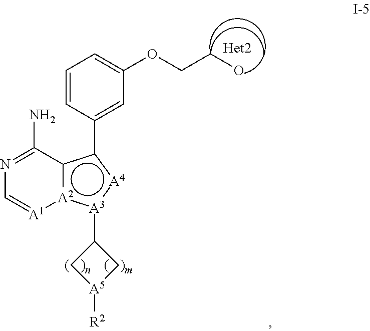

wherein the substituents are as defined herein; Het2 represents a bicyclic heterocyclyl as defined in formula I by R^{1a}, R^{1b}, R^{1c} together with the —CH—O— residue.

In a further advantageous embodiment, the present invention provides a compound of formula I, depicted by formula I-6

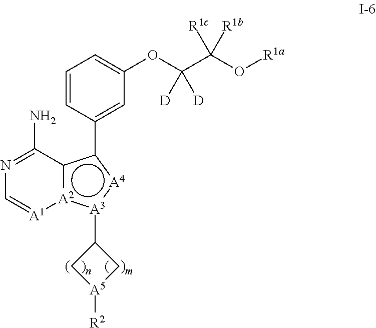

wherein the substituents are as defined herein. In a particularly advantageous embodiment, the present invention provides a compound of formula I-6, wherein A¹ represents N, A² represents C, A³ represents N, and A⁴ represents CH.

In an advantageous embodiment,
R^{1a} and R^{1b} together with the atoms to which they are attached form a monocyclic, saturated heterocyclyl, containing 3-12 ring forming atoms and 1-3 oxygen atoms, said heterocyclyl being optionally substituted, the substituents being selected from the group consisting of C₁₋₇alkyl, C₁₋₇alkoxy, halo, cyano, hydroxy, oxo, nitro, amino C₁₋₇alkylamino, di(C₁₋₇alkyl)amino, and
R^{1c} represents hydrogen.

In a further advantageous embodiment,
R^{1a}, R^{1b} and R^{1c} together with the atoms to which they are attached form a bicyclic heterocyclyl,
   said heterocyclyl being selected from the following moieties:

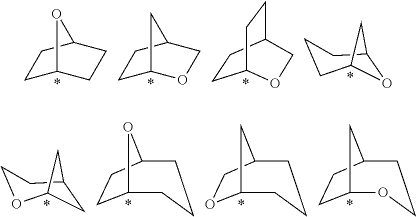

said heterocyclyl being bound to the molecule via the marked carbon atom,
said heterocyclyl being optionally substituted, the substituents being selected from the group consisting of C₁₋₇alkyl, C₁₋₇alkoxy halo, cyano hydroxy, oxo, nitro, amino C₁₋₇alkylamino, and di(C₁₋₇alkyl)amino.

In a further advantageous embodiment,

R^{1a} represents branched C₃₋₈alkyl or C₃₋₁₀cycloalkyl;
R^{1b} represents hydrogen; and
R^{1c} represents hydrogen.

In a further advantageous embodiment, m represents 1.

In a further advantageous embodiment, n represents 1.

In a further advantageous embodiment, m represents 2 and n represents 1.

In a further advantageous embodiment, A⁵-R² represents N—R².

In a further advantageous embodiment, A⁵-R² represents CHR² or CH—CH₂—R².

In a further advantageous embodiment, R² represents heterocyclyl, said heterocyclyl containing 5-6 ring forming atoms; containing 1-2 nitrogen atoms, 0-1 oxygen atoms, 0-1 sulfur atoms; being saturated; being optionally substituted by one or two substituents, the substituents being independently selected from the group consisting of fluoro, oxo, hydroxy, methyl, hydroxymethyl, ethyl, and aminocarbonyl.

In a further advantageous embodiment, R² represents heterocyclyl, said heterocyclyl containing 5-6 ring forming atoms; containing 1-2 nitrogen atoms, 0-1 oxygen atoms, and 0-1 sulfur atoms; being saturated and optionally substituted by one or two substituents, the substituents being independently selected from the group consisting of carboxy, methylcarbonyl, hydroxyethyl, ethyloxycarbonyl, methylsulfonyl, and hydroxymethyltrifluoromethyl.

In a further advantageous embodiment, R² represents OH, C₁₋₇alkoxy, SH, or C₁₋₇alkylthio.

In a further advantageous embodiment, R² represents SO₂R⁴; NHC(O)R⁵; NHR⁵; NHC(O)NHR⁵; NHC(O)OR⁵; NHSO₂R⁵; NHNHC(O)R⁴; imidazolyl optionally substituted with one methyl, CH₂OH or C(O)OR⁵; tetrazolyl optionally substituted with one methyl; or oxazoly.

In a particularly advantageous embodiment,
R¹ and R^{1b} together with the atoms to which they are attached form a heterocyclyl selected from the group consisting of (tetrahydro-2H-pyran)-2-yl and tetrahydrofuran-2-yl;
   said heterocyclyl being unsubstituted or substituted by one or two substituents, the substituents being selected from the group consisting of methyl and ethyl; and
R^{1c} represents hydrogen.

In a further particularly advantageous embodiment,
R^{1a}, R^{1b} and R^{1c} together with the atoms to which they are attached form a heterocyclyl selected from the group consisting of 8-oxabicyclo[3.2.1]octane-1-yl and 7-oxabicyclo[2.2.1]heptane-1y1,
   said heterocyclyl being unsubstituted or substituted by one or two substituents, the substituents being selected from the group consisting of methyl and ethyl.

In a further particularly advantageous embodiment,
R^{1a}, R^{1b} and R^{1c} together with the atoms to which they are attached form d₉-7-oxabicyclo[2.2.1]heptanyl unsubstituted or substituted by one or two substituents, the substituents being selected from the group consisting of methyl and ethyl.

In a further particularly advantageous embodiment,
R^{1a} represents iso-propyl, iso-butyl, tert-butyl, iso-pentyl, neo-pentyl, 3-pentyl, 2-ethylhexyl, cyclopropyl, cyclpentyl, cyclohexyl, cycloheptyl, or cyclooctyl;
R^{1b}b represents hydrogen; and
R^{1c} represents hydrogen.

In a further particularly advantageous embodiment, m and/or n represent 1.

In a further particularly advantageous embodiment, m and n represent 2.

In a further particularly advantageous embodiment, A⁵ represents N. In this embodiment, A⁵ may form, together with the carbon atoms to which it is attached, a piperidine, pyrrolidine or azetidine moiety.

In a further particularly advantageous embodiment, A⁵ represents N. In this embodiment, A⁵ may form, together with the carbon atoms to which it is attached, a oxetane, tetrahydropyran, or thietane moiety.

In a further particularly advantageous embodiment, A⁵ represents CH— or CH—CH₂—. In this embodiment, A⁵ may form, together with the carbon atoms to which it is attached, a cyclobutane-, cyclobutanemethylene-, cyclpentane-, cyclopentanemethylene-, cyclohexane-, or cyclohexanemethylene-moiety.

In a further particularly advantageous embodiment, R² represents heterocyclyl as defined herein, said heterocyclyl being bound to A⁵ via a nitrogen atom.

In a further particularly advantageous embodiment, R² represents a heterocyclyl selected from the following heterocyclic moieties:

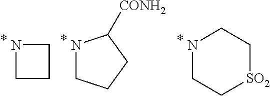

In a further particularly advantageous embodiment, R² represents a heterocyclyl selected from the following heterocyclic moieties:

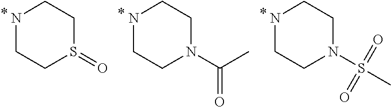

wherein the marked atom is bound to A⁵.

In a further particularly advantageous embodiment, A⁵-R² represents CR²R³ wherein R² and R³ together with the

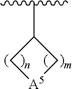

moiety form 5,7-dioxa-spiro[3.4]octanyl, 5-oxa-7-azaspiro[3.4]octanyl, or 5-oxa-8-aza-spiro[3.5]nonanyl, optionally substituted with one to three substituents each independently selected from the group consisting of: halo, cyano, oxo, hydroxy, amino, nitro, C₁₋₇alkyl, C₁₋₇alkoxy, hydroxy-C₁₋₇alkyl, aminocarbonyl, C₁₋₇alkylaminocarbonyl, and di(C₁₋₇alkyl)aminocarbonyl.

In a further particularly advantageous embodiment A⁵-R² represents CR²R³ wherein R² and R³ together with the

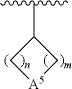

moiety form 5,7-dioxa-spiro[3.4]otanyl, 5-oxa-7-azaspiro[3.4]octanyl, or 5-oxa-8-aza-spiro[3.5]nonanyl, optionally substituted with one to three substituents each independently selected from the group consisting of methyl, ethyl, and oxo.

In a further particularly advantageous embodiment, R² represents hydroxy, methoxy, ethoxy, propoxy, iso-propoxy, thio, methylthio, ethylthio, propylthio, or iso-proylthio, particularly methylthio or hydroxy.

In a further particularly advantageous embodiment, R³ represents hydrogen, methyl, or hydroxy.

In a further particularly advantageous embodiment, R⁴ represents hydrogen, methyl, or ethyl.

In a further particularly advantageous embodiment, R⁵ represents hydrogen; methyl; ethyl; isopropyl; CD₃; hydroxy-C₁₋₃alkyl; C₁₋₃alkyl; halo; C₃₋₇ cycloalkyl optionally substituted with one or two C₁₋₃alkyl groups; piperazinly optionally substituted with one C₁₋₃alkyl; tetrahydropyranyl; pyridinyl optionally substituted with one methyl or cyano.

In a very particularly advantageous embodiment, the present invention provides a compound of formula I, depicted by formula I-7

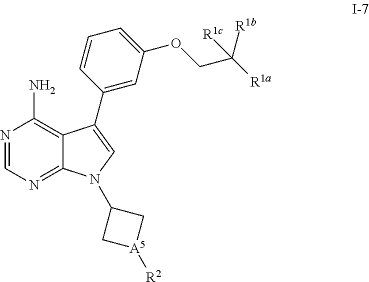

wherein
R^{1a} and R^{1b} together with the atoms to which they are attached form a tetrahydrofuranyl ring optionally substituted with one to three substituents each independently selected from the group consisting of C₁₋₇alkyl, C₁₋₇alkoxy halo, cyano, hydroxy, oxo, nitro, amino, C₁₋₇alkylamino, and di(C₁₋₇alkyl)amino; and
R^{1c} represents hydrogen or C₁₋₇alkyl; or
R^{1a} and R^{1b} and R^{1c} together with the atoms to which they are attached form 7-oxabicyclo[2.2.1]heptanyl or d₉-7-oxabicyclo[2.2.1]heptanyl either being optionally substituted with one to three substituents each independently selected from the group consisting of C₁₋₇alkyl, C₁₋₇alkoxy halo, cyano, hydroxy, oxo, nitro, amino C₁₋₇alkylamino, and di(C₁₋₇alkyl)amino;
A⁵-R² represents CR²R³ or CR³—CH₂—R²;
R³ represents hydrogen, C₁₋₇alkyl, or hydroxy; and
R² represents piperazinyl, thiomorpholinyl, or 2-thia-5-aza-bicyclo[2.2.1]heptanyl optionally substituted with one to four substituents each independently selected from the group consisting of halo, cyano, oxo, hydroxy, carboxy, amino, nitro, SO₂R⁴, COR⁵, C₁₋₇alkyl, C₁₋₇alkyl halo optionally substituted with one hydroxy; C₁₋₄alkoxy, hydroxy-C₁₋₇alkyl, piperazinly C₁₋₃alkyl, aminocarbonyl, C₁₋₇alkylaminocarbonyl, and di(C₁₋₇alkyl)aminocarbonyl; or
R² represents OH; or

R² and R³ together with the

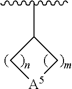

moiety form 5-oxa-7-aza-spiro[3.4]octanyl optionally substituted with one to three substituents each independently selected from the group consisting of: halo, cyano, oxo, hydroxy, amino, nitro, C₁₋₇alkyl, C₁₋₇alkoxy, hydroxy-C₁₋₇alkyl, aminocarbonyl, C₁₋₇alkylaminocarbonyl, and di(C₁₋₇alkyl)aminocarbonyl;

R⁴ represents hydrogen or C₁₋₇alkyl; and

R⁵ represents hydrogen; C₁₋₇alkyl; hydroxy-C₁₋₇alkyl; C₁₋₇alkyl; halo; C₃₋₇ cycloalkyl optionally substituted with one or two C₁₋₃alkyl groups; piperazinly optionally substituted with one C₁₋₃alkyl; tetrahydropyranyl; or pyridinyl optionally substituted with one methyl or cyano. In a very particularly advantageous embodiment, the present invention provides a compound of formula I-7 wherein:
R^{1a} and R^{1b} together with the atoms to which they are attached form tetrahydrofuranyl; and
R^{1c} represents hydrogen or C₁₋₇alkyl; or
R^{1a} and R^{1b} and R^{1c} together with the atoms to which they are attached form 7-oxabicyclo[2.2.1]heptanyl or d₉-7-oxabicyclo[2.2.1]heptanyl;
A⁵-R² represents CR²R³ or CR³—CH₂—R²;
R³ represents hydrogen or hydroxy; and
R² represents piperazinyl, thiomorpholinyl, or 2-thia-5-aza-bicyclo[2.2.1]heptanyl optionally substituted by one to four substituents each independently selected from the group consisting of oxo, carboxy, COR⁵, C₁₋₃alkyl, aminocarbonyl, C₁₋₃alkylaminocarbonyl, and di(C₁₋₃alkyl)aminocarbonyl; or
R² represents OH; or
R² and R³ together with the

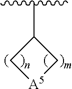

moiety form 5-oxa-7-aza-spiro[3.4]octanyl optionally substituted with one or two oxo groups; and
R⁵ represents hydrogen, C₁₋₃alkyl, or hydroxy-C₁₋₇alkyl.

In a very particularly advantageous embodiment, the present invention relates to a compound of formula I mentioned in the Examples, or a salt, especially a pharmaceutically acceptable salt, thereof.

In a very particularly advantageous embodiment, the present invention relates to a compound of formula I which is:
cis-7-{3-[(1,1-dioxidothiomorpholin-4-yl)methyl]cyclobutyl}-5-{3-[(2S)-tetrahydrofuran-2-ylmethoxy]phenyl}-7H-pyrrolo[2,3-d]pyrimidin-4-amine;
7-[3-(1,1-dioxo-1-thiomorpholin-4-ylmethyl)-cyclobutyl]-5-[3-(7-oxa-bicyclo[2.2.1]hept-1-ylmethoxy)-phenyl]-7H-pyrrolo[2,3-d]pyrimidin-4-ylamine;
d₂-7-[cis-3-(1,1-dioxo-1-thiomorpholin-4-ylmethyl)-cyclobutyl]-5-[3-(7-oxa-bicyclo[2.2.1]hept-1-ylmethoxy)-phenyl]-7H-pyrrolo[2,3-d]pyrimidin-4-ylamine;
d₉-7-[cis-3-(1,1-dioxo-1-thiomorpholin-4-ylmethyl)-cyclobutyl]-5-[3-(7-oxa-bicyclo[2.2.1]hept-1-ylmethoxy)-phenyl]-7H-pyrrolo[2,3-d]pyrimidin-4-ylamine; or
7-[cis-3-(1,1-dioxo-thiomorpholin-4-yl)-cyclobutyl]-5-[3-(7-oxa-bicyclo[2.2.1]hept-1-ylmethoxy)-phenyl]-7H-pyrrolo[2,3-d]pyrimidin-4-ylamine; or a salt thereof.

In a very particularly advantageous embodiment, the present invention relates to a compound of formula I which is:
(R)-1-(cis-3-{4-amino-5-[3-(7-oxa-bicyclo[2.2.1]hept-1-ylmethoxy)-phenyl]-pyrrolo[2,3-d]pyrimidin-7-yl}-cyclobutylmethyl)-pyrrolidine-2-carboxylic acid amide;
(S)-1-(trans-3-{(4-amino-5-[3-(7-oxa-bicyclo[2.2.1]hept-1-ylmethoxy)-phenyl]-pyrrolo[2,3-d]pyrimidin-7-yl}-cyclobutylmethyl)pyrrolidine-2-carboxylic acid amide; or
(R)-1-(trans-3-{(4-amino-5-[3-(7-oxa-bicyclo[2.2.1]hept-1-ylmethoxy)-phenyl]-pyrrolo[2,3-d]pyrimidin-7-yl}-cyclobutylmethyl)-pyrrolidine-2-carboxylic acid amide; or a salt thereof.

In a very particularly advantageous embodiment, the present invention relates to a compound of formula I which is:
1-}4-[cis-3-(4-amino-5-{3-(S)-1-(tetrahydrofuran-2-yl)methoxy]-phenyl}-pyrrolo[2,3-d]pyrimidin-7-yl)-cyclobutyl]-piperazin-1-yl}-ethanone;
7-[cis-3-(4-methanesulfonyl-piperazin-1-yl)-cyclobutyl]-5-[3-(7-oxa-bicyclo[2.2.1]hept-1-ylmethoxy)-phenyl]-7H-pyrrolo[2,3-d]pyrimidin-4-ylamine;
3-[3-(methyl-piperazin-1-yl)-cyclobutyl]-1-[3-(7-oxa-bicyclo[2.2.1]hept-1-ylmethoxy-phenyl]-imidazo[1,5-a]pyrazin-8-ylamine; or
1-[cis-4-(3-}4-Amino-5-[3-(7-oxa-bicyclo[2.2.1]hept-1-ylmethoxy)-phenyl]-pyrrolo[2,3-d]pyrimidin-7-yl}-cyclobutyl)-piperazin-1-yl]-ethanone; or a salt thereof.

In a very particularly advantageous embodiment, the present invention relates to a compound of formula I which is (3-}4-amino-5-[3-(7-oxa-bicyclo[2.2.1]hept-1-ylmethoxy)-phenyl]-pyrrolo[2,3-d]pyrimidin-7-yl}-cyclobutylmethyl)-carbamic acid methyl ester, or a salt thereof.

In a very particularly advantageous embodiment, the present invention relates to a compound of formula I which is: (endo)-5-[3-(7-oxa-bicyclo[2.2.1]hept-1-ylmethoxy)-phenyl]-7-[3-((1S,2S,4S)-2-oxo-2-thia-5-aza-bicyclo[2.2.1]hept-5-ylmethyl)-cyclobutyl]-7H-pyrrolo[2,3-d]pyrimidin-4-ylamine;
5-[cis-3-(7-oxa-bicyclo[2.2.1]hept-1-ylmethoxy)-phenyl]-7-[3-(1-oxo-thiomorpholin-4-ylmethyl)-cyclobutyl]-7H-pyrrolo[2,3-d]pyrimidin-4-ylamine; or
5-[3-(7-Oxa-bicyclo[2.2.1]hept-1-ylmethoxy)-phenyl]-7-[cis-3-(1-oxo-thiomorpholin-4-yl)-cyclobutyl]-7H-pyrrolo[2,3-d]pyrimidin-4-ylamine; or a salt thereof.

The invention relates in a second aspect to the manufacture of a compound of formula I. The compounds of formula I or salts thereof are prepared in accordance with processes known per se (see references cited above), though not previously described for the manufacture of the compounds of the formula I.

### General Reaction Processes:

In one embodiment, the invention relates to a process for manufacturing a compound of formula I (Method A) comprising the step of reacting a compound of formula II

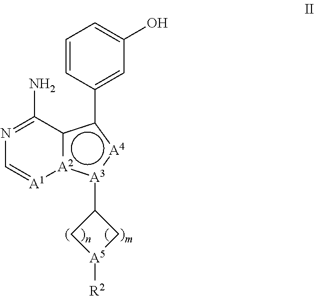

wherein the substituents are as defined above, with a compound of formula III,

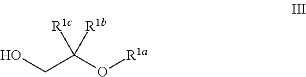

wherein the substituents are as defined above, optionally in the presence of one or more reaction aids, such as triphenylphosphine and DIAD, optionally in the presence of one or more diluents, particular polar solvents, e.g. THF. This type of reaction is also known as Mitsunobo reaction, typical reaction conditions are known in the field and may applied to the present process.

In a further embodiment, the invention relates to a process for manufacturing a compound of formula I (Method B) comprising the step of reacting a compound of formula IV

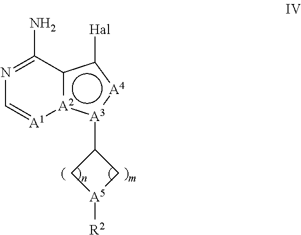

wherein the substituents are as defined above and Hal represents halogen, particularly iodo, with a compound of formula V,

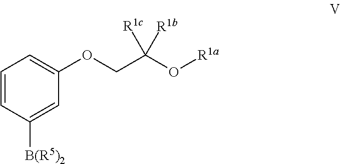

wherein the substituents are as defined above, and B(R⁵)₂ represents a cyclic or acyclic boronic acid, such as 4,4,5,5,-tetramethyl-1,3,2-diocoborolane, in the presence of a catalyst, such as a Pd(0) catalyst, e.g. Pd(PPh₃)₄, optionally in the presence of one or more reaction aids, such as a base, e.g. Na₂CO₃, optionally in the presence of one or more diluents, particularly polar solvents, e.g. H₂O/DMF. This type of reaction is also known as Suzuki reaction, typical reaction conditions are known in the field and may applied to the present process.

In a further embodiment, the invention relates to a process for manufacturing a compound of formula I (Method C) comprising the step of reacting a compound of formula VI

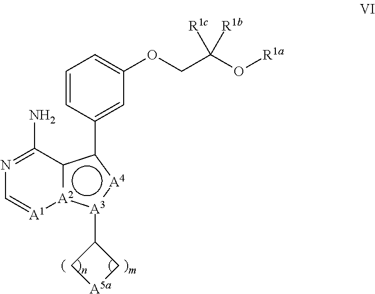

wherein the substituents are as defined above and A^{5a} represents CR³CHO, particularly CHCHO, with a compound of formula VII,

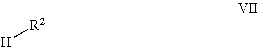

wherein R² are as defined above,
optionally in the presence of one or more reaction aids, such as a borohydride, e.g. triacetoxyborohydride, optionally in the presence of one or more diluents, particularly apolar solvents, e.g. dichloroethane. This type of reaction is also known as a reductive amination reaction, typical reaction conditions are known in the field and may applied to the present process. In this embodiment, the starting material, aldehyde VI may be formed in situ by oxidation of the corresponding alcohol, e.g. by using a hypervalent iodine reagent such as 2-iodoxybenzoic acid (IBX).

In a further embodiment, the invention relates to a process for manufacturing a compound of formula I (Method D) comprising the step of reacting a compound of formula IIX

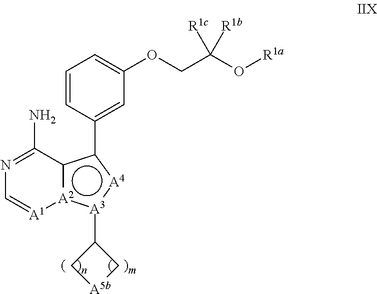

wherein the substituents are as defined above and A^{5b} represents CR³CH2O-FG (FG is a hydroxy activating group), particularly CHCH2OTs (Ts represents tosylate), with a compound of formula IX,

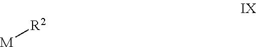

wherein R² is as defined above, particularly thio or alkylthio, and M represents an (earth) alkali metal, particularly sodium, optionally in the presence of one or more reaction aids, optionally in the presence of one or more diluents, particularly polar solvents, e.g. THF. Typical reaction conditions are known in the field and may applied to the present process.

In a further embodiment, the invention relates to a process for manufacturing a compound of formula I (Method E) comprising the step of reacting a compound of formula X

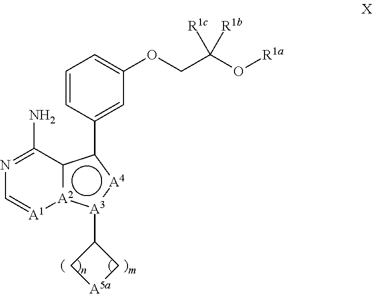

wherein the substituents are as defined above and A^{5a} represents N, with a compound of formula XI or XII,

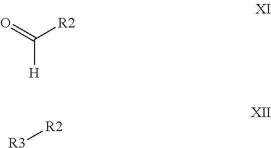

wherein R² are as defined above,

In the case of XI optionally in the presence of one or more reaction aids, such as a borohydride, e.g. triacetoxyborohydride, optionally in the presence of one or more diluents, particularly apolar solvents, e.g. dichloroethane. This type of reaction is also known as a reductive amination reaction, typical reaction conditions are known in the field and may be applied to the present process. In the case of XII (R³ is halogen, tosylate, mesylate or trifluoromethane sulphonate) optionally in the presence of one or more reaction aids, such as a base, eg sodium hydrogen carbonate or triethylamine in the presence of one or more diluents, eg. MeOH. This type of reaction is also known as an N-alkylation.

### Protecting Groups:

In the methods describe above, functional groups which are present in the starting materials and are not intended to take part in the reaction, are present in protected form if necessary, and protecting groups that are present are cleaved, whereby said starting compounds may also exist in the form of salts provided that a salt-forming group is present and a reaction in salt form is possible. In additional process steps, carried out as desired, functional groups of the starting compounds which should not take part in the reaction may be present in unprotected form or may be protected for example by one or more protecting groups. The protecting groups are then wholly or partly removed according to one of the known methods. Protecting groups, and the manner in which they are introduced and removed are described, for example, in “Protective Groups in Organic Chemistry”, Plenum Press, London, New York 1973, and in “Methoden der organischen Chemie”, Houben-Weyl, 4th edition, Vol. 15/1, Georg-Thieme-Verlag, Stuttgart 1974 and in Theodora W. Greene, “Protective Groups in Organic Synthesis”, John Wiley & Sons, New York 1981. A characteristic of protecting groups is that they can be removed readily, i.e. without the occurrence of undesired secondary reactions, for example by solvolysis, reduction, photolysis or alternatively under physiological conditions.

### Additional Process Steps:

In the methods described above, a compound of formula I thus obtained may be converted into another compound of formula I, a free compound of formula I is converted into a salt, an obtained salt of a compound of formula I is converted into the free compound or another salt, and/or a mixture of isomeric compounds of formula I is separated into the individual isomers. The end products of formula I may however also contain substituents that can also be used as protecting groups in starting materials for the preparation of other end products of formula I. Thus, within the scope of this text, only a readily removable group that is not a constituent of the particular desired end product of formula I is designated a “protecting group”, unless the context indicates otherwise.

A compound of formula I can be converted to a corresponding N-oxide. The reaction is carried out with a suitable oxidizing agent, preferably a peroxide, for example mchloroperbenzoic acid, in a suitable solvent, e.g. halogenated hydrocarbon, typically chloroform or dichloromethane, or in a lower alkanecarboxylic acid, typically acetic acid, preferably at a temperature between 0° C. and the boiling temperature of the reaction mixture, especially at about RT.

### General Process Conditions:

All process steps described here can be carried out under known reaction conditions, preferably under those specifically mentioned, in the absence of or usually in the presence of solvents or diluents, preferably those that are inert to the reagents used and able to dissolve them, in the absence or presence of catalysts, condensing agents or neutralizing agents, for example ion exchangers, typically cation exchangers, for example in the H⁺ form, depending on the type of reaction and/or reactants at reduced, normal, or elevated temperature, for example in the range from −100° C. to about 190° C., preferably from about −80° C. to about 150° C., for example at −80 to −60° C., at RT, at −20 to 40° C. or at the boiling point of the solvent used, under atmospheric pressure or in a closed vessel, if need be under pressure, and/or in an inert, for example an argon or nitrogen, atmosphere.

The invention relates also to those embodiments of the process in which one starts from a compound obtainable at any stage as an intermediate and carries out the missing steps, or breaks off the process at any stage, or forms a starting material under the reaction conditions, or uses said starting material in the form of a reactive derivative or salt, or produces a compound obtainable by means of the process according to the invention under those process conditions, and further processes the said compound in situ. In the preferred embodiment, one starts from those starting materials which lead to the compounds described hereinabove as preferred.

The compounds of formula I (or N-oxides thereof), including their salts, are also obtainable in the form of hydrates, or their crystals can include for example the solvent used for crystallisation (present as solvates). In the preferred embodiment, a compound of formula I is prepared according to the processes and process steps defined in the Examples.

### Starting Materials:

New starting materials and/or intermediates, as well as processes for the preparation thereof, are likewise the subject of this invention. In the preferred embodiment, such starting materials are used and reaction conditions so selected as to enable the preferred compounds to be obtained.

The starting materials used in the above described processes are known, capable of being prepared according to known processes (see references cited above), or commercially obtainable; in particular, they can be prepared using processes as described in the Examples.

In the preparation of starting materials, existing functional groups which do not participate in the reaction should, if necessary, be protected. Preferred protecting groups, their introduction and their removal are described above or in the examples. In place of the respective starting materials and transients, salts thereof may also be used for the reaction, provided that salt-forming groups are present and the reaction with a salt is also possible. Where the term starting materials is used hereinbefore and hereinafter, the salts thereof are always included, insofar as reasonable and possible.

The invention relates in a third aspect to the use of compounds of the present invention as pharmaceuticals. Particularly, the compounds of formula I have valuable pharmacological properties, as described hereinbefore and hereinafter. The invention thus provides:
a compound of the formula (I) as defined herein, as pharmaceutical/for use as pharmaceutical;
a compound of the formula (I) as defined herein, as medicament/for use as medicament;
compound of the formula (I) as defined herein, for the treatment of/for use in the treatment of one or more IGF-1R mediated disorders or diseases;
the use of a compound of formula (I) as defined herein, for the manufacture of a medicament for the treatment of an IGF-1R mediated disorder or disease;
the use of a compound of formula (I) as defined herein, for the treatment of an IGF-1R mediated disorder or disease;
the use of a compound of formula I as defined herein for the inhibition of the IGF-IR tyrosine kinase;
the use of a compound of formula (I) as defined herein, for the treatment of a disorder or disease selected from multiple myeloma, neuroblastoma, synovial, hepatocellular, Ewing's Sarcoma, adrenocotical carcinoma (ACC) or a solid tumor selected from osteosarcoma, melanoma, tumor of breast, renal, prostate, colorectal, thyroid, ovarian, pancreatic, lung, uterine or gastrointestinal tumor;
the use of a compound of formula (I) as defined herein, for the treatment of a disorder or disease selected from acute lung injury and pulmonary fibrosis;
the use of a compound of formula (I) as defined herein, for the treatment of diabetic retinopathy;
a method of modulating IGF-1R activity in a subject, comprising the step of administering to a subject a therapeutically effective amount of a compound of formula I as defined herein;
a method for the treatment of an IGF-1R mediated disorder or disease comprising the step of administering to a subject a therapeutically effective amount of a compound of formula (I) as defined herein;
a method for inhibition IGF-1R in a cell, comprising contacting said cell with an effective amount of a compound of formula I as defined herein.

A “Subject in need thereof” refers to any animal classified as a mammal, including humans, domestic and farm animals, and zoo, sports, or pet animals, such as dogs, cats, cattle, horses, sheep, pigs, goats, rabbits, etc. In particular examples, the mammal is human.

The term “administration” or “administering” of the subject compound means providing a compound of the invention and prodrugs thereof to a subject in need of treatment. Administration“in combination with” one or more further therapeutic agents includes simultaneous (concurrent) and consecutive administration in any order, and in any route of administration.

An “effective amount” of a compound is an amount sufficient to carry out a specifically stated purpose. An “effective amount” may be determined empirically and in a routine manner, in relation to the stated purpose.

The term “therapeutically effective amount” refers to an amount of a compound (e.g., an IGF-1R antagonist) effective to “treat” an IGF-1R-mediated disorder in a subject or mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See the definition herein of “treating”. To the extent the drug may prevent growth and/or kill existing cancer cells, it may be cytostatic and/or cytotoxic.

The term “cancer” refers to the physiological condition in mammals that is typically characterized by unregulated cell growth/proliferation. Examples of cancer include, but are not limited to: carcinoma, lymphoma, blastoma, and leukemia. More particular examples of cancers include, but are not limited to: chronic lymphocytic leukemia (CLL), lung, including non small cell (NSCLC), breast, ovarian, cervical, endometrial, prostate, colorectal, intestinal carcinoid, bladder, gastric, pancreatic, hepatic (hepatocellular), hepatoblastoma, esophageal, pulmonary adenocarcinoma, mesothelioma, synovial sarcoma, osteosarcoma, head and neck squamous cell carcinoma, juvenile nasopharyngeal angiofibromas, liposarcoma, thyroid, melanoma, basal cell carcinoma (BCC), adrenocotical carcinoma (ACC), medulloblastoma and desmoid.

The term “IGF-1R mediated disease” includes but is not limited to, multiple myeloma, neuroblastoma, synovial, hepatocellular, Ewing's Sarcoma, adrenocotical carcinoma (ACC), or a solid tumor selected from osteosarcoma, melanoma, tumor of breast, renal, prostate, colorectal, thyroid, ovarian, pancreatic, lung, uterine or gastrointestinal tumor.

It was further found that compounds of formula I are also useful in the treatment of acute lung injury and pulmonary fibrosis.

“Treating” or “treatment” or “alleviation” refers to both therapeutic treatment and prophylactic or preventative measures, wherein the object is to prevent or slow down (lessen) the targeted pathologic disease or condition or disorder. Those in need of treatment include those already with the disorder as well as those prone to having the disorder or those in whom the disorder is to be prevented (prophylaxis). When the IGF-1R-mediated disorder is cancer, a subject or mammal is successfully “treated” or shows a reduced tumor burden if, after receiving a therapeutic amount of an IGF-1R antagonist according to the methods of the present invention, the patient shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent the IGF-1R antagonist may prevent growth and/or kill existing cancer cells, it may be cytostatic and/or cytotoxic. Reduction of these signs or symptoms may also be felt by the patient.

The invention provides in further embodiments methods to treat, ameliorate or prevent a condition which responds to inhibition of IGF-1R in a mammal suffering from said condition, comprising administering to the mammal a therapeutically effective amount of a compound of formula I as defined herein, and optionally in combination with a second therapeutic agent. The compounds of the invention may be administered, for example, to a mammal suffering from an autoimmune disease, a transplantation disease, an infectious disease or a cell proliferative disorder. In particular examples, the compounds of the invention may be used alone or in combination with a chemotherapeutic agent to treat a cell proliferative disorder.

The efficacy of the compounds of the invention (i.e. a compound of formula I as defined herein) as inhibitors of IGF-IR tyrosine kinase activity can be demonstrated using a cellular “Capture ELISA”. In this assay the activity of the compounds of the invention against Insulin-like growth factor I (IGF-I) induced autophosphorylation of the IGF-IR was determined. The assay was conducted as follows: Compound-mediated inhibition of IGF1R and INSR phosphorylation in Hek293 cells transduced with the corresponding receptors was assessed in a capture ELISA format using the MSD (Meso Scale Discovery) platform. Briefly, 30,000 cells washed and diluted in starvation medium (DMEM high glucose supplemented with 0.1% BSA) were seeded in 90 μL per well into 96-well plates pre-coated with poly-D-lysine (0.1 mg/mL in PBS/O). After 24 h incubation at 37° C. and 5% CO2, dose-response effects were determined with 3-fold serial compound dilutions, starting at 10 μM. The final vehicle concentration is 0.1% DMSO in all wells. Following pre-incubation with compounds for 1 h, receptor phosphorylation was triggered by a 10 min exposure to 1.0 ng/μL IGF for Hek293-IGF1R cells, and 5.0 ng/μL insulin for Hek293-InsR cells. Cell lysis was achieved by addition of 80 μL MSD lysis buffer per aspirated well, incubation on ice for 20 min, and a freeze-thaw cycle. Target phosphorylation was then assessed by transferring volumes corresponding to approx. 6 μg Hek293-IGF1R or 0.6 μg Hek293-InsR lysates to MSD assay plates pre-coated with total-IGF1R or total-InsR Abs, respectively. After incubation for 2 h at rt, wells were exposed for 1 hr to a rabbit monoclonal antibody (CST #3024, 1:1000) detecting pIGF1R(Tyr1135/1136) as well as pINSR(Tyr1150/1151). Immune complexes were detected by a SULFO-Tag™-coupled anti-rabbit IgG antibody in the presence of 150 μL MSD read-buffer. Light emission at 620 nm triggered by application of electric current was recorded on a MSD Sectorlmager 6000. Acquired raw data (mean Ru-ECL units) were processed in an Excel analysis template. The plate blank (MSD lysis buffer) was subtracted from all data points. The effect of a particular test compound concentration on receptor phosphorylation was expressed relative to the window defined by ligand-stimulated vs unstimulated control cells (set as 100%). IC50 values [nM] were determined using 4-parametric curve-fitting (XLfit software, V4.3.2).

Alternatively, the assay can be conducted with a slightly different format; Compound-mediated inhibition of IGF-1R and InsR phosphorylation in HEK293 cells overexpressing the corresponding receptors were assessed by quantitative Western blot using an Odyssey infrared imager as readout.

Briefly, 1,200,000 cells washed and diluted in starvation medium (DMEM high glucose supplemented with 0.1% BSA) were seeded in 2 mL per well into 6-well plates. After 6-8 hrs incubation at 37° C. and 5% CO2, dose-response effects were determined with 3-fold serial dilutions. The final vehicle concentration was =<0.1% DMSO in all wells. Following pre-incubation with compounds for 1 hr, receptor phosphorylation was triggered by a 10-min exposure to 500 ng/mL IGF1 for HEK293-IGF1R cells, and 5 μg/mL insulin for HEK293-INSR cells. Whole cell extracts were prepared by addition of 200 μL ice-cold lysis buffer for 10 min and a freeze-thaw cycle of 30 min, and 20 μg were loaded onto 48-well 8% acrylamide E-PAGE gels, then proteins were separated by electrophoresis for 36 min and transferred onto PVDF membranes using the iBlot transfer system for 7 min. Target phosphorylation was then assessed by incubating the membranes with a rabbit mAb (CST #3024, 1:1000) detecting pIGF1R (Tyr1135/1136) as well as pINSR (Tyr1150/1151) overnight at 4° C. followed by a 3 hr incubation at room temperature with a mouse mAb detecting Tubulin (loading control) and an additional 1 hr incubation in the dark at room temperature with both Alexa fluor 680 conjugated anti-mouse IgG and IRDye 800CW conjugated anti-rabbit IgG as secondary antibodies. Quantification was performed by densitometry using an Odyssey infrared imager and raw data were processed in an Excel analysis template. The effect of a particular test compound concentration on receptor phosphorylation was expressed relative to the ligand-stimulated control cells (set as 100%), after protein loading normalization assessed by the Tubilin signal. IC50 values were determined using a 4-parametric curve-fitting (XLfit software, v4.3.2; model 205).

In a preferred embodiment, the invention relates to compounds of formula I, which in the above-described “Capture ELISA” assay have an IC₅₀ value of less than 500 nM, most preferably those having an IC₅₀ value of less than 100 nM.

The efficacy of the compounds of the invention (i.e. a compound of formula I as defined herein) as inhibitors of IGF-IR tyrosine kinase activity can be demonstrated as follows:

### In Vivo Activity in the Nude Mouse Xenotransplant Model:

For in vivo efficacy experiments, cells were resuspended in HBSS were injected subcutaneously (0.05 ml/mouse) into female nude (HsdNpa:athymic/nu) mice 6-8 weeks of age. Treatments were initiated when the mean tumor volumes were approximately 200 mm³. Body weights and tumor volumes were recorded three times a week. Tumor volumes were measured with calipers and determined according to the formula length×diameter²×π/6. In addition to presenting fractional changes of tumor volumes over the course of treatments, antitumor activity is expressed as TIC % (mean change of tumor volume of treated animals/mean change of tumor volume of control animals)×100. Efficacy of candidate IGF-1R inhibitors was determined by initiating oral dosing on day 17-18 post-cell injection following randomization of the mice so that each group has similar mean tumor size. Dosing with an appropriate schedule continued for 7 days based on the general health condition of the animals. All candidate IGF-1R inhibitors were formulated in a suitable vehicle, eg NMP/PEG300 (10:90) and applied daily by gavage. Vehicle consisted of, eg NMP/PEG300 (10:90). All application volumes were 5 ml/kg. After the last measurement a final dose of the compound was given and animals in each treatment group were sacrificed after different time points for terminal PK on blood, liver and other organs, as well as determination of phosphorylated IGF-1R (pIGF-1R) levels in tumor and phosphorylated InsR (pinsR) in liver samples. Plasma insulin levels were assessed using a commercial available ELISA kit (Mercodia). Blood glucose levels were assessed using a glucometer (One Touch Ultra®, LifeScan).

These studes can be used to show that a compound of formula I, or a salt thereof, has therapeutic efficacy, especially against proliferative diseases, responsive to an inhibition of the IGF-IR tyrosine kinase.

In a further embodiment, the invention relates to a process or a method for the treatment of one of the pathological conditions mentioned hereinabove, especially a disease which responds to an inhibition of the IGF-IR tyrosine kinase or of the IGF-IR-dependent cell proliferation, especially a corresponding neoplastic disease. The compounds of formula I, or a pharmaceutically acceptable salt thereof, can be administered as such or in the form of pharmaceutical compositions, prophylactically or therapeutically, preferably in an amount effective against the said diseases, to a warm-blooded animal, for example a human, requiring such treatment, the compounds especially being used in the form of pharmaceutical compositions. In the case of an individual having a bodyweight of about 70 kg the daily dose administered is from approximately 0.1 g to approximately 5 g, preferably from approximately 0.5 g to approximately 2 g, of a compound of the present invention.

In a further embodiment, the invention relates to use of a compound of formula I, or a pharmacuetically acceptable salt thereof, especially a compound of formula I which is said to be preferred, or a pharmaceutically acceptable salt thereof, as a medicament.

In a further embodiment, the invention relates to the use of a compound of formula I, or a pharmaceutically acceptable salt thereof, especially a compound of formula I which is said to be preferred, or a pharmaceutically acceptable salt thereof, as such or in the form of a pharmaceutical composition with at least one pharmaceutically acceptable carrier, for the therapeutic and also prophylactic management of one or more of the diseases mentioned hereinabove, preferably a disease which responds to an inhibition of the IGF-IR tyrosine kinase or of the IGF-IR-dependent cell proliferation, especially a neoplastic disease, in particular if the said disease responds to an inhibition of the IGF-IR tyrosine kinase or of the IGF-IR-dependent cell proliferation.

In a further embodiment, the invention relates to the use of a compound of formula I, or a pharmaceutically acceptable salt thereof, especially a compound of formula I which is said to be preferred, or a pharmaceutically acceptable salt thereof, for the manufacture of a medicament for the therapeutic and also prophylactic management of one or more of the diseases mentioned hereinabove, especially a neoplastic disease, in particular if the disease responds to an inhibition of the IGF-IR tyrosine kinase or of the IGF-IR-dependent cell proliferation.

The invention relates in a fourth aspect to pharmaceutical compositions comprising a compound of the present invention. The invention thus provides
a pharmaceutical composition comprising a compound of formula I, or a pharmaceutically acceptable salt thereof, and one or more carriers/excipients;
a pharmaceutical composition comprising a therapeutically effective amount of a compound of formula I as defined herein, and one or more pharmaceutically acceptable carriers/excipients.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

Suitable excipients/carriers may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like.

Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

Compressed gases may be used to disperse a compound of the formula (I) in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, I8th ed., I990).

The dosage of the active ingredient depends upon the disease to be treated and upon the species, its age, weight, and individual condition, the individual pharmacokinetic data, and the mode of administration. The amount of the compound in a formulation can vary within the full range employed by those skilled in the art. Typically, the formulation will contain, on a weight percent (wt %) basis, from about 0.01-99.99 wt % of a compound of formula (I) based on the total formulation, with the balance being one or more suitable pharmaceutical excipients. Preferably, the compound is present at a level of about 1-80 wt %. Unit dose forms are, for example, coated and uncoated tablets, ampoules, vials, suppositories or capsules. Examples are capsules containing from about 0.05 g to about 1.0 g of active substance.

Compositions for enteral administration, such as nasal, buccal, rectal or, especially, oral administration, and for parenteral administration, such as intravenous, intramuscular or subcutaneous administration, to warm-blooded animals, especially humans, are especially preferred. The compositions contain the active ingredient alone or, preferably, together with a pharmaceutically acceptable carrier.

Pharmaceutical compositions comprising a compound of formula I as defined herein in association with at least one pharmaceutical acceptable carrier (such as excipient a and/or diluent) may be manufactured in conventional manner, e.g. by means of conventional mixing, granulating, coating, dissolving or lyophilising processes.

In a further embodiment, the invention relates to a pharmaceutical composition for administration to a warm-blooded animal, especially humans or commercially useful mammals suffering from a disease which responds to an inhibition of the IGF-IR tyrosine kinase or of the IGF-IR-dependent cell proliferation, especially a neoplastic disease, comprising an effective quantity of a compound of formula I for the inhibition of the IGF-IR tyrosine kinase or of the IGF-IR-dependent cell proliferation, or a pharmaceutically acceptable salt thereof, together with at least one pharmaceutically acceptable carrier.

In a further embodiment, the invention relates to a pharmaceutical composition for the prophylactic or especially therapeutic management of neoplastic and other proliferative diseases of a warm-blooded animal, especially a human or other mammal requiring such treatment, especially suffering from such a disease, comprising as active ingredient in a quantity that is prophylactically or especially therapeutically active against said diseases a compound of formula I, or a pharmaceutically acceptable salt thereof, is likewise preferred.

The invention relates in a fifth aspect to combinations comprising a compound of formula I and one or more additional active ingredients. The invention thus provides
a combination, in particular, a pharmaceutical combination comprising a therapeutically effective amount of a compound of formula I and one or more therapeutically active agents, particularly antiproliferative agents;
a combined pharmaceutical composition, adapted for simultaneous or sequential administration, comprising a therapeutically effective amount of a compound of formula (I) as defined herein; therapeutically effective amount(s) of one or more combination partners, particularly antiproliferative agents; one or more pharmaceutically acceptable excepients;
a combined pharmaceutical composition as defined herein (i) as pharmaceutical, (ii) for use in the treatment of a IGF-1R mediated disease, (iii) in a method of treatment of a IGF-1R mediated disease.
a kit comprising two or more separate pharmaceutical compositions, at least one of which contains a compound of formula (I). In one embodiment, the kit comprises means for separately retaining said compositions, such as a container, divided bottle, or divided foil packet. An example of such a kit is a blister pack, as typically used for the packaging of tablets, capsules and the like. The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

The term “pharmaceutical combination” or “combined pharmaceutical composition”, as used herein, refers to a product obtained from mixing or combining active ingredients, and includes both fixed and non-fixed combinations of the active ingredients. The term “fixed combination” means that the active ingredients, e.g. a compound of formula I and a co-agent, are both administered to a patient simultaneously in the form of a single entity or dosage. The term “non-fixed combination” means that the active ingredients, e.g. a compound of formula I and a co-agent, are both administered to a patient as separate entities either simultaneously, concurrently or sequentially with no specific time limits, wherein such administration provides therapeutically effective levels of the active ingredients in the body of the patient. The latter also applies to cocktail therapy, e.g. the administration of three or more active ingredients.

In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

The term “antiproliferative agent” includes, but are not limited to, aromatase inhibitors, antiestrogens, topoisomerase I inhibitors, topoisomerase II inhibitors, microtubule active agents, alkylating agents, histone deacetylase inhibitors, farnesyl transferase inhibitors, COX-2 inhibitors, MMP inhibitors, compounds decreasing the lipid kinase activity, e.g. PI3 kinase inhibitors, antineoplastic antimetabolites, platin compounds, compounds decreasing the protein kinase activity, e.g. mTOR inhibitors, Raf inhibitors, MEK inhibitors, and further anti-angiogenic compounds, gonadorelin agonists, anti-androgens, bengamides, bisphosphonates, trastuzumab, and radiotherapy.

The term “aromatase inhibitors” as used herein relates to compounds which inhibit the estrogen production, i.e. the conversion of the substrates androstenedione and testosterone to estrone and estradiol, respectively. The term includes, but is not limited to steroids, especially exemestane and formestane and, in particular, non-steroids, especially aminoglutethimide, vorozole, fadrozole, anastrozole and, very especially, letrozole. Exemestane can be administered, e.g., in the form as it is marketed, e.g. under the trademark AROMASIN™. Formestane can be administered, e.g., in the form as it is marketed, e.g. under the trademark LENTARON™. Fadrozole can be administered, e.g., in the form as it is marketed, e.g. under the trademark AFEMA™. Anastrozole can be administered, e.g., in the form as it is marketed, e.g. under the trademark ARIMIDEX™. Letrozole can be administered, e.g., in the form as it is marketed, e.g. under the trademark FEMARA™ or FEMAR™. Aminoglutethimide can be administered, e.g., in the form as it is marketed, e.g. under the trademark ORIMETEN™.

A combination of the invention comprising an antineoplastic agent which is an aromatase inhibitor is particularly useful for the treatment of hormone receptor positive breast tumors.

The term “antiestrogens” as used herein relates to compounds which antagonize the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX™. Raloxifene hydrochloride can be administered, e.g., in the form as it is marketed, e.g. under the trademark EVISTA™. Fulvestrant can be formulated as disclosed in U.S. Pat. No. 4,659,516 or it can be administered, e.g., in the form as it is marketed, e.g. under the trademark FASLODEX″.

The term “topoisomerase I inhibitors” as used herein includes, but is not limited to topotecan, irinotecan, 9-nitrocamptothecin and the macromolecular camptothecin conjugate PNU-166148 (compound A1 in WO99/17804). Irinotecan can be administered, e.g., in the form as it is marketed, e.g. under the trademark CAMPTOSAR™. Topotecan can be administered, e.g., in the form as it is marketed, e.g. under the trademark HYCAMTIN™.

The term “topoisomerase II inhibitors” as used herein includes, but is not limited to the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide. Etoposide can be administered, e.g., in the form as it is marketed, e.g. under the trademark ETOPOPHOS™. Teniposide can be administered, e.g., in the form as it is marketed, e.g. under the trademark VM 26-BRISTOL™. Doxorubicin can be administered, e.g., in the form as it is marketed, e.g. under the trademark ADRIBLASTIN™. Epirubicin can be administered, e.g., in the form as it is marketed, e.g. under the trademark FARMORUBICIN™. Idarubicin can be administered, e.g., in the form as it is marketed, e.g. under the trademark ZAVEDOS™. Mitoxantrone can be administered, e.g., in the form as it is marketed, e.g. under the trademark NOVANTRON™.

The term “lipid kinase inhibitors’ relates to PI3 kinase inhibitors, PI4 kinase inhibitors, Vps34 inhibitors. Specific examples include: NVP-BEZ235, NVP-BGT226, NVP-BKM120, AS-604850, AS-041164, AS-252424, AS-605240, GDC0941, PI-103, TGX221, YM201636, ZSTK474, examples described in WO 2009/080705 and US 2009/163469.

The term “microtubule active agents” relates to microtubule stabilizing and microtubule destabilizing agents including, but not limited to the taxanes paclitaxel and docetaxel, the vinca alkaloids, e.g., vinblastine, especially vinblastine sulfate, vincristine especially vincristine sulfate, and vinorelbine, discodermolide and epothilones, such as epothilone B and D. Docetaxel can be administered, e.g., in the form as it is marketed, e.g. under the trademark TAXOTERE™. Vinblastine sulfate can be administered, e.g., in the form as it is marketed, e.g. under the trademark VINBLASTIN R.P.™. Vincristine sulfate can be administered, e.g., in the form as it is marketed, e.g. under the trademark FARMISTIN™. Discodermolide can be obtained, e.g., as disclosed in U.S. Pat. No. 5,010,099.

The term “alkylating agents” as used herein includes, but is not limited to cyclophosphamide, ifosfamide and melphalan. Cyclophosphamide can be administered, e.g., in the form as it is marketed, e.g. under the trademark CYCLOSTIN™. Ifosfamide can be administered, e.g., in the form as it is marketed, e.g. under the trademark HOLOXAN™.

The term “histone deacetylase inhibitors” relates to compounds which inhibit the histone deacetylase and which possess antiproliferative activity.

The term “farnesyl transferase inhibitors” relates to compounds which inhibit the farnesyl transferase and which possess antiproliferative activity.

The term “COX-2 inhibitors” relates to compounds which inhibit the cyclooxygenase type 2 enyzme (COX-2) and which possess antiproliferative activity such as celecoxib (Celebrex®) and rofecoxib (Vioxx®).

The term “MMP inhibitors” relates to compounds which inhibit the matrix metalloproteinase (MMP) and which possess antiproliferative activity.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578. The term “antineoplastic antimetabolites” includes, but is not limited to 5-fluorouracil, 5-fluorouracil, tegafur, capecitabine, cladribine, cytarabine, fludarabine phosphate, fluorouridine, gemcitabine, 6-mercaptopurine, hydroxyurea, methotrexate, edatrexate and salts of such compounds, and furthermore ZD 1694 (RALTITREXED™), LY231514 (ALIMTA ™) LY264618 (LOMOTREXOL™) and OGT719.

The term “platin compounds” as used herein includes, but is not limited to carboplatin, cisplatin and oxaliplatin. Carboplatin can be administered, e.g., in the form as it is marketed, e.g. under the trademark CARBOPLAT™. Oxaliplatin can be administered, e.g., in the form as it is marketed, e.g. under the trademark ELOXATIN™.

The term “compounds decreasing the protein kinase activity and further anti-angiogenic compounds” as used herein includes, but is not limited to compounds which decrease the activity of e.g. the Vascular Endothelial Growth Factor (VEGF), the Epidermal Growth Factor (EGF), and c-Src and anti-angiogenic compounds having another mechanism of action than decreasing the protein kinase activity.

Compounds which decrease the activity of VEGF are especially compounds which inhibit the VEGF receptor, especially the tyrosine kinase activity of the VEGF receptor, and compounds binding to VEGF, and are in particular those compounds, proteins and monoclonal antibodies generically and specifically disclosed in WO 98/35958 (describing compounds of formula 0, WO 00/09495, WO 00/27820, WO 00/59509, WO 98/11223, WO 00/27819, WO 01/55114, WO 01/58899 and EP 0 769 947; those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, vol. 27, no. 1, pp 14-21, 1999; in WO 00/37502 and WO 94/10202; Angiostatin, described by M. S. O'Reilly et al, Cell 79, 1994, 315-328; and Endostatin, described by M. S. O'Reilly et al, Cell 88, 1997, 277-285; sorefanib (Nexavar), Sutent (sunitinib), BAY 43-9006.

Compounds which decrease the activity of EGF are especially compounds which inhibit the EGF receptors, especially the tyrosine kinase activity of the EGF receptors, and compounds binding to EGF, and are in particular those compounds generically and specifically disclosed in WO 97/02266 (describing compounds of formula IV), EP 0 564 409, WO 99/03854, EP 0520722, EP 0 566 226, EP 0 787 722, EP 0 837 063, WO 98/10767, WO 97/30034, WO 97/49688, WO 97/38983 and, especially, WO 96/33980. Specific EGF receptor inhibitor examples include, but not limited to; Tarceva (erlotinib), Iressa (Gefitinib), Tykerb (lapatanib). Erbitux (cetuximab), Avastin (bevacizumab), Herceptin (trastuzamab), Rituxan (rituximab), Bexxar (tositumomab), and panitumumab.

Compounds which decrease the activity of c-Src include, but are not limited to, compounds inhibiting the c-Src protein tyrosine kinase activity as defined below and to SH2 interaction inhibitors such as those disclosed in WO97/07131 and WO97/08193; compounds inhibiting the c-Src protein tyrosine kinase activity include, but are not limited to, compounds belonging to the structure classes of pyrrolopyrimidines, especially pyrrolo[2,3-d]pyrimidines, purines, pyrazopyrimidines, especially pyrazo[3,4-d]pyrimidines, pyrazopyrimidines, especially pyrazo[3,4-d]pyrimidines and pyridopyrimidines, especially pyrido[2,3-d]pyrimidines. Preferably, the term relates to those compounds disclosed in WO 96/10028, WO 97/28161, WO97/32879 and WO97/49706;

Compounds which decrease the activity of Raf kinases include, but are not limited to: Raf265, sorefanib, and BAY 43-9006.

Compounds which inhibit downstream effectors of Raf kinases, such as MEK. Examples of MEK inhibitors include; PD 98059, AZD6244 (ARRY-886), CI-1040, PD 0325901, and u0126.

Anti-angiogenic compounds having another mechanism of action than decreasing the protein kinase activity include, but are not limited to e.g. thalidomide (THALOMID™), SU5416, and celecoxib (Celebrex™)

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX™. Abarelix can be formulated, eg. as disclosed in U.S. Pat. No. 5,843,901. The term “anti-androgens” as used herein includes, but is not limited to, bicalutamide (CASODEX™), which can be formulated, e.g. as disclosed in U.S. Pat. No. 4,636,505.

The term “bengamides” relates to bengamides and derivatives thereof having aniproliferative properties and includes, but is not limited to, the compounds generically and specifically disclosed in WO00/29382, preferably, to the compound disclosed in Example 1 of WO00/29382.

The term “bisphosphonates” as used herein includes, but is not limited to etridonic acid, clodronic acid, tiludronic acid, pamidronic acid, alendronic acid, ibandronic acid, risedronic acid and zoledronic acid. “Etridonic acid” can be administered, e.g., in the form as it is marketed, e.g. under the trademark DIDRONEL™. “Clodronic acid” can be administered, e.g., in the form as it is marketed, e.g. under the trademark BONEFOS™. “Tiludronic acid” can be administered, e.g., in the form as it is marketed, e.g. under the trademark SKELID™. “Pamidronic acid” can be administered, e.g., in the form as it is marketed, e.g. under the trademark AREDIA™. “Alendronic acid” can be administered, e.g., in the form as it is marketed, e.g. under the trademark FOSAMAX™. “Ibandronic acid” can be administered, e.g., in the form as it is marketed, e.g. under the trademark BONDRANAT™. “Risedronic acid” can be administered, e.g., in the form as it is marketed, e.g. under the trademark ACTONEL™. “Zoledronic acid” can be administered, e.g., in the form as it is marketed, e.g. under the trademark ZOMETA™.

“Trastuzumab” can be administered, e.g., in the form as it is marketed, e.g. under the trademark HERCEPTIN™.

In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

Accordingly, the invention provides the use of a compound of formula I for treating a disease or condition mediated by IGF-1R, wherein the medicament is prepared for administration with another therapeutic agent. The invention also provides the use of another therapeutic agent for treating a disease or condition mediated by IGF-1R, wherein the medicament is administered with a compound of formula I.

The invention also provides a compound of formula I for use in a method of treating a disease or condition mediated by IGF-1R, wherein the compound of formula I is prepared for administration with another therapeutic agent. The invention also provides another therapeutic agent for use in a method of treating a disease or condition mediated by IGF-1R, wherein the other therapeutic agent is prepared for administration with a compound of formula I. The invention also provides a compound of formula I for use in a method of treating a disease or condition mediated by IGF-1R, wherein the compound of formula I is administered with another therapeutic agent. The invention also provides another therapeutic agent for use in a method of treating a disease or condition mediated, wherein the other therapeutic agent is administered with a compound of formula I.

The invention also provides the use of a compound of formula I for treating a disease or condition mediated by IGF-1R, wherein the patient has previously (e.g. within 24 hours) been treated with another therapeutic agent. The invention also provides the use of another therapeutic agent for treating a disease or condition mediated by IGF-1R, wherein the patient has previously (e.g. within 24 hours) been treated with a compound of formula I.

For the treatment of acute myeloid leukemia (AML), compounds of formula I can be used in combination with standard leukemia therapies, especially in combination with therapies used for the treatment of AML. In particular, compounds of formula I can be administered in combination with e.g. farnesyltransferase inhibitors and/or other drugs used for the treatment of AML, such as Daunorubicin, Adriamycin, Ara-C, VP-16, Teniposide, Mitoxantrone, Idarubicin and Carboplatinum.

The structure of the active agents identified by code nos., generic or trade names may be taken from the actual edition of the standard compendium “The Merck Index” or from databases, e.g. Patents International (e.g. IMS World Publications).

The above-mentioned compounds, which can be used in combination with a compound of formula I, can be prepared and administered as described in the art such as in the documents cited above.

In one further embodiment, the additional active ingredient is a hormonal medicine.

In another further embodiment, the additional active ingredient is a PI3 kinase inhibitor, for example NVP-BEZ235 and NVP-BKM120.

In another further embodiment, the additional active ingredient is an mTOR inhibitor, for example everolimus.

### EXAMPLES

The following Examples serve to illustrate the invention without limiting its scope. Abbreviations used are those conventional in the art and as given below.

### ABBREVIATIONS

AcOH acetic acid
Br₂ bromine
brine saturated solution of NaCl in water
DCM dichloromethane
DIEA diisopropylethylamine
DMF dimethyl formamide
DMSO dimethylsulfoxide
eq equivalent(s)
EtOH ethanol
EtOAc ethyl acetate
h hour(s)
H₂O water
HPLC high pressure liquid chromatography
K₃PO₄ potassium phosphate
l liter(s)
MeCN acetonitrile
MEOH methanol
ml milliliter(s)
min minute(s)
MPLC medium pressure liquid chromatography
MS mass spectrum
Na₂CO₃ sodium carbonate
NaHCO₃ sodium bicarbonate
NaHCO₃^{sat} aqueous saturated solution of sodium bicarbonate
Na₂SO₄ sodium sulfate
NIS N-iodosuccinimide
NMR Nuclear Magnetic Resonance
PdCl₂(dppf) [1,1′-bis(diphenylphosphino) ferrocene]dichloropalladium(II)
Pd(PPh₃)₄ tetrakis(triphenylphosphine) palladium(0)
R_{f} ratio of fronts (TLC)
rt room temperature
TFA trifluoroacetic acid
THF tetrahydrofuran
TLC thin layer chromatography
t_{R} time of retention

### Analytical Methods

Temperatures are measured in degrees Celsius. Unless otherwise indicated, the reactions take place at RT. The following HPLC, HPLC/MS and MS methods are used in the preparation of the Intermediates and Examples.

### HPLC Methods:

### Method A

HPLC linear gradient between A=H₂O/TFA 1000:1 and B=acetonitrile/TFA 1000:1 Grad 1: 2-100% Bin 4.5 min and 1 min at 100% B; column: Chromolith Performance 100 mm×4.5 mm (Merck, Darmstadt, Germany); flow rate 2 ml/min. Detection at 215 nM.

### Method B

### Column: Speed ROD RP18e, 50×4.6 mm.

Flow rate: 1.3 ml/min
Mobile phase: A) TFA/water (0.1/100, v/v), B) TFA/acetonitrile (0.1/100, v/v)
Gradient: linear gradient from 0% B to 100% B in 6 min then 2 min 100% B

### Detection: UV at 215 nm

### Method C

Linear gradient 20-100% solvent A in 5 min+1.5 min 100% solvent A; detection at 215 nm, flow rate 1 mL/min at 30° C. Column: Nucleosil 100-3 C18 (70×4.0 mm). Solvent A=CH₃CN+0.1% TFA; Solvent B=H₂O+0.1% TFA.

### Method D

Column: Nucleodur C18 Gravity, 70×4.0 mm., flow rate: 2.0 ml/min
Mobile phase: A) TFA/water (0.1/100, v/v), B) TFA/acetonitrile (0.1/100, v/v)
Gradient: 1% B -->20% B (1 min), -->100% B (2 min), 100% B (1 min).

### Detection: UV at 215 nm

### MS Methods:

### Method L

### Micromass Platform II

### Range DA 200-900

### Cone+30 V and −30 V

Pump Agilent 1100 Quat, 2 min, 0.05 ml/min 1:1 methanol: 15% methanol in water, containing 0.2% ammonium hydroxide (25%).

### Injector, CTC PAL

### Method M

### Agilent G1379A Degasser

### Agilent G1312A Binary Pump

### Agilent G1367A Well Plate Auto Sampler

### Agilent G1316A Column Heater

### Agilent G1315B Diode Array Detector

### Agilent G1496C MSD

### Sedex 75 Evaporative Light Scattering Detector

### Mobile Phase: H2O+0.05% TFA and Acetonitrile+0.035% TFA

Gradient: 1 mL/minute, initial X % ACN to final X % ACN in 3 minutes, 100% B for 0.49 mlnutes, 100% B to initial X % B in 0.1 minute. The column is re-equilibrated in the ˜45 seconds between injections.
MS Scan: 150 to 1000 amu in 1 second
Diode Array Detector: monitors 220 nm, 254 nm, and 280 nm

### Preparative HPLC Methods:

### Method R

Gilson preparative HPLC system, with UV-triggered collection system
Column, Sunfire Prep C18 OBD 5 microm 30×100 mm, temperature 25° C.
Eluent, gradient from 5-100% acetonitrile in 0.05% aqueous trifluoroacetic acid over 20 minutes, flow rate 30 ml/min.

### Detection UV 254 nm

### Method S

### Instrument: Waters 2525 Binary Pump, Waters 515 Make Up Pump, Waters 2767 Auto Sampler/Fraction Collector, Waters 2487 Dual Wavelength UV Detector, Waters ZQ Mass Spectrometer

Mass triggered collection system.

### Mobile Phase: H2O+0.05% TFA (A), Acetonitrile+0.035% TFA (B)

### UV Detector: 220 nm and 254 nm

MS Scan: 180 to 800 amu in 0.5 seconds

### Gradient:

| Time (min) | Flow Rate (mL/min) | % B |
|---|---|---|
| 0 | 20 | 10 |
| 1.4 | 20 | 10 |
| 1.45 | 100 | 10 |
| 3.99 | 100 | 40 |
| 4 | 100 | 100 |
| 4.15 | 100 | 100 |
| 4.16 | 100 | 10 |
| 4.2 | 10 | 10 |
| 4.25 | 10 | 10 |

### HPLC/MS Methods:

### Method X

### ZQ 2000

Range Da 100-900 (positive) and 120-900 (negative)

### Cone+17V and −17 V

Pump Agilent 1100 Bin, 3.5 minute run time, channel A water with 5% acetonitrile, channel B acetonitrile, containing 0.5-1.0% formic acid

| Time (min) | Flow Rate (mL/min) | % B |
|---|---|---|
| 0 | 1.2 | 10 |
| 1.4 | 1.4 | 95 |
| 1.45 | 2.4 | 95 |
| 3.99 | 2.4 | 10 |

Injector, CTC PAL, 5 microl

### Oven Agilent 1100, 50° C.

Column, Waters XBridge, 3×30 mm, 2.5 microm, C18

### Detector, Agilent 1100 DAD, 210-350 nm

### Method Y

Instrument: Agilent G1379A Degasser, Agilent G1312A Binary Pump, Agilent G1367A Well Plate Auto Sampler, Agilent G1316A Column Heater, Agilent G1315B Diode Array Detector, Agilent G1496C MSD, Sedex 75 Evaporative Light Scattering Detector

### Eluent:

A: Water+0.05% Formic Acid+0.05% Ammonium acetate (7.5 Msolution)

### B: Acetonitrile+0.04% Formic Acid

### Column

### Ascentis Express RP-Amide 2.7 um 2.1×30 mm @ 50° C.

### Gradient

Flow: 1.2 ml/min

### Time % B

0 2
1.7 98
2.15 98
2.19 2
UV detection, DAD 210-350 nm
MS detection, 100-900 m/z

### Chemical Synthesis

### Intermediates

### Intermediate A

### cis-3-[4-amino-7-(3-azetidin-1-ylmethyl-cyclobutyl)-7H-pyrrolo[2,3-d]pyrimidin-5-yl]-phenol

cis-7-(3-azetidin-1-ylmethyl-cyclobutyl)-5-(3-benzyloxy-phenyl)-7H-pyrrolo[2,3-d]pyrimidin-4-ylamine (J. Slade et al., Organic Process Research & Development (2007), 11, p. 825, 2.0 g, 4.55 mmol) was shaken for 24 h in presence of Pd/C 10% (400 mg) in MeOH/THF 1:1 (30 ml) under H₂ atmosphere (1.1 bar). Then the RM was filtered over Celite; the catalyst was washed with MeOH and the whole solution evaporated under vacuum to give the title compound as a white solid. HPLC t_{R} 2.12 min (Method A); MS M+H=350 and M−H=348 (Method L).

### Intermediate B

### cis-3-{4-amino-7-[3-(1,1-dioxothiomorpholin-4-ylmethyl)-cyclobutyl]-7H-pyrrolo[2,3-d]pyrimidin-5-yl}-phenol

The title compound was synthesized in a similar manner as described for Intermediate A starting from cis-5-(3-benzyloxy-phenyl)-7-[3-(1,1-dioxothiomorpholin-4-ylmethyl)-cyclobutyl]-7H-pyrrolo[2,3-d]pyrimidin-4-ylamine stage B.2 to give the title compound as an off-white solid. HPLC: t_{R} 2.07 min (Method A); MS M+H=428 (Method L).

### Stage B.2

### cis-5-(3-benzyloxy-phenyl)-7-[3-(1,1-dioxothiomorpholin-4-ylmethyl)-cyclobutyl]-7H-pyrrolo[2,3-d]pyrimidin-4-ylamine

To a mixture of cis-5-(3-benzyloxy-phenyl)-7-(3-thiomorpholin-4-ylmethyl-cyclobutyl)-7H-pyrrolo[2,3-d]pyrimidin-4-ylamine (Stage B.3, 2.28 g, 4.69 mmol) in THF (50 ml) cooled to 0° C. was added potassium monopersulfate triple salt (Oxone) (8.67 g, 14.1 mmol) dissolved in water (50 ml). The RM was stirred at 0° C. for 1 h and then at rt for 2 h. Then the RM was cooled with an ice bath, buffered with NaOAc aq. (1.16 g, 14.1 mmol in 10 ml water) and treated with Na₂S₂O₅ (1.82 g) until KI paper stays white and stirred again for 20 min. Then the RM was extracted with EtOAc and saturated NaHCO₃ solution. The organic layer was dried over Na₂SO₄ and evaporated. The residue was purified by flash chromatography (DCM/MeOH 1% to 6%). The fractions containing product were evaporated together to give the title compound as an off-white foam. HPLC: t_{R} 2.83 min (Method A); MS M+H=518 (Method L).

### Stage B.3

### cis-5-(3-benzyloxy-phenyl)-7-(3-thiomorpholin-4-ylmethyl-cyclobutyl)-7H-pyrrolo[2,3-d]pyrimidin-4-ylamine

To a solution of thiomorpholine (ABCR, Karlsruhe, Germany, 7.44 g, 72.1 mmol) in DMF heated at 70° C. was added over 2 h in portion toluene-4-sulfonic acid cis-3-[4-amino-5-(3-benzyloxy-phenyl)-pyrrolo[2,3-d]pyrimidin-7-yl]-cyclobutylmethyl ester (stage B.4, 4 g, 7.21 mmol). The reaction mixture was stirred 4 h at 70° C., then cooled, poured into ice/water and extracted with EtOAc (2×). The combined organic layers were washed with water (2×) and brine, dried over Na₂SO₄, filtered and evaporated. The residue was purified by flash chromatography (DCM/MeOH 1% to 6%). The fractions containing product were evaporated together to give the title compound as an white foam. HPLC: t_{R} 2.91 min (Method A); MS M+H=486 (Method L).

### Stage B.4

### toluene-4-sulfonic acid cis-3-[4-amino-5-(3-benzyloxy-phenyl)-pyrrolo[2,3-d]pyrimidin-7-yl]-cyclobutylmethyl ester

The title compound is synthesized in a similar manner as described in (step 62.1, WO 02/092599) starting from the cis isomer obtained as described in (Example 47(b), WO 97/28161). HPLC: t_{R} 3.80 min (Method A); MS M+H=555 (Method L).

### Intermediate C

### (5-methyl-tetrahydro-furan-2-yl)methanol

According to the procedure described in J. Org. Chem. 1981, 46, 5 p. 938, to a mixture of 5-hexen-2-ol (ChemSampCo, Dallas, USA, 5 g, 49.9 mmol) in DCM (75 ml) was added dropwise at rt a solution of 3-chloroperbenzoic acid (Aldrich, Buchs, Switzerland, 8.61 g, 49.9 mmol) in DCM (125 ml) over 1 h. The RM was stirred at rt for 24 h. Then the RM was diluted with DCM (100 ml) and washed with 2×50 ml sat. aqueous Na₂CO₃ and 50 ml brine, dried over Na₂SO₄ and K₂CO₃, filtered and evaporated to dryness to give the title compound as a liquid. ¹H-NMR (CDCl₃, 400 MHz): 4.20-3.90 (m, 2H), 3.75-3.56 (m, 1H), 3.53-3.39 (m, 1H), 2.11-1.81 (m, 3H), 1.81-1.58 (m, 1H), 1.57-1.35 (m, 1H), 1.29-1.15 (m, 3H).

### Intermediate D

### 4-{3-[4-amino-5-(3-hydroxy-phenyl)-pyrrolo[2,3-d]pyrimidin-7-yl]-cyclobutylmethyl}-1-methyl-piperazin-2-one

The title compound was synthesized in a similar mariner as described for Intermediate A and Stage B.3. starting from commercially available 1-methyl-piperazin-2-one (3B Scientific, Libertyville, USA) to give the title compound as an off-white foam. HPLC: t_{R} 2.06 min (Method A); MS M+H=407 (Method L).

### Intermediate E

### (5,5-dimethyl-tetrahydro-furan-2-yl)-methanol

The following example was synthesized in a similar manner as described for Intermediate C using as replacement for the 5-hexen-2-ol the starting material 2-methyl-hex-5-en-2-ol (Beta Pharma, New Haven, USA) ¹H-NMR (CDCl₃, 400 MHz): 4.15-4.05 (m, 1H), 3.72-3.64 (m, 1H), 3.52-3.44 (m, 1H), 2.02-1.92 (m, 1H), 1.91-1.69 (m, 3H), 1.31-1.23 (m, 6H).

### Intermediate F

### ((S)-5,5-dimethyl-tetrahydro-furan-2-yl)-methanol

According to the procedure described in Tet. Lett. 1994, p. 7467, to a mixture of toluene-4-sulfonic acid (R)-5-oxo-tetrahydro-furan-2-ylmethyl ester (Aldrich, Buchs, Switzerland, 500 mg, 1.85 mmol) in THF (9 ml) cooled to −90° C. with a hexane/liquid nitrogen bath, was added dropwise a solution of methyllithium in diethyl ether 1.6 M (2.31 ml, 3.7 mmol). The RM was stirred at −90° C. for 2 h and then the RM was allowed to reach rt over 4 h. Then the RM was quenched with brine (20 ml) and 1 Maqueous HCl (3 ml). The RM was saturated with NaCl and extracted with diethyl ether. The combined organic layers were dried over Na₂SO₄, filtered and evaporated to dryness to give the title compound as an oil. ¹H-NMR (CDCl₃, 400 MHz): 4.14-4.08 (m, 1H), 3.68 (dd, 1H), 3.48 (dd, 1H), 2.01-1.93 (m, 1H), 1.86-1.73 (m, 3H), 1.31-1.23 (m, 6H).

### Intermediate G

### ((R)-5,5-dimethyl-tetrahydro-furan-2-yl)-methanol

The title compound was synthesized as described for Intermediate F starting from (S)-5-oxo-tetrahydro-furan-2-ylmethyl ester (Aldrich, Buchs, Switzerland).

### Intermediate H

### cis-3-{4-amino-7-[3-(4,4-difluoro-piperidin-1-ylmethyl)-cyclobutyl]-7H-pyrrolo[2,3-d]pyrimidin-5-yl}-phenol

The title compound was synthesized in a similar manner as described for stage B.3 using

4,4-difluoropiperidine (ChemCollect, Remscheid, Germany) to give the title compound as an off-white solid. HPLC: t_{R} 2.22 min (Method A); MS M+H=414 (Method L).

### Intermediate I

### 2-(1-ethyl-propoxy)-ethanol

To a solution of [2-(1-ethyl-propoxy)-ethoxymethyl]-benzene (Stage I.1, 180 mg, 0.802 mmol) in EtOAc (8 ml) was added Pd/C 10% (80 mg, 0.752 mmol) and the RM was stirred under H₂ atmosphere at rt for 5.5 h. The RM was then filtered over Celite and washed several times with EtOAc. The filtrate was evaporated. It was then dried under vacuum for 10 min to afford the title product as an oil. ¹H-NMR (CDCl₃, 400 MHz): 3.74-3.69 (m, 2H), 3.56-3.53 (m, 2H), 3.18 (qt, 1H), 2.07-2.03 (m, 1H), 1.55-1.48 (m, 4H), 0.90 (t, 6H).

### Stage I.1

### [2-(1-ethyl-propoxy)-ethoxymethyl]-benzene

In a microwave vial, pentan-3-ol (Aldrich, Buchs, Switzerland; 5 ml, 46.3 mmol) was dissolved in DMF (5 ml) under argon atmosphere. NaH in oil (55%, 401 mg, 9.20 mmol) was added in 2 portions and the RM was stirred at rt. After 15 min, (2-bromo-ethoxymethyl)-benzene (Aldrich, Buchs, Switzerland; 1.36 g, 6.13 mmol) was added and then more DMF (3 ml) was added to get a less viscous solution. The RM then was heated at 115° C. for 1.5 h under microwave irradiation. The RM was evaporated to dryness and the residue was diluted with water and extracted with EtOAc. The organic phase was washed with brine, dried over Na₂SO₄, filtered and evaporated. The crude was then dissolved in DMF and purified by Prep.HPLC(H₂O (0.1% TFA)/CH₃CN 95:5 to 0:100) in 4 runs. The fractions containing product were collected together and basified with NaHCO₃, before being concentrated and extracted with EtOAc (1×). The organic layer was washed with brine, before being dried over Na₂SO₄, filtered and evaporated to dryness to give after 15 min under vacuum the title compound as an oil. HPLC: t_{R} 3.80 min (Method A); MS M+H=223 (Method L).

### Intermdiate J

### 2-cyclopentyloxy-ethanol

The title compound was obtained by analogy to Intermediate I, starting with cyclopentanol (Aldrich, Buchs, Switzerland) instead of pentan-3-ol in Stage I.1. ¹H-NMR (CDCl₃, 400 MHz): 3.96-3.91 (m, 1H), 3.73-3.68 (m, 2H), 3.51-3.48 (m, 2H), 2.05-1.99 (m, 1H), 1.77-1.62 (m, 6H), 1.55-1.50 (m, 2H).

### Intermediate K

### 2-(3((7-oxa bicyclo[2.2.1]heptan-1-ylmethoxy)phenyl)-4,4,5,5-tetramethyl-1,3,2-dioxaborolane

A mixture of 1-(iodomethyl)-7-oxabicyclo[2.2.1]heptanes (Stage K.1, 2.24 g, 9.4 mmol), 3-(4,4,5,5-tetramethyl-1,3,2-dioxaborolan-2-yl)phenol (4.14 g, 18.8 mmol) and K₂CO₃ (5.19 g, 37.6 mmol) in dry acetonitrile (24 mL) were heated at 150° C. in a pressure vessel for 18 h. The reaction was cooled to room temperature and filtered, the filtrate was concentrated in vacuo, followed by silica gel chromatography (EtOAc I hexanes: 1-20% gradient) to afford the title compound. ¹H-NMR (CDCl₃, 400 MHz): 7.42-7.36 (m, 2H), 7.29 (t, 1H), 7.11-7.06 (m, 1H), 4.64-4.59 (m, 1H), 4.29 (s, 2H), 1.94-1.75 (m, 4H), 1.65-1.56 (m, 4H), 1.34 (s, 12H).

### Step K.1

### 1-(iodomethyl)-7-oxabicyclo[2.2.1]heptane

A solution of 4-methylenecyclohexanol (Step K.2, 2.4 g, 21.4 mmol) and N-iodosuccinimide (8.8 g, 37.6 mmol) in dry acetonitrile (100 mL) was stirred at room temperature in the dark overnight. The resulting mixture was poured into water and extracted with ether. The extract was washed successively with sat. aq. Na₂S₂O₄, sat. aq. NaHCO₃ and brine, then dried with Na₂SO₄. After concentration at 200 mbar and 30° C., the residue was purified by silica gel chromatography (EtOAc/Hexane: 0-20% gradient) to afford the title compound.

¹H-NMR (CDCl₃, 400 MHz): 4.66-4.62 (m, 1H), 3.55 (s, 2H), 1.97-1.60 (m, 8H).

### Step K.2

### 4-methylenecyclohexanol

To a solution of 4-methylenecyclohexanone (Step K.3, 2.8 g, 25.45 mmol) in MeOH (100 mL) was added NaBH₄ (1.93 g, 50.9 mmol) at 0° C. The reaction was stirred at room temperature for 2 h, and quenched with sat. aq. NH₄Cl. The reaction was extracted with DCM, the collected organic extracts were dried (Na₂SO₄), concentrated at 200 mbar and 30° C. to afford the title compound which was used without further purification.

### Step K.3

### 4-methylenecyclohexanone

To a solution of 8-methylene-1,4-dioxaspiro[4.5]decane (Step K.4, 5.12 g, 33.2 mmol) in acetone (15 mL) and water (15 mL) was added oxalic acid dihydride (8.33 g, 66.1 mol), the reaction was stirred at room temperature for 3 h. Solid NaHCO₃ was added slowly to the reaction, the solid was filtered and washed thoroughly with diethylether. The combined organic extracts were concentrated at 200 mbar and 30° C. to afford the title compound which was used without further purification. ¹H-NMR (CDCl₃, 400 MHz): 4.88 (5, 2H), 2.52 (t, 4H), 2.43 (t, 4H).

### Step K.4

### 8-methylene-1,4-dioxaspiro[4.5]decane

A solution of n-BuLi (2.5 M in hexanes, 30 mL, 75 mmol) was slowly added to a suspension of methyltriphenylphosphonium bromide (28.07 g, 79 mmol) in THF (150 mL) at −10° C. After stirring for 1 h, 1,4-dioxaspiro[4.5]decan-8-one (8.01 g, 51.3 mmol) was added. The reaction was warmed to room temperature and stirred for 4 h. The reaction was quenched with sat. aq. NH₄Cl, extracted by diethyl ether. The combined organic extracts were dried (Na₂SO₄), concentrated at 200 mbar and 30° C. The residue was diluted with DCM and hexanes (1:1), and the solid was filtered. The organic extracts were concentrated at 200 mbar and 30° C., followed by silica gel chromatography (EtOAc/hexanes: 0-10%-20% gradient) to afford the title compound. ¹H-NMR (CDCl₃, 400 MHz): 4.69 (s, 2H), 3.99 (s, 4H), 2.30 (t, 4H), 1.72 (t, 4H).

### Intermediate L

### benzoic acid cis-3-(4-chloro-5-iodo-pyrrolo[2,3-d]pyrimidin-7-yl)-cyclobutylmethyl ester

A mixture of benzoic acid 3-(4-chloro-pyrrolo[2,3-d]pyrimidin-7-yl)-cyclobutylmethyl ester (Staep L.1, 1.7 g, 4.97 mmol), N-iodosuccinimide (1.23 g, 5.47 mmol) and DMF (9 ml) was stirred at room temperature for 48 hours. Ethyl acetate and water were added and the title compound collected by filtration. HPLC/MS t_{R} 3.46 min, M+H 468.2 and M−H 467.0 (Method Y).

### Step L.1

### benzoic acid cis-3-(4-chloro-pyrrolo[2,3-d]pyrimidin-7-yl)-cyclobutylmethyl ester

A mixture of (4,6-dichloro-pyrimidin-5-yl)-acetaldehyde (Astatech, 1.40 g, 7.31 mmol), benzoic acid 3-amino-cyclobutylmethyl ester (prepared as described in Org. Process Res. Dev. 2007, 11, 825-835., 1.5 g, 7.31 mmol), diisopropylethylamine (0.95 g, 7.31 mmol) and ethanol (15 ml) were heated at reflux for 5.5 hours under an argon atmosphere. The reaction mixture was evaporated, taken up in THF (10 ml), aqueous HCl (4 ml, 4M) added and stood at room temperature for 1 hour. The volume of the mixture was then reduced under vacuum, made neutral with aqueous sodium bicarbonate solution, extracted 3× with DCM, the organic layers dried over sodium sulphate and evaporated. Purification by flash column chromatography, eluting with a DCM/EtOAc gradient gave the title compound. HPLC/MS t_{R} 1.52 min, M+H 342.1 (Method X).

### Intermediate M

### [cis-3-(4-amino-5-iodo-pyrrolo[2,3-d]pyrimidin-7-yl)-cyclobutyl]-methanol

The title compound is prepared as described in WO 2005/097800. Or alternatively as described below:

A mixture of [3-(4-Chloro-5-iodo-pyrrolo[2,3-d]pyrimidin-7-yl)-cyclobutyl]-methanol (Step N.1, 2.0 g, 5.50 mmol), 25% aqueous ammonia solution (10.4 ml) and 1,4-dioxane (5 ml) were heated in sealed tube at 80° C. for 15.5 hours. After cooling the reaction mixture was evaporated and purified by flash column chromatography, eluting with a gradient of DCM/methanol, to give the title compound. ¹H-NMR (d₆-DMSO, 400 MHz): 8.06 (s, 1H), 7.68 (s, 1H), 6.57 (broad s, 2H), 5.06-4.87 (m, 1H), 4.57 (t, 1H), 3.49-3.40 (m, 2H), 2.45-2.35 (m, 2H), 2.28-2.13 (m, 2H).

### Step M.1

### [cis-3-(4-Chloro-5-iodo-pyrrolo[2,3-d]pyrimidin-7-yl)-cyclobutyl]-methanol

A solution of DIBAL-H in toluene (0.73 ml, 0.73 mmol) was added dropwise to a stirred suspension of benzoic acid 3-(4-chloro-5-iodo-pyrrolo[2,3-d]pyrimidin-7-yl)-cyclobutylmethyl ester (Intermediate L, 170 mg, 0.36 mmol) in DCM (3 ml) cooled with a dry-ice I acetone bath. After 30 minutes the reaction mixture was warmed over 1 hour to 0° C., stirred 1 hour at 0° C., and silica gel (2 g) was added. The reaction mixture was evaporated and the residue purified by flash chromatography, to give the title compound. HPLC/MS t_{R} 1.09 min, M+H 365.8 (Method X).

### Intermediate N

### cis-3-{4-amino-5-[3-(7-oxa-bicyclo[2.2.1]hept-1-ylmethoxy)-phenyl]-pyrrolo[2,3-d]pyrimidin-7-yl}-cyclobutanecarbaldehyde

To a suspension of (3-{4-amino-5-[3-(7-oxa-bicyclo[2.2.1]hept-1-ylmethoxy)-phenyl]-pyrrolo[2,3-d]pyrimidin-7-yl}-cyclobutyl)-methanol (Example 22, 146 mg, 0.35 mmol) in AcCN (1 ml) was added 2-iodoxybenzoic acid (432 mg, 0.69 mmol) and the mixture was heated in a sealed vessel at 80° C. for 1 hour. After cooling the reaction mixture was evaporated and the residue eluted through a short plug of silica gel with DCM:methanol, the combined product containing fractions were combined and evaporated to give the title compound which was used without further purification.

### Intermediate O

### 2-[3-(4,4,5,5-tetramethyl-[1,3,2]dioxaborolan-2-yl)-phenoxymethyl]-tetrahydro-pyran

3-Hydroxyphenylboronic acid pinacol ester (5.0 g, 22.7 mmol), 2-(bromomethyl) tetrahydropyran (4.4 mL, 34.1 mmol) and potassium carbonate (12.6 g, 91.0 mmol) were suspended in DMF (25 mL) and stirred at 125° C. for 3 h. The reaction was allowed to cool and concentrated under reduced pressure. The remaining crude material was taken up in EtOAc, washed with brine and the organic layer were dried and concentrated. The remaining crude product was purified by normal phase chromatography, eluting with a hexanes/EtOAc gradient to give the title compound as colorless oil. MS M+H 319.1. ¹H-NMR (CDCl₃, 400 MHz): 7.38 (d, 1H), 7.33 (s, 1H), 7.28 (dd, 1H), 7.04 (d, 1H), 4.07-4.01 (m, 2H), 3.98 (dd, 1H), 3.90-3.84 (m, 1H), 3.51 (dd, 1H), 1.91-1.88 (m, 1H), 1.71-1.46 (m, 5H), 1.33 (s, 12H).

### Intermediate P

### (cis-3-{4-amino-5-[3-(tetrahydro-pyran-2-ylmethoxy)-phenyl]-pyrrolo[2,3-d]pyrimidin-7-yl}-cyclobutyl)-methanol

A mixture of [cis-3-(4-amino-5-iodo-pyrrolo[2,3-d]pyrimidin-7-yl)-cyclobutyl]-methanol (Intermediate M, 950 mg, 2.48 mmol), 2-[3-(4,4,5,5-tetramethyl-[1,3,2]dioxaborolan-2-yl)-phenoxymethyl]-tetrahydro-pyran (Intermediate O, 830 mg, 2.61 mmol), tetrakis(triphenylphosphine)palladium (287 mg, 0.25 mmol), sodium carbonate (527 mg, 4.97 mmol), DMF (10 ml) and water (5 ml) was heated in a sealed vessel at 80° C. for 18 hours under an argon atmosphere. After cooling, water was added and the mixture extracted 3× with DCM, dried over sodium sulphate and evaporated to give the crude product. Purification by normal phase chromatography, eluting with a DCM/methanol gradient gave the title compound. MS M+H 409.1 and M−H 407.2 (Method L).

### Intermediate Q

### cis-3-{4-amino-5-[3-(tetrahydro-pyran-2-ylmethoxy)-phenyl]-pyrrolo[2,3-d]pyrimidin-7-yl}-cyclobutanecarbaldehyde

To a mixture of (cis-3-(4-amino-5-(3-((tetrahydro-2H-pyran-2-yl)methoxy)phenyl)-7H-pyrrolo[2,3-d]pyrimidin-7-yl)-cyclobutyl)methanol (Intermediate P, 4 mg, 0.01 mmol) in acetonitrile (1 mL) was added IBX (5.5 mg, 0.02 mmol). The reaction vessel was sealed and the mixture heated at 80° C. for 1 h. The reaction mixture was cooled to room temperature and filtered to give the crude title compound which was used directly without further purification. MS m/z 407.2 (M+H⁺) and 425.2 (M+H₂O+H⁺) (Method M).

### Intermediate R

### [trans-3-(4-amino-5-iodo-pyrrolo[2,3-d]pyrimidin-7-yl)-cyclobutyl]-methanol

The title compound was prepared in a similar manner to Intermediate M. MS m/z 345 (M+H⁺) (Method M).

### Intermediate S

### (trans-3-{4-amino-5-[3-(tetrahydro-pyran-2-ylmethoxy)-phenyl]-pyrrolo[2,3-d]pyrimidin-7-yl}-cyclobutyl)-methanol

The title compound was prepared in a similar manner to Intermediate P starting from [trans-3-(4-amino-5-iodo-pyrrolo[2,3-d]pyrimidin-7-yl)-cyclobutyl]-methanol (intermediate R). MS m/z 409 (M+H⁺) (Method M).

### Intermediate T

### (cis-3-{8-amino-1-[3-(tetrahydro-pyran-2-ylmethoxy)-phenyl]-imidazo[1,5-a]pyrazin-3-yl}-cyclobutyl)-methanol

[3-(8-Amino-1-iodo-imidazol[1,5-a]pyrazin-3-yl-cyclobutyl]-methanol (prepared according to US20070129547 as 5:1 cis/trans mixture; 600 mg, 1.7 mmol) was dissolved in dioxane (10 mL). Water (10 mL), [3-(4,4,5,5-tetramethyl-[1,3,2]dioxaborolan-2-yl)-phenoxymethyl]-tetrahydro-pyran (832 mg, 2.6 mmol), K₃PO₄ (1.5 g, 6.9 mmol) and Pd (PPh₃)₄ (210 mg, 0.18 mmol) were added and the reaction mixture was flushed with argon and heated to 60° C. for 1 h. The reaction mixture was allowed to cool and diluted with EtOAc. The organic layer was washed with brine, dried and concentrated. The crude product was purified by flash column chromatography, eluting with a DCM/MeOH gradient to give the title compound as pure cis-isomer as the major fraction. M+H 409.2. ¹H-NMR (MeOH d₄, 400 MHz) cis-isomer 7.47-7.44 (m, 2H), 7.20 (d, 2H), 7.07 (d, 1H), 7.00 (d, 1H), 4.03-4.00 (m, 3H), 3.96-3.84 (m, 1H), 3.80-3.74 (m, 1H), 3.59 (d, 2H), 3.55-3.51 (m, 1H), 2.65-2.57 (m, 3H), 2.29-2.24 (m, 2H), 1.93-1.90 (m, 1H), 1.74 (d, 1H), 1.63-1.60 (m, 3H), 1.59-1.54 (m, 1H).

### Intermediate U

### 2-[3-(5,5-dimethyl-tetrahydro-furan-2-ylmethoxy)-phenyl]-4,4,5,5-tetramethyl-[1,3,2]dioxaborolane

A mixture of (5,5-dimethyltetrahydrofuran-2-yl)methyl bromide (Step U.1, 0.696 g, 3.63 mmol), 3-(4,4,5,5-tetramethyl-1,3,2-dioxaborolan-2-yl)phenol (0.532 g, 2.42 mmol) and K₂CO₃ (1.32 g, 9.57 mmol) in dry acetonitrile (6 mL) was heated at 100° C. in a pressure vessel for 4 h. An additional 0.696 g (3.63 mmol) of (5,5-dimethyltetrahydrofuran-2-yl)methyl bromide (Step U.1) was added to the reaction mixture and heating to 100° C. in a pressure vessel was resumed for 16 more hours. The reaction was cooled to room temperature and filtered, the filtrate was concentrated in vacuo, followed by silica gel chromatography (EtOAc/hexanes: 0-20% gradient) to afford the title compound. MS m/z 333.2 (M+H⁺) (Method M). ¹H-NMR (CDCl₃, 400 MHz): 7.40-7.24 (m, 3H), 7.06-7.01 (m, 1H), 4.39-4.31 (m, 1H), 4.04-3.89 (m, 2H), 2.19-2.10 (m, 1H), 1.95-1.73 (m, 3H), 1.34 (s, 12H), 1.30 (s, 3H), 1.28 (s, 31-1).

### Step U.1

### (5,5-dimethyltetrahydrofuran-2-yl)methyl bromide

(5,5-dimethyltetrahydrofuran-2-yl)methyl bromide was synthesized via a published synthetic procedure (Compound 9b in: Bloodworth, A. J. et al. J. Chem. Soc. Perkin Trans. 2. 1988, 575-582).

### Intermediate V

### (cis-3-{4-amino-5-[3-(5,5-dimethyl-tetrahydro-furan-2-ylmethoxy)-phenyl]-30 pyrrolo[2,3-d]pyrimidin-7-yl}-cyclobutylymethanol

The title compound was prepared in a similar manner to Intermediate P starting from 2-[3-(5,5-dimethyl-tetrahydro-furan-2-ylmethoxy)-phenyl]-4,4,5,5-tetramethyl-[1,3,2]dioxaborolane (Intermediate U). MS m/z 423.2 (M+H⁺) (Method M).

### Intermediate W

### (trans-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol

The title compound was prepared in a similar manner to Intermediate P starting from [trans-3-(4-amino-5-iodo-pyrrolo[2,3-d]pyrimidin-7-yl)-cyclobutyl]-methanol (Intermediate R) and 2-[3-(5,5-dimethyl-tetrahydro-furan-2-ylmethoxy)-phenyl]-4,4,5,5-tetramethyl-[1,3,2]dioxaborolane (Intermediate U). MS m/z 423.2 (M+H⁺) (Method M).

### Intermediate X

### -[3-(tetrahydro-furan-2-ylmethoxy)-phenyl]-4,4,5,5-tetramethyl-[1,3,2]dioxaborolane

A mixture of 3-(4,4,5,5-tetramethyl-1,3,2-dioxaborolan-2-yl)phenol (2.2 g, 10 mmol), potassium carbonate (5.52 mg, 40 mmol), 2-(bromomethyl)tetrahydrofuran (4.95 g, 30 mmol) in acetonitrile (20 mL) was heated at 100° C. in a sealed tube overnight. The mixture was cooled to room temperature, and diluted with ethyl acetate. After filtration, the filtrate was concentrated and purified with silica gel flash chromatography, eluent 10% ethyl acetate in hexanes to afford the title compound as colorless oil. MS m/z 305 (M+H⁺) (Method M).

### Intermediate Y

### (cis-3-{4-amino-5-[3-(tetrahydro-furan-2-ylmethoxy)-phenyl]-pyrrolo[2,3-d]pyrimidin-7-yl}-cyclobutyl)-methanol

The title compound was prepared in a similar manner to Intermediate P starting from 2-[3-(tetrahydro-furan-2-ylmethoxy)-phenyl]-4,4,5,5-tetramethyl-[1,3,2]dioxaborolane (Intermediate X). MS m/z 395 (M+H⁺) (Method M).

### Intermediate Z

### (trans-3-{4-amino-5-[3-(tetrahydro-furan-2-ylmethoxy)-phenyl]-pyrrolo[2,3-d]pyrimidin-7-yl}-cyclobutyl)-methanol

The title compound was prepared in a similar manner to Intermediate P starting from [trans-3-(4-amino-5-iodo-pyrrolo[2,3-d]pyrimidin-7-yl)-cyclobutyl]-methanol (Intermediate R) and -[3-(tetrahydro-furan-2-ylmethoxy)-phenyl]-4,4,5,5-tetramethyl-[1,3,2]dioxaborolane (Intermediate X). MS m/z 395 (M+H⁺) (Method M).

### Intermediate AA

### toluene-4-sulfonic acid cis-3-{4-amino-5-[3-(tetrahydro-pyran-2-ylmethoxy)-phenyl]-pyrrolo[2,3-d]pyrimidin-7-yl}-cyclobutylmethyl ester

To a solution of (cis-3-{4-amino-5-[3-(tetrahydro-pyran-2-ylmethoxy)-phenyl]-pyrrolo[2,3-d]pyrimidin-7-yl}-cyclobutyl)-methanol (Intermediate P) (320 mg, 0.78 mmol) in pyridine (3.5 ml) cooled at −20° C. was added portion wise p-toluenesulphonyl chloride (508 mg, 2.33 mmol). The reaction mixture was stirred at −20° C. for 18 hours, then quenched with ice-water and extracted 3×DCM. The combined organic extracts were washed with cold 1 Msulphuric acid, then saturated brine, dried over sodium sulphate and evaporated to give the title compound which was used without further purification.

### Intermediate AB

### (R)-2-[3-(4,4,5,5-tetramethyl-[1,3,2]dioxaborolan-2-yl)-phenoxymethyl]-tetrahydro-pyran and (S)-2-[3-(4,4,5,5-tetramethyl-[1,3,2]dioxaborolan-2-yl)-phenoxymethyl]-tetrahydro-pyran

A mixture of the enantiomers of 2-(3-bromo-phenoxymethyl)-tetrahydro-pyran (Step AB.2, 500 mg, 1.84 mmol), bis-(pinacolato)diboran (609 mg, 2.40 mmol), potassium acetate (452 mg, 4.61 mmol), 1,1′-bis(diphenylphosphino)ferrocene (135 mg, 0.24 mmol), 1,1′-bis(diphenylphosphino)ferrocenedichloro palladium(II) dichloromethane complex (135 mg, 0.18 mmol) and DMF (4.7 ml) were heated at 80° C. under an argon atmosphere. After 15 hours at 80° C. the reaction mixture was cooled, DCM added, the organic layers washed with water then brine, and dried over sodium sulphate. The crude material was prepabsorbed onto silica gel and then purified by normal phase flash column chromatography, eluting with a methanol/DCM gradient, to give the title compound. HPLC/MS t_{R} 1.61 min, M+H 319.0 (Method X).

### Step AB.1

### (R)-2-(3-bromo-phenoxymethyl)-tetrahydro-pyran and (S)-2-(3-bromo-phenoxymethyl)-tetrahydro-pyran

The racemic 2-(3-bromo-phenoxymethyl)-tetrahydropyran was separated on a Chiralpak OD-H column, 30×250 mm, superchritical CO₂/isopropanol 75:25, 150 bar; 120 ml/min, to give:

### Fraction 1: 1.59 min: R-enantiomer

### Fraction 2: 2.63 min: S-enantiomer

### Step AB.2

### 2-(3-bromo-phenoxymethyl)-tetrahydropyran

3-Bromophenol (10 g, 57.8 mmol), 2-(bromomethyl)-tetrahydropyran (25 g, 140 mmol) and potassium carbonate (24 g, 173 mmol) were suspended in DMF (60 mL) and heated with stirring to 125° C. for 3 h. The reaction mixture was then allowed to cool and concentrated under reduced pressure. The residual crude material was taken up with EtOAc and the organic phase was repeatedly washed with brine, dried and concentrated and further dried under high vacuum to give the title compound as a yellow oil. MS m/z 273 (M+H⁺). ¹H-NMR (MeOH d₄, 400 MHz) 7.19 (t, 1H), 7.14-7.09 (m, 2H), 6.84 (d, 1H), 3.99 (d, 1H), 3.64 (dd, 1H), 3.43 (d, 1H), 1.98-1.83 (m, 1H), 1.64-1.49 (m, 4H), 1.42-1.39 (m, 1H).

### Intermediate AC

### 4-(4-amino-5-(3-((5,5-dimethyltetrahydrofuran-2-yl)methoxy)phenyl)-7H-pyrrolo[2,3-d]pyrimidin-7-yl)-cyclohexanone

To a mixture of 5-(3-((5,5-dimethyltetrahydrofuran-2-yl)methoxy)phenyl)-7-(1,4-dioxaspiro[4.5]decan-8-yl)-7H-pyrrolo[2,3-d]pyrimidin-4-amine (Step AC.1, 90 mg, 0.188 mmol) in acetone (4 mL), was added HCl in ether (1N, 0.94 mL). The reaction mixture was heated at 50° C. for 5 hr. The reaction mixture was concentrated, and then diluted with ethyl acetate. The organic solution was washed with sodium carbonate aqueous solution, brine, and dried over sodium sulfate. After concentration, the title compound was obtained as a yellow solid, and is used directly without purification. MS m/z 435 (M+H⁺) (Method M).

### Step AC.1

### 5-(3-((5,5-dimethyltetrahydrofuran-2-yl)methoxy)phenyl)-7-(1,4-dioxaspiro[4.5]decan-8-yl)-7H-pyrrolo[2,3-d]pyrimidin-4-amine

The title compound was prepared in a similar manner to Intermediate P, utilizing Intermediate U and Step AC.2. MS m/z 479 (M+H⁺) (Method M).

### Step AC.2

### 5-iodo-7-(1,4-dioxaspiro[4.5]decan-8-yl)-7H-pyrrolo[2,3-d]pyrimidin-4-amine

A suspension of 4-chloro-5-iodo-7-(1,4-dioxaspiro[4.5]decan-8-yl)-7H-pyrrolo[2,3-d]pyrimidine (Step AC.3, 200 mg, 0.48 mmol) in ammonium hydroxide (3 mL) was heated in a microwave at 130° C. for 1 h. The reaction was cooled. The solid was collected by filtration to afford the title compound as a light yellow solid. MS m/z 401(M+H⁺) (Method M).

### Step AC.3

### 4-chloro-5-iodo-7-(1,4-dioxaspiro[4.5]decan-8-yl)-7H-pyrrolo[2,3-d]pyrimidine

To a mixture to 4-chloro-5-iodo-7H-pyrrolo[2,3-d]pyrimidine (280 mg, 1 mmol), 1,4-dioxaspiro[4.5]decan-8-ol (190 mg, 1.2 mmol), and PS-PPh₃ (loading 3 mmol/g, 670 mg, 2 mmol) in anhydrous THF (10 mL), was added DIAD (295 uL, 1.50 mmol). The mixture was put on a shaker for 15 hr. The resin was filtered, the filtrate was concentrated and purified with silica gel flash column chromatography (25-30% ethyl acetate in hexanes) to afford the title compound as a white solid, MS m/z 419.9 (M+H⁺) (Method M).

### Intermediate AD

### 5-(3-((5,5-dimethyltetrahydrofuran-2-yl)methoxy)phenyl)-7-(piperidin-4-yl)-7H-pyrrolo[2,3-d]pyrimidin-4-amine

To a solution of tert-butyl 4-(4-amino-5-(3-((5,5-dimethyltetrahydrofuran-2-yl)methoxy)phenyl)-7H-pyrrolo[2,3-d]pyrimidin-7-yl)piperidine-1-carboxylate (Step AD.1, 100 mg, 0.19 mmol) in DCM (3 mL), was slowly added TFA (1 mL). The mixture was stirred at room temperature for 1 hr. After evaporation of the solvents, the title compound was obtained as yellow oil, MS m/z 422.2 (M+H⁺) (Method M).

### Step AD.1

### tert-butyl 4-(4-amino-5-(3-((5,5-dimethyltetrahydrofuran-2-yl)methoxy)phenyl)-7H-pyrrolo[2,3-d]pyrimidin-7-yl)piperidine-1-carboxylate

The title compound was prepared in a similar manner to Intermediate P, utilizing Intermediate U and Step AD.2. MS m/z 522.2 (M+H⁺) (Method M).

### Step AD.2

### tert-butyl 4-(4-amino-5-iodo-7H-pyrrolo[2,3-d]pyrimidin-7-yl)piperidine-1-carboxylate

The title compound was obtained in a similar manner to Step AC.2, utilizing Step AD.3. MS m/z 444 (M+H⁺) (Method M).

### Step AD.3

### tert-butyl 4-(4-chloro-5-iodo-7H-pyrrolo[2,3-d]pyrimidin-7-yl)piperidine-1-carboxylate

The title compound was obtained in a similar manner to Step AC.3, utilizing tert-butyl 4-hydroxypiperidine-1-carboxylate. MS m/z 463 (M+H⁺) (Method M).

### Intermediate AE

### (2S,4R)-4-fluoropyrrolidine-2-carboxamide hydrochloride salt

A solution of (2S,4R)-tert-butyl 2-carbamoyl-4-fluoropyrrolidine-1-carboxylate (Step AE.1, 76 mg, 0.33 mmol) in 4 N HCl in dioxane (2 mL) was stirred at room temperature for 1.5 h. The solvent was evaporated and the residue was coevaporated twice with toluene to give the title compound as a white solid. MS m/z 133.2 (M+H⁺) (Method M).

### Step AE.1

### (2S,4R)-tert-butyl 2-carbamoyl-4-fluoropyrrolidine-1-carboxylate

To a solution of (2S,4R)-4-fluoropyrrolidine-2-carboxylic acid (250 mg, 1.08 mmol) in anhydrous MeCN (2.5 mL) at 0° C. was added EDCI (249 mg, 1.30 mmol, 1.22 eq) and HOBt (200 mg, 1.30 mmol, 1.22 eq). The reaction was allowed to slowly warm up to room temperature and stirred overnight. The reaction was then cooled in an ice bath and concentrated NH₄OH (0.28 mL) was added. After stirring at 0° C. for 1 h, the ice bath was removed and the reaction continued for an additional hour. The reaction was diluted with MeCN (2.7 mL) and the solid was removed by filtration. The filtrate was concentrated and the resulting residue was purified by flash chromatography (SiO₂, EtOAc:hex/0-100%) to give the title compound as a white solid. MS m/z 233.2 (M+H⁺) (Method M).

### Intermediate AF

### (1R,3R,4S)-2-azabicyclo[2.2.1]heptane-3-carboxamide hydrochloride salt.

The title compound was prepared in a similar manner to Intermediate AE starting from (1R,3R,4S)-tert-butyl 3-carbamoyl-2-azabicyclo[2.2.1]heptane-2-carboxylate. MS m/z 141.2 (M+1) (Method M).

### Intermediate AG

### (S)-morpholine-3-carboxamide

To a mixture of (S)-4-(tert-butoxycarbonyl)morpholine-3-carboxylic acid (60 mg, 0.26 mmol) and diisopropylethylamine (92 uL, 0.52 mmol) in THF (2 mL), was added HATU (99 mg, 0.26 mmol). After stirring for 15 min, NH₃ in 1,4-dioxane (0.5 M, 1 mL) was added dropwise. The mixture was stirred for 2 hours and concentrated. The residue was dissolved in DCM (2 mL). To the resulting solution was added TFA (0.4 mL). The mixture was stirred for 1 hour. After concentration, the residue was used directly for further reactions without purification. MS m/z 131.0 (M+H⁺) (Method M).

### Intermedaite AH

### (2S,4S)-4-fluoropyrrolidine-2-carboxamide hydrochloride salt

The title compound was prepared in a manner similar to Intermediate AE starting from (2S,4S)-1-(tert-butoxycarbonyl)-4-fluoropyrrolidine-2-carboxylic acid. MS m/z 133.2 (M+H⁺) (Method M).

### Intermediate AI

### (S)-6,6-dimethyl morpholine-3-carboxamide

The title compound was prepared in a similar manner to Intermediate AG starting from (S)-4-(tert-butoxycarbonyl)-6,6-dimethylmorpholine-3-carboxylic acid. MS m/z 159.1 (M+H⁺) (Method M).

### Intermediate AJ

### (S)-6,6-dimethyl morpholine-3-carboxamide

The title compound was prepared in a similar manner to Intermediate AG starting from (S)-4-(tert-butoxycarbonyl)-morpholine-3-carboxylic acid and methyl amine. MS m/z 145.1 (M+H⁺) (Method M).

### Intermediate AK

### (2R)-methyl 1-(((1r,3R)-3-(4-amino-5-(3-((tetrahydro-2H-pyran-2-yl)methoxy)phenyl)-7H-pyrrolo[2,3-d]pyrimidin-7-yl)cyclobutyl)methyl)pyrrolidine-2-carboxylate

The title compound was prepared in a similar manner to Example 27 starting from cis-3-{4-amino-5-[3-(tetrahydro-pyran-2-ylmethoxy)-phenyl]-pyrrolo[2,3-d]pyrimidin-7-yl}-cyclobutanecarbaldehyde (Intermediate Q) and (R)-methylpyrrolidine-2-carboxylate hydrochloride. MS m/z 520.3 (M+H⁺) (Method M).

### Intermediate AL

### (1R,2S,5S)-3-azabicyclo[3.1.0]hexane-2-carboxamide

The title compound was prepared in a similar manner to Intermediate AG starting from (1R,2S,5S)-3-(tert-butoxycarbonyl)-3-azabicyclo[3.1.0]hexane-2-carboxylic acid. MS m/z 127.1 (M+H⁺) (Method M).

### Intermediate AM

### Perhydro-1,4-thiazepine 1,1-dioxide

To a solution of 1,1-Dioxo-1λ6-perhydro-1,4-thiazepine-4-carboxylic acid tert-butyl ester (Step AM.1, 100 mg, 0.4 mmol) in DCM (2 mL) was added TFA (0.5 mL). The mixture was stirred at room temperature for 1 hour. After concentration, the crude title compound was used directly for next step without further purification.

### Step AM.1

### 1,1-Dioxo-1λ6-perhydro-1,4-thiazepine-4-carboxylic acid tert-butyl ester

To a solution of tert-butyl 1,4-thiazepane-4-carboxylate (Step AM.2, 500 mg, 2.3 mmol) in DCM (10 mL) was added mCPBA (1.3 g, 5.75 mmol). After stirring for 2 hours, the reaction was quenched with saturated sodium carbonate aqueous solution. The mixture was extracted with DCM. The organic layers were combined, washed with saturated aqueous NaCl, and concentrated to afford the title compound as a white solid. MS m/z 194.1 (M−56+H⁺) (Method M).

### Step AM.2

### tert-butyl 1,4-thiazepane-4-carboxylate

1,4-thiazepan-5-one (prepared as described in WO2006/056875, 910 mg, 6.94 mmol) was dissolved in anhydrous THF (27 mL) and cooled to 0° C. LAH (1 MTHF solution, 1.05 equiv., 7.28 mmol, 7.28 mL) was added dropwise over 10 minutes. Following the completion of LAH addition, the reaction was allowed to stir at 0° C. for an additional 10 minutes at which point the cooling bath was removed and the reaction was stirred at room temperature for 2 hours. The reaction was carefully quenched by the sequential addition of H₂O (0.3 mL) followed by 1 N aqueous NaOH (1.5 mL). The resulting solids were removed by filtration through a celite plug followed by washing of the celite plug with Et₂O (400 mL). The combined filtrate was concentrated to generate crude 1,4-thiazepane which was redissolved in DCM (70 mL). TFA (˜1.2 equiv, 8.4 mmol, 1.2 mL) was added followed by di-tert-butyl dicarbonate (˜1.05 equiv, 7.35 mmol, 1.604 g). The reaction mixture was allowed to stir at room temperature for 12 hours. The crude reaction mixture was concentrated and purified with silica gel flash column chromatography (0˜30% ethyl acetate in hexanes) to afford the title compound as a clear oil, MS m/z 240.1 (M+Na⁺) (Method M).

### Intermediate AN

(1R,3S,4S)-2-azabicyclo[2.2.1]heptane-3-carboxamide hydrochloride salt was prepared in a similar manner to Intermediate AE from (1R,3S,4S)-2-(tert-butoxycarbonyl)-2-azabicyclo[2.2.1]heptane-3-carboxylic acid. MS m/z 141.1 (M+H⁺) (Method M).

### Intermediate AO

### 2,2-Dimethyl-thiomorpholine 1,1-dioxide

To a solution of 2,2-Dimethyl-1,1-dioxo-1λ6-thiomorpholine-4-carboxylic acid tert-butyl ester (Step AO.1, 80 mg, 0.3 mmol) in DCM (2 mL) was added TFA (0.5 mL). The mixture was stirred at room temperature for 1 hour. After concentration, the crude title compound was used directly for next step without further purification.

### Step AO.1

### 2,2-Dimethyl-1,1-dioxo-1λ6-thiomorpholine-4-carboxylic acid tert-butyl ester

To a solution of tert-butyl 1,4-thiazepane-4-carboxylate (Step AO.2, 150 mg, 0.65 mmol) in DCM (5 mL) was added mCPBA (224 mg, 1.3 mmol). After stirring for 2 hours, the reaction was quenched with saturated sodium carbonate aqueous solution. The mixture was extracted with DCM. The organic layers were combined, washed with saturated aqueous NaCl, and concentrated to afford the title compound as a white solid. MS m/z 208.1 (M-56+H⁺) (Method M).

### Step AO.2

### tert-butyl 2,2-dimethylthiomorpholine-4-carboxylate

2-Amino-ethanethiol (20 mmol, 2.27 g) was suspended in EtOH (150 mL) at 0° C. KOH (2 equiv., 40 mmol, 2.25 g) was added followed by Ethyl-2-bromoisobutyrate (1 equiv, 20 mmol, 3.90 g, 2.97 mL). The cooling bath was removed and the reaction mixture was stirred at room temperature for 15 minutes. The reaction was heated to reflux for 24 hours after which point the reaction was concentrated, n-BuOH (150 mL) was added, and the reaction heated to reflux for an additional 40 hours. The solids were removed by filtration. The filtrate was concentrated, redissolved in DCM, sequentially washed with 1 N aqueous HCl (3×), saturated aqueous NaCl (1×), dried over Na₂SO₄, concentrated and partially purified with silica gel flash column chromatography (0˜75% ethyl acetate in hexanes) to afford partially purified 2,2-dimethylthiomorpholin-3-one, MS m/z 146.1 (M+H⁺) (Method M). The partially purified 2,2-dimethylthiomorpholin-3-one was continued to the next step although it contained an impurity overlaid in both TLC and LCMS. Partially purified 2,2-dimethylthiomorpholin-3-one (˜5 mmol) was dissolved in anhydrous THF (35 mL) and Borane-DMS complex (2 M in THF, ˜2 equiv, 10 mmol, 5 mL) was slowly added at room temperature. Following completion of the addition, the reaction was heated to 50° C. for 4 hours and then allowed to sit at room temperature overnight. The reaction was quenched by the careful addition of MeOH until gas evolution ceased. The reaction mixture was concentrated to generate crude 2,2-dimethylthiomorpholine (MS m/z 132.1 (M+H⁺) (Method M)) which was redissolved in DCM (50 mL). TEA (˜1.2 equiv, 6.06 mmol, 0.84 mL) was added followed by di-tert-butyl dicarbonate (˜1.05 equiv, 5.30 mmol, 1.157 g). The reaction mixture was allowed to stir at room temperature for 2 hours. The crude reaction mixture was concentrated and purified with silica gel flash column chromatography (0˜20% ethyl acetate in hexanes) to afford the title compound as a clear oil, MS m/z 132.1 (M-Boc+H⁺) (Method M).

### Intermediate AP

### ((1S,3S)-3-(5-(3-(7-oxabicyclo[2.2.1]heptan-1-ylmethoxy)phenyl)-4-amino-7H-pyrrolo[2,3-d]pyrimidin-7-yl)-cyclobutyl)methyl 4-methylbenzenesulfonate

Tosyl chloride (72 mg, 0.37 mmol) was added to a solution of (3-{4-amino-5-[3-(7-oxa-bicyclo[2.2.1]hept-1-ylmethoxy)-phenyl]-pyrrolo[2,3-d]pyrimidin-7-yl}-cyclobutyl)-methanol (Example 22, 100 mg, 0.24 mmol) in pyridine (1 mL) at 0° C. The mixture was stirred at room temperature for 6 h. The reaction was diluted with EtOAc (50 mL), washed with water (2×5 mL), saturated aqueous NaCl (5 mL), dried over Na₂SO₄ and evaporated. The residue was purified by flash chromatorgraphy (SiO₂, EtOAc:hex/0-100%) to give the title compound as a white solid. MS m/z 575.1 (M+H⁺) (Method M).

### Intermediate AQ

### 5-(3-(7-oxabicyclo[2.2.1]heptan-1-ylmethoxy)phenyl)-7-((1S,3S)-3-(aminomethyl)cyclobutyl)-7H-pyrrolo[2,3-d]pyrimidin-4-amine

A mixture of 2-(((1S,3S)-3-(5-(3-(7-oxabicyclo[2.2.1]heptan-1-ylmethoxy)phenyl)-4-amino-7H-pyrrolo[2,3-d]pyrimidin-7-yl)cyclobutyl)methyl)isoindoline-1,3-dione (Example 108, 20 mg, 0.036 mmol) and hydrazine monohydrate (0.05 mL) in EtOH (2 mL) was stirred at 60° C. overnight. The solvent was evaporated and the residue was purified by flash chromatography (SiO₂, MeOH:DCM/10-15%) to give the title compound. MS m/z 420.2 (M+H⁺) (Method M).

### Intermediate AR

### 4-(5-(3-(7-oxabicyclo[2.2.1]heptan-1-ylmethoxy)phenyl)-4-amino-7H-pyrrolo[2,3-d]pyrimidin-7-yl)-cyclohexanone

The title compound was prepared in a similar manner to Intermediate AC, utilizing intermediate K and Step AC.2. MS m/z 433.2 (M+H⁺) (Method M).

### Intermediate AS

### ((1S,4S)-4-(4-amino-5-iodo-7H-pyrrolo[2,3-d]pyrimidin-7-yl)-cyclohexyl)methanol

((1S,4S)-4-(4-chloro-5-iodo-7H-pyrrolo[2,3-d]pyrimidin-7-yl)-cyclohexyl)methanol (Step AS.1, 4.8 mmol) was stirred in EtOH:NH₄OH (8:8 mL) in a sealed reaction vial at 100° C. overnight. The EtOH was removed under reduced pressure. The resulting precipitate was collected by filtration. The solid was suspended in MeOH (15 mL) and stirred at room temperature for several hours. The solid was collected by filtration. The trituration was repeated one more time (in 10 mL MeOH) to give the product as an off white solid. The filtrate was concentrated and purified by reversed phase preparative HPLC (Method S) to offer additional product. MS m/z 373.0 (M+H⁺) (Method M).

### Step AS.1

### ((1S,4S)-4-(4-chloro-5-iodo-7H-pyrrolo[2,3-d]pyrimidin-7-yl)-cyclohexyl)methanol

A mixture of ((1S,4S)-4-(4-chloro-7H-pyrrolo[2,3-d]pyrimidin-7-yl)-cyclohexyl)methanol (Step AS.2, 5.10 g, 19.2 mmol) and N-iodosuccinimide (4.75 g, 21.1 mmol) in anhydrous DMF (30 mL) was stirred at room temperature overnight. Water (30 mL) was added. A brown orange solid was collected by filtration, thoroughly washed with water and dried in vacuo to give the title compound. MS m/z 392.0 (M+H⁺) (Method M).

### Step AS.2

### ((1S,4S)-4-(4-chloro-7H-pyrrolo[2,3-d]pyrimidin-7-yl)-cyclohexyl)methanol

To a solution of 2-(4,6-dichloropyrimidin-5-yl)acetaldehyde (2.80 g, 14.66 mmol) and ((1S,4S)-4-aminocyclohexyl)methanol (Step AS.3, 14.66 mmol) in anhydrous EtOH (30 mL) was added DIEA. The reaction was stirred in a sealed vial at 60° C. for 20 h. The reaction was diluted with EtOAc (150 mL), washed with water (10 mL), saturated aqueous NaCl (10 mL), dried over Na₂SO₄ and evaporated. The residue was purified by flash chromatography (SiO₂, EtOAc:hex/0-100%) to give the title compound as a yellow solid. MS m/z 266.1 (M+H⁺) (Method M).

### Step AS.3

### ((1S,45)-4-aminocyclohexyl)methanol

(1S,4S)-4-aminocyclohexanecarboxylic acid (5.11 g, 35.7 mmol) was added portionwise to a solution of BH₃-THF (1 M, 214 mL) at room temperature. The suspension was stirred at room temperature overnight. MeOH (100 mL) was added slowly to the reaction at room temperature. After stirring for 2 h, the solvent was evaporated to give a clear oily residue. The crude oil residue was redissolved in MeOH (80 mL). The remaining excess BH₃ was scavaged by stirring with Pd/C (10% wet, 250 mg) at room temperature for 60 h. The reaction mixture was filtered through a layer of celite, washed with MeOH and concentrated. The residue was dried further in vacuo. The crude product obtained was used for the next step without further purification. MS m/z 130.1 (M+H⁺) (Method M).

### Intermediate AT

### (1S,45)-4-(5-(3-(7-oxabicyclo[2.2.1]heptan-1-ylmethoxy)phenyl)-4-amino-7H-pyrrolo[2,3-d]pyrimidin-7-yl)-cyclohexanecarbaldehyde

A suspension of ((1S,4S)-4-(5-(3-(7-oxabicyclo[2.2.1]heptan-1-ylmethoxy)phenyl)-4-amino-7H-pyrrolo[2,3-d]pyrimidin-7-yl)-cyclohexyl)methanol (Example 110, 94 mg, 0.209 mmol) and 1-hydroxy-1,2-benziodoxol-3(1H)-one 1-oxide (117 mg, 0.42 mmol) in anhydrous acetonitrile (4 mL) was stirred at 80° C. for 15 min. The solvent was evaporated and the crude product obtained was used in the next step without further purification. MS m/z 447.2 (M+H⁺) (Method M).

### Intermediate AU

### 5-(3-(7-oxabicyclo[2.2.1]heptan-1-ylmethoxy)phenyl)-7-(piperidin-4-yl)-7H-pyrrolo[2,3-d]pyrimidin-4-amine

The title compound was obtained in a similar manner to Intermediates P and AD, utilizing Intermediate K and Step AD.2. MS m/z 420.2 (M+H⁺) (Method M).

### Intermediate AV

### 5-(3-(7-oxabicyclo[2.2.1]heptan-1-ylmethoxy)phenyl)-7-((3R,6R)-1-oxaspiro[2.5]octan-6-yl)-7H-pyrrolo[2,3-d]pyrimidin-4-amine

To a solution of 5-(3-(7-oxabicyclo[2.2.1]heptan-1-ylmethoxy)phenyl)-7-(4-methylenecyclohexyl)-7H-pyrrolo[2,3-d]pyrimidin-4-amine (Step AV.1) (49 mg, 0.1 mmol) in DCM (2 mL) at 0° C., was added mCPBA (71 mg, 0.42 mmol). The reaction mixture was stirred at room temperature for 2 h, followed by concentration and purification on reversed phase preparative HPLC (Method S) to afford the title compound. MS m/z 447.2 (M+(Method M).

### Step AV.1

### 5-(3-(7-oxabicyclo[2.2.1]heptan-1-ylmethoxy)phenyl)-7-(4-methylenecyclohexyl)-7H-pyrrolo[2,3-d]pyrimidin-4-amine

The title compound was obtained in a similar manner to Intermediate P, utilizing Intermediate K and Step AV.2. MS m/z 431.2 (M+H⁺) (Method M).

### Step AV.2

### 5-iodo-7-(4-methylenecyclohexyl)-7H-pyrrolo[2,3-d]pyrimidin-4-amine

The title compound was obtained in a similar manner to Intermediate M starting from Step AV.3. MS m/z 355.0 (M+H⁺) (Method M).

### Step AV.3

### 4-chloro-5-iodo-7-(4-methylenecyclohexyl)-7H-pyrrolo[2,3-d]pyrimidine

The title compound was obtained in a similar manner to Example 1 starting from 4-chloro-5-iodo-7H-pyrrolo[2,3-d]pyrimidine (Step AV.4) and Step K.2. MS m/z 374.0 (M+H⁺) (Method M).

### Step AV.4

### 4-chloro-5-iodo-7H-pyrrolo[2,3-d]pyrimidine

To a solution of 4-chloro-7H-pyrrolo[2,3-d]pyrimidine (2.6 g, 17.4 mmol) in DMF (50 mL) was added NIS (3.9 g, 17.4 mmol). The reaction was stirred at room temperature overnight. Water (125 mL) was added to the reaction mixture, and the precipitate was filtered and washed with water. The solid was dried in vacuo to afford the title compound. MS m/z 280.0 (M+H⁺) (Method M).

### Intermediate AW

### 5-(3-(7-oxabicyclo[2.2.1]heptan-1-ylmethoxy)phenyl)-7-((1S,4S)-4-aminocyclohexyl)-7H-pyrrolo[2,3-d]pyrimidin-4-amine

To a solution of 5-(3-(7-oxabicyclo[2.2.1]heptan-1-ylmethoxy)phenyl)-7-((1S,4S)-4-azidocyclohexyl)-7H-pyrrolo[2,3-d]pyrimidin-4-amine (Step AW.1, 141 mg, 0.09 mmol) in THF (2 mL) was sequentially added Ph₃P (45 mg, 0.18 mmol), aqueous NaOH (0.1 N in water, 0.3 mL, 0.03 mmol). The reaction mixture was stirred at room temperature overnight. The reaction mixture was partitioned between DCM and water, the organic extracts were dried (Na₂SO₄), concentrated in vacuo, and purified by silica gel flash column chromatography (MeOH/DCM: 0-10%) to afford the title compound. MS m/z 434.3 (M+H⁺) (Method M).

### Step AW.1

### 5-(3-(7-oxabicycl o[2.2.1]heptan-1-ylmethoxy)phenyl)-7-((1S,4S)-4-azidocyclohexyl)-7H-pyrrolo[2,3-d]pyrimidin-4-amine

To the solution of (1R,4R)-4-(5-(3-(7-oxabicyclo[2.2.1]heptan-1-ylmethoxy)phenyl)-4-amino-7H-pyrrolo[2,3-d]pyrimidin-7-yl)-cyclohexyl methanesulfonate (Step AW.2, 44 mg, 0.09 mmol) in DMF (2.0 mL) was added sodium azide (6.4 mg, 0.1 mmol). The reaction mixture was heated at 80° C. for 16 h. The mixture was cooled to room temperature, partitioned between EtOAc and water, the collected organic extracts were dried (Na₂SO₄), concentrated in vacuo, and purified by silica gel flash column chromatography (MeOH/DCM: 0-10%) to afford the title compound. MS m/z 460.3 (M+H⁺) (Method M).

### Step AW. 2

### (1R,4R)-4-(5-(3-(7-oxabicyclo[2.2.1]heptan-1-ylmethoxy)phenyl)-4-amino-7H-pyrrolo[2,3-d]pyrimidin-7-yl)-cyclohexyl methanesulfonate

To a solution of (1R,4R)-4-(5-(3-(7-oxabicyclo[2.2.1]heptan-1-ylmethoxy)phenyl)-4-amino-7H-pyrrolo[2,3-d]pyrimidin-7-yl)-cyclohexanol (Step AW.3, 1.3 g, 3.0 mmol) and triethylamine (0.84 mL, 6.0 mmol) in THF (25 mL) at 0° C., was added methanesulfonyl chloride (445 mg, 3.9 mmol) dropwise. The mixture was stirred for 4 hours at room temperature. The reaction was quenched with saturated aqueous ammonium chloride and extracted with EtOAc. The combined organic extracts were washed with saturated aqueous NaCl, dried with sodium sulfate and concentrated. The residue was purified with silica gel chromatography (5% MeOH in DCM containing 0.1 N NH₃) to afford the title compound as light yellow solid. MS m/z 513.2 (M+H⁺) (Method M).

### Step AW. 3

### (1R,4R)-4-(5-(3-(7-oxabicyclo[2.2.1]heptan-1-ylmethoxy)phenyl)-4-amino-7H-pyrrolo[2,3-d]pyrimidin-7-yl)-cyclohexanol

The title compound was obtained in a similar manner to Intermediate P, utilizing Step AW.4 and Intermediate K. MS m/z 435.2 (M+H⁺) (Method M).

### Step AW.4

### (1R,4R)-4-(4-amino-5-iodo-7H-pyrrolo[2,3-d]pyrimidin-7-yl)-cyclohexanol

The title compound was prepared in a similar manner to Step AC.2 using Step AW.5 as starting material. MS m/z 359.0 (M+H⁺) (Method M).

### Step AW. 5

### (1R,4R)-4-(4-chloro-5-iodo-7H-pyrrolo[2,3-d]pyrimidin-7-yl)-cyclohexanol

To a solution of (1R,4R)-4-(4-chloro-7H-pyrrolo[2,3-d]pyrimidin-7-yl)-cyclohexanol (Step AW.6, 4.5 g, 17.4 mmol) in DMF (50 mL) was added NIS (3.9 g, 17.4 mmol). The reaction was stirred at room temperature overnight. Water (125 mL) was added to the reaction mixture, the precipitate was filtered and washed with water. The solid collected was dried in vacuo to afford the title compound. MS m/z 378.0 (M+H⁺) (Method M).

### Step AW.6

### (1R,4R)-4-(4-chloro-7H-pyrrolo[2,3-d]pyrimidin-7-yl)-cyclohexanol

To a mixture of 2-(4,6-dichloropyrimidin-5-yl)acetaldehyde (5.0 g, 26.3 mmol) and (1R,4R)-4-aminocyclohexanol (3.0 g, 26.3 mmol) in EtOH (66 mL) was added DIEA (5.5 mL, 31.6 mmol). The reaction was heated at 80° C. overnight. The reaction mixture was cooled to room temperature, concentrated in vacuo, and purified by silica gel flash column chromatography (MeOH/DCM: 0-10%) to afford the title compound. MS m/z 252.0 (M+H⁺) (Method M).

### Intermediate AX

### 5-(3-(7-oxabicyclo[2.2.1]heptan-1-ylmethoxy)phenyl)-7-((1s,4s)-4-(azido methyl)cyclohexyl)-7H-pyrrolo[2,3-d]pyrimidin-4-a mine

A mixture of ((1S,4S)-4-(5-(3-(7-oxabicyclo[2.2.1]heptan-1-ylmethoxy)phenyl)-4-amino-7H-pyrrolo[2,3-d]pyrimidin-7-yl)-cyclohexyl)methyl methanesulfonate (Step AX.1, 113 mg 0.21 mmol) and sodium azide (28 mg, 0.42 mmol) in anhydrous DMF (1 mL) was stirred at 80° C. for 5 h. Additional sodium azide (20 mg, 0.3 mmol) was added and the reaction continued for 1 h. The solvent was evaporated under reduced pressure. The residue was diluted with DCM (50 mL), washed with water (10 mL), saturated aqueous NaCl (10 mL), dried over Na₂SO₄ and evaporated to afford the title compound. MS m/z 474.3 (M+H⁺) (Method M).

### Step AX. 1

### a1S,4S)-4-(5-(3-(7-oxabicyclo[2.2.1]heptan-1-ylmethoxy)phenyl)-4-amino-7H-pyrrolo[2,3-d]pyrimidin-7-yl)-cyclohexyl)methyl methanesulfonate

To a solution of ((1S,4S)-4-(5-(3-(7-oxabicyclo[2.2.1]heptan-1-ylmethoxy)phenyl)-4-amino-7H-pyrrolo[2,3-d]pyrimidin-7-yl)-cyclohexyl)methanol (Example 110, 224 mg, 0.50 mmol) in DCM (10 mL) at 0° C. was added methanesulfonyl anhydride (218 mg, 1.25 mmol) followed by slow addition of TEA (209 uL, 1.5 mmol). After stirring at 0° C. for 1 h, the reaction was quenched with water (10 mL). The mixture was stirred vigorously, then extracted with DCM (50 mL). DCM was washed with saturated aqueous NaCl (5 mL), dried over Na₂SO₄ and evaporated to give an off-white solid which was purified by flash chromatography (SiO₂, MeOH:DCM/0-10%) to offer the title compound as off-white solid. MS m/z 527.2 (M+H⁺) (Method M).

### Intermediate AY

### 3,3-Dimethyl-thiomorpholine 1,1-dioxide

The 3,3-Dimethyl-1,1-dioxo-thiomorpholine-4-carboxylic acid tert-butyl ester (Step AY.1, 106 mg, 0.40 mmol) in 4 N HCl in dioxane (4 mL) was stirred at room temperature for two hours. The solvent was evaporated and dried in vacuo to afford the title compound as its HCl salt. MS m/z 164.1 (M+H⁺) (Method M).

### Step AY. 1

### 3,3-Dimethyl-1,1-dioxo-thiomorpholine-4-carboxylic acid tert-butyl ester

5,5-dimethylthiomorpholin-3-one (Step AY.2, 8.24 mmol, 1.196 g) was dissolved in anhydrous THF (60 mL) and Borane-DMS complex (2 M in THF, 2 equiv, 16.5 mmol, 8.25 mL) was slowly added at room temperature. Following completion of the addition, the reaction was heated to 50° C. for 5 hours and then cooled to room temperature. The reaction was quenched by the careful addition of MeOH until gas evolution ceased. The reaction mixture was concentrated to generate crude 3,3-dimethylthiomorpholine which was redissolved in DCM (80 mL). TFA (˜1.2 equiv, 9.89 mmol, 1.38 mL) was added followed by di-tert-butyl dicarbonate (˜1.05 equiv, 8.65 mmol, 1.89 g). The reaction mixture was allowed to stir at room temperature for 3 days. The crude reaction mixture was concentrated and partially purified with silica gel flash column chromatography (0˜15% ethyl acetate in hexanes) to afford partially purified tert-butyl 3,3-dimethylthiomorpholine-4-carboxylate as a clear oil, MS m/z 132.1 (M-Boc+H⁺) (Method M). The partially purified tert-butyl 3,3-dimethylthiomorpholine-4-carboxylate (1.35 g) was continued to the next step without further purification and dissolved in DCM (60 mL) at 0° C. mCPBA (77% pure, ˜2.1 equiv, 12.25 mmol, 2.75 g) was added to the solution, the cooling bath was removed and the reaction mixture was allowed to stir at room temperature for 3 hours. Starting material remained (LCMS, Method M) so additional mCPBA (˜77% pure, ˜2.1 equiv., 12.25 mmol, 2.75 g) was added and the reaction mixture stirred for an additional 2 hours. The reaction mixture was diluted with DCM, washed with saturated aqueous NaHCO₃ (3×), saturated NaCl (1×), and dried over Na₂SO₄. The crude reaction mixture was concentrated and purified with silica gel flash column chromatography (0-50% ethyl acetate in hexanes) to afford the title compound as a clear oil, MS m/z 264.1 (M+H⁺) (Method M).

### Step AY.2

### 5,5-dimethylthiomorpholin-3-one

2-amino-2-methylpropane-1-thiol hydrochloride salt (20 mmol, 2.833 g) was suspended in EtOH (150 mL) at 0° C. KOH (2 equiv., 40 mmol, 2.25 g) was added followed by ethyl bromoacetate (1 equiv, 20 mmol, 3.34 g, 2.21 mL). The cooling bath was removed and the reaction mixture was stirred at room temperature for 15 minutes. The reaction was heated to reflux for 3 hours after which point the reaction was concentrated, toluene (150 mL) was added, and the reaction heated to reflux for an additional 40 hours. The reaction mixture was diluted with EtOAc (150 mL) and sequentially washed with 1 N aqueous HCl (3×), saturated aqueous NaCl (1×), dried over Na₂SO₄, and concentrated to afford 5,5-dimethylthiomorpholin-3-one, MS m/z 146.1 (M-FH⁺) (Method M).

### Intermediate AZ

### 4-(5-(3-(7-oxabicyclo[2.2.1]heptan-1-ylmethoxy)phenyl)-4-amino-7H-pyrrolo[2,3-d]pyrimidin-7-yl)-1-(aminomethyl)cyclohexanol

Under nitrogen, to a solution of 4-(5-(3-(7-oxabicyclo[2.2.1]heptan-1-ylmethoxy)phenyl)-4-amino-7H-pyrrolo[2,3-d]pyrimidin-7-yl)-1-(aminomethyl)cyclohexanol (Step AZ.1, 26.5 mg, 0.5 mmol) in toluene (2.0 mL) was added Red-Al (65% wt in toluene, 0.37 mL, 1.2 mmol) slowly. After addition, the reaction mixture was heated at 70° C. for 1.5 h, then cooled to room temperature, and stirred overnight. Aqueous NaOH (1.0 N, 1.0 mL) was added to the solution slowly, and the mixture was partitioned between EtOAc and saturated aqueous NaCl. The combined organic extracts were dried over Na₂SO₄ and concentrated in vacuo. The residue was purified by silica gel chromatography (MeOH/DCM: 0-10%) to afford the title compound. MS m/z 464.3 (M+H⁺) (Method M).

### Step AZ.1

### 4-(5-(3-(7-oxabicyclo[2.2.1]heptan-1-ylmethoxy)phenyl)-4-amino-7H-pyrrolo[2,3-d]pyrimidin-7-yl)-1-(trimethylsilyloxy)cyclohexanecarbonitrile

Under nitrogen, to a solution of LiOMe (1.0 M in MeOH, 0.044 mL, 0.044 mmol) in THF (1.0 mL) was added TMSCN (0.142 mmol, 1.07 mL) slowly. The mixture was stirred at room temperature for 10 min. To the mixture, was added a solution of 4-(5-(3-(7-oxabicyclo[2.2.1]heptan-1-ylmethoxy)phenyl)-4-amino-7H-pyrrolo[2,3-d]pyrimidin-7-yl)-cyclohexanone (Intermediate AR, 38.4 mg, 0.89 mmol) in THF (2.0 mL) slowly. The reaction mixture was stirred overnight. The reaction was quenched by saturated aqueous NaHCO₃ (2.0 mL), and partitioned between EtOAc and saturated aqueous NaCl. The combined organic extracts were dried over Na₂SO₄ and concentrated in vacuo. The resulting residue was purified by silica gel column chromatography (MeOH/DCM: 0-5%) to afford the title compound. MS m/z 532.3 (M+H⁺) (Method M).

### Intermediate BA

### toluene-4-sulphonic acid cis-3-(4-amino-5-{3-[(R)-1-(tetrahydro-pyran-2-yl)methoxy]-phenyl}-pyrrolo[2,3-d]pyrimidin-7-yl)-cyclobutylmethyl ester

To a solution of [cis-3-(4-amino-5-{3[(R)-1-(tetrahydro-pyran-2-yl)methoxy]-phenyl}-pyrrolo[2,3-d]pyrimidin-7-yl)-cyclobutyl]-methanol (Step BA.1, 1.00 mmol) in dry pyridine (1 ml) cooled at −20° C. was added p-toluenesulphonyl chloride portionwise over 45 minutes (764 mg, 4.01 mmol) and the reaction mixture allowed to stand overnight at −20° C. The reaction mixture was partitioned between ice/water and dichloromethane, extracting 3× with dichloromethane. The combined organic layers were then washed with cold 1 Msulphuric acid then saturated brine, dried over sodium sulphate and evaporated to give the title compound as a pale green glass. MS m/z 536.0 (M+H⁺) (Method L).

### Step BA.1

### [cis-3-(4-amino-5-{3-[(R)-1-(tetrahydro-pyran-2-yl)methoxy]-phenyl}-pyrrolo[2,3-d]pyrimidin-7-yl)-cyclobutyl]-methanol

A mixture of [cis-3-(4-amino-5-iodo-pyrrolo[2,3-d]pyrimidin-7-yl)-cyclobutyl]-methanol (Intermediate M, 540 mg, 1.57 mmol), (R)-2-[3-(4,4,5,5-tetramethyl-[1,3,2]dioxaborolan-2-yl)-phenoxymethyl]-tetrahydro-pyran (Intermediate AB, 749 mg, 1.65 mmol), sodium carbonate (333 mg, 3.14 mmol), tetrakis(triphenylphosphine)palladium (181 mg, 0.16 mmol), water (2.5 ml) and DMF (5 ml) was purged with argon, sealed under argon and then heated at 80° C. for 15 hours. The cooled reaction mixture was then partitioned between water and DCM, extracting 2× with DCM, the DCM layers dried over sodium sulphate and evaporated. Purification of the residue by normal phase chromatography with a DCM/methanol gradient followed by preparative reversed phase chromatography (Method R) gave the title compounde as a colourless glass. MS m/z 409.2 (M+H⁺) (Method L).

### Intermediate BB

### 1-methyl-4-[3-(4,4,5,5-tetramethyl[1,3,2]dioxaborolan-2-yl)-phenoxymethyl]-7-oxa-bicyclo[2.2.1]heptane

A mixture of 1-(3-bromo-phenoxymethyl)-4-methyl-7-oxa-bicyclo[2.2.1]heptane (Step BB. 1, 1.66 g, 5.59 mmol), bis-(pinacolato)diboron (1.84 g, 7.26 mmol), potassium acetate (1.37 g, 13.96 mmol) and 1,1′-bis(diphenylphosphino)ferrocenedicholoro palladium (II) dichloromethane complex (0.41 g, 0.56 mmol) in DMF (12 ml) was purged with argon and then heated for 15 hours at 80° C. in a sealed vessel under an argon atmosphere. The cooled reaction mixture was then partitioned between water and DCM, extracting 2× with DCM, the DCM layers dried over magnesium sulphate and evaporated. Purification of the residue by normal phase chromatography with a DCM/methanol gradient to give the title compound as an orange solid. ¹H NMR (400 MHz, CDCl₃) 3 ppm 1.20-1.30 (m, 6H), 1.34 (5, 6H), 1.51 (s, 3 H), 1.57-1.81 (m, 6H), 1.84-2.00 (m, 2H), 4.24 (s, 2H), 7.02-7.11 (m, 1H), 7.27-7.32 (m, 1H), 7.35-7.43 (m, 2H).

### Step BB. 1

### 1-(3-bromo-phenoxymethyl)-4-methyl-7-oxa-bicyclo[2.2.1]heptane

A mixture of 1-iodomethyl-4-methyl-7-oxa-bicyclo[2.2.1]heptane (Step BB.2, 1.72 g, 6.82 mmol), 3-bromophenol (1.30 g, 7.51 mmol) and potassium carbonate (1.89 g, 13.7 mmol) in acetonitrile (4.6 ml) was heated at 150° C. for 48 hours in a sealed reaction vessel with stirring under an argon atmosphere. After standing a further 5 days at room temperature the reaction mixture was diluted with DCM and washed with 1 N aqueous sodium hydroxide solution, then water and saturated brine. The organic layer was dried over magnesium sulphate, evaporated and the residue purified by flash column chromatography, eluenting with a hexane ethyl acetate gradient, to give the title compound as a clear yellow oil. ¹H NMR (400 MHz, CDCl₃) δ ppm 1.52 (s, 3H), 1.59-1.81 (m, 6H), 1.83-1.98 (m, 2H), 4.18 (s, 2H), 6.84-6.95 (m, 1H), 7.04-7.18 (m, 3H).

### Step BB.2

### 1-iodomethyl-4-methyl-7-oxa-bicyclo[2.2.1]heptane

Iodine (33.9 g, 134 mmol) was added portion-wise to a suspension of sodium carbonate (4.95 g, 46.7 mmol) in a solution of 1-methyl-4-methylene-cyclohexanol (Step BB.3, 4.68 g, 33.4 mmol) in acetonitrile (300 ml) at room temperature. After stirring for 1 hour at room temperature the reaction mixture was diluted with diethyl ether and washed with a 10% aqueous solution of Na₂S₂O₃, the aqueous layers were then back extracted with further diethyl ether followed by washing of the combined organic layers with saturated brine. The organic layers were dried over magnesium sulphate and evaporated under a vacuum of 200 mbar at 30° C. and the residue purified by normal phase chromatography, eluting with a hexane/ethyl acetate gradient to give the title compound as a pale yellow oil. ¹H NMR (400 MHz, CDCl₃) δ ppm 1.46 (s, 3H), 1.64 (d, J=4.7 Hz, 2H), 1.70-1.82 (m, 4H), 1.85 (dd, J=9.4 and 5.5 Hz, 2H), 3.50 (s, 2H).

### Step BB. 3

### 1-methyl-4-methylene-cyclohexanol

p-Toluenesulphonic acid monohydrate (64 mg, 0.34 mmol) was added to a solution of 2-(1-methyl-4-methylene-cyclohexyloxy)-tetrahydro-pyran (Step BB.4, 7.06 g, 33.6 mmol) in methanol (67 ml) at room temperature. After stirring for 24 hours at room temperature the reaction mixture was evaporated under a vacuum of 200 mbar at 30° C., the residue partitioned between aqueous NaHCO₃/DCM, extracting with further DCM and the organic layers then dried over magnesium sulphate. Evaporation of the organic layers under a vacuum of 150 mbar at 30° C. gave the title compound as a pale yellow oil which crystallised on standing. ¹H NMR (400 MHz, CDCl₃) δ ppm 1.25 (s, 3H), 1.48-1.58 (m, 2H), 1.63-1.73 (m, 2H), 2.07-2.17 (m, 2H), 2.29-2.41 (m, 2H), 3.40 (s, 1H), 4.64 (s, 2H).

### Step BB.4

### 2-(1-methyl-4-methylene-cyclohexyloxy)-tetrahydro-pyran

A solution of n-butyl lithium in hexanes (26 ml, 2.5 M, 65.0 mmol) was added to a suspension of methyltriphenylphosphonium bromide (19.1 g, 53.6 mmol) in THF (185 ml) cooled at −10° C. After stirring 1 hour at −10° C. 4-methyl-4-(tetrahydro-pyran-2-yloxy)-cyclohexanone (Step BB.5, 8.6 g, 38. 3 mmol) was added and the reaction mixture allowed to warm to room temperature over a period of 3 hours. Acetone (20 ml) was added and the reaction mixture allowed to stand overnight at room temperature. The reaction volume was reduced under a vacuum of 120 mbar with a bath temperature of 30° C., silica gel (40 g) added and the mixture filtered through a plug of silica gel (60 g), eluting with a 1:1 mixture of ether and heptane (900 ml), and the filtrate evaorated at 30° C. under a 120 mbar vacuum to give the title compound as a pale yellow oil. ¹H NMR (400 MHz, CDCl₃) δ ppm 1.22 (s, 3H), 1.46-1.60 (m, 6H), 1.63-1.74 (m, 1H), 1.81-1.93 (m, 3H), 2.06 (ddd, J=13.4, 4.6, 4.3 Hz, 2 H), 2.22-2.35 (m, 1H), 2.41-2.52 (m, 1H), 3.47 (ddd, J=11.1, 5.9, 5.7 Hz, 1H), 3.91-4.02 (m, 1H), 4.60 (s, 2H) 4.78 (t, J=4.3 Hz, 1H).

### Step BB.5

### 4-methyl-4-(tetrahydro-pyran-2-yloxy)-cyclohexanone

para-Toluene sulphonic acid (90 mg, 0.47 mmol) was added to a mixture of 4-hydroxy-4-methyl-cyclohexanone (6.06 g, 47.3 mmol) and 3,4-dihydro-2H-pyran (9.94 g, 118 mmol) cooled at room temperature. After stirring for 1 hour at room temperature the reaction mixture was partitioned between aqueous sodium bicarbonate solution and diethyl ether, extracting 2× with diethyl ether, the organic layers dried over magnesium sulphate and then evaporated with a bath temperature of 30° C. under a 60 mbar vacuum. The crude product was purified by flach column chromatography, eluting with an ethyl acetate/hexane gradient, to give the title compound as a pale yellow oil. ¹H NMR (400 MHz, CDCl₃) δ ppm 1.32 (s, 3H), 1.47-1.64 (m, 4H), 1.66-1.80 (m, 3H), 1.88 (br. s., 1H), 2.15-2.26 (m, 4H), 2.62 (td, J=14.1, 6.3 Hz, 1H), 2.77-2.90 (m, 1H), 3.50 (dt, J=11.3, 5.7 Hz, 1H), 3.93-4.02 (m, 1H), 4.80-4.87 (m, 1H).

### Step BB.6

### 4-hydroxy-4-methyl-cyclohexanone

A mixture of 8-methyl-1,4-dioxa-spiro[4.5]decan-8-ol (Step BB.7, 9.35 g, 54.3 mmol), water (400 ml) and hydrochloric acid (0.3 ml) was stirred at room temperature for 48 hours and then extracted 3× with ethyl acetate. The combined organic layers were dried over magnesium sulphate and evaporated at 40° C. under a vacuum of 170 mbar to give the title compound as a pale yellow oil. ¹HNMR (400 MHz, CDCl₃) δ ppm 1.29-1.40 (m, 1H), 1.58 (s, 1H), 1.85 (td, 1H), 1.97 (qd, 1H), 2.24 (d, J=14.8 Hz, 1H), 2.72 (td, J=13.7, 6.3 Hz, 1H).

### Step BB.7

### 8-methyl-1,4-dioxa-spiro[4.5]decan-8-ol

A solution of methylmagnesium bromide in diethyl ether (3 molar, 32 ml, 96 mmol) was added to a solution of 1,4-cyclohexanedione-monoethyleneacetal (10 g, 64.0 mmol) in diethyl ether (330 ml) at room temperature with cooling. After stirring for 4.5 hours at room temperature water was added, the layers separated and the aqueous layer extracted a further 2× with diethyl ether, the combined organic layers dried over magnesium sulphate and evaporated to give the tile compound as a white solid.

### Intermediate BC

### 5-bromo-7-[3-(1,1-dioxo-1-thiomorpholin-4-ylmethyl)-cyclobutyl]-7H-pyrrolo[2,3-d]pyrimidin-4-ylamine

A mixture of 5-bromo-4-chloro-7-[cis-3-(1,1-dioxo-1-thiomorpholin-4-ylmethyl)-cyclobutyl]-7H-pyrrolo[2,3-d]pyrimidine (Step BC.1, 1.53 g, 3.54 mmol), aqueous ammonium hydroxide solution (25%, 20 ml) and dioxane (20 ml) were heated in a sealed vessel for 17 hours at 100° C. The cooled reaction mixture was then filtered, washing with water, to give the title compound as a white solid. HPLC/MS t_{R} 0.65 min, M+H 414.3 & 416.3 (Method X).

### Step BC. 1

### 5-bromo-4-chloro-7-[cis-3-(1, 1-dioxo-1-thiomorpholin-4-ylmethyl)-cyclobutyl]-7H-pyrrolo[2,3-d]pyrimidine

N-Bromosuccinimide (1.12 g, 6.20 mmol) was added to a mixture of 4-chloro-7-[cis-3-(1,1-dioxo-1-thiomorpholin-4-ylmethyl)-cyclobutyl]-7H-pyrrolo[2,3-d]pyrimidine (Step BC.2, 2.0 g, 5.64 mmol) in DMF (20 ml) at room temperature. After stirring 4 hours at room temperature the reaction mixture was diluted with DCM, washed with water, then saturated brine, dried over sodium sulphate and evaporated. Purification by flash column chromatography, eluting with a gradient of methanol in DCM, gave the title compound. HPLC/MS t_{R} 1.08 min, M+H 433.3 & 435.3 (Method X).

### Step BC.2

### 4-chloro-7-[cis-3-(1,1-dioxo-1-thiomorpholin-4-ylmethyl)-cyclobutyl]-7H-pyrrolo[2,3-d]pyrimidine

(4,6-Dichloro-pyrimidin-5-yl)-acetadehyde (1.26 g, 6.37 mmol) was added to a solution of ci33-(1,1-dioxo-1-thiomorpholin-4-ylmethyl)-cyclobutylamine (Step BC.3, 1.21 g, 5.54 mmol) in diisopropylethylamine (1.98 ml 11.1 mmol) and ethanol (28 ml) at room temperature and the reaction mixture was then heated for 5 hours at reflux. After cooling to room temperature the reaction mixture was diluted with ethyl acetate, washed with aqueous sodium bicarbonate, then saturated brine, dried over sodium sulphate and evaporated. Purification by flash column chromatography, eluting with a gradient of methanol in DCM, gave the title compound. HPLC/MS t_{R} 0.89 min, M+H 355.4 (Method X).

### Step BC.3

### cis-3-(1,1-dioxo-1-thiomorpholin-4-ylmethyl)-cyclobutylamine

A suspension of 10% palladium on charcoal (5.40 g) in a solution of [3-(1,1-dioxo-1-thiomorpholin-4-ylmethyl)-cyclobutyl]-carbamic acid benzyl ester (Step BC.4, 13 g, 36.9 mmol) in ethanol was stirred under an atmosphere of hydrogen for 4 days. After flushing the apparatus with nitrogen the reaction mixture was filtered and evaporated to give the title compound. ¹H NMR (400 MHz, DMSO) δ ppm 1.20-1.35 (m, 2H), 1.82-1.99 (m, 1H), 2.20-2.31 (m, 2H), 2.41-2.53 (m, 2H), 2.70-2.86 (m, 4H), 3.02-3.14 (m, 5H).

### Step BC.4

### [cis-3-(1,1-dioxo-1-thiomorpholin-4-ylmethyl)-cyclobutyl]-carbamic acid benzyl ester

Sodium triacetoxy borohydride (37.6 g, 159 mmol) was added potion-wise to a mixture of (cis-3-formyl-cyclobutyl)-carbamic acid benzyl ester (Step BC.5, 12.92 g, 53.2 mmol), thiomorpholine-1,1-dioxide (14.7 g, 106 mmol) and THF (150 ml) at room temperature. After stirring for 2 hours at room temperature the reaction mixture was partitioned between aqueous sodium bicarbonate solution and DCM, the organic layer washed with water and brine, dried over sodium sulphate and evaporated. Purification of the residue by flash column chromatography, eluting with a gradient of methanol in DCM, gave the title compound. HPLC/MS t_{R} 0.88 min, M+H 353.4 (Method X).

### Step BC.5

### (cis-3-formyl-cyclobutyl)-carbamic acid benzyl ester

Oxalyl chloride (5.84 ml, 64.1 mmol) in DCM (150 ml) was added dropwise over 15 minutes to a solution of DMSO (11.4 ml, 160 mmol) in DCM (30 ml) cooled at −78° C. After stirring for 20 minutes at −78° C. a solution of (cis-3-Hydroxymethyl-cyclobutyl)-carbamic acid benzyl ester (Step BC.6, 12.56 g, 53.4 mmol) in DCM (70 ml) was added dropwise over 15 minutes and 30 minutes later a solution of triethylamine (26.1 ml, 187 mmol) in DCM (30 ml) was added. The reaction mixture was stirred for a further 1 hour at −78° C., allowed to warm to 0° C. over 1 hour and then partitioned between aqueous sodium hydrogen carbonate solution and DCM. The organic layer was washed with water and brine, dried over sodium sulphate and evaporated to give the title compound. HPLC/MS t_{R} 0.92 min, M+H 234.1 (Method X).

### Step BC.6

### (cis-3-Hydroxymethyl-cyclobutyl)-carbamic acid benzyl ester

An aqueous solution of lithium hydroxide (179 ml, 1 M) was added to a mixture of benzoic acid cis-3-benzyloxycarbonylamino-cyclobutylmethyl ester (Step BC.7, 20.2 g, 59.5 mmol) and THF (500 ml) and the reaction mixture stirred for 16 hours at 50° C. After cooling to room temperature the reaction mixture was diluted with ethyl acetate and washed with water followed by brine. The organic layer was then dried over sodium sulphate, evaporated and recrystallised from a mixture of DCM/diethyl ether/heptane to give the title compound. ¹H NMR (400 MHz, DMSO)S ppm 1.51-1.64 (m, 2H), 1.88-2.05 (m, 1H), 2.13-2.21 (m, 2H), 3.25-3.36 (m, 3H), 3.76-3.89 (m, 1H), 4.46 (t, 1H), 4.99 (s, 2H), 7.26-7.39 (m, 4H), 7.43-7.52 (m, 1H).

### Step BC.7

### benzoic acid cis-3-benzyloxycarbonylamino-cyclobutylmethyl ester

Benzyl chloroformate (15.7 ml, 110 mmol) was added dropwise to a mixture of benzoic acid 3-amino-cyclobutylmethyl ester (prepared according to the procedure of: J. Slade Organic Process Research & Development 2007, 11, 825-835., 15 g, 73.1 mmol), diisopropylethylamine (25.5 ml, 146 mmol) and DCM (225 ml) cooled at 0° C. After stirring for 20 hours at room temperature the reaction mixture was diluted with DCM, washed with 5% aqueous potassium hydrogen phosphate solution, aqueous sodium bicarbonate solution, water and brine, dried over sodium sulphate and evaporated. Purification of the residue by flash column chromatography, eluting with a gradient of methanol in DCM, gave the title compound. HPLC/MS t_{R} 1.17 min, M+H 340.1 (Method X).

### Intermediate BD

### 4,4,5,5-tetramethyl-2-(3-[(S)-1-(tetrahydrofuran-2-yl)methoxy]-phenyl)-[1,3,2]dioxaborolane

A mixture of (S)-2-(3-bromo-phenoxymethyl)-tetrahydrofuran (Step BD.1, 1.75 g, 6.81 mmol), bis(pinacolato)diboron (1.73 g, 6.81 mmol), potassium acetate (2.0 g, 20.4 mmol), [1,1-bis(diphenylphosphino)ferrocene]dichloropalladium(II) (0.15 g, 0.03 mmol) and DMF (13 ml) was heated under an argon atmosphere at 100° C. for 90 minutes. The cooled reaction mixture was partitioned between DCM and water, extracted 3× with DCM, the organic layers dried over sodium sulphate and evaporated. Purification of the residue by flash column chromatography, eluting with a methanol in DCM gradient, gave the title compound as viscous clear-yellow oil.

### Step BD.1

### (S)-2-(3-bromo-phenoxymethyl)-tetrahydrofuran

Diisopropyl azodicarboxylate (2.17 ml, 11.2 mmol) was added dropwise over 5 minutes to a mixture of 3-bromophenol (1.93 g, 11.2 mmol), (S)-2-tetrahydrofurylmethanol (1.3 ml, 13.4 mmol), triphenylphosphine (4.11 g, 15.7 mmol) and THF (22 ml) cooled at room temperature. After standing 18 hours at room temperature, volatiles were removed under reduced pressure, the residue partitioned between 1 N aqueous sodium hydroxide and DCM, extracted 2× with DCM, the combined organic layers, washed with water, dried over sodium sulphate and evaporated. Purification of the residue by flash column chromatography, eluting with DCM, gave the title compound as a clear pale-yellow oil. ¹H NMR (400 MHz, CDCl₃) δ ppm 1.41-1.59 (m, 1H), 1.78-1.99 (m, 2H), 2.02-2.24 (m, 1H), 3.57-3.71 (m, 1H), 3.79-3.93 (m, 3H), 4.43-4.58 (m, 1H), 6.66-6.74 (m, 1H), 7.09-7.28 (m, 3H).

### Intermediates BE

### cis-toluene-4-sulphonic acid 3-{4-amino-5-[3-(4-methyl-7-oxa-bicyclo[2.2.1]hept-1-ylmethoxy)-phenyl]-pyrrolo[2,3-d]pyrimidin-7-yl}-cyclobutylmethyl ester

A solution of para-toluene sulphonyl chloride (115 mg, 0.604 mmol) in pyridine (0.7 ml) was added portion-wise over 1 hour to a solution of (cis-3-{4-amino-5-[3-(4-methyl-7-oxa-bicyclo[2.2.1]hept-1-ylmethoxy)-phenyl]-pyrrolo[2,3-d]pyrimidin-7-yl}-cyclobutyl)-methanol (Example 153, 87.5 mg, 0.201 mmol) in pyridine (0.8 ml) cooled at −25° C. After 18 hours at −25° C. the reaction mixture was partitioned between 1 N sulphuric acid and DCM cooled ar 0° C., extracted 2× with DCM, the combined organic layers dried over sodium sulphate and evaporated to give the title compound as a yellow glass. HPLC/MS t_{R} 1.21 min, M+H 589.2 (Method X).

### Intermediate BF

### 1-{4-[cis-3-(4-amino-5-iodo-pyrrolo[2,3-d]pyrimidin-7-yl)-cyclobutyl]-piperazin-1-yl}-ethanone

Sodium triacetoxyborohydride (707 mg, 3.34 mmol) was added portionwise over 5 minutes to a mixture of 3-(4-amino-5-iodo-pyrrolo[2,3-d]pyrimidin-7-yl)-cyclobutanone (Intermediate BG, 730 mg, 2.23 mmol), N-acetylpiperazine (342 mg, 2.67 mmol), acetic acid (401 mg, 6.67 mmol) and 1,2-dichloroethane (4.5 ml) at room temperature. After stirring for 3.5 hours at room temperature the reaction mixture was diluted with aqueous sodium hydrogen carbonate solution (10 ml), stirred for a further 10 minutes and then filtered, washing with water, to give the title compound as a beige solid after drying at 60° C. under high vacuum. ¹H NMR (400 MHz, CDCl₃) δ ppm 1.96 (s, 3H), 2.21-2.33 (m, 6H), 2.44-2.66 (m, 3H), 3.38-3.44 (m, 4H), 4.76-4.84 (m, 1H), 6.57 (s, br, 2H), 7.61 (s, 1H), 8.07 (s, 1H).

### Intermediate BG

### 3-(4-amino-5-iodo-pyrrolo[2,3-d]pyrimidin-7-yl)-cyclobutanone

Sodium periodate (7.1 g, 33.3 mmol, 1.5 eq) was added to a stirred suspension of intermediate BH (8 g, 22.2 mmol) in 400 mL of THF/H₂O (3/1, v:v). the reaction mixture was stirred for 18 h at rt, diluted with ethyl acetate/NaHCO₃^{sat} and extracted with ethyl acetate. The combined organic extracts were washed with H₂O and brine, dried (Na₂SO₄), filtered and concentrated. The residue was purified by silica gel column chromatography (DCM/MeOH, 95:5) followed by trituration in diethyl ether to afford 4.3 g of the title compound as a yellow solid: ES-MS: 329.1 [M+H]⁺; R_{f}=0.17 (DCM/MeOH, 95:5).

### Intermediate BH

### 3-(4-amino-5-iodo-pyrrolo[2,3-d]pyrimidin-7-yl)-1-hydroxymethyl-cyclobutanol

A mixture (2:1) of E- and Z-3-(4-chloro-5-iodo-pyrrolo[2,3-d]pyrimidin-7-yl)-1-hydroxymethyl-cyclobutanol (Step BH.1, 6 g, 15.8 mmol) was suspended in a mixture of dioxane (30 mL) and aqueous NH₃ (25%, 60 mL) and transferred into three glass autoclaves vessels (50 mL) (Büchi, Flawil) and stirred at 100° C. for 19 hours. The combined reaction mixtures were concentrated under reduced pressure to give the title compound as a mixture of geometric isomers. MS (Method L) M+H=361 (100%). HPLC (Method B): t_{R} 1.83 minutes. TLC (NH₃/MeOH/CH₂Cl₂=1:10:89): R^{F}=0.33 and 0.31, ¹H-NMR (600 MHz, DMSO-d₆): δ ppm (peak intensities Z/E=1:2) 8.08(E)/8.07(Z) (s/s, 1H), 7.74(Z)/7.58(E) (s/s, 1H), 6.65 (s/broad, 2H), 5.30/4.80 (t/t, 1H), 5.26/5.13 (s/s, 1H), 4.84 (m, 1H), 3.30 (m, 2H), 2.70/2.30 (tit, 2H, Z-isomer), 2.60/2.25 (t/t, 2H, E-isomer).

### Step BH1

### 3-(4-chloro-5-iodo-pyrrolo[2,3-d]pyrimidin-7-yl)-1-hydroxymethyl-cyclobutanol

3-(4-Chloro-pyrrolo[2,3-d]pyrimidin-7-yl)-1-hydroxymethyl-cyclobutanol (Step BH.2, 6.94 g, 27.4 mmol) and N-iodosuccinimide (7.39 g, 32.8 mmol) was dissolved in DMF (110 mL) and the mixture stirred at 60° C. under argon. After 2.5 hours, N-iodosuccinimide (0.25 g, 1.1 mmol) was added and the reaction mixture stirred for a further 1 hour at 60° C. After concentration of the reaction mixture under reduced pressure, sodium bicarbonate solution (15 mL) was added and the resulting suspension was extracted with AcOEt (30 mL, 8×). The combined organic phases were washed with Na₂SO₃ solution (10 mL, 2×) and brine (5 mL, 2×), dried (MgSO₄), and concentrated under reduced pressure to give a beige solid, which was further suspended in hexane and washed, and then dried under vacuum to give the title compound as a beige solid. MS (Method L) M+H=380/382. HPLC (Method B): t_{R} 2.53 min. ¹H-NMR (600 MHz, DMSO-d₆): δ ppm (peak intensities Z/E=1:2) 8.61(E)/8.59(Z) (sls, 1H), 8.25 (Z)/8.12(E) (sls, 1H), 5.40/4.86 (quint/quint, 1H), 5.29/5.16 (s/s, 1H), 4.80 (m, 1H), 3.39/3.30 (d/d, 2H), 2.70/2.30 (t/t, 2H/Z-isomer), 2.60/2.25 (t/t, 2H/E-isomer).

### Step BH.2

### 3-(4-chloro-pyrrolo[2,3-d]pyrimidin-7-yl)-1-hydroxymethyl-cyclobutanol

(2,4-Dichloro-pyrimidin-5-yl)-acetaldehyde (7.21 g, 37.7 mmol), 3-amino-1-hydroxymethyl-cyclobutanol (Step BH.3, 4.42 g, 37.7 mmol), and DIPEA (13.18 mL, 75 mmol) were dissolved in EtOH (190 mL) and stirred under reflux (oil bath at 90° C.) for 4.5 hours. After cooling to room temperature, TFA (260 mmol, 20 mL) was added and the reaction mixture stirred under reflux for a further 1 hour. After cooling to room temperature, conc. NaHCO₃ solution (0.5 L) was added, the alcohol evaporated under reduced pressure, and the reaction mixture was then extracted with AcOEt (4×, 100 mL). The combined organic phases were washed with conc. NaHCO₃ solution (50 mL) and brine (40 mL), dried (MgSO₄), concentrated under reduced pressure, purified by normal phase chromaography on a 120 g RediSept silica gel column, and fractioned by means of a Sepacore Control chromatography system (Büchi, Flawil, Switzerland) (eluent: 1 to 10% MeOH (10% NH₃) in CH₂Cl₂) to give the title compound as a beige solid. MS (Method L) M+H=254/256 (100%). HPLC (Method B): t_{R} 2.24 min. ¹H-NMR (600 MHz, DMSO-d₆): 6 ppm (peak intensities Z/E=1:2) 8.63 (E)/8.60(Z) (s/s, 1H), 8.02 (Z)/7.89(E) (did, 1H), 6.72 (Z)/6.68 (E) (d/d, 1H), 3.41/2.78 (quint/quint, 1H), 3.30 (S/broad, 4H), 3.21/3.14 (did, 2H), 2.29/1.50 (m/m, 2H/Z-isomer), 1.95/1.70 (t/t, 2H1E-isomer).

### Step BH.3

### 3-amino-1-hydroxymethyl-cyclobutanol

(3-Hydroxy-3-hydroxymethyl-cyclobutyl)-carbamic acid benzyl ester (Step BH.4, _{—}9.49 g, 37.8 mmol) was dissolved in THF/MeOH (1:1, 150 mL) and hydrogenated under 1 atmosphere of hydrogen for 1 hour in the presence of Pd/C Engelhard 4505 (1.5 g). The reaction mixture was then filtered and the solvent evaporated under reduced pressure to give the title compound as a brown oil. ¹H-NMR (400 MHz, DMSO-d₆): δ ppm (peak intensities Z/E=1:2) 5.40/4.86 (quint/quint, 1H), 5.29/5.16 (s/s, 1H,), 4.85 (m, 1H), 3.41/3.31 (d/d, 2H), 2.75/2.40 (t/t, 2H/Z-isomer), 2.68/2.30 (t/t, 2H/E-isomer).

### Step BH.4

### 3-hydroxy-3-hydroxymethyl-cyclobutyl)-carbamic acid benzyl ester

3-Methylene-cyclobutyl)-carbamic acid benzyl ester (Step BH.5, 9.92 g, 45.6 mmol) dissolved in tert.-butanol/H₂O (40 mL, 1:1) was added to a solution of AD-Mix alpha (70 g, 50.2 mmmol) in tert.-butanol/H₂O (360 mL, 1:1) at 0° C. After stirring at room temperature for 16 hours, the reaction mixture was cooled to 0° C. and Na₂SO₃ (40 g) was added and the reaction mixture was stirred for a further 1 hour at room temperature. After adding H₂O (150 mL), the reaction mixture was extracted with AcOEt (150 mL, 3×). The combined organic phases were washed with brine, dried (MgSO₄), and concentrated under reduced pressure to give the title compound as white solid. MS (Method L): M+H=252. HPLC (Method B): t_{R} 2.32 minutes. TLC(NH₃/MeOH/CH₂Cl₂=1:10:89): R^{f}=0.25. ¹H-NMR (600 MHz, DMSO-d₆): S ppm (peak intensities Z/E=1:2) 7.51 (Z)/7.44(E) (s/s, 1H), 7.35 (m, 5H), 4.95 (s, 2H), 4.80 (Z)/4.70(E) (s/s, 1H), 4.65/4.62 (t/t, 1H), 4.12 (E)/3.52(Z) (sextet/sextet, 1H), 3.25 (Z)/3.20(E) (d/d, 2H), 2.30/1.80 (t/t, 2H/Z-isomer), 1.96 (t, 2H/E-isomer).

### Step BH.5

### 3-methylene-cyclobutyl)-carbamic acid benzyl ester

Diphenylphosphoryl azide (25.3 g, 89 mmol) was added to 3-methylene cyclobutyl carboxylic acid (10 g, 89 mmol) and NEt₃ (15 mL, 105 mmol) dissolved in dioxane/MeCN (15 mL/35 mL) over 15 minutes. The temperature of the reaction mixture then increased to 75° C. with the evolution of gas. After heating the reaction mixture for a further 1 hour at 100° C., benzyl alcohol (20 mL) was added and the reaction mixture was then stirred for 19 hours at 100° C. After cooling and evaporation of the solvent, the residue was taken up in AcOEt (250 mL) and extracted with half conc. NH₄Cl solution (80 mL), half con. NaHCO₃ solution (80 mL), and brine (40 mL), dried (MgSO₄), and concentrated under vacuum. The residue was purified by means of a 120 g RediSept silica gel column using a Sepacore Control chromatographic separator (Büchi) (eluent: hexane/AcOEt=1:9 to 4:6) to give the title compound as a white solid. MS (Method L) M+H=218. HPLC (Method B): t_{R} 3.12 minutes. TLC (AcOEt/hexane=1:4): R^{F}=0.30. ¹H-NMR (400 MHz, DMSO-d₆): δ ppm 7.64 (d, 1H), 7.32 (m, 5H), 4.99 (s, 2H), 4.76 (s, 2H), 3.95 (sextet, 1H), 2.85 (m, 2H), 2.62 (m, 2H).

### Intermediate BI

### d₂-1-[3-(4,4,5,5-tetramethyl-[1,3,2]dioxaborolan-2-yl)-phenoxymethyl]-7-oxa-bicyclo[2.2.1]heptane

A mixture of d₂-1-(3-bromo-phenoxymethyl)-7-oxa-bicyclo[2.2.1]heptane (Step B1.1, 281 mg, 0.985 mmol), bis(pinacolato)diboron (325 mg, 1.28 mmol), potassium acetate (242 mg, 2.46 mmol), [1,1-bis(diphenylphosphino)ferrocene]dichloropalladium(II) dichloromethane complex (72 mg, 0.099 mmol), 1,1-bis(diphenylphosphino)ferrocene (72 mg, 0.130 mmol) and DMF (2.5 ml) was heated under an argon atmosphere at 80° C. for 20 hours. The cooled reaction mixture was diluted with DCM, washed with water and brine, dried over sodium sulphate and evaporated. Purification of the residue by flash column chromatography, eluting with a methanol in DCM gradient, gave the title compound as viscous clear-red/brown oil.

### Step B1.1

### d₂-1-(3-bromo-phenoxymethyl)-7-oxa-bicyclo[2.2.1]heptane

A mixture of d₂-1-iodomethyl-7-oxa-bicyclo[2.2.1]heptane (Step B1.2, 253 mg, 1.05 mmol), 3-bromophenol (201 mg, 1.16 mmol), potassium carbonate (291 mg, 2.11 mmol) and acetonitrile (1 ml) were heated for 24 hours at 150° C. in a sealed vessel. After cooling the reaction mixture was diluted with DCM, washed with 1 N aqueous sodium hydroxide solution and water, dried over magnesium sulphate and evaporated to the title compound as a yellow oil. ¹H NMR (400 MHz, CDCl₃) δ ppm 1.50-1.58 (m, 1H), 1.58-1.70 (m, 3H), 1.70-1.85 (m, 2 H), 1.85-1.98 (m, 2H), 4.59-4.67 (m, 1H), 6.91 (d, J=8.2 Hz, 1H), 7.05-7.15 (m, 3H).

### Step B1.2

### d₂-1-iodomethyl-7-oxa-bicyclo[2.2.1]heptane

Iodine (5.04 g, 19.9 mmol) was added to a mixture of d₂-4-methylene-cyclohexanol (Step BI.3, 480 mg, 2.94 mmol), sodium carbonate (437 mg, 4.1 mmol) and acetonitrile (28 ml) at room temperature. After stirring for 1 hour at room temperature in the dark the reaction mixture was diluted with diethyl ether, washed with 10% aqueous sodium thiosulphate solution and brine, the combined organic layers dried over magnesium sulphate and evaporated under a reduced pressure of 200 mbar at 40° C. The residue was purified by flash column chromatography, eluting with a gradient of ethyl acetate in hexane, to give the title compound as a pale yellow oil.

### Step B1.3

### d₂-4-methylene-cyclohexanol

para-Toluenesulphonic acid (10 mg, 0.053 mmol) was added to a solution of d₂-2-(4-methylene-cyclohexyloxy)-tetrahydropyran (Step B1.4, 973 mg, 4.86 mmol) in methanol (10 ml) and the mixture stirred for 17 hours at room temperature. Volatiles were then removed under a reduced pressure of 300 mbar at 40° C., the residue taken up in DCM, the organic phase washed with water, dried over sodium sulphate and evaporated to give the title compound a clear colourless oil. ¹H NMR (400 MHz, CDCl₃) δ ppm 1.50 (s, 2H), 1.87-1.97 (m, 2H), 2.02-2.12 (m, 2H), 2.34 (dt, J=14.0 & 5.1 Hz, 2H), 3.78-3.86 (m, 1H).

### Step B1.4

### d₂-2-(4-methylene-cyclohexyloxy)-tetrahydropyran

A solution of n-butyllitium in hexanes (2.5 M, 3.05 ml, 7.62 mmol) was added to a mixture of d3-methyltriphenylphosphonium bromide (2.94 g, 8.16 mmol) and THF (25 ml) cooled at −10° C. After stirring for 90 minutes a solution of 4-(tetrahydropyran-2-yloxy)-cyclohexanone (Step B1.5, 1.18 g, 5.44 mmol) in THF (5 ml) was added and the reaction mixture stirred for a further 3 hours as it warmed to room temperature. Acetone was then added and the reaction mixture allowed to stand for 18 hours at room temperature. Partial removal of volatiles under vacuum was then followed by elution through a plug of silica gel with a 1:1 mixture of heptane and diethyl ether and evaporation to the title compound as a clear colourless oil. ¹H NMR (400 MHz, CDCl₃) δ ppm 1.39-1.66 (m, 6H), 1.66-1.77 (m, 1H), 1.77-1.97 (m, 3H), 1.98-2.14 (m, 2H), 2.26-2.43 (m, 2H), 3.50 (ddd, J=11.0, 5.4 & 5.1 Hz, 1H), 3.72-3.84 (m, 1H), 3.87-3.97 (m, 1H), 4.70-4.75 (m, 1H).

### Step B1.5

### 4-(tetrahydropyran-2-yloxy)-cyclohexanone

A mixture of 4-hydroxycyclohexanone (1.0 g, 8.76 mmol), 3,4-dihydro-2H-pyran (2.5 ml, 29.5 mmol) and para-toluenesulphonic acid (20 mg, 0.11 mmol) was stirred at room temperature for 45 minutes and then diluted with diethyl ether. The mixture washed with aqueous sodium hydrogen carbonate solution, dried over magnesium sulphate and evaporated. Purification of the residue by flash column chromatography, eluting with a gradient of ethyl acetate in heptane, gave the title compounds a clear colourless oil.

### Intermediate BJ

### 7-(3-aminomethyl-cyclobutyl)-5-iodo-7H-pyrrolo[2,3-d]pyrimidin-4-ylamine

Triphenylphosphine (833 mg, 3.18 mmol) was added to a mixture of 7-(3-azidomethyl-cyclobutyl)-5-iodo-7H-pyrrolo[2,3-d]pyrimidin-4-ylamine (Step BJ.1,920 mg, 2.12 mmol), ammonium hydroxide solution (25%, 1.32 ml, 8.47 mmol), water (1.4 ml), methanol (7 ml) and THF (7 ml). The reaction mixture was stirred overnight at room temperature, then diluted with water, extracted 2× with ethyl acetate, the combined organic phases washed with brine, dried over sodium sulphate and evaporated. Purification of the residue by flash column chromatography, eluting with a gradient of methanol in DCM containing 1% concentrated ammonia solution, gave the title compoundas a white solid. ¹H NMR (400 MHz, DMSO) 5 ppm 1.74 (s, br, 2H), 2.06-2.18 (m, 3H), 2.32-2.39 (m, 2H), 2.57-2.60 (m, 2H), 4.95-5.02 (m, 1H), 6.59 (s, br, 2H), 7.68 (s, 1H), 8.08 (s, 1H).

### Step BJ.1

### 7-(3-azidomethyl-cyclobutyl)-5-iodo-7H-pyrrolo[2,3-d]pyrimidin-4-ylamine

A mixture of toluene-4-sulfonic acid 3-(4-amino-5-iodo-pyrrolo[2,3-d]pyrimidin-7-yl)-cyclobutylmethyl ester (18.0 g, 18.1 mmol), sodium azide (4.70 g, 72.2 mmol) and DMF (60 ml) was heated at 65° C. for 1 hour. The cooled reaction mixture was diluted with water, extracted 3× with ethyl acetate, the combined organic phases washed with brine, dried over magnesium sulphate and evaporated to give the title compound as a yellow solid. HPLC/MS t_{R} 0.97 min, M+H 369.9 (Method X).

### Step BJ.2

### toluene-4-sulfonic acid 3-(4-amino-5-iodo-pyrrolo[2,3-d]pyrimidin-7-yl)-cyclobutylmethyl ester

para-toluene sulphonyl chloride (11.52 g, 60.4 mmol) was added portion-wise over 45 minutes to a solution of [cis-3-(4-amino-5-iodo-pyrrolo[2,3-d]pyrimidin-7-yl)-cyclobutyl]-methanol (Intermediate M, 7.0 g, 20.14 mmol) in pyridine (20 ml) cooled at −20° C. After 18 hours at −25° C. the reaction mixture was partitioned between 1 N sulphuric acid and DCM cooled at 0° C., extracted 2× with DCM, the combined organic layers dried over sodium sulphate and evaporated to give the title compound as a yellow solid. HPLC/MS t_{R} 1.12 min, M+H 498.9 (Method X).

### Intermediate BK

### d₉-1-[3-(4,4,5,5-tetramethyl-[1,3,2]dioxaborolan-2-yl)-phenoxymethyl]-7-oxa-bicyclo[2.2.1]heptane

A dispersion of sodium hydride in mineral oil (60%, 178 mg, 4.46 mmol) was added to a mixture of d_{g}-1-iodomethyl-7-oxa-bicyclo[2.2.1]heptane (Step BK.1, 1.24 g, 3.27 mmol), 3-hydroxyphenyl boronic acid pinacol ester (667 mg, 2.97 mmol), tetrabutylammonium iodide (56 mg, 0.15 mmol) and DMF (12 ml) and stirred for 45 minutes. The reaction mixture was then heated at 100° C. for 4 hours. After cooling to room temperature the reaction mixture was diluted with water, extracted 2× with ethyl acetate, the combined organic layers washed with brine, dried over sodium sulphate and evaporated. Purification of the residue by flash column chromatography, eluting with a gradient of methanol in DCM, gave the title compound as an orange oil.

### Step BK.1

### d₉-1-iodomethyl-7-oxa-bicyclo[2.2.1]heptane

Iodine (170 g, 168 mmol) was added to a mixture of d₉-4-methylene-cyclohexanol (Step BK.2, 25.4 g, 168 mmol), sodium carbonate (31.1 g, 293 mmol) and acetonitrile (1.7 I) at room temperature. After stirring for 1 hour at room temperature in the dark the reaction mixture was diluted with 10% aqueous sodium thiosulphate solution, extracted 2× with ethyl acetate, washed with brine, the combined organic layers dried over magnesium sulphate and evaporated under a reduced pressure of 200 mbar at 40° C. to give the title compound as a pale yellow oil. ¹H NMR (400 MHz, CDCl₃) δ ppm 3.54 (s, 2H).

### Step BK.2

### d₉-4-methylene-cyclohexanol

Sodium borodeuteride (27.4 g, 654 mmol) was added portion-wise to a solution of d₈-4-methylene-cyclohexanone (Step BK.3, 38.68 g, 327 mmol), in THF (82 ml) at 0° C. The reaction mixture was stirred for 1 hour at room temperature, diluted with diethyl ether, washed with water, dried over sodium sulphate and evaporated. Purification of the residue by flash column chromatography, eluting with a gradient of DCM in hexane, gave the title compound as a yellow oil. ¹H NMR (400 MHz, CDCl₃) δ ppm 4.65 (s, 2H).

### Step BK.3

### d₈-4-methylene-cyclohexanone

A mixture of d₄-4-methylene-cyclohexanone (Step BK.3, 48.0 g, 420 mmol), triethylamine (13.5 ml, 97 mmol), d₁-ethanol (180 ml) and deuterium oxide (20 ml) was stirred for 17 hours at room temperature. The volume was then reduced under a vacuum of 120 mbar at 40° C., then extracted with diethyl ether, the combined organic layers washed with water, dried over magnesium sulphate and evaporated to give a clear yellow oil. Exposure of the isolated material to the above procedure for a second time gave the title compound as a yellow oil. ¹H NMR (400 MHz, CDCl₃) δ ppm 4.89 (s, 2H).

### Step BK.3

### d₄-4-methylene-cyclohexanone

Oxalic acid (53.4 g, 594 mmol) was added to a mixture of d₄-8-methylene-1,4-dioxaspiro[4.5]decane (Step BK.4, 43.0 g, 245 mmol), acetone (300 ml) and water (150 ml) at room temperature. After 8 hours sodium bicarbonate was added, the reaction mixture filtered, washing with diethyl ether and the filtrate extracted with diethyl ether. The combined organic layers were then washed with brine, dried over magnesium sulphate and evaporated under a reduced pressure of 200 mbar at 30° C. Exposure of the isolated material to the above procedure for a second time gave the title compound as a colourless oil. ¹H NMR (400 MHz, CDCl₃) δ ppm 2.39 (s, 4H), 4.89 (s, 2H).

### Step BK.4

### d₄-8-methylene-1,4-dioxa-spiro[4.5]decane

A solution of n-butyllitium in hexanes (2.5 M, 164 ml, 411 mmol) was added to a mixture of methyltriphenylphosphonium bromide (161 g, 440 mmol) and THF (1 l) cooled at −10° C. After 85 minutes d₄-1,4-dioxa-spiro[4.5]decan-8-one (58.9 g, 294 mmol) was added. After stirring for a further 2 hours at room temperature acetone was added followed by partial removal of volatiles under vacuum. Elution of the remaining reaction mixture through a plug of silica gel with a 1:1 mixture of heptane and diethyl ether and evaporation under a reduced pressure of 200 mbar at 35° C. gave the title compound as a clear yellow oil. ¹H NMR (400 MHz, CDCl₃) δ ppm 1.69 (s, 4H), 3.96 (s, 4H), 4.67 (s, 2H).

### Step BK.5

### d₄-1,4-dioxa-spiro[4.5]decan-8-one

A mixture of 1,4-dioxa-spiro[4.5]decan-8-one (52 g, 323 mmol), triethylamine (10 ml, 71.7 mmol), d₁-ethanol (152 ml) and deuterium oxide (8 ml) was stirred for 24 hours at room temperature. The volume was then reduced under vacuum at 35° C., benzene added and the mixture evaporated to give a clear yellow oil. Exposure of the isolated material to the above procedure for a second time gave the title compound. ¹H NMR (400 MHz, CDCl₃) δ ppm 2.00 (s, 4H), 4.16 (s, 4H).

### Intermediate BL

### 1-aminomethyl-(E)-3-{4-amino-5-[3-(7-oxa-bicyclo[2.2.1]hept-1-ylmethoxy)-phenyl]-pyrrolo[2,3-d]pyrimidin-7-yl}-cyclobutanol

3-{(4-Amino-5-[3-(7-oxa-bicyclo[2.2.1]hept-1-ylmethoxy)-phenyl]-pyrrolo[2,3-d]pyrimidin-7-yl}-1-azidomethyl-cyclobutanol (280 mg, 0.546 mmol; Step BL.1) was dissolved in a mixture of THF (1.35 mL), H₂O (0.34 mL), MeOH (1.35 mL), and aqueous ammonia (25%, 0.340 mL) and stirred at RT for 10 min under Ar. After adding triphenylphosphine (212 mg, 0.819 mmol), the reaction mixture was stirred at RT for 4.5 h. After adding H₂O (15 mL), the reaction mixture was extracted with AcOEt (20 mL, 3×), the combined organic phases were extracted with H₂O (10 mL) and brine (10 mL), dried (Na₂SO₄), and concentrated under reduced pressure. The resulting yellow foam (428 mg) taken up in Isolute (International Sorbent Technology) and purified by column chromatography using a 12 g silica gel column (RediSept (Isco)) and a Sepacore Control chromatography system from Büchi (DCM/MeOH (10% NH₃ (25%))=99:1 (10 min) -->DCM/MeOH (10% NH₃ (25%))=9:1 in 30 min at a flow rate of 30 mL/min) to give the title compound as a white solid (185 mg). HPLC/MS t_{R}=−0.64 min, M+H=436.3 (method X). ¹H NMR (600 MHz, DMSO-d₆) δ ppm 8.14 (s, 1H, pyrimidinyl), 7.60 (s, 1H, pyrrolyl), 7.37 (t, 1H, phenyl), 7.08 (s, 1H, phenyl), 7.05/6.96 (d/d, 1H/1H, phenyl), 6.15 (s/b, 2H, NH₂), 5.40 (quintet, 1H, CH-cyclobutyl), 5.01 (s, 1H, OH), 4.51 (s, 1H, CH-oxabicycloheptanyl), 4.30 (s, 2H, CH₂—O), 2.65 (s, 2H, CH₂—N), 2.65/2.30 (m/m, 4H, CH₂-cyclobutyl), 1.70/1.55 (m/m, 10H, CH₂-oxabicycloheptanyl, NH₂-CH₂).

### Step BL.1

### 3-{4-Amino-5-[3-(7-oxa-bicyclo[2.2.1]hept-1-ylmethoxy)-phenyl]-pyrrolo[2,3-d]pyrimidin-7-yl}-1-azidomethyl-cyclobutanol

To a solution of toluene-4-sulfonic acid 3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl ester (Step BL.2, 400 mg, 0.67 mmol) in DMF (3.3 ml), NaN₃ (178 mg, 2.71 mmol) was added and the reaction mixture was stirred at 65° C. for 3 h under Ar. The reaction mixture was cooled down to RT, added to H₂O (15 mL), and extracted with AcOEt (20 mL, 3×). The combined organic phases were washed with H₂O (10 mL) and brine (10 mL, 2×), dried (Na₂SO₄), and concentrated under reduced pressure to give a yellow oil (414 mg). The material was purified by column chromatography using a 40 g silica gel column (RediSept (Isco)) and a Sepacore Control chromatography system from Büchi: DCM (5 min) -->DCM/MeOH (10% NH₃ (25%))=95:1 in 30 min at a flow rate of 40 ml/min) to give the title compound as a colorless foam (283 mg). HPLC/MS t_{R}=0.99 min, M+H=462.3 (method X). ¹H NMR (600 MHz, DMSO-d₆) 8 ppm 8.12 (s, 1H, pyrimidinyl), 7.63 (s, 1H, pyrrolyl), 7.36 (t, 1H, phenyl), 7.07 (s, 1H, phenyl), 7.04/6.96 (d/d, 1H/1H, phenyl), 6.15 (s/b, 2H, NH₂), 5.63 (s, 1H, OH), 5.44 (quintet, 1H, CH-cyclobutyl), 4.51 (s, 1H, CH-oxabicycloheptanyl), 4.29 (s, 2H, CH₂—O), 3.43 (s, 2H, CH₂—N), 2.58/2.44 (m/m, 4H, CH₂-cyclobutyl), 1.68/1.56 (m/m, 8H, CH₂-oxabicycloheptanyl).

### Step BL.2

### Toluene-4-sulfonic acid (E)-3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl ester

A 1:2 mixture of cis and trans 3-{4-Amino-5-[3-(7-oxa-bicyclo[2.2.1]hept-1-ylmethoxy)-phenyl]pyrrolo2,3-d]pyrimidin-7-yl}-1-hydroxymethyl-cyclobutanol (Intermediate BN, 1.5 g, 3.26 mmol) and dibutyltin oxide (0.894 g, 3.59 mmol) were stirred in DCM/CHCl₃,/MeOH (86 mL, 3:6:1) at 65° C. for 2 h under Ar. After evaporation of the solvent and drying the remaining solid under vacuum at 50° C. for 4 h, DCM (75 mL) and Ts-Cl (0.685 g, 3.59 mmol) were added and the reaction mixture was stirred at RT for 90 min. After adding H₂O (0.118 mL, 6.53 mmol), the reaction suspension is vigorously stirred at RT for 30 min. After evaporation of the solvent, the remaining residue (3.23 g) is purified by means of Flash-Master chromatograph using a 100 g silica gel column (RediSept (Isco)): A=DCM, B=MeOH with 10% NH₃ (25%); A: 5 min, -->5% B in 30 min, remaining at 5% B for 28 min, —>10% Bin 15 min, remaining at 10% B for 10 min; flow rate of 40 mL/min) to give the title compound (the trans-isomer) as a white solid (1.03 g, second eluting geometric isomer). HPLC/MS t_{R}=1.00 min, M+H=591.3 (method X). ¹H NMR (600 MHz, DMSO-d₆) δ ppm 8.08 (s, 1H, pyrimidinyl), 7.80 (d, 2H, Ts), 7.55 (s, 1H, pyrrolyl), 7.45 (d, 2H, Ts), 7.36 (t, 1H, phenyl), 7.08 (s, 1H, phenyl),7.03/6.97 (did, 2H, phenyl), 6.15 (s/b, 2H, NH₂), 5.65 (s, 1H, OH), 5.34 (quintet, 1H, CH-cyclobutyl), 4.51 (s, 1H, CH-oxabicycloheptanyl),), 4.29 (s, 2H, CH₂—O-phenyl), 3.30 (s, 3H, CH₃-Ts), 2.54/2.35 (m/m, 4H, CH₂-cyclobutyl), 2.40 (s, 2H, CH₂-OTs),1.70/1.55 (m/m, 8H, CH₂-oxabicycloheptanyl).

### Step BL.3

### Toluene-4-sulfonic acid (Z)-3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl ester

Isolated from Step BL.2 (first eluting geometric isomer) as a colorless solid (0.55 g).

HPLC/MS t_{R}=1.02 min, M+H=591.3 (method X). ¹H NMR (600 MHz, DMSO-d₆) 8 ppm 8.13 (s, 1H, pyrimidinyl), 7.86 (d, 2H, Ts), 7.61 (s, 1H, pyrrolyl), 7.53 (d, 2H, Ts), 7.38 (t, 1H, phenyl), 7.08 (s, 1H, phenyl),7.03/6.97 (did, 2H, phenyl), 6.15 (s/b, 2H, NH₂), 5.77 (s, 1H, OH), 4.82 (quintet, 1H, CH-cyclobutyl), 4.50 (s, 1H, CH-oxabicycloheptanyl),), 4.29 (s, 2H, CH₂—O-phenyl), 4.11 (s, 2H, CH₂—OTs), 2.65/2.45 (m/m, 4H, CH₂-cyclobutyl), 2.40 (s, 3H, CH₃-Ts), 1.70/1.55 (m/m, 8H, CH₂-oxabicycloheptanyl).

### Intermediate BM

### 3-{4-amino-5-[3-(7-oxa-bicyclo[2.2.1]hept-1-ylmethoxy)-phenyl]-pyrrolo[2,3-d]pyrimidin-7-yl}-cyclobutanone

Sodium periodate (1.135 g, 5.31 mmol, 1.3 eq) was added to a stirred suspension of intermediate BN (1.80 g, 4.08 mmol) in 164 mL of THF/H₂O (3/1, v:v). the reaction mixture was stirred for 17 h at rt, diluted with ethyl acetate/brine and extracted with ethyl acetate. The combined organic extracts were washed with brine, dried (Na₂SO₄), filtered and concentrated. The residue was purified by silica gel column chromatography (DCM/MeOH/NH4OH c., 200:10:1) to afford 1.62 g of the title compound as a beige foam: HPLC/MS t_{R}: 0.94 min, M+H=405.3 (methode X). ¹H NMR (600 MHz, DMSO-d₆) δ ppm 8.16 (s, 1H), 7.73 (s, 1H), 7.38 (t, 1H), 7.10 (s, 1H), 7.05 (d, 1H), 6.97 (d, 1H), 6.22 (s/b, 2H), 5.46 (m, 1H), 4.51 (m, 1H), 4.31 (5, 2H), 3.77 (m, 2H), 3.57 (m, 2H), 1.67 (m, 4H), 1.57 (m, 4H),

### Intermediate BN.1

### 3-{4-amino-5-[3-(7-oxa-bicyclo[2.2.1]hept-1-ylmethoxy)-phenyl]-pyrrolo[2,3-d]pyrimidin-7-yl}-1-hydroxymethyl-cyclobutanol

Intermediate BH (3.0 g, 7.5 mmol), Intermediate BB (3.13 g, 9.0 mmol), Pd(PPh3)4 (0.433 g, 0.375 mmol), K₃PO₄ (3.18 g, 15 mmol), and soda (1.589 g, 15 mmol) were dissolved in DMF (50 mL) and H₂O (2.5 mL) under Ar and stirred at 100° C. for 45 min. After cooling down to RT, the reaction mixture was filtered over Hyflo and the solvent was evaporated concentrated under vacuum. The residue was partitioned between AcOEt (50 mL) and H₂O (50 mL). The aqueous phase was extracted twice with AcOEt (60 mL). The combined organic phases were washed with H₂O (20 mL) and brine (20 mL), dried (MgSO₄), concentrated under reduced pressure. Precipitation from AcOEt/hexane and THF/hexane yielded to the title compound as a 1:2 mixture of the cis and trans isomers (2.14 g). HPLC/MS t_{R}=0.66 min, M+H=437.3 (method X). ¹H NMR (600 MHz, DMSO-d₆) δ ppm 8.08 (s, 1H, pyrimidinyl), 7.80 (d, 2H, Ts), 7.55 (s, 1H, pyrrolyl), 7.40 (d, 2H, Ts), 7.38 (t, 1H, phenyl), 7.07 (s, 1H, phenyl), 7.04/6.97 (d/d, 1H/1H, phenyl), 6.20 (s/b, 2H, NH₂), 5.65 (s, 1H, OH), 5.32 (quintet, 1H, CH-cyclobutyl), 5.25/5.08 (s/s, 1H, HO-cycciobutyl, cis/trans),4.85 (m, 1H, CH₂—OH), 4.50 (5, 1H, CH-oxabicycloheptanyl), 4.29 (s, 2H, CH₂-phenyl), 3.60/3.45 (d/d, 2H, CH₂—OH, cis/trans), 2.65-2.25 (m, 4H, CH₂-cyclobutyl), 1.74-1.45 (m, 8H, CH2-oxabicycloheptanyl).

### Intermediate BO

### 7-[3-(2,5-diaza-bicyclo[2.2.1]hept-2-yl)-cyclobutyl]-5-[3-(7-oxa-bicyclo[2.2.1]hept-1-ylmethoxy)-phenyl]-7H-pyrrolo[2,3-d]pyrimidin-4-ylamine

5-(3-{4-Amino-5-[3-(7-oxa-bicyclo[2.2.1]hept-1-ylmethoxy)-phenyl]-pyrrolo[2,3-d]pyrimidin-7-yl}-cyclobutyl)-2,5-diaza-bicyclo[2.2.1]heptane-2-carboxylic acid tert-butyl ester (Step BO.1, 200 mg, 0.341 mmol) was stirred with DCM (3 ml) and trifluoroacetic acid (0.026 ml, 341 ml) for 18 hours at room temperature. The reaction mixture was then partitioned between aqueous sodium bicarbonate solution and DCM, the organic layer dried over sodium sulphate and evaporated to give the title compound as a 2:1 cis:trans mixture. HPLC/MS t_{R} 0.73 min, M+H 487.2 (Method X).

### Step B0.1

### 5-(3-{4-amino-5-[3-(7-oxa-bicyclo[2.2.1]hept-1-ylmethoxy)-phenyl]-pyrrolo[2,3-d]pyrimidin-7-yl}-cyclobutyl)-2,5-diaza-bicyclo[2.2.1]heptane-2-carboxylic acid tert-butyl ester

Sodium triacetoxyborohydride (118 mg, 0.556 mmol) was added portionwise over 5 minutes to a mixture of 3-(4-amino-5-iodo-pyrrolo[2,3-d]pyrimidin-7-yl)-cyclobutanone (Intermediate BG, 150 mg, 0.371 mmol), N-Boc-2,5-diazabicyclo[2.2.1]heptane (90 mg, 0.445 mmol), acetic acid (0.021 ml, 0.371 mmol) and 1,2-dichloroethane (2 ml) at room temperature. After stirring for 2 hours at room temperature the reaction mixture was diluted with water, extracted 2× with DCM, the combined organic layers dried over sodium sulphate and evaporated to give the title compound as a clear yellow glass. HPLC/MS t_{R} 0.96 min, M+H 587.4 (Method X).

### Intermediate BP

### cis-7-(3-Azidomethyl-cyclobutyl)-5-[3-(4-methyl-7-oxa-bicyclo[2.2.1]hept-1-ylmethoxy)-phenyl]-pyrrolo[2,3-d]pyrimidin-4-ylamine

The title compound was prepared in a similar manner as described for Intermediate BE starting from Intermediate BE and sodium azide. NMR (400 MHz, CDCl₃) δ ppm 8.31 (s, 1H), 8.02 (s, 1H), 7.39 (dd, 1H), 7.18 (s, 1H), 7.11 (d, 1H), 7.01 (d, 1H), 5.31 (bs, 2H, NH₂), 5.22 (quin, 1H), 4.64 (quin, 1H), 4.33 (s, 2H), 3.48 (d, 2H), 2.76-2.70 (m, 2H), 2.66-2.45 (m, 1H), 2.38-2.31 (m, 4H), 1.93-1.84 (m, 6H).

### Intermediate BQ

### 3-{8-Amino-1-[3-(7-oxa-bicyclo[2.2.1]hept-1-ylmethoxy)-phenyl]-imidazo[1,5-a]pyrazin-3-yl}-cyclobutanone

3-{8-Amino-1-[3-(7-oxa-bicyclo[2.2.1]hept-1-ylmethoxy)-phenyl]-imidazo[1,5-a]pyrazin-3-yl}-1-hydroxymethyl-cyclobutanol (Intermediate BR; E/Z-mixture; 0.8 g, 1.8 mmol) was dissolved in THF (10 mL) and cooled to 0° C. Water (3 mL) and sodium periodide (0.51 g, 2.4 mmol) were added and the reaction mixture slowly warmed to room temperature and stirred for 1 h. It was then subjected to aqueous workup and the crude producted purified by flash chromatography (SiO₂; DCM/MeOH; gradient 0-20% MeOH) to give the title as a yellow solid. ¹H NMR (400 MHz, MeOH-d₄) δ ppm 1.57-1.68 (m, 4H), 1.77-1.97 (m, 4H), 3.56-3.68 (m, 2 H), 3.76-3.94 (m, 3H), 4.31-4.37 (m, 2H), 4.64 (t, J=5.1 Hz, 1H), 7.06-7.11 (m, 2H), 7.17-7.21 (m, 1H), 7.23 (d, J=7.4 Hz, 1H), 7.40-7.46 (m, 1H).

### Intermediate BR

### E-3-{8-Amino-1[3-(7-oxa-bicyclo[2.2.1]hept-1-ylmethoxy)-phenyl]-imidazo[1,5-a]pyrazin-3-yl}-1-hydroxymethyl-cyclobutanol and Z-3-{8-Amino-1-[3-(7-oxa-bicyclo[2.2.1]hept-1-ylmethoxy)-phenyl]-imidazo[1,5-a]pyrazin-3-yl}-1-hydroxymethyl-cyclobutanol

3-(8-Amino-iodo-imidazo[1,5-a]pyrazin-3-yl)-1-hydroxymethyl-cyclobutanol (E/Z mixture, Intermediate BS, 3.0 g, 8.33 mmol) and 1-methyl-4-[3-(4,4,5,5-tetramethyl-[1,3,2]dioxaborolan-2-yl)-phenoxymethyl]-7-oxa-bicyclo[2.2.1]heptane (intermediate BB; 3.30 g, 10.0 mmol) were dissolved in DMF (40 mL). Water (2 mL), K₃PO₄ (3.5 g, 16.6 mmol) Na₂CO₃ (1.76 g, 16.6 mmol) and tetrakis(triphenylphosphine)palladium (0.9 g, 0.83 mmol) were added and the reaction mixture purged with Argon. It was then heated to 100° C. and stirred at this temperature for 2 h. After cooling to room temperature. It was subjected to aqueous workup and the crude product purified by flash chromatography (SiO₂; DCM/MeOH; gradient 0-20% MeOH) to give the title as a yellow solid as a mixture of E- and Z-isomers. M+H 437.2 (Method X). ¹H NMR (400 MHz, MeOH-d₄) S ppm 1.63-1.73 (m, 4H), 1.78-1.90 (m, 4H), 2.45-2.54 (m, 2H), 2.70-2.78 (m, 2H), 3.47 (t, J=8.8 Hz, 1H), 3.67 (s, 2H), 4.36 (s, 2H), 4.54-4.61 (m, 1H), 6.99 (d, J=5.1 Hz, 1H), 7.12 (dd, J=8.4, 2.5 Hz, 1H), 7.20-7.28 (m, 2 H), 7.40-7.49 (m, 2H), (data for Z-isomer).

### Intermediate BS

### (Z)-3-(8-Amino-1-iodo-imidazo[1,5-a]pyrazin-3-yl)-1-hydroxymethyl-cyclobutanol and (E)-3-(8-amino-1-iodo-imidazo[1,5-a]pyrazin-3-yl)-1-hydroxymethyl-cyclobutanol

A mixture of (Z)-3-(8-chloro-1-iodo-imidazo[1,5-a]pyrazin-3-yl)-1-hydroxymethyl-cyclobutanol and (E)-3-(8-chloro-1-iodo-imidazo[1,5-a]pyrazin-3-yl)-1-hydroxymethyl-cyclobutanol (prepared as described in WO 2005/097800, 8.8 g, 23.18 mmol), 25% aqueous ammonia solution (46.1 ml) and 1,4-dioxane (30 ml) were heated in sealed tube at 100° C. for 20 hours. After cooling the reaction mixture the title compound was collected, as a yellow solid, by filtration and trituration with methanol. MS: M+H 362.2 (Method X). ¹H NMR (400 MHz, DMSO-d₆) δ ppm 7.43 (d, 1H), 6.94 (d, 1H), 6.57 (bs, 2H), 3.85-3.76 (m, 1H), 3.15 (s, 2H), 2.49-2.42 (m, 2H), 2.25-2.21 (m, 2H).

### Intermediate BT and BU

### (E)-3-(4-amino-5-iodo-pyrrolo[2,3-d]pyrimidin-7-yl)-1-hydroxymethyl-cyclobutanol and (Z)-3-(4-amino-5-iodo-pyrrolo[2,3-d]pyrimidin-7-yl)-1-hydroxymethyl-cyclobutanol

The mixture (2:1) of the geometric isomeres E- and Z-3-(4-amino-5-iodo-pyrrolo[2,3-d]pyrimidin-7-yl)-1-hydroxymethyl-cyclobutanol (intermediate BH: 34.5 g, 82.5 mmol) was recrystallized serveral times from methanol to afford Intermediate BT (E-isomer) as yellow crystals. The mother liquids were combined, concentrated and dried in vacuo to afford the enriched Intermediate BU (Z-isomer) as beige crystals.

Alternatively, recrystallisation of the E:Z mixture (Intermediate BH) from acetic acid gives (E)-3-(4-amino-5-iodo-pyrrolo[2,3-d]pyrimidin-7-yl)-1-hydroxymethyl-cyclobutanol (Intermediate BT) as a white solid. ¹H NMR (400 MHz, DMSO) δ ppm 8.06 (s, 1H), 7.56 (s, 1H), 6.57 (s, br, 2H), 5.29 (pent, 1H), 5.06 (s, 1H), 4.84 (t, 1H), 3.27 (d, 2H), 2.58-2.50 (m, 2H), 2.26-2.19 (m, 2H).

### Intermediate BV

### (trans-4-{4-Amino-5-[3-(7-oxa-bicyclo[2.2.1]hept-1-ylmethoxy)-phenyl]-pyrrolo[2,3-d]pyrimidin-7-yl}-cyclohexyl)-carbamic acid tert-butyl ester

A mixture of the intermediate prepared in step BV.1 (88 mg, 0.205 mmol), 2-(3-((7-oxabicyclo[2.2.1]heptan-1-ylmethoxy)phenyl)-4,4,5,5-tetramethyl-1, 3,2-dioxaborolane (Intermediate K, 303 mg, 0.918 mmol, 1.2 eq), Pd(PPh₃)₄ (44.2 mg, 38 μmol, 0.05 eq), K₃PO₄ (325 mg, 1.53 mmol, 2 eq), Na₂CO₃ (162 mg, 1.53 mmol, 2 eq) in DMF (5 mL) and H₂O (0.5 mL) was stirred at 100° C. for 30 min under an argon atmosphere, allowed to cool to rt and diluted with ethyl acetate/H₂O. The aqueous layer was extracted with ethyl acetate. The combined organic extracts were washed with H₂O and brine, dried (Na₂SO₄), filtered and concentrated. The residue was purified by silica gel column chromatography (ethyl acetate/MeOH, 97.5:2.5) to afford 300 mg of the title compound as a white foam: ES-MS: 534.3 [M+H]⁺; t_{R}=4.50 min (Method C); R_{f}=0.24 (ethyl acetate/MeOH, 97.5:2.5).

### Step BV.1

### [4-(4-Amino-5-iodo-pyrrolo[2,3-d]pyrimidin-7-yl)-cyclohexyl]-carbamic acid tert-butyl ester

A mixture of the intermediate prepared in step BV.2 (404 mg, 0.847 mmol), NH₄OH (30% NH₃ aqueous solution, 2 mL) and EtOH (2 mL) was stirred at 120° C. for 16 h in a sealed vessel, allowed to cool to rt and diluted in ethyl acetate/H₂O. The aqueous layer was extracted with ethyl acetate. The combined organic extracts were washed with H₂O and brine, dried (Na₂SO₄), filtered and concentrated to afford 360 mg of the title compound as a white solid: ES-MS: 458.1 [M+H]⁺; t_{R}=3.69 min (Method C).

### Step BV.2

### [trans-4-(4-Chloro-5-iodo-pyrrolo[2,3-d]pyrimidin-7-O-cyclohexyl]-carbamic acid tert-butyl ester

A mixture of the intermediate prepared in step BV.3 (344 mg, 0.980 mmol) and NIS (331 mg, 1.47 mmol, 1.1 eq) in DMF (4 mL) was stirred for 18 h at rt. Then, NIS (80 mg) was added. The mixture was stirred for additional 4 h at rt and then diluted with ethyl acetate 1H₂O. The aqueous layer was extracted with ethyl acetate. The combined organic extracts were washed with H₂O and brine, dried (Na₂SO₄), filtered and concentrated. The residue was purified by silica gel column chromatography (hexane/ethyl acetate, 80:20) to afford 408 mg of the title compound as a white solid: ES-MS: 477.0 [M+H]⁺; t_{R}=5.82 min (Method C); R_{f}=0.14 (hexane/ethyl acetate, 80:20).

### Step BV.3

### [trans-(4-Chloro-pyrrolo[2,3-d]pyrimidin-7-yl)-cyclohexyl]-carbamic acid tert-butyl ester

A mixture of (4,6-dichloro-pyrimidin-5-yl)-acetadehyde (555 mg, 2.91 mmol), N-(trans-4-aminocyclohexyl)-carbamic acid 1,1-dimethylethyl ester (623 mg, 2.91 mmol) and DIEA (508 μL, 2.91 mmol) in EtOH (5 mL) was stirred for 18 h at reflux, allowed to cool at rt, concentrated and diluted with DCM/H₂O. The aqueous layer was extracted with DCM. The combined organic extracts were dried (Na₂SO₄), filtered and concentrated. The residue was purified by silica gel column chromatography (hexane/ethyl acetate, 70:30) to afford to afford 348 mg of the title compound as a white soild: ES-MS: 351.1 [M+H]⁺; t_{R}=4.86 min (Method C), R_{f}=0.29 (hexane/ethyl acetate, 70:30).

### Intermediate BW

### 1-[cis-3-(4-Amino-5-bromo-pyrrolo[2,3-d]pyrimidin-7-yl)-cyclobutylmethyl]-piperidin-4-ol

The title compound was prepared in analogy to the procedure described in step BV.1 but using the intermediate prepared in step BW.1 and DCM as the solvent for dilution and extraction. The crude material was purified by silica gel column chromatography (DCM/MeOH/NH₃^{aq}, 91.5:7.5:1) to afford 56 mg of the title compound as a white foam: ES-MS: 380/382 [M+H]⁺; R_{f}=0.06 (DCM/MeOH/NH₃^{aq}, 91.5:7.5:1).

### Step BW. 1

### 1-[cis-3-(5-Bromo-4-chloro-pyrrolo[2,3-d]pyrimidin-7-yl)-cyclobutylmethyl]-piperidin-4-ol

A mixture of the intermediate prepared in step BW.2 (185 mg, 0.577 mmol), bromine (0.036 mL, 0.692 mmol, 1.2 eq) and AcOH (1 mL) was stirred for 30 min at rt, concentrated, diluted with NaHCO₃^{sat}/DCM and extracted with DCM. The combined organic layers were dried (Na₂SO₄), filtered and concentrated. The residue was purified by silica gel column chromatography (DCM/MeOH/NH₃^{aq}, 94:5:1) to afford 96 mg of the title compound: ES-MS: 399/401 [M+H]⁺; R_{f}=0.19 (DCM/MeOH/NH₃″, 94:5:1).

### Step BW.2

### 1-[cis-3-(4-Chloro-pyrrolo[2,3-d]pyrimidin-7-yl)-cyclobutylmethyl]-piperidin-4-ol

A mixture of (4,6-dichloro-pyrimidin-5-yl)-acetadehyde (612 mg, 3.20 mmol), the intermediate prepared in step BW.3 (590 mg, 3.20 mmol) and DIEA (559 μL, 3.20 mmol) in EtOH (10 mL) was stirred for 2 h at reflux, allowed to cool at rt, concentrated, diluted with a 6N aqueous solution of HCl, stirred for 10 min, basified by addition of NaHCO₃ and extracted with DCM. The residue was purified by silica gel column chromatography (DCM/MeOH/NH₃^{aq}, 94:5:1) to afford 702 mg of the title compound: ES-MS: 321 [M+H]⁺; R_{f}=0.14 (DCM/MeOH/NH₃^{aq}, 94:5:1).

### Step BW.3

### 1-(cis-3-Amino-cyclobutylmethyl)-piperidin-4-ol

A mixture of the intermediate prepared in step BW.4 (1.03 g, 3.2 mmol) and palladium on carbon (200 mg) in MeOH (20 mL) was stirred for 16 h at rt and under a hydrogen atmosphere. The reaction mixture was filtered through a pad of celite and concentrated to afford 595 mg of the title compounds as a white solid: API-ES: 185 [M+H]⁺.

### Step BW.4

### [cis-3-(4-Hydroxy-piperidin-1-ylmethyl)-cyclobutyl]-carbamic acid benzyl ester

Sodium triacetoxyborohydride (1 g, 4.7 mmol, 1.1 eq) was added to a mixture of the intermediate prepared in step BC.5 (1 g, 4.3 mmol) and 4-hydroxypiperidine (0.87 g, 8.6 mmol, 2 eq) in DCM (20 mL), under an argon atmosphere. The resulting mixture was stirred for 10 min at rt, quenched by addition of NaHCO₃^{sat} and extracted with DCM. The combined organic layers were washed with H₂O, brine, dried (Na₂SO₄), filtered and concentrated. The residue was purified by silica gel column chromatography (DCM/MeOH/NH₃^{aq}, 91.5:7.5:1) to afford 1 g of the title compound as a white solid: ES-MS: 319.2 [M+H]+; R_{f}=0.15 (DCM/MeOH/NH₃^{aq}, 91.5:7.5:1).

### Intermediate BX

### 8-[cis-3-(4-Amino-5-bromo-pyrrolo[2,3-d]pyrimidin-7-yl)-cyclobutylmethyl]-8-aza-bicyclo[3.2.1]octan-(3-exo)-ol

The title compound was prepared in analogy to the procedure described for intermediate BW but with the following modifications. Regarding the procedure of intermediate BW, the crude product was purified by silica gel column chromatography (DCM/MeOH/NH₃^{aq}, 89:10:1). In step BW.1, 1 eq of Br₂ was used. In step BW.2, the reaction mixture was stirred for 1 h after addition of 6 N HCl. In step BW.3, the reaction time was 1 h and the intermediate prepared in step BX.1 was used. The title compound: ES-MS: 406/408 [M+H]⁺; R_{f}=0.14 (DCM/MeOH/NH₃^{aq}, 89:10:1).

### Step BX.1

### [cis-3-((3-exo)-Hydroxy-8-aza-bicyclo[3.2.1]oct-8-ylmethyl)-cyclobutyl]-carbamic acid benzyl ester

Sodium triacetoxyborohydride (1.8 g, 8.6 mmol, 2 eq) was added to a mixture of the intermediate prepared in step BC.5 (1 g, 4.3 mmol) and 8-aza-bicyclo[3.2.1]octan-(3-exo)-ol (Baeckvall, J. E.; Renko, Z. D.; Bystroem, S. E.; Tetrahedron Letters (1987), 28(36), 4199-4202) (0.91 g, 5.6 mmol, 1.3 eq) in DCM (20 mL), under an argon atmosphere. The resulting mixture was stirred for 2 h at rt, quenched by addition of NaHCO₃^{sat} and extracted with DCM. The combined organic layers were dried (Na₂SO₄), filtered and concentrated. The residue was purified by silica gel column chromatography (DCM/MeOH/NH₃^{aq}, 94:5:1) to afford 1.1 g of the title compound as a white solid: ES-MS: 345.3 [M+H]⁺; R_{f}=0.07 (DCM/MeOH/NH₃^{aq}, 94:5:1).

### Intermediate BY

### 1-{4[cis-3-(4-Amino-5-bromo-pyrrolo[2,3-d]pyrimidin-7-yl)-cyclobutyl]-piperazin-1-yl}-ethanone

The title compound was prepared in analogy to the procedure described in step BV.1 but with the following modifications. The intermediate prepared in step BY.1 was used. The reaction mixture was stirred for 18 h at 120° C., concentrated and the residue was purified by silica gel column chromatography (DCM/MeOH/NH₃^{aq}, 98:1:1 then 95:4:1) to afford the title compound: ES-MS: 393/395 [M+H]⁺; R_{f}=0.44 (DCM/MeOH, 9:1).

### Step BY.1

### 1-{4-[cis-3-(5-Bromo-4-chloro-pyrrolo[2,3-d]pyrimidin-7-yl)-cyclobutyl]-piperazin-1-yl}-ethanone

A mixture of the intermediate prepared in step BY.2 (960 mg, 2.88 mmol), bromine (0.148 mL, 2.88 mmol) and AcOH (6 mL) was stirred for 30 min at rt, quenched by addition of NaHCO₃^{sat} and extracted with DCM. The combined organic layers were washed with NaHCO₃^{sat}, dried (Na₂SO₄), filtered and concentrated. The residue was purified by silica gel column chromatography (DCM/MeOH/NH₃^{aq}, 98:1:1 then 97:2:1) to afford 114 mg of the pure (cis isomer) title compound and 735 mg of an isomeric (cis/trans, 3:7) mixture (step BZ.1). The title compound (cis isomer): ES-MS: 412.1/414.1 [M+H]⁺; R_{f}=0.43 (DCM/MeOH, 9:1).

### Step BY.2

### 1-{4-[3-(4-Chloro-pyrrolo[2,3-d]pyrimidin-7-yl)-cyclobutyl]-piperazin-1-yl}-ethanone

The title compound was prepared in analogy to the procedure described in step BW.2 but with the following modifications. The intermediate prepared in step BY.3 and 2.2 eq of DIEA were used. The reaction mixture was stirred for 18 h at 80° C., concentrated, diluted with a 6N aqueous solution of HCl, stirred for 10 min, basified by addition of NaHCO₃ and extracted with DCM. The residue was purified by silica gel column chromatography (DCM/MeOH/NH₃^{aq}, 99:0:1 then 97:2:1) to afford the title compound: ES-MS: 334.2 [M+H]⁺; R_{f}=0.47 (DCM/MeOH, 9:1).

### Step BY.3

### 1-[4-(3-Amino-cyclobutyl)-piperazin-1-yl]-ethanone

A mixture of the intermediate prepared in step BY.4 (1.49 g, 5.0 mmol) and TFA (3.9 mL, 50 mmol, 10 eq) in DCM (4 mL) was stirred for 1 h at rt, concentrated, diluted with a 7 N solution of NH₃ in MeOH and concentrated. The residue was purified by silica gel column chromatography (DCM/MeOH/NH₃^{aq}, 96:3:1 then 92:7:1) to afford 843 mg of the title compound as a white solid: ES-MS: 198.2 [M+H]⁺; R_{f}=0.02 (DCM/MeOH, 9:1).

### Step BY.4

### [3-(4-Acetyl-piperazin-1-yl)-cyclobutyl]-carbamic acid tert-butyl ester

A mixture of (3-oxo-cyclobutyl)-carbamic acid tert-butyl ester (1 g, 5.40 mmol, purchased from PrincetonBio), 1-acetyl-piperazine (0.830 g, 6.48 mmol, 1.2 eq) and sodium triacetoxyborohydride (3.43 g, 16.20 mmol, 3 eq) in DCM (10 mL) was stirred for 1 h at rt, quenched by addition of NaHCO₃^{sat} and extracted with DCM. The combined organic layers were washed with NaHCO₃^{sat}, dried (Na₂SO₄), filtered and concentrated. The residue was purified by silica gel column chromatography (DCM/MeOH/NH₃^{aq}, 98:1:1 then 95:4:1) to afford 1.49 g of the title compound as a white solid: ES-MS: 298.3 [M+H]+; R_{f}=0.34 (DCM/MeOH, 9:1).

### Intermediate BZ

### 1-{4-[trans-(4-Amino-5-bromo-pyrrolo[2,3-d]pyrimidin-7-yl)-cyclobutyl]-piperazin-1-yl}-ethanone

The title compound was prepared in analogy to the procedure described in step BV.1 but with the following modifications. The intermediate prepared in step BZ.1 was used. The reaction mixture was stirred for 17 h at 120° C., concentrated and the residue was purified by silica gel column chromatography (DCM/MeOH/NH₃^{aq}, 98:1:1 then 95:4:1) to afford the title compound: ES-MS: 393/395 [M+H]⁺; R_{f}=0.29 (DCM/MeOH, 9:1).

### Step BZ. 1

### 1-{trans-4-[3-(5-Bromo-4-chloro-pyrrolo[2,3-d]pyrimidin-7-yl)-cyclobutyl]-piperazin-1-yl}-ethanone

The procedure is described in step BY.1. The title compound was obtained as an isomeric (cis/trans, 3:7) mixture: ES-MS: 412.1/414.1 [M+H]⁺; R_{f}=0.37 (DCM/MeOH, 9:1).

### Intermediate CA

### (S)-1-[cis-(4-Amino-5-bromo-pyrrolo[2,3-d]pyrimidin-7-yl)-cyclobutyl]-pyrrolidine-2-carboxylic acid amide

The title compound was prepared in analogy to the procedure described in step BV.1 but with the following modifications. The intermediate prepared in step CA.1 was used. The reaction mixture was stirred for 18 h at 120° C. DCM was used as the solvent for dilution and extraction. The crude product was not purified. The title compound: ES-MS: 379/381 [M+H]⁺.

### Step CA.1

### (S)-1-[cis-3-(5-Bromo-4-chloro-pyrrolo[2,3-d]pyrimidin-7-yl)-cyclobutyl]-pyrrolidine-2-carboxylic acid amide

Bromine (0.150 mL, 2.91 mmol) was slowly added to a solution of the intermediate prepared in step CA.2 (930 mg, 2.91 mmol) in AcOH (15 mL). The reaction mixture was stirred for 30 min at it, diluted with DCM/NaHCO₃^{sat} and extracted with DCM. The combined organic layers were washed with H₂O and brine, dried (Na₂SO₄), filtered and concentrated. The residue was purified by silica gel column chromatography (DCM/MeOH/NH₃^{aq}, 94:5:1) to afford 350 mg of the cis isomer (title compound) and 288 mg of the trans isomer (step CB.1). The title compound: ES-MS: 398/400 [M+H]⁺; R_{f}=0.34 (DCM/MeOH/NH₃^{aq}, 94:5:1).

### Step CA.2

### (S)-1-[-3-(4-Chloro-pyrrolo[2,3-d]pyrimidin-7-yl)-cyclobutyl]-pyrrolidine-2-carboxylic acid amide

A mixture of (4,6-dichloro-pyrimidin-5-yl)-acetadehyde (746 mg, 3.91 mmol), intermediate CA.3 (716 mg, 3.91 mmol) and DIEA (682 μL, 3.91 mmol) in EtOH (8 mL) was stirred for 16 h at reflux and allowed to cool at rt. Then, a 6 N aqueous solution of HCl (2 mL) was added. The resulting mixture was stirred for 2 h at rt, concentrated and diluted with DCM/NaHCO₃^{sat} and extracted with DCM. The combined organic extracts were dried (Na₂SO₄), filtered and concentrated. The residue was purified by silica gel column chromatography (DCM/MeOH/NH₃^{aq}, 94:5:1) to afford 932 mg of the title compound as a white solid: ES-MS: 320.2 [M+H]⁺; R_{f}=0.37 (DCM/MeOH/NH₃^{aq}, 94:5:1).

### Step CA.3

### (S)-1-(3-Amino-cyclobutyl)-pyrrolidine-2-carboxylic acid amide

A mixture of intermediate CA.4 (1.43 g, 5.1 mmol), TFA (2 mL) and DCM (10 mL) was stirred for 72 h at rt, concentrated, diluted with MeOH and loaded onto a Varian Mega BE-SCX column (10 g), washed with MeOH, eluted with a 7 N solution of NH₃ in MeOH to afford 740 mg of the title compound as a white solid: ES-MS: 184.1 [M+H]⁺.

### Step CA.4

### [3-((S)-2-Carbamoyl-pyrrolidin-1-yl)-cyclobutyl]-carbamic acid tert-butyl ester

Sodium triacetoxyborohydride (1.6 g, 7.6 mmol, 1.4 eq) was added to (3-oxo-cyclobutyl)-carbamic acid tert-butyl ester (1 g, 5.40 mmol, purchased from PrincetonBio) and L-prolinamide (0.74 g, 6.48 mmol, 1.2 eq) in DCM (20 mL). The resulting mixture was stirred for 2 h at rt, quenched by addition of NaHCO₃^{sat} and extracted with DCM. The combined organic layers were dried (Na₂SO₄), filtered and concentrated. The residue was purified by sillca gel column chromatography (DCM/MeOH/NH₃^{aq}, 91.5:7.5:1) to afford 1.4 g of the title compound as a white solid: ES-MS: 284.3 [M+H]⁺; R_{f}=0.40 (DCM/MeOH/NH₃^{aq}, 91.5:7.5:1).

### Intermediate CB

### (S)-1-[trans-3-(4-Amino-5-bromo-pyrrolo[2,3-d]pyrimidin-7-yl)-cyclobutyl]-pyrrolidine-2-carboxylic acid amide

The title compound was prepared in analogy to the procedure described in step BV.1 but with the following modifications. The intermediate prepared in step CB.1 (0.715 mmol), 6 mL of NH₄OH and 6 mL of EtOH were used. The reaction mixture was stirred for 18 h at 120° C. The crude product was not purified. The title compound: ES-MS: 379/381 [M+H]⁺.

### Step CB. 1

### (S)-1-[trans-3-(5-Bromo-4-chloro-pyrrolo[2,3-d]pyrimidin-7-yl)-cyclobutyl]-pyrrolidine-2-carboxylic acid amide

The title compound (trans isomer) was obtained after the purification of the crude material in step CA.1. The title compound: ES-MS: 398/400 [M+H]⁺; R_{f}=0.32 (DCM/MeOH/NH₃^{aq}, 94:5:1).

### Intermediate CC

### 5-Bromo-7-{cis-3-[(6-methyl-pyridin-2-ylamino)-methyl]-cyclobutyl}-7H-pyrrolo[2,3-d]pyrimidin-4-ylamine

A mixture of the intermediate prepared in step CC.1 (330 mg, 0.811 mmol), NH₄OH (30% NH₃ aqueous solution, 6 mL) and EtOH (6 mL) was stirred at 120° C. for 18 h in a sealed vessel, allowed to cool to it and diluted in ethyl acetate/H₂O. The aqueous layer was extracted with ethyl acetate. The combined organic extracts were washed with H₂O and brine, dried (Na₂SO₄), filtered and concentrated to afford 290 mg of the title compound as a yellow foam: ES-MS: 387/389 [M+H]⁺.

### Step CC.1

### [cis-3-(5-Bromo-4-chloro-pyrrolo[2,3-d]pyrimidin-7-yl)-cyclobutylmethyl]-(6-methyl-pyridin-2-yl)-amine

A solution of bromine (0.035 mL, 0.686 mmol, 0.5 eq) in AcOH (1 mL) was added slowly to a solution of the intermediate prepared in step CC.2 (450 mg, 1.37 mmol) in AcOH (9 mL) at rt. The mixture was concentrated, diluted with NaHCO₃^{sat} and extracted with DCM. The combined organic layers were washed with H₂O and brine, dried (Na₂SO₄), filtered and concentrated. The residue was purified by silica gel column chromatography (hexane/ethyl acetate, 40:60) to afford 313 mg of title compound as a colorless oil: ES-MS: 406/408 [M+H]⁺; R_{f}=0.27 (hexane/ethyl acetate, 40:60).

### Step CC.2

### [cis-3-(4-Chloro-pyrrolo[2,3-d]pyrimidin-7-yl)-cyclobutylmethyl]-(6-methyl-pyridin-2-y0-amine

A mixture of (4,6-dichloro-pyrimidin-5-yl)-acetadehyde (599 mg, 3.14 mmol), the intermediate prepared in step CC.3 (600 mg, 3.14 mmol) and DIEA (548 4, 3.14 mmol) in EtOH (6 mL) was stirred for 16 h at reflux, allowed to cool at rt, diluted with a 6 N aqueous solution of HCl, stirred for 2 h, diluted with NaHCO₃^{sat} and extracted with DCM. The combined organic layers were washed with H₂O and brine, dried (Na₂SO₄), filtered and concentrated. The residue was purified by silica gel column chromatography (DCM/ethyl acetate 50:50) to afford 700 mg of title compound as a yellow oil: ES-MS: 328.2 [M+H]⁺; R_{f}=0.31 (DCM/ethyl acetate 50:50).

### Step CC.3

### (cis-3-Amino-cyclobutylmethyl)-(6-methyl-pyridin-2-yl)-amine

The title compound was prepared in analogy to the procedure described in step BW.3 but with the following modifications. The intermediate prepared in step CC.3 was used; the reaction time was 2 h; the crude product was purified by silica gel column chromatography (DCM/MeOH/NH₃^{aq}, 94:5:1). The title compound: ES-MS: 192.2 [M+H]⁺; R_{f}=0.12 (DCM/MeOH/NH₃^{aq}, 94:5:1).

### Step CC.4

### {cis-3-[(6-Methyl-pyridin-2-ylamino)-methyl]-cyclobutyl}-carbamic acid benzyl ester

Sodium triacetoxyborohydride (1.36 g, 6.4 mmol, 1.5 eq) was added to a mixture of the intermediate prepared in step BC.5 (1 g, 4.3 mmol) and 6-methyl-2-aminopyridine (0.56 g, 5.1 mmol, 1.2 eq) in DCM (20 mL), under an argon atmosphere. The resulting mixture was stirred for 16 h at rt, quenched by addition of NaHCO₃^{sat} and extracted with DCM. The combined organic layers were washed with H₂O and brine, dried (Na₂SO₄), filtered and concentrated. The residue was purified by silica gel column chromatography (DCM/MeOH, 97.5:2.5) to afford 1.3 g of the title compound as a yellow oil: ES-MS: 326.3 [M+H]⁺; R_{f}=0.29 (DCM/MeOH, 97.5:2.5).

### Intermediate CD

### 4-[3-(4-Amino-5-bromo-pyrrolo[2,3-d]pyrimidin-7-yl)-cyclobutyl]-1-methyl-piperazin-2-one

The title compound was prepared in analogy to the procedure described in step BV.1 but with the following modifications. The intermediate prepared in step CD.1 was used. The reaction mixture was stirred for 18 h at 120° C. and concentrated. The crude product was purified by silica gel column chromatography (DCM/MeOH/NH₃^{aq}, 98:1:1 then 96:3:1). The title compound: ES-MS: 379/381 [M+H]⁺; R_{f}=0.35 (DCM/MeOH, 9:1).

### Step CD.1

### 4-[3-(5-Bromo-4-chloro-pyrrolo[2,3-d]pyrimidin-7-yl)-cyclobutyl]-1-methyl-piperazin-2-one

A solution of bromine (0.122 mL, 2.38 mmol) and the intermediate prepared in step CD.2 (760 mg, 2.38 mmol) in AcOH (5 mL) was stirred for 30 min at rt. The mixture was diluted with NaHCO₃^{sat} and extracted with DCM. The combined organic layers were washed with NaHCO₃^{aq}, dried (Na₂SO₄), filtered and concentrated. The residue was purified by silica gel column chromatography (DCM/MeOH/NH₃^{aq}, 99:0:1 then 97:2:1) to afford 650 mg of title compound: ES-MS: 398/400 [M+H]⁺; R_{f}=0.27 (DCM/MeOH, 9:1).

### Step CD.2

### 4-[3-(4-Chloro-pyrrolo[2,3-d]pyrimidin-7-yl)-cyclobutyl]-1-methyl-piperazin-2-one

The title compound was prepared in analogy to the procedure described in step BV.3 but with the following modifications. The intermediate prepared in step CD.3, 2 eq of DIEA and 2.6 mL of EtOHI1 mmol of reactant were used. The reaction mixture was quenched by addition of NaHCO₃^{sat} and extracted with DCM. The crude material was purified by silica gel column chromatography (DCM/MeOH/NH₃^{aq}, 99:0:1 then 96:3:1) to afford the title compound:

ES-MS: 320.2 [M+H]⁺; R_{f}=0.29 (DCM/MeOH, 9:1).

### Step CD.3

### 4-(3-Amino-cyclobutyl)-1-methyl-piperazin-2-one

A mixture of the intermediate prepared in step 16.5 (1.46 g, 5.2 mmol), TFA (10 mL) and DCM (10 mL) was stirred for 1 h at rt, quenched by addition of a 7 N solution of NH₃ in MeOH and concentrated. The residue was purified by silica gel column chromatography (DCM/MeOH/NH₃^{aq}, 97:2:1 then 93:6:1) to afford 706 mg of the title compound as a yellow oil: ES-MS: 184.2 [M+H]⁺; R_{f}=0.03 (DCM/MeOH, 9:1).

### Step CD.4

### [3-(4-Methyl-3-oxo-piperazin-1-yl)-cyclobutyl]-carbamic acid tert-butyl ester

Sodium triacetoxyborohydride (3.4 g, 16 mmol, 3 eq) was added to (3-oxo-cyclobutyl)-carbamic acid tert-butyl ester (1 g, 5.40 mmol, purchased from PrincetonBio) and 1-methyl-piperazin-2-one (0.74 g, 6.48 mmol, 1.2 eq) in DCM (10 mL). The resulting mixture was stirred for 1 h at rt, quenched by addition of NaHCO₃^{sat} and extracted with DCM. The combined organic layers were dried (Na₂SO₄), filtered and concentrated. The residue was purified by silica gel column chromatography (DCM/MeOH/NH₃″, 98:1:1 then 96:3:1) to afford 1.47 g of the title compound as a white solid: ES-MS: 284.3 [M+H]+; R_{f}=0.56 (DCM/MeOH, 9:1).

Intermediate CE: (Z)-3-(4-Amino-5-[3-(7-oxa-bicyclo[2.2.1]hept-1-ylmethoxy)-phenyl]-pyrrolo[2,3-d]pyrimidin-7-yl}-1-azidomethyl-cyclobutanol

The title compound was synthesized according to the synthesis of Intermediate BL.1 starting from toluene-4-sulfonic acid (Z)-3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl ester (Intermediate BL.3). Colorless solid. HPLC/MS t_{R}=0.99 min, M+H=462.3 (method X). ¹H NMR (600 MHz, DMSO-dₑ) δ ppm 8.12 (s, 1H, pyrimidinyl), 7.63 (s, 1H, pyrrolyl), 7.36 (t, 1H, phenyl), 7.07 (s, 1H, phenyl), 7.04/6.96 (d/d, 1H/1H, phenyl), 6.15 (s/b, 2H, NH₂), 5.61 (s, 1H, OH), 5.44 (quintet, 1H, CH-cyclobutyl), 4.50 (s, 1H, CH-oxabicycloheptanyl), 4.29 (s, 2H, CH₂—O), 3.43 (s, 2H, CH₂—N), 2.58/2.44 (m/m, 4H, CH₂-cyclobutyl), 1.68/1.56 (m/m, 8H, CH₂-oxabicycloheptanyl).

### Intermediate CF

### Toluene-4-sulfonic acid 3-{4-amino-5-[3-(7-oxa-bicyclo[2.2.1]hept-1-ylmethoxy)-phenyl]-pyrrolo[2,3-d]pyrimidin-7-yl}-cyclobutylmethyl ester

The title compound was prepared in an analagous manner to Intermediate AA using (cis3-{4-amino-5-[3-(7-oxa-bicyclo[2.2.1]hept-1-ylmethoxy)-phenyl]pyrrolo[2,3-d]pyrimidin-7-yl}-cyclobutyl)-methanol (Example 22) in place of (cis-3-{4-amino-5-[3-(tetrahydro-pyran-2-ylmethoxy)-phenyl]-pyrrolo[2,3-d]pyrimidin-7-yl}-cyclobutyl)-methanol (Intermediate P).

### Intermediate CG

### (R)-5-[3-(7-oxa-bicyclo[2.2.1]hept-1-ylmethoxy)-phenyl]-7-pyrrolidin-3-yl-7H-pyrrolo[2,3-d]pyrimidin-4-ylamine

To the stirred solution of (R)-3-{4-amino-5-[3-(7-oxa-bicyclo[2.2.1]hept-1-ylmethoxy)-phenyl]-pyrrolo[2,3-d]pyrimidin-7-yl}-pyrrolidine-1-carboxylic acid tert-butyl ester (Step CG.1, 1.15 g, 2.15 mmol) and DCM (18 ml) was added trifluoroacetic acid (3.6 ml) at 0° C. The reaction mixture was stirred for 17 hours at room temperature. The reaction mixture was then partitioned between NaOH 2 M (cold) and EtOAc. The combined organic layers were washed with brine, dried (Na₂SO₄), filtered and concentrated. The residue was purified by silica gel column chromatography (DCM/MeOH/NH₃^{aq}, 40:10:1) to afford 0.82 g of the title compound as beige foam: HPLC-MS: M+H=406.1 (Rₜ=0.74) (Method X) (:TLC R_{f}=0.18 (DCM/MeOH/NH₃^{aq}, 200:20:1).

### Step CG.1

### (R)-3-{(4-amino-5-[3-(7-oxa-bicyclo[2.2.1]hept-1-ylmethoxy)-phenyl]-pyrrolo[2,3-d]pyrimidin-7-yl}-pyrrolidine-1-carboxylic acid tert-butyl ester

(R)-3-(4-Amino-5-iodo-pyrrolo[2,3-d]pyrimidin-7-yl)-pyrrolidine-1-carboxylic acid tert-butyl ester (Step CG.2, 1.07 g, 2.49 mmol) and 1-methyl-4-[3-(4,4,5,5-tetramethyl-[1,3,2]dioxaborolan-2-yl)-phenoxymethyl]-7-oxa-bicyclo[2.2.1]heptane (intermediate BB; 1.04 g, 2.99 mmol, 1.2 eq) were dissolved in DMF (23.5 mL). Water (1.18 mL), K₃PO₄ (1.06 g, 4.99 mmol, 2 eq), Na₂CO₃ (0.53 g, 4.99 mmol, 2 eq) and tetrakis (triphenylphosphine) palladium (0.144 g, 0.125 mmol, 0.05 eq) were added and the reaction mixture purged with argon. It was then heated to 100° C. and stirred at this temperature for 1 h. The reaction mixture was then partitioned between brine and EtOAc. The combined organic layers were washed with brine, dried (Na₂SO₄), filtered and concentrated. The residue was purified by silica gel column chromatography (DCM/MeOH, 20:1) to afford 1.18 g of the title compound as beige foam: HPLC-MS: M+H=506.6 (Rₜ=1.02) (Method X): TLC Rf=0.22 (DCM/MeOH, 20:1).

### Step CG.2

### (R)-3-(4-amino-5-iodo-pyrrolo[2,3-d]pyrimidin-7-yl)-pyrrolidine-1-carboxylic acid tert-butyl ester

A mixture of (R)-3-(4-chloro-5-iodo-pyrrolo[2,3-d]pyrimidin-7-yl)-pyrrolidine-1-carboxylic acid tert-butyl ester (Step CG.3, 5.24 g, 11.68 mmol), 25% aqueous ammonia solution (40 mL) and 1,4-dioxane (40 mL) was heated in sealed tube at 100° C. for 16 hours. After cooling the reaction mixture was concentrated. The residue was triturated (water-ethanol, 4:1) and filtration, afforded the title compound, as beige solid: HPLC-MS: M+H=430.3 (Rₜ=1.05) (methode X): TLC R_{f}=0.26 (DCM/MeOH, 20:1).

### Step CG.3

### (R)-3-(4-chloro-5-iodo-pyrrolo[2,3-d]pyrimidin-7-yl)-pyrrolidine-1-carboxylic acid tert-butyl ester

A mixture of (R)-3-(4-chloro-pyrrolo[2,3-d]pyrimidin-7-yl)-pyrrolidine-1-carboxylic acid tert-butyl ester (Step CG.4, 6.60 g, 20.45 mmol) and NIS (5.06 g, 22.49 mmol, 1.1 eq) in DMF (82 mL) was stirred for 19 h at rt. The reaction mixture was then partitioned between water and EtOAc. The combined organic layers were washed with water and brine, dried (Na₂SO₄), filtered and concentrated. The residue was purified by silica gel column chromatography (DCM/MeOH, 30:1) to afford 5.25 g of the title compound as grey crystals: ES-MS: M+H=448.9; TLC R_{f}=0.56 (DCM/MeOH, 20:1).

### Step CG.4

### (R)-3-(4-Chloro-pyrrolo[2,3-d]pyrimidin-7-yl)-pyrrolidine-1-carboxylic acid tert-butyl ester

To the stirred solution of (R)-3-amino-pyrrolidine-1-carboxylic acid tert-butyl ester (CAS 147081-49-0; 4.68 g, 25.1 mmol, 1.0 eq) and EtOH (50 mL) was added N,N-diisopropylamine (4.30 mL, 25.1 mmol, 1.0 eq) and 4,6-dichloro-pyrimidin-5-yl)-acetaldehyde (CAS16019-33-3; 4.80 g, 25.1 mmol,). The reaction mixture was stirred for 8 h at reflux temperature and then concentrated. The residue was dissolved in THF (37.5 mL) and HCl 4M (12.5 mL) was added at 0° C. and stirred for an additional hour at 0° C. The reaction mixture was partitioned between Na₂CO₃ 1 Mand EtOAc. The combined organic layers were washed with brine, dried (Na₂SO₄), filtered and concentrated. The residue was purified by silica gel column chromatography (EtOAc-nheptane 1:1) to afford 6.70 g of the title compound as a yellow oil: HPLC-MS: M+H=323.4 (Rₜ=1.27) (Method X): TLC; R_{f}=0.19 (EtOAc-n heptane 1:2).

### Intermediate CH

### (E)-3-{4-amino-5-[3-(7-oxa-bicyclo[2.2.1]hept-1-ylmethoxy)-phenyl]-pyrrolo[2,3-d]pyrimidin-7-yl}-1-(1H-tetrazol-5-ylmethyl)-cyclobutanol

The mixture of (E)-(3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutyl)-acetonitrile (Step CH.1, 84 mg, 0.187 mmol), tributyltin azide (127 mg, 0.373 mmol, 2 eq) and xylole (1.5 mL) was stirred at 145° C. for 15 h. The reaction mixture was concentrated. The residue was triturated (n-hexane), filtered to afford 121 mg of the title compound (crude), as beige solid: HPLC-MS: M+H=489.4 (Rₜ=0.80) (Method X).

### Step CH.1

### (E)-(3-{(4-amino-5-[3-(7-oxa-bicyclo[2.2.1]hept-1-ylmethoxy)-phenyl]-pyrrolo[2,3-d]pyrimidin-7-yl}-1-hydroxy-cyclobutyl)-acetonitrile

The mixture of toluene-4-sulfonic acid (E)-3-}(4-amino-5-[3-(7-oxa-bicyclo[2.2.1]hept-1-yl methoxy)-phenyl]-pyrrolo[2,3-d]pyrimidin-7-yl}-1-hydroxy-cyclobutyl methyl ester (Intermediate BL.2; 179 mg, 0.30 mmol), KCN (195 mg, 3.0 mmol, 10 eq) and DMF (1.8 mL) was stirred for 1 h at 65° C. The reaction mixture was partitioned between water and EtOAc. The combined organic layers were washed with water and brine, dried (Na₂SO₄), filtered and concentrated. The residue was purified by silica gel column chromatography (DCM/MeOH/NH₃^{aq}, 200:20:1) to afford 86 mg of the title compound as beige foam: HPLC-MS: M+H=446.3 (Rₜ=0.90) (Method X).

### Intermediate CI.1

### (E)-N-(3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl)-2-chloro-acetamide

To the stirred mixture of 1-aminomethyl-(E)-3-{4-amino-5-[3-(7-oxa-bicyclo[2.2.1]hept-1-ylmethoxy)-phenyl]-pyrrolo[2,3-d]pyrimidin-7-yl}-cyclobutanol (Intermediate BL; 66 mg, 0.15 mmol), DCM (1.5 mL) and NaOH 4 M (0.225 mL, 0.90 mmol, 6 eq) was added chloro-acetyl chloride (0.017 mL, 0.216 mmol, 1.44 eq) at 0° C. The reaction mixture was stirred for an additional hour at 0° C. and then partitioned between NaHCO₃ 1 Mand EtOAc. The combined organic layers were washed with brine, dried (Na₂SO₄), filtered and concentrated. The residue was purified by silica gel column chromatography (DCM/MeOH/NH₃^{aq}, 200:20:1) to afford 55 mg of the title compound as white crystals: HPLC-MS: M+H=512.3 (Rₜ=0.86) (Method X); TLC; R_{f}=0.26 (DCM/MeOH/NH₃^{aq}, 200:20:1).

### Intermediate CJ

### (Z)-3-{4-Amino-5-[3-(7-oxa-bicyclo[2.2.1]hept-1-ylmethoxy)-phenyl]-pyrrolo[2,3-d]pyrimidin-7-yl}-cyclobutanol

To the stirred solution of (Z)-7-(3-azido-cyclobutyl)-5-[3-(7-oxa-bicyclo[2.2.1]hept-1-ylmethoxy)-phenyl]-7H-pyrrolo[2,3-d]pyrimidin-4-ylamine (Step CJ.1, 75 mg, 0.138 mmol) and THF (0.30 mL) was added subsequently water (0.075 mL), MeOH (0.30 mL), ammonium hydroxide (25% in water; 0.085 mL, 0.551 mmol, 4 eq) and triphenylphosphine (54 mg, 0.206 mmol, 1.5 eq). The reaction mixture was stirred for 16 h at rt and then partitioned between water and EtOAc. The combined organic layers were washed with brine, dried (Na₂SO₄), filtered and concentrated. The residue was purified by silica gel column chromatography (DCM/MeOH/NH_{3′, 200:20:1} and DCM/MeOH/NH₃^{aq}, 40:10:1) to afford 51 mg of the title compound as beige foam: HPLC-MS: M+H=406.3 (Rₜ=0.68) (Method X); TLC; R_{f}=0.15 (DCM/MeOH/NH₃^{aq}, 200:20:1).

### Step CJ. 1

### (Z)-7-(3-azido-cyclobutyl)-5-[3-(7-oxa-bicyclo[2.2.1]hept-1-ylmethoxy)-phenyl]-7H-pyrrolo[2,3-d]pyrimidin-4-ylamine

To the stirred solution of (E)-3-{4-amino-5-[3-(7-oxa-bicyclo[2.2.1]hept-1-ylmethoxy)-phenyl]-pyrrolo[2,3-d]pyrimidin-7-yl}-cyclobutanol (Example 222, 100 mg, 0.244 mmol) and pyridine (2.44 mL) was added tosyl chloride (163 mg, 0.852 mmol, 3.5 eq) in portions at 0° C. over a period of 4 h. The reaction mixture was stirred for 20 h at 0° C. and then partitioned between brine and EtOAc. The combined organic layers were washed with brine, dried (Na₂SO₄), filtered and concentrated. The residue (tosylate intermediate) was dissolved in DMF (1.22 mL) and then sodium azide was added. The mixture was stirred for 8 h at 65° C. and then partitioned between brine and EtOAc. The combined organic layers were washed with brine, dried (Na₂SO₄), filtered and concentrated. The residue was purified by silica gel column chromatography (DCM/MeOH/NH₃^{aq}, 200:10:1) to afford 78 mg of the title compound as a beige foam: HPLC-MS: M+H=432.3 (Rₜ=1.16) (Method X); TLC; R_{f}=0.63 (DCM/MeOH/NH₃^{aq}, 200:20:1).

### Intermediate CK

### 7-azetidin-3-yl-5-[3-(7-oxa-bicyclo[2.2.1]hept-1-ylmethoxy)-phenyl]-7H-pyrrolo[2,3-d]pyrimidin-4-ylamine

The title compound was prepared in a similar manner to Intermediate CG starting from 3-amino-azetidine-1-carboxylic acid tert-butyl ester and 4,6-dichloro-pyrimidin-5-yl)-acetaldehyde (CAS16019-33-3;). ¹H NMR (600 MHz, DMSO-d₆) δ ppm 8.12 (s, 1H), 7.69 (s, 1H), 7.37 (t, 1H), 7.09 (s, 1H), 7.06 (d, 1H), 6.97 (d, 1H), 6.15 (bs, 2H), 5.49 (m, 1H), 4.50 (t, 1H), 4.30 (s, 2H), 3.98 (t, 2H), 3.84 (t, 2H), 1.68 (m, 4H), 1.56 (m, 4H).

### Intermediate CL

### 5-lodo-7-{3-[(1S,4S)-1-(2-thia-5-aza-bicyclo[2.2.1]hept-5-yl)methyl]-cyclobutyl}-7H pyrrolo[2,3-d]pyrimidin-4-ylamine

A mixture of 4-chloro-5-iodo-7-{3-[(15,45)-1-(2-thia-5-aza-bicyclo[2.2.1]hept-5-yl)methyl]-cyclobutyl}-7H-pyrrolo[2,3-d]pyrimidine (Step CL.1, 60 mg, 0.094 mmol), 25% aqueous ammonia solution (0.29 mL) and 1,4-dioxane (0.94 mL) was heated in sealed tube for 15 h at 100° C. After cooling the reaction mixture was concentrated and the residue was purified by silica gel column chromatography (DCM/MeOH/NH₃^{aq}, 200:20:1) to afford 33 mg of the title compound as beige foam: HPLC-MS: M+H=442.1 (Rₜ=0.53) (Method X); TLC; R_{f}=0.44 (DCM/MeOH/NH₃^{aq}, 200:20:1).

### Step CL.1

### 4-chloro-5-iodo-7-{3-[(1S,4S)-1-(2-thia-5-aza-bicyclo[2.2.1]hept-5-yl)methyl]-cyclobutyl}-7H-pyrrolo[2,3-d]pyrimidine

To the stirred solution of 3-(4-chloro-5-iodo-pyrrolo[2,3-d]pyrimidin-7-yl)-cyclobutane carbaldehyde (Step CL.2, 212 mg, 0.587 mmol) and 1,2-dichloroethane (5.9 mL) was added subsequently N-ethyldiisopropylamine (1.05 mL, 5.87 mmol, 10 eq) and (1S,4S)-2-thia-5-aza bicyclo[2.2.1]heptane (Bioorganic & Medicinal Chemistry Letters (2009) 19(15) 4130-3) (98 mg, 0.646 mmol, 1.1 eq). After stirring 0.5 h at rt, sodium triacetoxyborohydride (328 mg, 1.469 mmol, 2.5 eq) was added and stirring was continued for 2 h. The reaction mixture was then partitioned between NaHCO₃ 1 Mand DCM. The combined organic layers were washed with brine, dried (Na₂SO₄), filtered and concentrated. The residue was purified by silica gel column chromatography (DCM/MeOH/NH₃^{aq}, 200:20:1) to afford 193 mg of the title compound as white foam: HPLC-MS: M+H=461.0 (Rₜ=0.83) (Method X); TLC; R_{f}=0.60 (DCM/MeOH/NH₃^{aq}, 200:20:1).

### Step CL.2

### 3-(4-chloro-5-iodo-pyrrolo[2,3-d]pyrimidin-7-yl) cyclobutane carbaldehyde

To a stirred solution of (Z)-3-(4-chloro-5-iodo-pyrrolo[2,3-d]pyrimidin-7-yl)-cyclobutanecarboxylic acid methyl ester (prepared as described in WO2005097800, 2.77 g, 7.07 mmol) and DCM (40 mL) was added slowly DIBAL-H (1.7 M in toluene) (5.41 mL, 9.20 mmol, 1.3 eq) at −78° C. Stirring was continued for 4 h at −78° C. The reaction mixture was quenched with sat. ammonium chlorid (5 mL). The reaction mixture was partitioned between HCl 1 Mand DCM. The combined organic layers were washed with brine, dried (Na₂SO₄), filtered and concentrated. The residue was purified by silica gel column chromatography (EtOAc-n hexane 1:1) to afford 2.01 g of the title compound as white solid: HPLC-MS: M+H=362.0 (Rₜ=1.13. ¹H NMR (400 MHz, DMSO-d₆) δ ppm 9.70 (s, 1H), 8.62 (s, 1H), 8.26 (s, 1H), 5.29 (m, 1H), 3.08 (m, 1H), 2.73 (m, 2H), 2.61 (m, 2H).

### Intermediate CM

### 5-iodo-7-[cis-3-(1-oxo-thiomorpholin-4-ylmethyl)-cyclobutyl]-7H-pyrrolo[2,3-d]pyrimidin-4-ylamine

To the stirred solution of [cis-3-(4-amino-5-iodo-pyrrolo[2,3-d]pyrimidin-7-yl)-cyclobutyl]methanol (Intermediate M: 348 mg, 1.0 mmol) and acetonitrile (70 mL) was added IBX (Atlantic SciTech 86900: 561 mg, 2.0 mmol, 2 eq). The reaction mixture was stirred for 1 h at 80° C. The reaction mixture was filtered at 40° C. and the filtrate was concentrated. To the residue was added subsequently DCM (50 mL), ethyl-diisopropyl-amine (3.43 mL, 20 mmol, 20 eq), 1-oxide thiomorpholin hydrochloride (312 mg, 2.0 mmol, 2 eq) and sodium triacetoxyborohydride (637 mg, 3.0 mmol, 3 eq) with stirring at rt. The reaction mixture was stirred for 1 h at rt and then partitioned between NaHCO₃ 1 Mand EtOAc. The combined organic layers were washed with water and brine, dried (Na₂SO₄), filtered and concentrated. The residue was purified by silica gel column chromatography (DCM/MeOH/NH₃^{aq}, 200:20:1) to afford 238 mg of the title compound as pale yellow crystals: HPLC-MS: M+H=446.2 (Rₜ=0.41) (Method X); TLC; R_{f}=0.26 (DCM/MeOH/NH₃^{aq}, 200:20:1).

### Intermediate CN

### 5-iodo-7-[cis-3-(1-oxo-thiomorpholin-4-yl)-cyclobutyl]-7H-pyrrolo[2,3-d]pyrimidin-4-ylamine

To the stirred solution of 3-(4-amino-5-iodo-pyrrolo[2,3-d]pyrimidin-7-yl)-cyclobutanone (Intermediate BG; 680 mg, 2.05 mmol), 1,2-dichloroethane (55 ml) and diisopropylethylamine (1.79 ml, 10.25 mmol) was subsequently added 1-oxo-thiomorpholine hydrochloride (638 mg, 4.10 mmol) and sodium triacetoxyborohydride (652 mg, 3.08 mmol) at 0° C. The reaction mixture was stirred for 1 h at room temperature and then poured into the stirred mixture of water (150 ml) and EtOAc (150 ml). The precipitate was filtered and washed with water and EtOAc. The solid collected was dried in vacuo to afford the title compound as beige crystals. HPLC-MS: M+H=432.1 (Rₜ=0.43); TLC; R_{f}=0.36 (DCM/MeOH/NH₃^{aq}, 200:20:1).

### Intermediate CO

### 2-(4-Amino-5-iodo-pyrrolo[2,3-d]pyrimidin-7-yl)-5-oxa-7-aza-spiro[3.4]octan-6-one

3-(4-Amino-5-iodo-pyrrolo[2,3-d]pyrimidin-7-yl)-1-aminomethyl-cyclobutanol (837 mg) was suspended in THF/DMF (5/1) (24 ml) and CDI (453 mg) was added. The reaction mixture was stirred at ambient temperature for 150 minutes. The reaction was left over night in solution. The THF was removed and water (40 mL) was added and a precipitate was obtained which was filtered off to give the title compound as a white solid (642 mg). HPLC/MS t_{R} 0.55 min, M+H 386.0 (Method X). ¹H NMR (400 MHz, DMSO-d₆) δ ppm 2.73-2.96 (m, 4H), 3.56 (s, 2H), 5.22 (m, 1H), 6.61 (bs, 1H), 7.53 (s, 1H), 7-71 (s, 1H), 8.08 (s, 1H).

### Step CO.1

### 3-(4-Amino-5-iodo-pyrrolo[2,3-d]pyrimidin-7-yl)-1-aminomethyl-cyclobutanol

The title compound was prepared in similar manner to intermediate BL using 3-(4-Amino-5-iodo-pyrrolo[2,3-d]pyrimidin-7-yl)-1-azidomethyl-cyclobutanol (step CO.2) as the starting material. HPLC/MS t_{R} 0.37 min, M+H 360.1 (Method X). ¹H NMR (400 MHz, DMSO-d₆) δ ppm 2.16-2.33 (m, 2H), 2.39-2.54 (m, 2H), 2.58 (s, 2H), 5.29 (m, 1H), 6.55 (bs, 1H), 7.66 (s, 1H), 8.06 (s, 1H).

### Step CO.2

### 3-(4-Amino-5-iodo-pyrrolo[2,3-d]pyrimidin-7-yl)-1-azidomethyl-cyclobutanol

The title compound was prepared in similar manner to step BL.1 using Toluene-4-sulfonic acid 3-(4-amino-5-iodo-pyrrolo[2,3-d]pyrimidin-7-yl)-1-hydroxy-cyclobutylmethyl ester as the starting material. MS M+H 386.1. ¹H NMR (400 MHz, DMSO-d₆) δ ppm 2.23-2.46 (m, 2H), 2.46-2.62 (m, 2H), 3.41 (s, 2H), 5.35 (t, 1H), 5.61 (s, 1H), 6.57 (bs, 1H), 7.72 (s, 1H), 8.07 (s, 1H).

### Step CO.2

### Toluene-4-sulfonic acid 3-(4-amino-5-iodo-pyrrolo[2,3-d]pyrimidin-7-yl)-1-hydroxy-cyclobutylmethyl ester

The title compound was prepared using a method similar to step BL.2 using (E)-3-(4-amino-5-iodo-pyrrolo[2,3-d]pyrimidin-7-yl)-1-hydroxymethyl-cyclobutanol (Intermediate BT (trans)) as the starting material. HPLC/MS t_{R} 0.87 min, M+H 515.0 (Method X). ¹H NMR (400 MHz, DMSO-d₆) δ ppm 2.21-2.36 (m, 2H), 2.36-2.57 (m, 2H), 2.40 (s, 3H), 4.06 (s, 1H), 5.26 (t, 1H), 5.57 (s, 1H), 6.58 (bs, 1H), 7.46 (d, 2H), 7.60 (s, 1H), 7.79 (d, 2H), 8.07 (s, 1H).

### Chemical Synthesis

### Compounds of the Invention

### Example 1

### (rac)-cis-7-(3-azetidin-1-ylmethyl-cyclobutyl)-5-[3-(tetrahydro-pyran-2-ylmethoxy)-phenyl]-7H-pyrrolo[2,3-d]pyrimidin-4-ylamine

In a 3 ml vial were introduced under argon cis-3-[4-amino-7-(3-azetidin-1-ylmethyl-cyclobutyl)-7H-pyrrolo[2,3-d]pyrimidin-5yl]-phenol (Intermediate A, 100 mg, 0.286 mmol), triphenylphosphine (120 mg, 0.458 mmol), THF (1.4 ml) and (rac)-2-tetrahydropyranylmethanol (Aldrich, Buchs, Switzerland; 39.9 mg, 0.343 mmol). DIAD (46.3 mg, 0.229 mmol) was added dropwise. The RM was stirred 15 h at rt then the solvent was blown off with N₂ and the residue was taken in EtOAc and extracted with 1 Maqueous HCl (2×). The aqueous layers were neutralized with 10 Maqueous NaOH and basified with 10% aqueous NaHCO₃, then extracted with EtOAc (2×). The organic layers are dried over Na₂SO₄, filtered and evaporated. The crude product was dissolved in DMA and MeOH, filtered and purified by prep HPLC. The fraction containing pure product was basified with NaHCO₃ (10 mg/ml), concentrated and extracted with EtOAc (3×). The organic layers were dried over Na₂SO₄, filtered and evaporated to give the title compound as a film. HPLC: t_{R} 2.63 min (Method A); M+H=448 MS-ES.

### Examples 2 & 3

### (R)- & (S)-cis-7-(3-azetidin-1-ylmethyl-cyclobutyl)-5-[3-(tetrahydro-pyran-2-ylmethoxy)-phenyl]-7H-pyrrolo[2,3-d]pyrimidin-4-ylamine

The racemic sample of cis-7-(3-azetidin-1-ylmethyl-cyclobutyl)-5-[3-(tetrahydro-pyran-2-ylmethoxy)-phenyl]-7H-pyrrolo[2,3-d]pyrimidin-4-ylamine (Example 1) was separated by chiral Prep.HPLC (Chiracel OD, EtOH/MeOH 1:1/diethylamine 0.1%) to give:

Example 2 as first eluting enantiomer (R)-cis-7-(3-azetidin-1-ylmethyl-cyclobutyl)-5-[3-(tetrahydro-pyran-2-ylmethoxy)-phenyl]-7H-pyrrolo[2,3-d]pyrimidin-4-ylamine (HPLC: t_{R} 2.63 min (Method A); M+H=448 MS-ES).
Example 3 as the second eluting enantiomer (S)-cis-7-(3-azetidin-1-ylmethyl-cyclobutyl)-5-[3-(tetrahydro-pyran-2-ylmethoxy)-phenyl]-7H-pyrrolo[2,3-d]pyrimidin-4-ylamine (HPLC: t_{R} 2.63 min (Method A); M+H=448 MS-ES.
NMR data were identical for Example 1, Example 2 & Example 3: ¹H NMR (d₆-DMSO, 400 MHz): 8.10 (s, 1H), 7.61 (s, 1H), 7.37-7.33 (m, 1H), 7.03-7.01 (m, 2H), 6.91-6.88 (m, 1H), 5.03 (qt, 1H), 3.98-3.86 (m, 3H), 3.66-3.60 (m, 1H), 3.41-3.34 (m, 1H), 3.07 (t, 4H), 2.50-2.44 (m, 4H), 2.18-2.02 (m, 3H), 1.92 (t, 2H), 1.83-1.77 (m, 1H), 1.66-1.63 (m, 1H), 1.53-1.43 (m, 3H), 1.37-1.27 (m, 1H)).

### Example 4

### cis-7-(3-[(1,1-dioxidothiomorpholin-4-yl)methyl]cyclobutyl}-5-{3-[(2S)-tetrahydrofuran-2-ylmethoxy]phenyl}-7H-pyrrolo[2,3-d]pyrimidin-4-amine

The title compound was synthesized in a similar manner as described for Example 1 using cis-3-{4-amino-7-[3-(1,1-dioxothiomorpholin-4-ylmethyl)-cyclobutyl]-7H-pyrrolo[2,3-d]pyrimidin-5-yl}-phenol (Intermediate B, 30 mg, 0.070 mmol) and (5)-tetrahydrofurfurylalcohol (3B Scientific, Libertyville, USA, 8.6 mg, 0.084 mmol) to give the title compound as a film. HPLC: t_{R} 2.45 min (Method A); M+H=512 MS-ES; ¹H-NMR (d₆-DMSO, 500 MHz): 8.15-8.11 (m, 1H), 7.68-7.64 (m, 1H), 7.41-7.35 (m, 1H), 7.08-7.02 (m, 2H), 6.95-6.90 (m, 1H), 6.4-5.85 (br, 2H), 5.10 (qt, 1H), 4.21-4.14 (m, 1H), 4.05-3.95 (m, 2H), 3.83-3.75 (m, 1H), 3.73-3.65 (m, 1H), 3.13-3.03 (m, 4H), 2.97-2.87 (m, 4H), 2.73-2.67 (m, 2H), 2.58-2.48 (m, 2H), 2.36-2.29 (m, 1H), 2.24-2.16 (m, 2H), 2.04-1.97 (m, 1H), 1.93-1.79 (m, 2H), 1.73-1.65 (m, 1H).

### Example 5

### cis-7-(3-azetidin-1-ylmethyl-cyclobutyl)-5-[3-(5-methyl-tetrahydro-furan-2-ylmethoxy)-phenyl]-7H-pyrrolo[2,3-d]pyrimidin-4-ylamine

The title compounds were synthesized in a similar manner as described for Example 1 using the diastereomeric mixture of 5-methyl-tetrahydro-furan-2-yl)-methanol (intermediate C).

### Examples 6, 7, 8 & 9

### cis-(RR, RS, SR and SS)-7-(3-azetidin-1-ylmethyl-cyclobutyl)-5-[3-(5-methyl-tetrahydro-furan-2-ylmethoxy)-phenyl]-7H-pyrrolo[2,3-d]pyrimidin-4-ylamine

The diastereoisomeric mixture Example 5 was separated by chiral Prep.HPLC on a Chiracel OD-H column (scCO₂/MeOH (1% 2-propylamine) 20% to 45%) to give:

Example 6 and Example 7: first and last eluting products being cis-(RR and SS)-7-(3-azetidin-1-ylmethyl-cyclobutyl)-5-[3-(5-methyl-tetrahydro-furan-2-ylmethoxy)-phenyl]-7H-pyrrolo[2,3-d]pyrimidin-4-ylamine enantiomers (HPLC t_{R} 2.64 min (Method A); M+H=448 MS-ES; ¹H-NMR (d₆-DMSO, 600 MHz): 8.11 (s, 1H), 7.61 (s, 1H), 7.37-7.35 (m, 1H), 7.04-7.02 (m, 2H), 6.92-6.90 (m, 1H), 6.3-5.9 (NH₂), 5.04 (qt, 1H), 4.16-4.12 (m, 1H), 4.02-3.91 (m, 3H), 3.08 (t, 4H), 2.50-2.43 (m, 4H), 2.17-1.90 (m, 6H), 1.79-1.71 (m, 1H), 1.46-1.41 (m, 1H), 1.16 (d, 3H)).
Example 8 and Example 9: second and third eluting products being cis-(RS and SR)-7-(3-Azetidin-1-ylmethyl-cyclobutyl)-5-[3-(5-methyl-tetrahydro-furan-2-ylmethoxy)-phenyl]-7H-pyrrolo[2,3-d]pyrimidin-4-ylamine (HPLC t_{R} 2.61 min (Method A); M+H=448 MS-ES; ¹H-NMR (d₅-DMSO, 600 MHz): 8.11 (s, 1H), 7.61 (s, 1H), 7.37-7.35 (m, 1H), 7.04-7.02 (m, 2H), 6.92-6.90 (m, 1H), 6.3-5.9 (NH₂), 5.04 (qt, 1H), 4.32-4.28 (m, 1H), 4.07-4.03 (m, 1H), 3.98-3.94 (m, 2H), 3.08 (t, 4H), 2.50-2.44 (m, 4H), 2.17-2.01 (m, 5H), 1.93 (t, 2H), 1.74-1.68 (m, 1H), 1.45-1.38 (m, 1H), 1.15 (d, 3H)).

### Example 10

### cis-7-[3-(1,1-dioxothiomorpholin-4-ylmethyl)-cyclobutyl]-5-[3-(5-methyl-tetrahydro-furan-2-ylmethoxy)-phenyl]-7H-pyrrolo[2,3-d]pyrimidin-4-ylamine

The title compound was prepared in a similar manner to Example 1 starting from cis-3-{4-amino-7-[3-(1,1-dioxothiomorpholin-4-ylmethyl)-cyclobutyl]-7H-pyrrolo[2,3-d]pyrimidin-5-yl}-phenol (Intermediate B) and (5-methyl-tetrahydro-furan-2-yl)-methanol (Intermediate C). HPLC t_{R} 2.57 min (Method A); M+H=526 MS-ES.

### Example 11

### 4-(3-{4-amino-5-[3-(5,5-dimethyl-tetrahydro-furan-2-ylmethoxy)-phenyl]-pyrrolo[2,3-d]pyrimidin-7-yl}-cyclobutylmethyl)-1-methyl-piperazin-2-one

The title compound was synthesized in a similar manner as described for Example 1 starting from 4-{3-[4-amino-5-(3-hydroxy-phenyl)-pyrrolo[2,3-d]pyrimidin-7-yl]-cyclobutylmethyl}-1-methyl-piperazin-2-one (Intermediate D) and 5,5-dimethyl-tetrahydro-furan-2-yl)-methanol (Intermediate E) to give the title compound as a film. HPLC: t_{R} 2.68 min (Method A); M+H=519 MS-ES; ¹H NMR (dₑ-DMSO, 400 MHz): 8.11-8.10 (m, 1H), 7.65-7.62 (m, 1H), 7.38-7.33 (m, 1H), 7.05-7.01 (m, 2H), 6.92-6.88 (m, 1H), 5.08 (qt, 1H), 4.27-4.20 (m, 1H), 4.01-3.91 (m, 2H), 3.33-3.30 (m, 1H), 3.26-3.20 (m, 2H), 2.96 (s, 2H), 2.79 (s, 3H), 2.65-2.60 (m, 2H), 2.55-2.46 (m, 4H), 2.23-2.03 (m, 3H), 1.83-1.68 (m, 3H), 1.19 (s, 3H), 1.17 (s, 3H).

### Example 12

### cis-7-(3-azetidin-1-ylmethyl-cyclobutyl)-5-[3-((S)-5,5-dimethyl-tetrahydro-furan-2-ylmethoxy)-phenyl]-7H-pyrrolo[2,3-d]pyrimidin-4-ylamine

The title compound was prepared in a similar manner to Example 1 starting from ((S)-5,5-dimethyl-tetrahydro-furan-2-yl)-methanol (Intermediate F). HPLC t_{R} 2.74 min (Method A); M+H=462 MS-ES.

### Example 13

### cis-7-(3-azetidin-1-ylmethyl-cyclobutyl)-5-[3-((R)-5,5-dimethyl-tetrahydro-furan-2-ylmethoxy)-phenyl]-7H-pyrrolo[2,3-d]pyrimidin-4-ylamine

The title compound was prepared in a similar manner to Example 1 starting from ((R)-5,5-dimethyl-tetrahydro-furan-2-yl)-methanol (Intermediate G). HPLC t_{R} 2.74 min (Method A); M+H=462 MS-ES.

### Example 14

### 5-[3-(5,5-dimethyl-tetrahydro-furan-2-ylmethoxy)phenyl]-7-[3-(1,1-dioxothiomorpholin-4-ylmethyl)-cyclobutyl]-7H-pyrrolo[2,3-d]pyrimidin-4-ylamine

The title compound was prepared in a similar manner to Example 1 starting from cis-3-O-amino-7-[3-(1,1-dioxothiomorpholin-4-ylmethyl)-cyclobutyl]-7H-pyrrolo[2,3-d]pyrimidin-5-yl}-phenol (Intermediate B) and (5,5-dimethyl-tetrahydro-furan-2-yl)-methanol (Intermediate E). HPLC t_{R} 2.70 min (Method A); M+H=540 MS-ES.

### Example 15

### 4-(3-{(4-amino-5-[3-(5-methyl-tetrahydro-furan-2-ylmethoxy)-phenyl]-pyrrolo[2,3-d]pyrimidin-7-yl}-cyclobutylmethyl)-1-methyl-piperazin-2-one

The title compound was prepared in a similar manner to Example 1 starting from 4-{3-[4-amino-5-(3-hydroxy-phenyl)-pyrrolo[2,3-d]pyrimidin-7-yl]-cyclobutylmethyl}-1-methyl-piperazin-2-one (Intermediate D) and (5-methyl-tetrahydro-furan-2-yl)-methanol (Intermediate C). HPLC t_{R} 2.55 min (Method A); M+H=505 MS-ES.

### Example 16

### cis-7-[3-(4,4-Difluoro-piperidin-1-ylmethyl)-cyclobutyl]-5-{3-[(S)-1-(tetrahydrofuran-2-yl)methoxy]-phenyl}-7H-pyrrolo[2,3-d]pyrimidin-4-ylamine

The title compound was synthesized in a similar manner as described for Example 1 using cis-3-{4-amino-7-[3-(1,1-dioxothiomorpholin-4-ylmethyl)-cyclobutyl]-7H-pyrrolo[2,3-d]pyrimidin-5-yl}-phenol (Intermediate H) and (S)-tetrahydrofurfurylalcohol (3B Scientific, Libertyville, USA, 8.6 mg, 0.084 mmol) to give the title compound as a film. HPLC: t_{R} 2.60 min (Method A); M+H=498 MS-ES; ¹H-NMR (d₆-DMSO, 400 MHz): 8.11-8.10 (m, 1H), 7.65-7.62 (m, 1H), 7.38-7.32 (m, 1H), 7.05-7.01 (m, 2H), 6.92-6.88 (m, 1H), 5.07 (qt, 1H), 4.19-4.14 (m, 1H), 4.03-3.93 (m, 2H), 3.80-3.74 (m, 1H), 3.70-3.63 (m, 1H), 2.57-2.46 (m, 9H), 2.22-2.12 (m, 2H), 2.04-1.75 (m, 7H), 1.71-1.62 (m, 1H).

### Example 17

### cis-7-(3-azetidin-1-ylmethyl-cyclobutyl)-5-{3-[(S)-1-(tetrahydro-furan-2-yl)methoxy]-phenyl}-7H-pyrrolo[2,3-d]pyrimidin-4-ylamine

The title compound was prepared in a similar manner to Example 1 starting from (S)-tetrahydrofurfurylalcohol (3B Scientific, Libertyville, USA). HPLC t_{R} 2.50 min (Method A); M+H=434 MS-ES.

### Example 18

### 7-[3-(1,1-dioxothiomorpholin-4-ylmethyl)-cyclobutyl]-5-[3-(tetrahydro-pyran-2-ylmethoxy)-phenyl]-7H-pyrrolo[2,3-d]pyrimidin-4-ylamine

The title compound was prepared in a similar manner to Example 1 starting from cis-3-{4-amino-7-[3-(1,1-dioxothiomorpholin-4-ylmethyl)-cyclobutyl]-7H-pyrrolo[2,3-d]pyrimidin-5-yl}-phenol (Intermediate B).

### Example 19

### cis-4-[3-(4-amino-5-{3-[(S)-1-(tetrahydro-furan-2-yl)methoxy]-phenyl}-pyrrolo[2,3-d]pyrimidin-7-yl)-cyclobutylmethyl]-1-methyl-piperazin-2-one

The title compound was prepared in a similar manner to Example 1 starting from 4-{3-[4-amino-5-(3-hydroxy-phenyl)-pyrrolo[2,3-d]pyrimidin-7-yl]-cyclobutylmethyl}-1-methyl-piperazin-2-one (Intermediate D) and (S)-tetrahydrofurfurylalcohol (3B Scientific, Libertyville, USA). HPLC t_{R} 2.44 min (Method A); M+H=491 MS-ES.

### Example 20

### cis-7-(3-azetidin-1-ylmethyl-cyclobutyl)-5-{3-[2-(1-ethyl-propoxy)-ethoxy]-phenyl}-7H-pyrrolo[2,3-d]pyrimidin-4-ylamine

The title compound was prepared in a similar manner to Example 1 starting from 2-(1-ethyl-propoxy)-ethanol (Intermediate I). HPLC t_{R} 2.95 min (Method A); M+H=464 MS-ES.

### Example 21

### cis-7-(3-azetidin-1-ylmethyl-cyclobutyl)-5-[3-(2-cyclopentyloxy-ethoxy)-phenyl]-7H-pyrrolo[2,3-d]pyrimidin-4-ylamine

The title compound was prepared in a similar manner to Example 1 starting from 2-cyclopentyloxy-ethanol (Intermediate J). HPLC t_{R} 2.84 min (Method A); M+H=462 MS-ES.

### Example 22

### (3-{4-amino-5-[3-(7-oxa-bicyclo[2.2.1]hept-1-ylmethoxy)-phenyl]-pyrrolo[2,3-d]pyrimidin-7-yl}-cyclobutyl)-methanol

A mixture of 2-(3-(7-oxabicyclo[2.2.1]heptan-1-ylmethoxy)phenyl)-4,4,5,5-tetramethyl-1,3,2-dioxaborolane (Intermediate K, 155 mg, 0.47 mmol), [3-(4-amino-5-iodo-pyrrolo[2,3-d]pyrimidin-7-yl)-cyclobutyl]-methanol (Intermediate M, 154 mg, 0.45 mmol), tetrakis(triphenylphosphine)palladium (52 mg, 0.05 mmol), sodium carbonate (99 mg, 0.94 mmol), water (2 ml) and DMF (4 ml) was heated at 80° C. for 16 hours under an argon atmosphere in the dark. After cooling water was added and the mixture extracted 3× with DCM, dried over sodium sulphate and the organic layers evaporated. Purification of the residue by flash chromatography, eluting with a gradient of methanol in DCM, gave the title compound. HPLC/MS t_{R} 0.91 min, M+H 421.1 (Method X); ¹H-NMR (CDCl₃, 400 MHz): 8.31 (s, 1H), 7.37 (t, 1H), 7.12 (s, 1H), 7.09-7.05 (m, 2H), 6.98 (dd, 1H), 5.29 (broad s, 1H), 5.15-5.09 (m, 1H), 4.61 (t, 1H), 4.30 (s, 2H), 3.73 (d, 2H), 2.70-2.58 (m, 4H), 2.52-2.44 (m, 1H), 1.93-1.78 (m, 4H), 1.67-1.57 (m, 4H).

### Example 23

### 7-(3-azetidin-1-ylmethyl-cyclobutyl)-5-[3-(7-oxa-bicyclo[2.2.1]hept-1-ylmethoxy)-phenyl]-7H-pyrrolo[2,3-d]pyrimidin-4-ylamine

Sodium triacetoxyborohydride (30 mg, 0.14 mmol) was added to a mixture of 3-{4-amino-5-[3-(7-oxa-bicyclo[2.2.1]hept-1-ylmethoxy)-phenyl]-pyrrolo[2,3-d]pyrimidin-7-yl}-cyclobutanecarbaldehyde (Intermediate N, 15 mg, 0.04 mmol), azetidine (2.7 mg, 0.05 mmol), acetic acid (5.4 mg, 0.09 mmol) and DCE (1 ml) at room temperature. After stirring for 1 hour the reaction mixture was evaporated, taken up in DMF and purified by preparative reversed phase chromatography. Product containing fractions were eluted through a VARIAN Bond Elut SCK cartridge (300 mg), then released by elution with ammonia in methanol (1 ml, 7M) and evaporated to give the title compound. HPLC/MS t_{R} 0.74 min, M+H 460.0 (Method X).

### Example 24

### 7-[3-(1,1-dioxo-1-thiomorpholin-4-ylmethyl)-cyclobutyl]-5-[3-(7-oxa-bicyclo[2.2.1]hept-1-ylmethoxy)-phenyl]-7H-pyrrolo[2,3-d]pyrimidin-4-ylamine

The title compound was prepared in a similar manner to Example 23 starting from thiomorpholine-1,1-dioxide. HPLC/MS t_{R} 0.86 min, M+H 538.0 and M−H 581.8 (Method X). Alternatively, a mixture of 1-methyl-4-[3-(4,4,5,5-tetramethyl-[1,3,2]dioxaborolan-2-yl)-phenoxymethyl]-7-oxa-bicyclo[2.2.1]heptane (Intermediate BB, 992 mg, 2.70 mmol), 5-bromo-7-[3-(1,1-dioxo-1-thiomorpholin-4-ylmethyl)-cyclobutyl]-7H-pyrrolo[2,3-d]pyrimidin-4-ylamine (Intermediate BC, 800 mg, 1.93 mmol), K₃PO₄ (845 mg, 3.86 mmol), Na₂CO₃ (409 mg, 3.86 mmol), DMF (19 ml) and water (0.8 ml) was purged with argon, tetrakis(triphenylphosphine)palladium (180 mg, 0.15 mmol) added, the reaction vessel sealed under argon and heated for 5 hours at 100° C. The cooled reaction mixture was diluted with ethyl acetate and washed with water then brine, the organic layers dried over Na₂SO₄ and evaporated. Purification of the residue by normal phase column chromatography, eluting with a gradient of methanol in DCM, gave the title compound as a white solid.

### Example 25

### [(S)-1-(3-{4-amino-5-[3-(7-oxa-bicyclo[2.2.1]hept-1-ylmethoxy)-phenyl]-pyrrolo[2,3-d]pyrimidin-7-yl}-cyclobutylmethyl)-pyrrolidin-2-yl]-methanol

The title compound was prepared in a similar manner to Example 23 starting from L-prolinol. HPLC/MS t_{R} 0.80 min, M+H 504.0 (Method X).

### Example 26

### 1-(3-{4-amino-5-[3-(7-oxa-bicyclo[2.2.1]hept-1-ylmethoxy)-phenyl]-pyrrolo[2,3-d]pyrimidin-7-yl}-cyclobutylmethyl)azetidin-3-ol

The title compound was prepared in a similar manner to Example 23 starting from azetidine-3-ol. HPLC/MS t_{R} 0.75 min, M+H 475.9 (Method X).

### Example 27

### 7-[3-((R)-3-fluoro-pyrrolidin-1-ylmethyl)-cyclobutyl]-5-[3-(tetrahydro-pyran-2-ylmethoxy)-phenyl]-7H-pyrrolo[2,3-d]pyrimidin-4-ylamine

To a mixture of 3-{4-chloro-5-[3-(tetrahydro-pyran-2-ylmethoxy)-phenyl]-pyrrolo[2,3-d]pyrimidin-7-yl}-cyclobutanecarbaldehyde (Intermediate Q, 0.01 mmol), 3-(R)-fluoropyrrolidine hydrochloride (5 mg, 0.04 mmol), diisopropylethylamine (0.2 mmol, 27 uL) in dichloroethane (1 mL), was added sodium triacetoxyborohydride (6 mg, 0.03 mmol). The mixture was stirred at room temperature for 2 h and then concentrated to give the crude title compound which was purified by reversed phase preparative HPLC (Method S). MS m/z 480.3 (M+H⁺) (Method M).

### Example 28

### 1-(cis-3-{4-amino-5-[3-(tetrahydro-pyran-2-ylmethoxy)-phenyl]-pyrrolo[2,3-d]pyrimidin-7-yl}-cyclobutylmethyl)-azetidin-3-ol

The title compound was prepared in a similar manner to Example 27 starting from 3-hydroxyazetidine. MS m/z 464.3 (M+H⁺) (Method M).

### Example 29

### (R)-1-(cis-3-{4-amino-5-[3-(tetrahydro-pyran-2-ylmethoxy)-phenyl]-pyrrolo[2,3-d]pyrimidin-7-yl}-cyclobutylmethyl)-pyrrolidine-2-carboxylic acid amide

The title compound was prepared in a similar manner to Example 27 starting from D-proline amide. MS m/z 505.3 (M+H⁺) (Method M).

### Example 30

### 3-[cis-3-(1,1-Dioxothiomorpholin-4-ylmethyl)-cyclobutyl]-1-[3-(tetrahydro-pyran-2-ylmethoxy)-phenyl]-imidazo[1,5-a]pyrazin-8-ylamine

(cis-3-{8-Amino-1-[3-(tetrahydro-pyran-2-ylmethoxy)-phenyl]-imidazo[1,5-a]pyrazin-3-yl}-cyclobutyl)-methanol (Intermediate T, 95 mg, 0.23 mmol) was dissolved in acetonitrile (10 mL) and IBX (78 mg, 0.27 mmol) was added. The reaction mixture was heated with stirring in a sealed tube to 90° C. for 20 min. It was then allowed to cool to rt. Solids were filtered off through a plug of celite and the filtrate concentrated. The remaining solid was taken up in 1,2 dichloroethane (5 mL) and thiomorpholine-1,1-dioxide (173 mg, 1.2 mmol), acetic acid (146 μL, 2.5 mmol) and sodium triacetoxy borohydride (108 mg, 0.52 mmol) were added and the reaction mixture was stirred for 16 h at rt. It was then diluted with DCM and quenched by addition of aqueous NaHCO₃ solution. The aqueous layer was repeatedly extracted with DCM. Combined organic extracts were dried and concentrated. The remaining crude material was purified by normal phase preparative TLC (DCM/MeOH: 9:1) to give the title compound as a yellow solid. M+H 527.1. ¹H-NMR (DMSO d₆, 400 MHz) 7.46-7.34 (m, 2H), 7.20-7.06 (m, 2H), 7.04-6.96 (m, 2H), 6.02 (bs, 2H), 4.03-3.86 (m, 3H), 3.83-3.72 (m, 1H), 3.69-3.59 (m, 1H), 3.11-3.00 (m, 4H), 2.94-2.83 (m, 4H), 2.74-2.64 (m, 1H), 2.60-2.53 (m, 4H), 2.07-2.05 (m, 2H), 1.82-1.80 (m, 1H), 1.73-1.61 (m, 1H), 1.57-1.42 (m, 3H), 1.40-1.37 (m, 1H).

### Example 31

### 7-[cis-3-(4,4-difluoro-piperidin-1-ylmethyl)-cyclobutyl]-5-[3-(tetrahydro-pyran-2-ylmethoxy)-phenyl]-7H-pyrrolo[2,3-d]pyrimidin-4-ylamine

The title compound was prepared in a similar manner to Example 27 starting from 4,4-difluoropiperidine. MS m/z 512.3 (M+H⁺) (Method M).

### Example 32

### 7-[cis-3-(4-fluoro-piperidin-1-ylmethyl)-cyclobutyl]-5-[3-(tetrahydro-pyran-2-ylmethoxy)-phenyl]-7H-pyrrolo[2,3-d]pyrimidin-4-ylamine

The title compound was prepared in a similar manner to Example 27 starting from 4-fluoropiperidine. MS m/z 494.3 (M+H⁺) (Method M).

### Example 33

### (R)-1-(cis-3-{4-amino-5-[3-(tetrahydro-pyran-2-ylmethoxy)-phenyl]-pyrrolo[2,3-d]pyrimidin-7-yl}-cyclobutylmethyl)-pyrrolidin-3-ol

The title compound was prepared in a similar manner to Example 27 starting from (R)-pyrrolidin-3-ol. MS m/z 478.3 (M+H⁺) (Method M).

### Example 34

### (S)-1-(cis-3-{4-amino-5-[3-(tetrahydro-pyran-2-ylmethoxy)-phenyl]-pyrrolo[2,3-d]pyrimidin-7-yl}-cyclobutylmethyl)-pyrrolidin-3-ol

The title compound was prepared in a similar manner to Example 27 starting from (S)-pyrrolidin-3-ol. MS m/z 478.3 (M+H⁺) (Method M).

### Example 35

### (2S,3S)-1-(cis-3-{4-amino-5-[3-(tetrahydro-pyran-2-ylmethoxy)-phenyl]-pyrrolo[2,3-d]pyrimidin-7-yl}-cyclobutylmethyl)-3-hydroxy-pyrrolidine-2-carboxylic acid amide

The title compound was prepared in a similar manner to Example 27 starting from (2S,3S)-3-hydroxyproline amide. MS m/z 521.3 (M+H⁺) (Method M).

### Example 36

### 1-(trans-3-{4-amino-5-[3-(tetrahydro-pyran-2-ylmethoxy)-phenyl]pyrrolo[2,3-d]pyrimidin-7-yl}-cyclobutylmethyl)-azetidin-3-ol

The title compound was prepared in a similar manner to Example 27 starting from (trans-3-{4-amino-5-[3-(tetrahydro-pyran-2-ylmethoxy)-phenyl]-pyrrolo[2,3-d]pyrimidin-7-yl}-cyclobutyl)-methanol (Intermediate S) and 3-hydroxyazetidine. MS m/z 464.3 (M+H⁺) (Method M).

### Example 37

### 7-[trans-3-(3-fluoro-azetidin-1-ylmethyl)-cyclobutyl]-5-[3-(tetrahydro-pyran-2-ylmethoxy)-phenyl]-7H-pyrrolo[2,3-d]pyrimidin-4-ylamine

The title compound was prepared in a similar manner to Example 27 starting from (trans-3-{4-amino-5-[3-(tetrahydro-pyran-2-ylmethoxy)-phenyl]-pyrrolo[2,3-d]pyrimidin-7-yl}-cyclobutyl)-methanol (Intermediate S) and 3-fluoroazetidine. MS m/z 466.3 (M+H⁺) (Method M).

### Example 38

### (S)-1-(trans-3-{4-amino-5-[3-(tetrahydro-pyran-2-ylmethoxy)-phenyl]-pyrrolo[2,3-d]pyrimidin-7-yl}-cyclobutylmethyl)-pyrrolidine-2-carboxylic acid amide

The title compound was prepared in a similar manner to Example 27 starting from (trans-3-{4-amino-5-[3-(tetrahydro-pyran-2-ylmethoxy)-phenyl]-pyrrolo[2,3-d]pyrimidin-7-yl}-cyclobutyl)-methanol (Intermediate S) and L-proline amide. MS m/z 505.3 (M+H⁺) (Method M).

### Example 39

### 5-[cis-3-(5,5-dimethyl-tetrahydro-furan-2-ylmethoxy)-phenyl]-7-[3-((S)-3-fluoro-pyrrolidin-1-ylmethyl)-cyclobutyl]-7H-pyrrolo[2,3-d]pyrimidin-4-ylamine

The title compound was prepared in a similar manner to Example 27 starting from (cis-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate V) and (S)-3-fluoropyrrolidine. MS m/z 494.3 (M+H⁺) (Method M). ¹H NMR (MeOD-d₄) δ 8.03 (s, 1H), 7.34 (s, 1H), 7.29 (m, 1H), 6.97 (m, 2H), 6.86 (m, 1H), 5.01-5.14 (m, 2H), 4.28 (m, 1H), 3.90-3.99 (m, 2H), 2.82 (m, 2H), 2.61 (m, 4H), 2.34 (m, 2H), 2.14 (m, 4H), 1.92 (m, 3H), 1.75 (m, 2H), 1.19 (s, 3H), 1.18 (s, 3H).

### Example 40

### 5-(cis-3-((5,5-dimethyltetrahydrofuran-2-yl)methoxy)phenyl)-7-(3-((3-fluoroazetidin-1-yl)methyl)cyclobutyl)-7H-pyrrolo[2,3-d]pyrimidin-4-amine

The title compound was prepared in a similar manner to Example 27 starting from (cis-3-(4-amino-5-[3-(5,5-dimethyl-tetrahydro-furan-2-ylmethoxy)-phenyl]-pyrrolo[2,3-d]pyrimidin-7-yl)-cyclobutyl)-methanol (Intermediate V) and 3-fluoroazetidine. MS m/z 480.3 (M+H⁺) (Method M). ¹H NMR (MeOD-d₄) δ 8.33 (s, 1H), 7.73 (s, 1H), 7.45 (t, 1H), 7.07 (m, 2H), 7.04 (m, 1H), 5.30 (m, 1H), 4.39 (m, 2H), 4.04 (m, 2H), 3.52 (m, 2H), 2.80 (m, 2H), 2.51 (m, 4H), 2.18 (m, 2H), 1.94 (m, 2H), 1.87 (m, 3H), 1.29 (s, 6H).

### Example 41

### 1-(cis-3-{4-amino-5-[3-(5,5-dimethyl-tetrahydro-furan-2-ylmethoxy)-phenyl]-pyrrolo[2,3-d]pyrimidin-7-yl}-cyclobutylmethyl)-azetidin-3-ol

The title compound was prepared in a similar manner to Example 27 starting from (cis-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate V) and 3-hydroxyazetidine. MS m/z 478.3 (M+H⁺). (Method M). ¹HNMR (MeOD-d₄) δ 8.02 (s, 1H), 7.40 (s, 1H), 7.28 (t, 1H), 6.97 (m, 2H), 6.85 (m, 1H), 5.22 (m, 1H), 4.26 (m, 2H), 3.90 (m, 2H), 3.59 (m, 2H), 3.20 (m, 2H), 2.68 (m, 2H), 2.57 (m, 2H), 2.28 (m, 2H), 2.08 (m, 1H), 1.83 (m, 4H), 1.18 (s, 3H), 1.17 (s, 3H).

### Example 42

### (S)-1-(cis-3-{4-amino-5-[3-(tetrahydro-pyran-2-ylmethoxy)-phenyl]-pyrrolo[2,3-d]pyrimidin-7-yl}-cyclobutylmethyl)-pyrrolidine-2-carboxylic acid amide

The title compound was prepared in a similar manner to Example 27 starting from L-proline amide. MS m/z 505.3 (M+H⁺). (Method M).

### Examples 43 & 44

### (S)-1-[3-(4-amino-5-{3-[(R)-1-(tetrahydro-pyran-2-yl)methoxy]-phenyl}-pyrrolo[2,3-d]pyrimidin-7-yl)-cyclobutylmethyl]-pyrrolidine-2-carboxylic acid amide and (S)-1-[3-(4-amino-5-{3-[(S)-1-(tetrahydro-pyran-2-yl)methoxy]-phenyl}-pyrrolo[2,3-d]pyrimidin-7-yl)-cyclobutylmethyl]-pyrrolidine-2-carboxylic acid amide

(S)-1-(cis-3-{4-amino-5-[3-(tetrahydro-pyran-2-ylmethoxy)-phenyl]-pyrrolo[2,3-d]pyrimidin-7-yl}-cyclobutylmethyl)-pyrrolidine-2-carboxylic acid amide (Example 42) was separated into its pure optical isomers by means of chiral chromatography (Column: 21×250 mm ChiralCel OD-H; Conditions: 20 mL/min flow rate, 6:2:2 Hexane:EtOH:MeOH; Run Time: 35 minutes).

Analytical chiral HPLC retention times: 12.74 min. and 32.22 min. (Column: 4.6×250 mm ChiralCel OD-H; Conditions: 1mL/min flow rate, 70:15:15 Hexane:EtOH:MeOH modified with 0.1% DEA). MS m/z 505.3 (M+H⁺). (Method M).

### Example 45

### 5-[trans-3-(5,5-dimethyl-tetrahydro-furan-2-ylmethoxy)-phenyl]-7-[3-(S)-3-fluoro-pyrrolidin-1-ylmethyl)cyclobutyl]-7H-pyrrolo[2,3-d]pyrimidin-4-ylamine

The title compound was prepared in a similar manner to Example 27 starting from (trans-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate W) and (S)-3-fluoropyrrolidine. MS m/z 494.3 (M+H⁺) (Method M). ¹HNMR (MeOD-d₄) δ 8.13 (s, 1H), 7.53 (s, 1H), 7.39 (t, 1H), 7.01 (m, 2H), 6.96 (m, 1H), 5.34 (m, 1H), 5.10-5.25 (m, 1H), 4.38 (m, 1H), 4.03 (m, 2H), 2.93 (m, 2H), 2.78 (m, 2H), 2.68 (m, 4H), 2.45 (m, 3H), 2.19 (m, 2H), 1.96 (m, 2H), 1.85 (m, 2H), 1.29 (s, 3H), 1.28 (s, 3H).

### Example 46

### 5-[trans-3-(5,5-dimethyl-tetrahydro-furan-2-ylmethoxy)-phenyl]-7-[3-((R)-3-fluoro-pyrrolidin-1-ylmethyl)cyclobutyl]-7H-pyrrolo[2,3-d]pyrimidin-4-ylamine

The title compound was prepared in a similar manner to Example 27 starting from (trans-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate W) and (R)-3-fluoropyrrolidine. MS m/z 494.3 (M+H⁺). ¹H NMR (MeOD-d₄) δ 8.13 (s, 1H), 7.53 (s, 1H), 7.39 (t, 1H), 7.09 (m, 2H), 6.97 (m, 1H), 5.32 (m, 1H), 5.10-5.25 (m, 1H), 4.38 (m, 1H), 4.03 (m, 2H), 2.92 (m, 2H), 2.78 (m, 2H), 2.68 (m, 4H), 2.45 (m, 3H), 2.19 (m, 2H), 1.96 (m, 2H), 1.85 (m, 2H), 1.29 (s, 3H), 1.28 (s, 3H).

### Example 47

### 5-[trans-3-(5,5-dimethyl-tetrahydro-furan-2-ylmethoxy)-phenyl]-7-[3-(3-fluoro-azetidin-1-ylmethyl)-cyclobutyl]-7H-pyrrolo[2,3-d]pyrimidin-4-ylamine

The title compound was prepared in a similar manner to Example 27 starting from (trans-3-{(4-amino-5-[3-(5,5-dimethyl-tetrahydro-furan-2-ylmethoxy)-phenyl]-pyrrolo[2,3-d]pyrimidin-7-yl}cyclobutyl)-methanol (Intermediate W) and 3-fluoroazetidine. MS m/z 480.3 (M+H⁺) (Method M). ¹HNMR (MeOD-d₄) δ 8.02 (s, 1H), 7.40 (s, 1H), 7.28 (t, 1H), 6.98 (m, 2H), 6.85 (m, 1H), 5.22 (m, 1H), 4.97-5.10 (m, 1H), 4.27 (m, 1H), 3.90 (m, 2H), 3.58 (m, 2H), 3.20 (m, 2H), 2.70 (d, 2H), 2.56 (m, 2H), 2.28 (m, 2H), 2.08 (m, 1H), 1.80 (m, 4H), 1.18 (s, 3H), 1.17 (s, 3H).

### Example 48

### 1-(trans-3-{4-amino-5-[3-(5,5-dimethyl-tetrahydro-furan-2-ylmethoxy)-phenyl]-pyrrolo[2,3-d]pyrimidin-7-yl}-cyclobutylmethyl)-azetidin-3-ol

The title compound was prepared in a similar manner to Example 27 starting from (trans-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate W) and 3-hydroxyazetidine. MS m/z 478.3 (M+H⁺)

(Method M). ¹HNMR (MeOD-d₄) δ 8.02 (s, 1H), 7.40 (s, 1H), 7.28 (t, 1H), 6.97 (m, 2H), 6.85 (m, 1H), 5.22 (m, 1H), 4.26 (m, 2H), 3.90 (m, 2H), 3.59 (m, 2H), 3.20 (m, 2H), 2.68 (m, 2H), 2.57 (m, 2H), 2.28 (m, 2H), 2.08 (m, 1H), 1.83 (m, 4H), 1.18 (s, 3H), 1.17 (s, 3H).

### Example 49

### 1-(cis-3-{4-amino-5-[3-(5,5-dimethyl-tetrahydro-furan-2-ylmethoxy)-phenyl]-pyrrolo[2,3-d]pyrimidin-7-yl}-cyclobutylmethyl)-azetidine-3-carboxylic acid amide

The title compound was prepared in a similar manner to Example 27 starting from (cis-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate V) and azetidine-3-carboxylic acid amide. MS m/z 505.3 (M+H⁺) (Method M). ¹HNMR (MeOD-d₄) δ 8.14 (s, 1H), 7.46 (s, 1H), 7.40 (t, 1H), 7.09 (t, 2H), 6.98 (m, 1H), 5.10 (m, 1H), 4.39 (m, 1H), 4.02 (m, 3H), 3.57 (m, 2H), 2.67 (m, 4H), 2.25 (m, 4H), 1.90 (m, 5H), 1.30 (s, 3H), 1.29 (s, 3H).

### Example 50

### (S)-1-(cis-3-{4-amino-5-[3-(5,5-dimethyl-tetrahydro-furan-2-ylmethoxy)-phenyl]-pyrrolo[2,3-d]pyrimidin-7-yl}-cyclobutylmethyl)-pyrrolidine-2-carboxylic acid amide

The title compound was prepared in a similar manner to Example 27 starting from (cis-3-{4-amino-5-[3-(5,5-dimethyl-tetrahydro-furan-2-ylmethoxy)-phenyl]-pyrrolo[2,3-d]pyrimidin-7-yl}-cyclobutylymethanol (Intermediate V) and L-proline amide. MS m/z 519.3 (M+H⁺) (Method M). ¹HNMR (MeOD-d₄) δ 8.14 (s, 1H), 7.43 (s, 1H), 7.39 (t, 1H), 7.07 (m, 2H), 6.97 (m, 1H), 5.10 (m, 1H), 4.39 (m, 2H), 4.02 (m, 2H), 3.23 (m, 1H), 2.96 (m, 1H), 2.80 (m, 1H), 2.70 (m, 4H), 2.41 (m, 2H), 2.18 (m, 4H), 1.82 (m, 4H), 1.30 (s, 3H), 1.29 (s, 3H).

### Example 51

### (R)-1-(cis-3-{4-amino-5-[3-(5,5-dimethyl-tetrahydro-furan-2-ylmethoxy)-phenyl]-pyrrolo[2,3-d]pyrimidin-7-yl}-cyclobutylmethyl)-pyrrolidine-2-carboxylic acid amide

The title compound was prepared in a similar manner to Example 27 starting from (cis-3-{4-amino-5-[3-(5,5-dimethyl-tetrahydro-furan-2-ylmethoxy)-pheny]-pyrrolo[2,3-d]pyrimidin-7-yl}-cyclobutyl)-methanol (Intermediate V) and D-proline amide. MS m/z 519.3 (M+H⁺) (Method M). ¹HNMR (MeOD-d₄) δ 8.13 (s, 1H), 7.44 (s, 1H), 7.39 (t, 1H), 7.09 (m, 2H), 6.97 (m, 1H), 5.10 (m, 1H), 4.39 (m, 2H), 4.07 (m, 2H), 3.49 (m, 1H), 3.00 (m, 2H), 2.78 (m, 4H), 2.42 (m, 2H), 2.19 (m, 4H), 1.94 (m, 4H), 1.30 (s, 3H), 1.29 (s, 3H).

### Example 52

### 5-[cis-3-(5,5-dimethyl-tetrahydro-furan-2-ylmethoxy)-phenyl]-7-[3-((R)-3-fluoro-pyrrolidin-1-ylmethyl)cyclobutyl]-7H-pyrrolo[2,3-d]pyrimidin-4-ylamine

The title compound was prepared in a similar manner to Example 27 starting from (cis-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate V) and (R)-3-fluoropyrrolidine. MS m/z 494.3 (M+H⁺) (Method M). ¹HNMR (MeOD-d₄) δ 8.14 (s, 1H), 7.45 (s, 1H), 7.38 (t, 1H), 7.09 (m, 2H), 6.97 (m, 1H), 5.11 (m, 2H), 4.39 (m, 1H), 4.02 (m, 2H), 2.94 (m, 2H), 2.73 (m, 4H), 2.47 (m, 2H), 2.24 (m, 4H), 1.94 (m, 5H), 1.30 (s, 3H), 1.29 (s, 3H).

### Example 53

### 7-[cis-3-(4,4-difluoro-piperidin-1-ylmethyl)-cyclobutyl]-5-[3-(5,5-dimethyl-tetrahydro-furan-2-ylmethoxy)-phenyl]-7H-pyrrolo[2,3-d]pyrimidin-4-ylamine

The title compound was prepared in a similar manner to Example 27 starting from (cis-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate V) and 4,4-difluoropiperidine. MS m/z 526.3 (M+H⁺) (Method M).

### Example 54 & 55

### 7-[cis-3-(4,4-difluoro-piperidin-1-ylmethyl)-cyclobutyl]-5-[3-((R)-5,5-dimethyl-tetrahydro-furan-2-ylmethoxy)-phenyl]-7H-pyrrolo[2,3-d]pyrimidin-4-ylamine and 7-[cis-3-(4,4-difluoro-piperidin-1-ylmethyl)-cyclobutyl]-5-[3-((S)-5,5-dimethyl-tetrahydro-furan-2-ylmethoxy)-phenyl]-7H-pyrrolo[2,3-d]pyrimidin-4-ylamine

7-[cis-3-(4,4-difluoro-piperidin-1-ylmethyl)-cyclobutyl]-5-[3-(5,5-dimethyl-tetrahydro-furan-2-ylmethoxy)-phenyl]-7H-pyrrolo[2,3-d]pyrimidin-4-ylamine (Example 53) was separated into its pure optical isomers by means of chiral chromatography (Column: 21×250 mm ChiralCel OD-H; Conditions: 18 mL/min flow rate, 8:1:1 Hexane:EtOH:MeOH; Run Time: 14 minutes). Analytical chiral HPLC retention times: 2.62 min. and 4.10 min. (Column: 4.6×100 mm ChiralCel OD-H; Conditions: 2 mL/min flow rate, 7:3 CO₂:MeOH, 30° C.). MS m/z 526.3 (M+H⁺) (Method M).

### Example 56

### (2R,3S)-1-(cis-3-{4-amino-5-[3-(tetrahydro-pyran-2-ylmethoxy)-phenyl]-pyrrolo[2,3d]pyrimidin-7-yl}-cyclobutylmethyl)-3-hydroxy-pyrrolidine-2-carboxylic acid amide

The title compound was prepared in a similar manner to Example 27 starting from (2R,3S)-3-hydroxyproline amide. MS m/z 521.3 (M+H⁺) (Method M).

### Example 57

### (S)-1-(3-{4-amino-5-[3-(5,5-dimethyl-tetrahydro-furan-2-ylmethoxy)-phenyl]-5 pyrrolo[2,3-d]pyrimidin-7-yl}-cyclobutylmethyl)-pyrrolidin-3-ol

The title compound was prepared in a similar manner to Example 27 starting from (cis-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate V) and (S)-3-hydroxypyrrolidine. MS m/z 492.3 (M+H⁺) (Method M). ¹HNMR (MeOD-d₄) δ 8.14 (s, 1H), 7.45 (s, 1H), 7.40 (1, 1H), 7.08 (m, 2H), 6.97 (m, 1H), 5.10 (m, 1H), 4.37 (m, 2H), 4.02 (m, 2H), 2.87 (m, 1H), 2.70 (m, 4H), 2.60 (m, 1H), 2.51 (m, 1H), 2.43 (m, 1H), 2.17 (m, 5H), 1.88 (m, 3H), 1.72 (m, 1H), 1.30 (s, 3H), 1.29 (s, 3H).

### Example 58

### (R)-1-(cis-3-{4-amino-5-[3-(5,5-dimethyl-tetrahydro-furan-2-ylmethoxy)-phenyl]-pyrrolo[2,3-d]pyrimidin-7-yl}cyclobutylmethyl)-pyrrolidin-3-ol

The title compound was prepared in a similar manner to Example 27 starting from (cis-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate V) and (R)-3-hydroxypyrrolidine. MS m/z 492.3 (M+H⁺) (Method M). ¹H NMR (MeOD-d₄) δ 8.03 (s, 1H), 7.34 (s, 1H), 7.29 (t, 1H), 6.98 (m, 2H), 6.86 (m, 1H), 5.01 (m, 1H), 4.27 (m, 2H), 3.96 (m, 2H), 2.78 (m, 1H), 2.61 (m, 5H), 2.53 (m, 1H), 2.43 (m, 1H), 2.33 (m, 1H), 2.11 (m, 4H), 1.77 (m, 4H), 1.30 (s, 3H), 1.28 (s, 3H).

### Example 59

### 7-[cis-3-(4-fluoro-piperidin-1-ylmethyl)-cyclobutyl]-5-[3-(5,5-dimethyl-tetrahydro-furan-2-ylmethoxy)-phenyl]-7H-pyrrolo[2,3-d]pyrimidin-4-ylamine

The title compound was prepared in a similar manner to Example 27 starting from (cis-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate V) and 4-fluoropiperidine. MS m/z 508.3 (M+H⁺) (Method M). ¹H NMR (MeOD-d₄) δ 8.03 (s, 1H), 7.34 (s, 1H), 7.29 (t, 1H), 6.97 (m, 2H), 6.88 (m, 1H), 5.01 (m, 1H), 4.28 (m, 2H), 3.94 (m, 2H), 2.60 (m, 6H), 2.37 (m, 3H), 2.11 (m, 3H), 1.857 (m, 7H), 1.19 (s, 3H), 1.18 (s, 3H).

### Example 60

### 5-[3-(5,5-dimethyl-tetrahydro-furan-2-ylmethoxy)-phenyl]-7-(1′-methyl-[1,4′]bipiperidinyl-4-yl)-7H-pyrrolo[2,3-d]pyrimidin-4-ylamine

The title compound was prepared in a similar manner to Example 27 starting from Intermediate AD and 1-methylpiperidin-4-one. MS m/z 519.3 (M+H⁺) (Method M). ¹HNMR (MeOD-d₄) δ 8.15 (s, 1H), 7.38 (t, 1H), 7.33 (s, 1H), 7.08-7.06 (m, 2H), 6.98-6.95 (m, 1H), 4.68-4.60 (m, 1H), 4.40-4.34 (m, 1H), 4.07-3.96 (m, 2H), 3.14 (d, 2H), 2.97 (d, 2H), 2.50-2.37 (m, 2H), 2.27 (s, 3H), 2.21-2.07 (m, 1H), 2.13-2.07 (m, 4H), 1.98-1.81 (m, 4H), 1.68-1.58 (m,2H), 1.33-1.28 (m, 2H), 1.28 (d, 6H), 0.92-0.88 (m, 2H).

### Example 61

### 4-(cis-3-{4-amino-5-[3-(5,6-dimethyl-tetrahydro-furan-2-ylmethoxy)-phenyl]-pyrrolo[2,3-d]pyrimidin-7-yl}-cyclobutylmethyl)-piperazin-2-one

The title compound was prepared in a similar manner to Example 27 starting from (cis-3-{4-amino-5-[3-(5,6-dimethyl-tetrahydro-furan-2-ylmethoxy)-phenyl]-pyrrolo[2,3-d]pyrimidin-7-yl}-cyclobutyl)-methanol (Intermediate V) and piperazin-2-one. MS m/z 505.3 (M+H⁺) (Method M). ¹HNMR (MeOD-d₄) δ 8.04 (s, 1H), 7.37 (s, 1H), 7.30 (t, 1H), 6.99 (m, 2H), 6.87 (m, 1H), 5.04 (m, 1H), 4.29 (m, 2H), 3.92 (m, 2H), 3.24 (m, 1H), 3.04 (s, 2H), 2.62 (m, 6H), 2.37 (m, 1H), 2.16 (m, 3H), 1.77 (m, 4H), 1.20 (s, 3H), 1.18 (s, 3H).

### Example 62 and 63

### 5-[3-(5,5-dimethyl-tetrahydro-furan-2-ylmethoxy)-phenyl]-7-[trans-4-(4-methyl-piperazin-1-yl)-cyclohexyl]-7H-pyrrolo[2,3-d]pyrimidin-4-ylamine and 5-[3-(5,5-dimethyl-tetrahydro-furan-2-ylmethoxy)-phenyl]-7-[cis-4-(4-methyl-piperazin-1-yl)-cyclohexyl]-7H-pyrrolo[2,3-d]pyrimidin-4-ylamine

The title compounds were prepared in a similar manner to Example 27 starting from Intermediate AC and 1-methylpiperazine. The trans and cis isomers were separated with prep. TLC, eluting with 10% MeOH in DCM with 0.1 N ammonia. MS m/z 519.3 (M+H⁺) (Method M) and MS m/z 519.3 (M+H⁺) (Method M). ¹HNMR (Acetone-d₆) δ 8.05 (s, 1H), 7.27-7.23 (m, 2H), 6.97-6.96 (m, 2H), 6.82-6.79 (m, 1H), 4.59-4.51 (m, 1H), 4.24-4.18 (m, 1H), 3.91 (d, 2H), 2.50-2.48 (m, 2H), 2.37-2.30 (m, 1H), 2.24 (br, 2H), 2.06 (s, 3H), 2.03-2.00 (m, 2H), 2.06-1.88 (m, 2H), 1.88-1.86 (m, 2H), 1.77-1.66 (m, 2H), 1.49-1.43 (m, 2H), 1.18 (br, 4H), 1.11 (d, 6H), 0.78-0.75 (m, 2H).

### Example 64 and 65

### 5-[3-(5,5-dimethyl-tetrahydro-furan-2-ylmethoxy)-phenyl]-7-(trans-4-morpholin-4-yl-cyclohexyl)-7H-pyrrolo[2,3-d]pyrimidin-4-ylamine and 5-[3-(5,5-dimethyl-tetrahydro-furan-2-ylmethoxy)-phenyl]-7-(cis-4-morpholin-4-yl-cyclohexyl)-7H-pyrrolo[2,3-d]pyrimidin-4-ylamine

The title compounds were prepared in a similar manner to Example 27 starting from Intermediate AC and morpholine. The trans and cis isomers were separated with prep. TLC, eluting with 10% MeOH in DCM with 0.1 N ammonia. MS m/z 506.3 (M+H⁺) (Method M), and MS m/z 506.3 (M+H⁺) (Method M).

### Example 66 & 67

### 5-[(R)-3-(5,5-dimethyl-tetrahydro-furan-2-ylmethoxy)-phenyl]-7-[cis-3-(1,1-dioxothiomorpholin-4-ylmethyl)-cyclobutyl]-7H-pyrrolo[2,3-d]pyrimidin-4-ylamine and 5-[(S)-3-(5,5-dimethyl-tetrahydro-furan-2-ylmethoxy)-phenyl]-7-[cis-3-(1,1-dioxothiomorpholin-4-ylmethyl)-cyclobutyl]-7H-pyrrolo[2,3-d]pyrimidin-4-ylamine

The title compounds were prepared in a similar manner to Example 27 starting from (cis-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate V) and 1,1-dioxothiomorpholine to generate 5-[3-(5,5-dimethyl-tetrahydro-furan-2-ylmethoxy)-phenyl]-7-[cis-3-(1,1-dioxothiomorpholin-4-ylmethyl)-cyclobutyl]-7H-pyrrolo[2,3-d]pyrimidin-4-ylamine which was separated into its pure optical isomers by means of chiral chromatography (Column: 20×250 mm ChiralCel OD; Conditions: 20 mL/min flow rate, 6:2:2 Hexane:EtOH:MeOH; Run Time: 20 minutes). Analytical chiral HPLC retention times: 9.25 min. and 12.97 min. (Column: 4.6×250 mm ChiralCel OD-H; Conditions: 1 mL/min flow rate, 60:20:20 Hexane:EtOH:MeOH modified with 0.1% DEA). MS m/z 540.3 (M+H⁺) (Method M).

### Example 68

### (S)-1-(cis-3-(4-amino-5-[3-(7-oxa-bicyclo[2.2.1]hept-1-ylmethoxy)-phenyl]-pyrrolo[2,3-d]pyrimidin-7-yl)-cyclobutylmethyl)-pyrrolidine-2-carboxylic acid amide

The title compounds were prepared in a similar manner to Example 27 starting from cis-3-{4-amino-5-[3-(7-oxa-bicyclo[2.2.1]hept-1-ylmethoxy)-phenyl]-pyrrolo[2,3-d]pyrimidin-7-yl}-cyclobutanecarbaldehyde (Intermediate N) and L-proline amide. MS m/z 517.3 (M+H⁺) (Method M). ¹HNMR (MeOD-d₄) δ 8.03 (d, 1H), 7.70 (d, 1H), 7.30 (t, 1H), 7.02 (m, 2H), 6.90 (m, 1H), 5.00 (m, 1H), 4.50 (m, 1H), 4.24 (s, 2H), 3.13 (m, 1H), 2.88 (m, 1H), 2.68 (m, 1H), 2.58 (m, 3H), 2.29 (m, 3H), 2.10 (m, 3H), 1.73 (m, 6H), 1.58 (m, 4H).

### Example 69

### (S)-1-(4-{4-amino-5-[3-(5,5-dimethyl-tetrahydro-furan-2-ylmethoxy)-phenyl]-pyrrolo[2,3-d]pyrimidin-7-yl}-cyclohexyl)-pyrrolidine-2-carboxylic acid amide

The title compounds were prepared in a similar manner to Example 27 starting from Intermediate AC and L-prolinamide. The trans and cis isomers were separated with prep. TLC, eluting with 10% MeOH in DCM with 0.1 N ammonia. MS m/z 533.3 (M+H⁺) (Method M).

### Example 70

### 5-[3-(5,5-dimethyl-tetrahydro-furan-2-ylmethoxy)-phenyl]-7-(cis-3-thiazolidin-3-ylmethyl-cyclobutyl)-7H-pyrrolo[2,3-d]pyrimidin-4-ylamine

The title compound was prepared in a similar manner to Example 27 starting from (cis-3-{4-amino-6-[3-(5,5-dimethyl-tetrahydro-furan-2-ylmethoxy)-phenyl]-pyrrolo[2,3-d]pyrimidin-7-yl}-cyclobutyl)-methanol (Intermediate V) and thiazolidine. MS m/z 494.3 (M+H⁺) (Method M).

### Example 71

### (S)-1-(trans-3-{4-amino-5-[3-(5,5-dimethyl-tetrahydro-furan-2-ylmethoxy)-phenyl]-pyrrolo[2,3-d]pyrimidin-7-yl}-cyclobutylmethyl)-pyrrolidine-2-carboxylic acid amide

The title compound was prepared in a similar manner to Example 27 starting from (trans-3-{4-amino-5-[3-(5,5-dimethyl-tetrahydro-furan-2-ylmethoxy)-phenyl]-pyrrolo[2,3-d]pyrimidin-7-yl}cyclobutyl)-methanol (Intermediate W) and L-proline amide. MS m/z 519.3 (M+H⁺) (Method M). ¹H NMR (MeOD-d₄) δ 8.14 (d, 1H), 7.43 (s, 1H), 7.39 (m, 1H), 7.08 (m, 2H), 6.96 (m, 1H), 5.33 (m, 1H), 5.10 (m, 1H), 4.39 (m, 1H), 4.03 (m, 2H), 3.36 (s, 1H), 3.23 (m, 1H), 3.01 (m, 1H), 2.80 (m, 4H), 2.43 (m, 2H), 2.18 (m, 3H), 1.97 (m, 5H), 1.30 (s, 3H), 1.29 (s, 3H).

### Example 72

### (R)-1-(trans-3-{4-amino-5-[3-(5,5-dim ethyl-tetrahydro-furan-2-ylmethoxy)-phenyl]pyrrolo[2,3-d]pyrimidin-7-yl}-cyclobutylmethyl)-pyrrolidine-2-carboxylic acid amide

The title compound was prepared in a similar manner to Example 27 starting from (trans-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate W) and D-proline amide. MS m/z 519.3 (M+H⁺) (Method M). ¹H NMR (MeOD-d₄) δ 8.03 (d, 1H), 7.30 (m, 2H), 6.98 (m, 2H), 6.89 (m, 1H), 5.23 (m, 1H), 5.00 (m, 1H), 4.28 (m, 1H), 3.92 (m, 2H), 3.12 (m, 1H), 2.89 (m, 1H), 2.60 (m, 5H), 2.31 (m, 2H), 2.08 (m, 3H), 1.75 (m, 5H), 1.19 (s, 3H), 1.18 (s, 3H).

### Example 73

### 6-[3-(5,5-dimethyl-tetrahydro-furan-2-ylmethoxy)-phenyl]-7-[trans-3-(1,1-dioxothiomorpholin-4-ylmethyl)-cyclobutyl]-7H-pyrrolo[2,3-d]pyrimidin-4-ylamine

The title compound was prepared in a similar manner to Example 27 starting from (trans-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate W) and 1,1-dioxothiomorpholine. MS m/z 540.3 (M+H⁺) (Method M). ¹H NMR (MeOD-d₄) δ 8.14(d, 1H), 7.53 (s, 1H), 7.38 (dt, 1H), 7.09 (m, 1H), 6.97 (m, 1H), 5.36 (m, 1H), 5.12 (m, 1H), 4.39 (m, 1H), 4.02 (m, 2H), 3.10 (m, 3H), 3.08 (m, 3H), 2.79 (m, 1H), 2.75 (m, 1H), 2.68 (m, 3H), 2.42 (m, 2H), 2.18 (m, 2H), 1.86 (m, 4H), 1.30 (s, 3H), 1.29 (s, 3H).

### Example 74

### 5-[3-(5,5-dimethyl-tetrahydro-furan-2-ylmethoxy)-phenyl]-7-[cis-3-(4-fluoro-piperidin-1-ylmethyl)-cyclobutyl]-7H-pyrrolo[2,3-d]pyrimidin-4-ylamine

The title compound was prepared in a similar manner to Example 27 starting from (cis-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate V) and 4-fluoropiperidine. MS m/z 508.3 (M+H⁺) (Method M).

### Example 75

### (R)-1-(cis-3-{4-amino-5-[3-(7-oxa-bicyclo[2.2.1]hept-1-ylmethoxy)-phenyl]-pyrrolo[2,3-d]pyrimidin-7-yl}-cyclobutylmethyl)-pyrrolidine-2-carboxylic acid amide

The title compounds were prepared in a similar manner to Example 27 starting from cis-3-{4-amino-5-[3-(7-oxa-bicyclo[2.2.1]hept-1-ylmethoxy)-phenyl]-pyrrolo[2,3-d]pyrimidin-7-yl}-cyclobutanecarbaldehyde (Intermediate N) and D-proline amide. MS m/z 517.3 (M+H⁺) (Method M). ¹H NMR (MeOD-d₄) δ 8.03 (s, 1H), 7.34 (s, 1H), 7.30 (t, 1H), 7.02 (m, 2H), 6.91 (m, 1H), 5.00 (m, 1H), 4.50 (t, 1H), 4.24 (s, 2H), 3.14 (m, 1H), 2.89 (m, 1H), 2.69 (m, 1H), 2.58 (m, 3H), 2.31 (m, 3H), 2.11 (m, 3H), 1.74 (m, 6H), 1.69 (m, 4H).

### Example 76

### (S)-1-(cis-3-{4-amino-5-[3-(tetrahydro-furan-2-ylmethoxy)-phenyl]-pyrrolo[2,3-d]pyrimidin-7-yl}-cyclobutylmethyl)-pyrrolidine-2-carboxylic acid amide

The title compounds were prepared in a similar manner to Example 27 starting from (cis-3-{4-amino-5-[3-(tetrahydro-furan-2-ylmethoxy)-phenyl]-pyrrolo[2,3-d]pyrimidin-7-yl}-cyclobutyl)-methanol and L-proline amide. MS m/z 491.3 (M+H⁺) (Method M).

### Example 77

### (trans-3-{4-amino-5-[3-(7-oxa-bicyclo[2.2.1]hept-1-ylmethoxy)-phenyl]-pyrrolo[2,3-d]pyrimidin-7-yl}-cyclobutyl)-methanol

The title compounds were prepared in a similar manner to Example 22 starting from [trans-3-(4-amino-5-iodo-pyrrolo[2,3-d]pyrimidin-7-yl)-cyclobutyl]-methanol (Intermediate R) and 2-(3-((7-oxabicyclo[2.2.1]heptan-1-ylmethoxy)phenyl)-4, 4,5,5-tetramethyl-1,3,2-dioxaborolane (Intermediate K). MS m/z 421.2 (M+H⁺) (Method M).

### Example 78

### (S)-1-(trans-3-{4-amino-5-[3-(7-oxa-bicyclo[2.2.1]hept-1-ylmethoxy)-phenyl]-pyrrolo[2,3-d]pyrimidin-7-yl}cyclobutylmethyl)-pyrrolidine-2-carboxylic acid amide

The title compounds were prepared in a similar manner to Example 27 starting from (trans-3-{4-amino-5-[3-(7-oxa-bicyclo[2.2.1]hept-1-ylmethoxy)-phenyl]-pyrrolo[2,3-d]pyrimidin-7-yl}-cyclobutyl)-methanol (Example 77) and L-proline amide. MS m/z 517.3 (M+H⁺) (Method M). ¹H NMR (MeOD-d₄) 68.13 (s, 1H), 7.48 (d, 1H), 7.40 (m, 1H), 7.13 (m, 2H), 7.02 (m, 1H), 5.33 (m, 1H), 5.10 (m, 1H), 4.60 (m, 1H), 4.35 (s, 2H), 3.23 (m, 1H), 3.00 (m, 1H), 2.80 (m, 1H), 2.68 (m, 3H), 2.40 (m, 3H), 2.21 (m, 2H), 1.83 (m, 6H), 1.68 (m, 4H).

### Example 79

### (R)-1-(trans-3-{4-amino-5-[3-(7-oxa-bicyclo[2.2.1]hept-1-ylmethoxy)-phenyl]-pyrrolo[2,3-d]pyrimidin-7-yl}cyclobutylmethyl)-pyrrolidine-2-carboxylic acid amide

The title compounds were prepared in a similar manner to Example 27 starting from (trans-3-{4-amino-5-[3-(7-oxa-bicyclo[2.2.1]hept-1-ylmethoxy)-phenyl]-pyrrolo[2,3-d]pyrimidin-7-yl}-cyclobutyl)-methanol (Example 77) and D-proline amide. MS m/z 517.3 (M+H⁺) (Method M). ¹H NMR (MeOD-d₄) 68.02 (d, 1H), 7.36 (d, 1H), 7.29 (m, 1H), 7.02 (m, 2H), 6.92 (m, 1H), 5.23 (m, 1H), 5.00 (m, 1H), 4.50 (t, 1H), 4.24 (s, 2H), 3.14 (m, 1H), 2.93 (m, 1H), 2.80 (m, 1H), 2.59 (m, 3H), 2.32 (m, 3H), 2.11 (m, 2H), 1.73 (m, 6H), 1.59 (m, 4H).

### Example 80

### (2S,3R)-1-(trans-3-(4-amino-5-[3-(5,5-dimethyl-tetrahydro-furan-2-ylmethoxy)-phenyl]-pyrrolo[2,3-d]pyrimidin-7-yl}-cyclobutylmethyl)-3-hydroxy-pyrrolidine-2-carboxylic acid amide

The title compound was prepared in a similar manner to Example 27 starting from (trans-3-{4-amino-5-[3-(5,5-dimethyl-tetrahydro-furan-2-ylmethoxy)-phenyl]-pyrrolo[2,3-d]pyrimidin-7-yl}-cyclobutyl)-methanol (Intermediate W) and (2R,3S)-3-hydroxyproline amide. MS m/z 535.3 (M+H⁺) (Method M). ¹H NMR (MeOD-d₄) δ 8.02 (s, 1H), 7.42 (s, 1H), 7.29 (m, 1H), 6.98 (m, 2H), 6.86 (m, 1H), 5.23 (m, 1H), 4.38 (m, 1H), 4.29 (m, 1H), 3.92 (m, 2H), 2.95 (m, 1H), 2.80 (m, 1H), 2.57 (m, 4H), 2.31 (m, 3H), 2.09 (m, 3H), 1.85 (m, 2H), 1.82 (m, 4H), 1.20 (s, 3H), 1.18 (s, 3H).

### Example 81

### (R)-1-(cis-3-{4-amino-5-[3-(tetrahydro-furan-2-ylmethoxy)-phenyl]-pyrrolo[2,3-d]pyrimidin-7-yl}-cyclobutylmethyl)-pyrrolidine-2-carboxylic acid amide

The title compounds were prepared in a similar manner to Example 27 starting from (cis-3-{(4-amino-5-[3-(tetrahydro-furan-2-ylmethoxy)-phenyl]-pyrrolo[2,3-d]pyrimidin-7-yl}-cyclobutyl)-methanol and D-proline amide. MS m/z 491.3 (M+H⁺) (Method M). ¹H NMR (MeOD-d₄) δ 8.13 (s, 1H), 7.42 (s, 1H), 7.38 (t, 1H), 7.07 (m, 2H), 6.95 (m, 1H), 5.09 (m, 1H), 4.28 (m, 1H), 4.07 (m, 1H), 4.01 (m, 1H), 3.91 (m, 1H), 3.83 (m, 1H), 3.22 (m, 1H), 2.97 (m, 1H), 2.77 (m, 1H), 2.66 (m, 4H), 2.37 (m, 2H), 2.20 (m, 3H), 1.98 (m, 2H), 1.81 (m, 4H).

### Example 82

### (2S,3R)-1-(cis-3-{4-amino-5-[3-(tetrahydro-furan-2-ylmethoxy)-phenyl]-pyrrolo[2,3-d]pyrimidin-7-yl}-cyclobutylmethyl)-3-hydroxy-pyrrolidine-2-carboxylic acid amide

The title compounds were prepared in a similar manner to Example 27 starting from (cis-3-{4-amino-5-[3-(tetrahydro-furan-2-ylmethoxy)-phenyl]-pyrrolo[2,3-d]pyrimidin-7-yl}-cyclobutyl)-methanol and (2R,3S)-3-hydroxyproline amide. MS m/z 507.3 (M+H⁺) (Method M). ¹H NMR (MeOD-d₄) δ 8.03 (s, 1H), 7.32 (s, 1H), 7.28 (t, 1H), 6.97 (m, 2H), 6.86 (m, 1H), 5.00 (m, 1H), 4.34 (m, 1H), 4.20 (m, 1H), 3.99 (m, 2H), 3.92 (m, 1H), 3.81 (m, 1H), 3.73 (m, 1H), 2.91 (m, 1H), 2.70 (m, 1H), 2.60 (m, 2H), 2.45 (m, 1H), 2.29 (m, 2H), 2.11 (m, 3H), 1.98 (m, 1H), 1.87 (m, 2H), 1.71 (m, 2H).

### Example 83

### 7-[cis-3-(4,4-difluoro-piperidin-1-ylmethyl)-cyclobutyl]-5-[3-(tetrahydro-furan-2-ylmethoxy)-phenyl]-7H-pyrrolo[2,3-d]pyrimidin-4-ylamine

The title compounds were prepared in a similar manner to Example 27 starting from (cis-3-{(4-amino-5-[3-(tetrahydro-furan-2-ylmethoxy)-phenyl]-pyrrolo[2,3-d]pyrimidin-7-yl}-cyclobutyl)-methanol and 4,4-difluoropiperidine. MS m/z 498.3 (M+H⁺) (Method M).

### Example 84

### (cis-3-{8-amino-1-[3-(5,5-dimethyl-tetrahydro-furan-2-ylmethoxy)-phenyl]-imidazo[1,5-a]pyrazin-3-yl}-cyclobutyl)-methanol

[3-(8-Amino-1-iodo-imidazol[1,5-a]pyrazin-3-yl-cyclobutyl]-methanol (prepared according to US20070129547 as 5:1 cis/trans mixture; 103 mg, 0.3 mmol) was dissolved in dioxane (2 mL). Water (2 mL), [2-(3-(5,5)-dimethyltetrahydrofuran-2-ylmethoxy)phenyl]-4,4,5,5-tetramethyl-[1,3,2]dioxaborolane (139 mg, 0.4 mmol), K₃PO₄ (254 mg, 1.2 mmol) and Pd(PPh₃)₄ (69 mg, 0.06 mmol) were added and the reaction mixture was flushed with argon and heated to 60° C. for 1 h. The reaction mixture was allowed to cool and diluted with EtOAc. The organic layer was washed with brine, dried and concentrated. The remaining crude product was purified by flash column chromatography, eluting with a DCM/MeOH gradient to give the title compound as pure cis isomer as major fraction. M+H 424.4. ¹H-NMR (MeOH d₄, 400 MHz) cis-isomer 7.46-7.42 (m, 2H), 7.20 (s, 1H), 7.19 (d, 1H), 7.09 (d, 1H), 6.97 (d, 1H), 4.40-4.36 (m, 1H), 4.08-4.05 (m, 1H), 4.02-3.99 (m, 1H), 3.86-3.83 (m, 1H), 3.57 (d, 2H), 2.64-2.56 (m, 3H), 2.27-2.15 (m, 3H), 2.00-1.90 (m, 1H), 1.87-1.84 (m, 2H), 1.28 (s, 6H).

### Example 85

### 7-(cis-3-methylsulfanylmethyl-cyclobutyl)-5-[3-(tetrahydro-pyran-2-ylmethoxy)-phenyl]-7H-pyrrolo[2,3-d]pyrimidin-4-ylamine

Sodium thiomethoxide (40 mg, 0.57 mmol) was added to a mixture of toluene-4-sulfonic acid cis-3-{4-amino-5-[3-(tetrahydro-pyran-2-ylmethoxy)-phenyl]-pyrrolo[2,3-d]pyrimidin-7-yl}-cyclobutylmethyl ester (Intermediate AA, 100 mg, 0.18 mmol) and THF (1 ml) at room temperature. After stirring for 1 hour at room temperature water was added, the mixture extracted 2× with DCM, dried over sodium sulphate and evaporated to give the crude product. Purification by preparative reversed phase chromatography (Method R) and trituration with methanol gave the title compound. HPLC/MS t_{R} 1.12 min, M+H 439.2 (Method X).

### Example 86

### (S)-1-(trans-3-(4-amino-5-[3-(tetrahydro-furan-2-ylmethoxy)-phenyl]-pyrrolo[2,3-d]pyrimidin-7-yl}-cyclobutylmethyl)-pyrrolidine-2-carboxylic acid amide

The title compounds were prepared in a similar manner to Example 27 starting from (trans-3-}4-amino-5-[3-(tetrahydro-furan-2-ylmethoxy)-phenyl]-pyrrolo[2,3-d]pyrimidin-7-yl}-cyclobutyl)-methanol (Intermediate Z) and L-proline amide. MS m/z 491.3 (M+H⁺) (Method M).

### Example 87

### (R)-1-(trans-3-(4-amino-5-[3-(tetrahydro-furan-2-ylmethoxy)-phenyl]-pyrrolo[2,3-d]pyrimidin-7-yl}-cyclobutylmethyl)-pyrrolidine-2-carboxylic acid amide

The title compounds were prepared in a similar manner to Example 27 starting from (trans-3-(4-amino-5-[3-(tetrahydro-furan-2-ylmethoxy)-phenyl]-pyrrolo[2,3-d]pyrimidin-7-yl}-cyclobutyl)-methanol (intermediate Z) and D-proline amide. MS m/z 491.3 (M+H⁺) (Method M). ¹H NMR (MeOD-d₄) δ 8.14 (d, 1H), 7.47 (d, 1H), 7.39 (m, 1H), 7.08 (m, 2H), 6.96 (m, 1H), 5.10 (m, 1H), 4.29 (m, 1H), 4.07 (m, 1H), 4.03 (m, 1H), 3.91 (m, 1H), 3.84 (m, 1H), 3.22 (m, 1H), 2.98 (m, 1H), 2.78 (m, 1H), 2.68 (m, 3H), 2.41 (m, 2H), 2.19 (m, 4H), 1.98 (m, 2H), 1.82 (m, 4H).

### Example 88

### 7-[cis-3-(1,1-Dioxothiomorpholin-4-ylmethyl)-cyclobutyl]-5-[3-(tetrahydro-furan-2-ylmethoxy)-phenyl]-7H-pyrrolo[2,3-d]pyrimidin-4-ylamine

The title compounds were prepared in a similar manner to Example 27 starting from (cis-3-{4-amino-5-[3-(tetrahydro-furan-2-ylmethoxy)-phenyl]-pyrrolo[2,3-d]pyrimidin-7-yl}-cyclobutyl)-methanol and 1,1-dioxothiomorpholine. MS m/z 512.2 (M+H⁺) (Method M).

### Example 89

### 7-[trans-3-(1,1-Dioxothiomorpholin-4-ylmethyl)-cyclobutyl]-5-[3-(tetrahydro-furan-2-ylmethoxy)-phenyl]-7H-pyrrolo[2,3-d]pyrimidin-4-ylamine

The title compound was prepared in a similar manner to Intermediate Q and Example 27 starting from (trans-3-(4-amino-5-[3-(tetrahydro-furan-2-ylmethoxy)-phenyl]-pyrrolo[2,3-d]pyrimidin-7-yl)cyclobutyl)-methanol (Intermediate Z) and 1,1-dioxothiomorpholine. MS m/z 512.2 (M+H⁺) (Method M). NMR (MeOD-d₄) δ ppm 8.14 (d, 1H), 7.49 (d, 1H), 7.40 (m, 1H), 7.09 (m, 2H), 6.99 (m, 1H), 5.14 (m, 1H), 4.29 (m, 1H), 4.04 (m, 2H), 3.86 (m, 2H), 3.11 (m, 4H), 3.05 (m, 4H), 2.76 (m, 3H), 2.45 (m, 2H), 2.25 (m, 2H), 2.10 (m, 1H), 2.01 (m, 2H), 1.82 (m, 1H).

### Example 90

### 1-(cis-3-{4-amino-5-[3-(tetrahydro-pyran-2-ylmethoxy)-pheny]-pyrrolo[2,3-d]pyrimidin-7-yl}-cyclobutylmethyl)-pyrrolidine-2-carboxylic acid

The title compound was prepared in a similar manner to Example 27 starting from cis-3-{4-amino-5-[3-(tetrahydro-pyran-2-ylmethoxy)-phenyl]-pyrrolo[2,3-d]pyrimidin-7-yl}-cyclobutanecarbaldehyde (Intermediate Q) and D-proline. MS m/z 506.3 (M+H⁺) (Method M)

### Example 91

### (2S,3R)-1(trans-((1R,3S)-3-(4-amino-5-(3-(tetrahydrofuran-2-yl)methoxy)phenyl)-7H-pyrrolo[2,3-d]pyrimidin-7-yl)cyclobutyl)methyl-3-hydroxylpyrrolidine-2-carboxamide

The title compound was prepared in a similar manner to Intermediate Q and Example 27 starting from (trans-3-{4-amino-5-[3-(tetrahydro-furan-2-ylmethoxy)-phenyl]-pyrrolo[2,3-d]pyrimidin-7-yl}-cyclobutyl)-methanol (Intermediate Z) and (2S,3R)-3-hydroxypyrrolidine-2-carboxamide. MS m/z 507.2 (M+H⁺) (Method M). ¹H NMR (MeOD-d₄)₆ ppm 8.14 (m, 1H), 7.44 (m, 3H), 7.08 (m, 2H), 6.97 (m, 1H), 5.11 (m, 1H), 4.30 (m, 1H), 4.05 (m, 2H), 3.87 (m, 2H), 2.72 (m, 5H), 2.42 (m, 3H), 2.22 (m, 2H), 2.11 (m, 2H), 1.99 (m, 3H), 1.83 (m, 3H).

### Example 92

### 1-(cis-3-{4-amino-5-[3-(tetrahydro-pyran-2-ylmethoxy)-phenyl]-pyrrolo[2,3-d]pyrimidin-7-yl}-cyclobutylmethyl)-pyrrolidine-2-carboxylic acid

The title compound was prepared in a similar manner to Example 27 starting from cis-3-{4-amino-5-[3-(tetrahydro-pyran-2-ylmethoxy)-phenyl]-pyrrolo[2,3-d]pyrimidin-7-yl}-cyclobutanecarbaldehyde (Intermediate Q) and L-proline. MS m/z 506.3 (M+H⁺) (Method M)

### Example 93

### (2S)-1-(((1s,3R)-3-(4-amino-5-(3-((tetrahydro-2H-pyran-2-yl)methoxy)phenyl)-7H-pyrrolo[2,3-d]pyrimidin-7-yl)cyclobutyl)methyl)-2-methylpyrrolidine-2-carboxamide

The title compound was prepared in a similar manner to Example 27 starting from 3-{4-chloro-5-[3-(tetrahydro-pyran-2-ylmethoxy)-phenyl]-pyrrolo[2,3-d]pyrimidin-7-yl}-cyclobutanecarbaldehyde (Intermediate Q) and (S)-2-methylpyrrolidine-2-carboxamide. MS m/z 519.3 (M+H⁺) (Method M).

### Example 94

### (2S,4R)-1-(((1 S,3R)-3-(4-amino-5-(3-((tetrahydro-2H-pyran-2-yl)methoxy)phenyl)-7H-pyrrolo[2,3-d]pyrimidin-7-yl)cyclobutyl)methyl)-4-fluoropyrrolidine-2-carboxamide

The title compound was prepared in a similar manner to Example 27 starting from 3-{4-chloro-5-[3-(tetrahydro-pyran-2-ylmethoxy)-phenyl]-pyrrolo[2,3-d]pyrimidin-7-yl}-cyclobutanecarbaldehyde (Intermediate Q) and (2S,4R)-4-fluoropyrrolidine-2-carboxamide hydrochloride salt (Intermediate AE). MS m/z 523.3 (M+H⁺) (Method M).

### Example 95

### (1R,3R,45)-2-(((1s, 3S)-3-(4-amino-5-(3-((tetrahydro-2H-pyran-2-yl)methoxy)phenyl)-7H-pyrrolo[2,3-d]pyrimidin-7-yl)cyclobutyl)methyl)-2-azabicyclo[2.2.1]heptane-3-carboxamide

The title compound was prepared in a similar manner to Example 27 starting from 3-{4-chloro-5-[3-(tetrahydro-pyran-2-ylmethoxy)-phenyl]-pyrrolo[2,3-d]pyrimidin-7-yl}-cyclobutanecarbaldehyde (Intermediate Q) and (1R,3R,4S)-2-azabicyclo[2.2.1]heptane-3-carboxamide hydrochloride salt (Intermediate AF). MS m/z 531.2 (M+H⁺) (Method M).

### Example 96

### (3S)-cis-4-((3-(4-amino-5-(3-((tetrahydro-2H-pyran-2-yl)methoxy)phenyl)-7H-pyrrolo[2,3-d]pyrimidin-7-yl}-cyclobutyl)methyl)morpholine-3-carboxamide

The title compound was prepared in a similar manner to Example 27 starting from cis-3-{4-amino-5-[3-(tetrahydro-pyran-2-ylmethoxy)-phenyl]-pyrrolo[2,3-d]pyrimidin-7-yl}-cyclobutanecarbaldehyde (Intermediate Q) and (S)-morpholine-3-carboxamide (Intermediate AG). MS m/z 521.3 (M+H⁺) (Method M). ¹H-NMR (MeOH d₄, 400 MHz) δ ppm 8.09 (s, 1H), 7.37 (s, 1H), 7.33 (t, 1H), 7.04-7.01 (m, 2H), 6.92-6.89 (m, 1H), 5.07-5.00 (m, 1H), 3.97-3.94 (m, 3H), 3.84-3.76 (m, 2H), 2.72-3.66 (m, 1H), 3.61-3.55 (m, 1H), 3.50-3.45 (m, 2H), 2.93-2.87 (m, 2H), 2.74-2.62 (m, 3H), 1.86 (br, 1H), 1.68-1.65 (m, 1H), 1.56-1.51 (m, 2H), 1.47-1.40 (m, 1H).

### Example 97

### (2S,4S)-1-(((1S,3R)-3-(4-amino-5-(3-((tetrahydro-2H-pyran-2-yl)methoxy)phenyl)-7H-pyrrolo[2,3-d]pyrimidin-7-yl)cyclobutyl)methyl)-4-fluoropyrrolidine-2-carboxamide

The title compound was prepared in a similar manner to Example 27 starting from cis-3-{4-amino-5-[3-(tetrahydro-pyran-2-ylmethoxy)-phenyl]-pyrrolo[2,3-d]pyrimidin-7-yl}-cyclobutanecarbaldehyde (Intermediate Q) and (2S,4S)-4-fluoropyrrolidine-2-carboxamide hydrochloride salt (Intermediate AH). MS m/z 523.3 (M+H⁺) (Method M).

### Example 98

### (3S)-4-((3-(4-amino-5-(3-((tetrahydro-2H-pyran-2-yl)methoxy)phenyl)-7H-pyrrolo[2,3-d]pyrimidin-7-yl)cyclobutyl)methyl)-6,6-dimethylmorpholine-3-carboxamide

The title compound was prepared in a similar manner to Example 27 starting from cis-3-{4-amino-5-[3-(tetrahydro-pyran-2-ylmethoxy)-phenyl]-pyrrolo[2,3-d]pyrimidin-7-yl}-cyclobutanecarbaldehyde (Intermediate Q) and (S)-6,6-dimethylmorpholine-3-carboxamide (Intermediate Al). MS m/z 549.3 (M+H⁺). (Method M). ¹H-NMR (MeOH d₄, 400 MHz) δ ppm 8.13 (s, 1H), 7.74 (s, 1H), 7.38 (t, 1H), 7.09-7.06 (m, 2H), 6.97-6.95 (m, 1H), 5.13-5.05 (m, 1H), 4.02-4.00 (m, 3H), 3.80-3.67 (m, 3H), 3.56-3.50 (m, 1H), 2.84-2.81 (m, 2H), 2.76-2.66 (m, 3H), 2.52-2.42 (m, 1H), 2.40-2.37 (m, 1H), 2.29-2.18 (m, 2H), 2.04-19.97 (m, 1H), 1.91 (br, 1H), 1.74-1/1 (m, 1H), 1.64-1.56 (m, 3H), 1.52-1.44 (m, 1H), 1.38 (s, 3H), 1.18 (s, 3H).

### Example 99

### (3S)-4-((3-(4-amino-5-(3-((tetrahydro-2H-pyran-2-yl)methoxy)phenyl)-7H-pyrrolo[2,3-d]pyrimidin-7-yl)cyclobutyl)methyl)-N-methylmorpholine-3-carboxamide

The title compound was prepared in a similar manner to Example 27 starting from cis-3-{4-amino-5-[3-(tetrahydro-pyran-2-ylmethoxy)-phenyl]-pyrrolo[2,3-d]pyrimidin-7-yl}-cyclobutanecarbaldehyde (Intermediate Q) and (S)—N-methylmorpholine-3-carboxamide (Intermediate AJ). MS m/z 521.3 (M+H⁺) (Method M).

### Example 100

### (2R)-1-(((1R,3R)-3-(4-amino-5-(3-((tetrahydro-2H-pyran-2-yl)methoxy)phenyl)-7H-pyrrolo[2,3-d]pyrimidin-7-yl)cyclobutyl)methyl)-N-methylpyrrolidine-2-carboxamide

A mixture of (R)-methyl 1-(((1R,3R)-3-(4-amino-5-(3-((tetrahydro-2H-pyran-2-yl)methoxy)phenyl)-7H-pyrrolo[2,3-d]pyrimidin-7-yl)cyclobutyl)methyl)pyrrolidine-2-carboxylate (Intermediate AK, 30 mg, 0.058 mmol) and lithium hydroxide (7 mg, 0.29 mmol) in ethanol and water (5:1, 1 mL) was heated at 120° C. under microwave irridiation for 10 minutes. The mixture was concentrated and the resulting residue was dried under vacuum. Then the residue was suspended in DMF (1 mL). To the mixture was added diisopropylethylamine (50 uL, 0.29 mmol) and HATU (22 mg, 0.058 mmol). After stirring for 15 minutes, methylamine (1 M in THF, 0.3 mL) was added. The mixture was stirred for 2 hours and purified by reversed phase preparative HPLC (Method S) to afford the title compound. MS m/z 519.3 (M+H⁺) (Method M).

### Example 101

### (S)-1-(((1S,3R)-3-(5-(3-(7-oxabicyclo[2.2.1]heptan-1-ylmethoxy)phenyl)-4-amino-7H-pyrrolo[2,3-d]pyrimidin-7-yl)cyclobutyl)methyl)-2-methylpyrrolidine-2-carboxamide

The title compound was prepared in a similar manner to Example 27 starting from cis-3-{4-amino-5-[3-(7-oxa-bicyclo[2.2.1]hept-1-ylmethoxy)-phenyl]-pyrrolo[2,3-d]pyrimidin-7-yl}-cyclobutanecarbaldehyde (Intermediate N) and (S)-2-methylpyrrolidine-2-carboxamide. MS m/z 531.3 (M+H⁺) (Method M). ¹HNMR (MeOD-d₄) δ ppm 8.03 (s, 1H), 7.34 (m, 2H), 7.01 (m, 2H), 6.92 (m, 1H), 5.00 (m, 1H), 4.50 (m, 1H), 4.24 (s, 2H), 2.90 (s, 3H), 2.76 (s, 3H), 2.62 (m, 3H), 2.45 (m, 2H), 2.30 (m, 1H), 2.07 (m, 4H), 1.76 (m, 4H), 1.59 (m, 4H).

### Example 102

### (1R,2S,5S)-3-(((1S,3R)-3-(4-amino-5-(3-((tetrahydro-2H-pyran-2-yl)methoxy)phenyl)-7H-pyrrolo[2,3-d]pyrimidin-7-yl)cyclobutyl)methyl)-3-azabicyclo[3.1.0]hexane-2-carboxamide

The title compound was prepared in a similar manner to Example 27 starting from (cis-3-{4-amino-5-[3-(tetrahydro-pyran-2-ylmethoxy)-phenyl]-pyrrolo[2,3-d]pyrimidin-7-yl}-cyclobutanecarbaldehyde (Intermediate Q) and (1R,2S,5S)-3-azabicyclo[3.1.0]hexane-2-carboxamide (Intermediate AL). MS m/z 520.3 (M+H⁺) (Method M).

### Example 103

### 7-[cis-3-(1,1-Dioxo-1λ6-perhydro-1,4-thiazepin-4-ylmethyl)-cyclobutyl]-5-[3-(tetrahydro-pyran-2-ylmethoxy)-phenyl]-7H-pyrrolo[2,3-d]pyrimidin-4-ylamine

The title compound was prepared in a similar manner to Example 27 starting from cis-3-{4-amino-5[3-(tetrahydro-pyran-2-ylmethoxy)-phenyl]-pyrrolo[2,3-d]pyrimidin-7-yl}-cyclobutanecarbaldehyde (Intermediate Q) and perhydro-1,4-thiazepine 1,1-dioxide (Intermediate AM). MS m/z 538.2 (M+H⁺) (Method M).

### Example 104

### (1R,3S,4S)-2-(((1S,3R)-3-(5-(3-(7-oxabicyclo[2.2.1]heptan-1-ylmethoxy)phenyl)-4-amino-7H-pyrrolo[2,3-d]pyrimidin-7-yl)cyclobutyl)methyl)-2-azabicyclo[2.2.1]heptane-3-carboxamide

The title compound was prepared in a similar manner to Example 27 starting from (1S,3S)-3-5-(3-(7-oxabicyclo[2.2.1]heptan-1-ylmethoxy)phenyl)-4-amino-7H-pyrrolo[2,3-d]pyrimidin-7-yl)cyclobutanecarbaldehyde (intermediate N) and (1R,3S,4S)-2-azabicyclo[2.2.1]heptane-3-carboxamide hydrochloride salt (Intermediate AN). MS m/z 543.2 (M+H⁺) (Method M).

### Example 105

### 7-[cis-3-(2,2-Dimethyl-1,1-dioxo-1λ6-thiomorpholin-4-ylmethyl)-cyclobutyl]-5-[3-(tetrahydro-pyran-2-ylmethoxy)-phenyl]-7H-pyrrolo[2,3-d]pyrimidin-4-ylamine

The title compound was prepared in a similar manner to Example 27 starting from cis-3-{4-amino-5-[3-(tetrahydro-pyran-2-ylmethoxy)-phenyl]-pyrrolo[2,3-d]pyrimidin-7-yl}-cyclobutanecarbaldehyde (Intermediate Q) and 2,2-dimethyl-thiomorpholine 1,1-dioxide (Intermediate AO). MS m/z 552.3 (M+H⁺) (Method M). ¹H-NMR (MeOH d₄, 400 MHz) δ ppm 8.13 (s, 1H), 7.43 (s, 1H), 7.38 (t, 1H), 7.09-7.06 (m, 2H), 6.97-6.94 (m, 1H), 5.15-5.06 (m, 1H), 4.02-3.99 (m, 3H), 3.77-3.71 (m, 1H), 3.55-3.50 (m, 1H), 3.13 (br, 2H), 2.95 (br, 2H), 2.71-2.65 (m, 6H), 2.46-2.38 (m, 1H), 2.25-2.17 (m, 2H), 1.92 (br, 1H), 1.73-1.69 (m, 1H), 1.64-1.55 (m, 3H), 1.55-1.45 (m, 1H), 1.38 (s, 6H).

### Example 106

### 4-(((1S,3S)-3-(5-(3-(7-oxabicyclo[2.2.1]heptan-1-ylmethoxy)phenyl)-4-amino-7H-pyrrolo[2,3-d]pyrimidin-7-yl}-cyclobutyl)methyl)thiomorpholin-3-one

A mixture of sodium hydride (5.8 mg, 0.145 mmol, 4 eq) and thiomorpholin-3-one (40 mg, 0.34 mmol, 10 eq) in DMF (1 mL) was stirred at room temperature for 10 min. ((1S,3S)-3-(5-(3-(7-oxabicyclo[2.2.1]heptan-1-ylmethoxy)phenyl)-4-amino-7H-pyrrolo[2,3-d]pyrimidin-7-yl}-cyclobutyl)methyl 4-methylbenzenesulfonate (Intermediate AP, 20 mg, 0.035 mmol, 1 eq) was added. The reaction was stirred at 60° C. overnight. The reaction was diluted with EtOAc (50 mL), washed with water (2×5 mL), saturated aqueous NaCl (5 mL), dried over Na₂SO₄ and evaporated. The crude product was purified by flash chromatography (SiO₂, MeOH:EtOAc/0-10%) to give the title compound as a white solid. MS m/z 520.2 (M+H⁺) (Method M). ¹HNMR (CDCl₃) δ ppm 8.31 (s, 1H), 7.37 (t, J=7.6 Hz, 1H), 7.16 (s, 1H), 7.09-7.06 (m, 2H), 6.98 (dd, J=2.0, 8.4 Hz, 1H), 5.18-5.12 (m, 3H), 4.63 (t, J=4.8 Hz, 1H), 4.32 (s, 2H), 3.65 (m, 2H), 3.61 (d, J=6.8 Hz, 2H), 3.33 (s, 2H), 2.89 (m, 2H), 2.73 (m, 2H), 2.46 (m, 1H), 2.28 (ddd, J=2.4, 9.2, 18.8 Hz, 2H), 1.86 (m, 4H), 1.64 m, 4H).

### Example 107

### 1-methylcyclopropyl ((1S,3S)-3-(5-(3-(7-oxabicyclo[2.2.1]heptan-1-ylmethoxy)phenyl)-4-amino-7H-pyrrolo[2,3-d]pyrimidin-7-yl)cyclobutyl)carbamate

A mixture of 5-(3-(7-oxabicyclo[2.2.1]heptan-1-ylmethoxy)phenyl)-7-((1S,3S)-3-(aminomethyl)cyclobutyl)-7H-pyrrolo[2,3-d]pyrimidin-4-amine (Intermediate AQ, 11 mg, 0.026 mmol), 1-methylcyclopropyl 4-nitrophenyl carbonate (12 mg, 0.52 mmol) and TEA (50 uL) in DCM (1 mL) was stirred at room temperature overnight. The reaction was diluted with EtOAc (10 mL), washed with NaOH (1N, 2×10 mL), saturated aqueous NaCl (1 mL), dried over Na₂SO₄ and evaporated. The residue was purified by flash chromatography (SiO₂, MeOH:DCM/0-10%) to give the title compound as a white solid. MS m/z 518.2 (M+H⁺) (Method M). ¹HNMR (CD₃OD) δ ppm 8.15 (s, 1H), 7.40 (m, 2H), 7.13 (m, 1H), 7.1 (dt, J=7.6, 1.2 Hz, 1H), 7.02 (ddd, J=0.8, 2.4, 8.4 Hz, 1H), 5.09 (quintet, J=8.4 Hz, 1H), 4.60 (t, J=4.8 Hz, 1H), 4.35 (s, 2H), 3.27 (m, 2H), 2.62 (m, 2H), 2.40 (m, 1H), 2.31 (m, 2H), 1.85 (m, 4H), 1.70 (m, 4H), 1.50 (5, 3H), 0.82 (m, 2H), 0.60 (m, 2H).

### Example 108

### 2-(((1S,3S)-3-(5-(3-(7-oxabicyclo[2.2.1]heptan-1-ylmethoxy)phenyl)-4-amino-7H-pyrrolo[2,3-d]pyri nnidin-7-yl)cyclobutyl)methyl)isoindoline-1,3-dione

To a mixture of ((1S,3S)-3-(5-(3-(7-oxabicyclo[2.2.1]heptan-1-ylmethoxy)phenyl)-4-amino-7H-pyrrolo[2,3-d]pyrimidin-7-yl)cyclobutyl)methanol (Example 22, 100 mg, 0.24 mmol), tributylphosphine (72 mg, 0.36 mmol) and phthalimide (52 mg, 0.36 mmol) in anhydrous toluene (1 mL) at 0° C. was added DEAD (162 uL, 40% toluene, 1.5 eq). The reaction was stirred at room temperature overnight. After aqueous work up, the crude product was purified by flash chromatography (SiO₂, MeOH:EtOAc0-10%) to give the title compound. MS m/z 550.2 (M+H⁺) (Method M). ¹HNMR (CDCl₃) δ ppm 8.11 (s, 1H), 7.85 (m, 2H), 7.20 (m, 2H), 7.35 (t, J=7.6 Hz, 1H), 7.12 (s, 1H), 7.07-7.04 (m, 2H), 6.97 (ddd, J=0.8, 2.8, 8.4 Hz, 1H), 5.28 (br s, 2H), 5.07 (m, 1H), 4.62 (t, 4.8 Hz, 1H), 4.31 (s, 2H), 3.85 (d, J=6.8 Hz, 2H), 2.67 (m, 2H), 2.58 (m, 1H), 2.37 (m, 2H), 1.85 (m, 4H), 1.62 (m, 4H).

### Example 109

### 5-(3-(7-oxabicyclo[2.2.1]heptan-1-ylmethoxy)phenyl)-7-((1S,4S)-4-(4-methylpiperazin-1-yl)cyclohexyl)-7H-pyrrolo[2,3-d]pyrimidin-4-amine

The title compound was prepared in a similar manner to Example 27 starting from Intermediate AR and 1-methylpiperazine. The trans (upper band) and cis isomers (lower band) were separated with prep. TLC, eluting with 10% MeOH in DCM with 0.1 N ammonia. MS m/z 517.3 (M+H⁺) (Method M) and MS m/z 517.3 (M+H⁺) (Method M). cis isomer: ¹H-NMR (Acetone-d₆, 400 MHz) δ ppm 8.17 (s, 1H), 7.39-7.35 (m, 2H), 7.14-7.13 (m, 1H), 7.10-7.08 (m, 1H), 6.97-6.95 (m, 1H), 5.81 (br, 2H), 4.68-4.62 (m, 1H), 4.51-4.49 (m, 1H), 4.35 (s, 2H), 2.93 (br, 2H), 2.58 (br, 4H), 2.46-2.40 (m, 1H), 2.33 (br, 1H), 2.17 (s, 3H), 2.11-2.06 (m, 1H), 2.04-1.92 (m, 4H), 1.76-1.72 (m, 3H), 1.61-1.50 (m, 6H).

### Example 110

### ((1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexyl)methanol

A mixture of 2-(3-(7-oxabicyclo[2.2.1]heptan-1-ylmethoxy)phenyl)-4,4,5,5-tetramethyl-1,3,2-dioxaboroiane (Intermediate K, 1.78 g, 5.39 mmol), ((1S,4S)-4-(4-amino-5-iodo-7H-pyrrolo[2,3-d]pyrimidin-7-yl)cyclohexyl)methanol (Intermediate AS, 2.01 g, 5.39 mmol), tetrakis triphenylphosphine palladium (311 mg, 0.27 mmol, 0.05 eq) and sodium carbonate (2.86 g, 27 mmol) in DMF (26 mL) and water (13 mL) was degassed by a stream of Argon gas. The mixture was sealed and stirred at 90° C. for 2 h. The reaction was quenched with water (60 mL) and extracted with dichloromethane (4×50 mL). The dichloromethane layer was washed with water (20 mL), saturated aqueous NaCl (20 mL), dried over Na₂SO₄ and evaporated. The crude product was purified by Flash chromatography (SiO₂, MeOH:DCM/1:9) to give the title compound as an off-white solid. MS m/z 449.2 (M+H⁺) (Method M). ¹HNMR (CD₃OD) δ ppm 8.14 (s, 1H), 7.37 (m, 2H), 7.10 (m, 2H), 7.00 (m, 1H), 4.66 (m, 1H), 4.59 (t, J=4.0 Hz, 1H), 4.34 (s, 2H), 3.71 (d, J=7.2 Hz, 2H), 2.10-1.70 (m, 13H), 1.70 (m, 4H).

### Example 111

### 7-[4-(1,1-Dioxo-thiomorpholin-4-ylmethyl)-cyclohexyl]-5-[3-(7-oxa-bicyclo[2.2.1]hept-1-ylmethoxy)-phenyl]-7H-pyrrolo[2,3-d]pyrimidin-4-ylamine

To a mixture of (1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexanecarbaldehyde (Intermediate AT, 0.105 mmol), 1,1-dioxide-thiomorpholine (57 mg, 0.42 mmol) and diisopropylethylamine (145 uL, 0.84 mmol) in dichloroethane (2 mL) was added sodium triacetoxyborohydride (89 mg, 0.42 mmol). After stirring at room temperature for 1 h, the reaction was quenched with water (15 mL), extracted with EtOAc (3×30 mL). The EtOAc layer was washed with water (10 mL), saturated aqueous NaCl (5 mL), dried over Na₂SO₄ and evaporated. The resulting residue was purified by flash chromatography (SiO₂, MeOH:DCM/0-10%) to give the title compound as off-white solid. MS m/z 566.2 (M+H⁺) (Method M). ¹HNMR (CD₃OD) δ ppm 8.14 (s, 1H), 7.39 (m, 2H), 7.10 (m, 2H), 7.00 (dd, J=1.6, 8.0 Hz, 1H), 4.60 (m, 2H), 4.33 (s, 2H), 3.11 (m, 4H), 3.04 (m, 4H), 2.66 (d, J=8.0 Hz, 2H), 1.99 (m, 3H), 1.90-1.74 (m, 10H), 1.80 (m, 4H).

### Example 112

### 5-(3-(7-oxabicyclo[2.2.1]heptan-1-ylmethoxy)phenyl)-7-((1S,4S)-4-(thiomorpholinomethyl)cyclohexyl)-7H-pyrrolo[2,3-d]pyrimidin-4-amine

The title compound was prepared in a similar manner to Example 111 starting from (1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexanecarbaldehyde (Intermediate AT) and thiomorpholine. MS 534.2 (M+H⁺) (Method M). ¹HNMR (CD₃OD) δ ppm 8.13 (s, 1H), 7.39 (m, 2H), 7.10 (m, 2H), 7.00 (dd, J=2.4, 8.0 Hz, 1H), 4.61 (m, 2H), 4.34 (s, 2H), 2.76 (m, 4H), 2.67 (m, 4H), 2.51 (d, J=7.6 Hz, 2H), 2.01 (m, 3H), 1.92-1.72 (m, 10H), 1.68 (nri, 4H).

### Example 113

### 5-(3-(7-oxabicyclo[2.2.1]heptan-1-ylmethoxy)phenyl)-7-((1S,4S)-4-((4-methylpiperazin-1-yl)methyl)cyclohexyl)-7H-pyrrolo[2,3-d]pyrimidin-4-amine

The title compound was prepared in a similar manner to Example 111 starting from (1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexanecarbaldehyde (Intermediate AT) and methylpiperazine. MS m/z 531.3 (M+H⁺) (Method M). ¹HNMR (CD₃OD) δ ppm 8.13 (s, 1H), 7.39 (m, 2H), 7.10 (m, 2H), 6.99 (dd, J=2.4, 8.0 Hz, 1H), 4.60 (m, 2H), 4.33 (s, 2H), 2.80-2.30 (m, 9H), 2.29 (s, 3H), 2.01 (m, 3H), 1.89-1.70 (11H), 1.67 (m, 4H).

### Example 114

### 5-(3-(7-oxabicyclo[2.2.1]heptan-1-ylmethoxy)phenyl)-7-(1-(2-(methylsulfonyl)ethyl)piperidin-4-yl)-7H-pyrrolo[2,3-d]pyrimidin-4-amine

To the solution of 5-(3-(7-oxabicyclo[2.2.1]heptan-1-ylmethoxy)phenyl)-7-(piperidin-4-yl)-7H-pyrrolo[2,3-d]pyrimidin-4-amine (Intermediate AU, 20 mg, 0.04 mmol) in DMF (1.0 mL) was sequentially added methylsulfonylethene (43 mg, 0.4 mmol) and triethylamine (0.05 mL, 0.4 mmol). The reaction was heated at 120° C. in a microwave reactor for 10 min. and the crude product was purified by reverse phase preparative HPLC (Method 5) to obtain the title compound. MS m/z 526.2 (M+H⁺) (Method M). ¹HNMR (MeOD-d₄) δ ppm 8.15 (s, 1H), 7.39 (dt, 1H), 7.35 (s, 1H), 7.10 (m, 2H), 7.00 (m, 1H), 4.67 (m, 1H), 4.59 (m, 1H), 3.34 (m, 2H), 3.32 (m, 3H), 3.16 (m, 2H), 3.10 (s, 3H), 2.93 (m, 2H), 2.34 (m, 2H), 2.07 (m, 4H), 1.82 (m, 3H), 1.68 (m, 3H).

### Example 115

### 5-[3-(7-Oxa-bicyclo[2.2.1]hept-1-ylmethoxy)-phenyl]-7-cis-[4-(1-oxo-1λ4-thiomorpholin-4-ylmethyl)-cyclohexyl]-7H-pyrrolo[2,3-d]pyrimidin-4-ylamine

To a solution of 5-(3-(7-oxabicyclo[2.2.1]heptan-1-ylmethoxy)phenyl)-7-((1S,4S)-4-(thiomorpholinomethyl)cyclohexyl)-7H-pyrrolo[2,3-d]pyrimidin-4-amine (Example 112, 18 mg, 0.034 mmol) in MeCN (2 mL) was added oxone (8 mg) solution in water (0.4 mL). After stirring at room temperature overnight, the reaction mixture was purified by flash chromatography (SiO₂, MeOH:DCM/0-10%) to give the title compound. MS m/z 550.2 (M+H⁺) (Method M). ¹HNMR (CD₃OD) δ ppm 8.14 (s, 1H), 7.41 (s, 1H), 7.38 (d, J=7.6 Hz, 1H), 7.10 (m, 2H), 6.99 (dd J=2.4, 8.4 Hz, 1H), 4.68-4.58 (m, 3H), 4.33 (s, 2H), 3.02 (m, 4H), 2.84 (m, 4H), 2.58 (d, J=7.6 Hz, 2H), 2.01 (m, 3H), 1.90-1.75 (m, 9H), 1.68 (m, 4H).

### Example 116

### 5-(3-(7-oxabicyclo[2.2.1]heptan-1-ylmethoxy)phenyl)-7-((1S,4S)-4-(4,4-difluoropiperidin-1-yl)cyclohexyl)-7H-pyrrolo[2,3-d]pyrimidin-4-amine

The title compound was prepared in a similar manner to Example 27 starting from Intermediate AR and 4,4-difluoropiperidine hydrochloride. The trans isomer (upper band) and cis isomer (lower band) were separated with prep. TLC, eluting with 6% MeOH in DCM with 0.1 N ammonia MS m/z 538.3 (M+H⁺) (Method M) and MS m/z 538.3 (M+H⁺) (Method M). cis isomer: ¹H-NMR (MeOH d₄, 400 MHz) δ ppm 8.14 (s, 1H), 7.39 (t, 1H), 7.33 (s, 1H), 7.10-7.06 (m, 2H), 7.01-6.99 (m, 1H), 4.61-4.58 (m, 2H), 4.33 (s, 2H), 2.80-2.78 (m, 4H), 2.71-2.66 (m, 1H), 2.16-1.94 (m, 10H), 1.85-1.82 (m, 4H), 1.71-1.63 (m, 6H).

### Example 117

### (1R,4R)-4-(5-(3-(7-oxabicyclo[2.2.1]heptan-1-ylmethoxy)phenyl)-4-amino-7H-pyrrolo[2,3-d]pyrimidin-7-yl)-1-((cyclopropylamino)methyl)cyclohexanol

A mixture of 5-(3-(7-oxabicyclo[2.2.1]heptan-1-ylmethoxy)phenyl)-7-((3R,6R)-1-oxaspiro[2.5]octan-6-yl)-7H-pyrrolo[2,3-d]pyrimidin-4-amine (Intermediate AV) (45 mg, 0.1 mmol) and cyclopropaneamine (1.0 mL) in EtOH (1 mL) was heated at 80° C. overnight. The reaction mixture was cooled to room temperature and concentrated in vacuo. The residue was purified by reverse phase preparative HPLC (Method S) to afford the title compound. MS m/z 503.2 (M+H⁺) (Method M). ¹HNMR (MeOD-d₄) δ ppm 8.04 (s, 1H), 7.27 (m, 2H), 7.01 (m, 2H), 6.90 (m, 2H), 4.62 (m, 2H), 4.53 (m, 2H), 4.49 (m, 1H), 4.24 (m, 2H), 3.49 (m, 1H), 2.87 (m, 1H), 2.01 (m, 6H), 1.73 (m, 5H), 1.59 (m, 5H).

### Example 118

### 5-(3-(7-oxabicyclo[2.2.1]heptan-1-ylmethoxy)phenyl)-7-(1-(oxetan-3-yl)piperidin-4-yl)-7H-pyrrolo[2,3-d]pyrimidin-4-amine

The title compound was prepared in a similar manner to Example 27 starting from 5-(3-(7-oxabicyclo[2.2.1]heptan-1-ylmethoxy)phenyl)-7-(piperidin-4-yl)-7H-pyrrolo[2,3-d]pyrimidin-4-amine (Intermediate AU) and oxetan-3-one. MS m/z 476.3 (M+H⁺) (Method M).

### Examples 119 and 121

### 5-(3-(((S)-5,5-dimethyltetrahydrofuran-2-yl)methoxy)phenyl)-7-((1S,4S)-4-(4-methylpiperazin-1-yl)cyclohexyl)-7H-pyrrolo[2,3-d]pyrimidin-4-amine and 5-(3-(((R)-5,5-dimethyltetrahydrofuran-2-yl)methoxy)phenyl)-7-((1S,4S)-4-(4-methylpiperazin-1-yl)cyclohexyl)-7H-pyrrolo[2,3-d]pyrimidin-4-amine

The racemic sample of 5-[3-(5,5-dimethyl-tetrahydro-furan-2-ylmethoxy)-phenyl]-7-[cis-4-(4-methyl-piperazin-1-yl)-cyclohexyl]-7H-pyrrolo[2,3-d]pyrimidin-4-ylamine (Example 63) was separated into pure enantiomers by chiral preparative HPLC (Column: 20×250 mm ChiralCel OD; Conditions: 25 mL/min flow rate, 65/17.5/17.5 Hexane:EtOH:MeOH; Run Time: 16 minutes). Analytical chiral HPLC retention times: 6.10 min. and 14.67 min. (Column: 4.6×250 mm ChiralCel OD-H; Conditions: 1mL/min flow rate, 70:15:15 Hexane:EtOH:MeOH to give:

Example 119 as the second eluting enanantimer 5-(3-(((S)-5,5-dimethyltetrahydrofuran-2-yl)methoxy)phenyl)-7-((1S,4S)-4-(4-methylpiperazin-1-yl)cyclohexyl)-7H-pyrrolo[2,3-d]pyrimidin-4-amine. MS m/z 519.3 (M+H⁺) (Method M).
Example 121 as the first eluting enantiomer 5-(3-(((R)-5,5-dimethyltetrahydrofuran-2-yl)methoxy)phenyl)-7-(1S,4S)-4-(4-methylpiperazin-1-yl)cyclohexyl)-7H-pyrrolo[2,3-d]pyrimidin-4-amine. MS m/z 519.3 (M+H⁺) (Method M).
LCMS & NMR data were identical for Example 63, Example 119 & Example 121.

### Example 120

### (1S)-1-((2S)-1-(((1S,3R)-3-(4-amino-5-(3-((tetrahydro-2H-pyran-2-yl)methoxy)phenyl)-7H-pyrrolo[2,3-d]pyrimidin-7-yl)cyclobutyl)methyl)pyrrolidin-2-yl)-2,2,2-trifluoroethanol

The title compound was prepared in a similar manner to Example 27 starting from (S)-2,2,2-trifluoro-1((S)-pyrrolidin-2-yl)pethanol (ref.: Tetrahedron 2008 (64), 7353-7361). MS m/z 560.3 (M+H⁺) (Method M). ¹HNMR (MeOD-d₄) δ ppm 8.14 (s, 1H), 7.46 (s, 1H), 7.39 (m, 1H), 7.07 (m, 2H), 6.96 (m, 1H), 5.11 (m, 1H), 4.16 (m, 1H), 4.00 (m, 3H), 3.76 (m, 1H), 3.54 (m, 1H), 3.15 (m, 1H), 2.98 (m, 1H), 2.72 (m, 3H), 2.47 (m, 2H), 2.27 (m, 3H), 2.09 (m, 1H), 1.92 (m, 1H), 1.77 (m, 3H), 1.60 (m, 3H).

### Example 122

### (Z)-3-{(8-Amino-1-[3-(7-oxa-bicyclo[2.2.1]hept-1-ylmethoxy)-phenyl]-imidazo[1,5-a]pyrazin-3-yl}-1-methyl-cyclobutanol

A solution of methylmagnesium bromide in diethyl ether (3 M, 0.128 ml, 383 mmol) was added dropwise to a solution of 3-{8-amino-1-[3-(7-oxa-bicyclo[2.2.1]hept-1-ylmethoxy)-phenyl]-imidazo[1,5-a]pyrazin-3-yl}-cyclobutanone (Intermediate BQ, 86 mg, 0.213 mmol) in THF (2 ml) at −78° C. The reaction mixture was stirred for 10 minutes at −78° C., for 1 hour at 0° C. and then further methylmagnesium bromide in diethyl ether (0.035 ml) added. After 1 hour at 0° C. the reaction mixture was partitioned between water and ethyl acetate, the organic layers washed with brine, dried over sodium sulphate and evaporated. Purification of the residue by normal phase chromatography, eluting with a gradient of methanol in DCM, gave the title compound as a yellow solid. HPLC/MS t_{R} 0.78 min, M+H 421.1 (Method X).

### Example 123

### (Z)-3-{4-Amino-5-[3-(7-oxa-bicyclo[2.2.1]hept-1-ylmethoxy)-phenyl]-pyrrolo[2,3-d]pyrimidin-7-yl}-1-(4-methyl-piperazin-1-ylmethyl)-cyclobutanol

Compound of Step BL.3 (30 mg, 0.051 mmol), methylpiperazine (0.028 mL, 0.254 mmol), K₂CO₃ (70.9 mg, 0.508 mmol), and NEt₃ (0.072 mL, 0.508 mmol) were suspended in DMF (1 mL) and stirred at 90° C. for 2 h under Ar. The reaction mixture was concentrated under reduced pressure and partitined between AcOEt (40 mL) and H₂O (40 mL). The aqueous phase was extracted with AcOEt (20 mL, 2×). The combined organic phases were washed with H₂O (10 mL) and brine (10 mL, 2×), dried (Na₂SO₄), and concentrated under reduced pressure to give a white solid (29 mg). The material was purified by column chromatography using a 12 g silica gel column (RediSept (Isco)) and a Sepacore Control chromatography system from Buchi: DCM (10 min) -->DCM/MeOH (10% NH₃ (25%))=9:1 in 30 min at a flow rate of 30 ml/min) to give the title compound as a white solid (19 mg). HPLC t_{R}=2.151 min (method D), M+H=519.3 (method L). ¹H NMR (600 MHz, DMSO-d₆) δ ppm 8.12 (s, 1H, pyrimidinyl), 7.63 (s, 1H, pyrrolyl), 7.36 (t, 1H, phenyl), 7.09 (s, 1H, phenyl), 7.0716.96 (d/d, 1H/1H, phenyl), 6.15 (s/b, 2H, NH₂), 5.20 (s, 1H, OH), 4.71 (quintet, 1H, CH-cyclobutyl), 4.50 (s, 1H, CH-oxabicycloheptanyl), 4.30 (s, 2H, CH₂—O), 2.7012.35 (tit, 4H, CH₂-cyclobutyl), 2.70-2.30 (m, 8H, CH₂-piperazinyl), 2.41 (s, 2H, CH₂—N), 2.24 (s, 3H, CH₃-piperazinyl), 1.68/1.62 (m/m, 8H, CH₂-oxabicycloheptanyl).

### Example 124

### (R)-1-(((1S,4S)-4-(5-(3-(7-oxabicyclo[2.2.1]heptan-1-yl(nnethoxy)phenyl)-4-amino-7H-pyrrolo[2,3-d]pyrimidin-7-cyclohexyl)methyl)pyrrolidin-3-ol

The title compound was prepared in a similar manner to Example 111 starting from (1S,4s)-4-(5-(3-(7-oxabicyclo[2.2.1]heptan-1-ylmethoxy)phenyl)-4-amino-7H-pyrrolo[2,3-d]pyrimidin-7-yl)cyclohexanecarbaldehyde (Intermediate AT) and (R)-pyrrolidin-3-ol. MS m/z 518.3 (M+H⁺) (Method M).

### Example 125

### 5-(3-(7-oxabicyclo[2.2.1]heptan-1-ylmethoxy)phenyl)-7-((1R,4S)-4-((R)-3-fluoropyrrolidin-1-yl)cyclohexyl)-7H-pyrrolo[2,3-d]pyrimidin-4-amine

The title compound was prepared in a similar manner to Example 27 starting from Intermediate AR and (R)-3-fluoropyrrolidine. The trans and cis isomers were separated with preparative TLC, eluting with 10% MeOH in DCM with 0.1 N ammonia. MS m/z 506.7 (M+H⁺) (Method M). ¹HNMR (MeOD-d₄) δ ppm 8.15 (s, 1H), 7.40 (m, 1H), 7.33 (s, 1H), 7.10 (m, 2H), 7.02 (m, 1H), 5.30 (m, 1H), 4.67 (m, 1H), 4.60 (m, 1H), 4.34 (s, 2H), 3.18 (m, 2H), 3.00 (m, 1H), 2.82 (m, 1H), 2.59 (m, 1H), 2.25 (m, 6H), 1.96 (m, 3H), 1.83 (m, 4H), 1.68 (m, 5H).

### Example 126

### (S)-1-(((1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexyl)methyl)pyrrolidin-3-ol

The title compound was prepared in a similar manner to Example 111 starting from (1S,4s)-4-(5-(3-(7-oxabicyclo[2.2.1]heptan-1-ylmethoxy)phenyl)-4-amino-7H-pyrrolo[2,3-d]pyrimidin-7-yl)cyclohexanecarbaldehyde (Intermediate AT) and (S)-pyrrolidin-3-ol. MS m/z 518.2 (M+H⁺) (Method M).

### Example 127

### (S)-1-((1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexyl)pyrrolidin-3-ol

The title compound was prepared in a similar manner to Example 27 starting from Intermediate AR and (S)-pyrrolidin-3-ol. The trans and cis isomers were separated with preparative TLC, eluting with 10% MeOH in DCM with 0.1 N ammonia. MS m/z 504.3 (M+H⁺) (Method M). ¹HNMR (MeOD-d₄) δ ppm 8.16 (s, 1H), 7.40 (m, 1H), 7.33 (s, 1H), 7.10 (m, 2H), 7.01 (m, 1H), 4.65 (m, 1H), 4.60 (m, 1H), 4.50 (m, 1H), 4.34 (s, 2H), 3.44 (m, 2H), 3.23 (m, 1H), 3.06 (m, 1H), 2.35 (m, 2H), 2.21 (m, 3H), 1.83 (m, 4H), 1.68 (m, 4H), 1.32 (m, 3H).

### Example 128

### (R)-1-((1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexyl)pyrrolidin-3-ol

The title compound was prepared in a similar manner to Example 27 starting from Intermediate AR and (R)-pyrrolidin-3-ol. The trans and cis isomers were separated with preparative TLC, eluting with 10% MeOH in DCM with 0.1 N ammonia. MS m/z 504.3 (M+H⁺) (Method M). ¹HNMR (MeOD-d₄) δ ppm 8.16 (s, 1H), 7.40 (m, 1H), 7.33 (s, 1H), 7.10 (m, 2H), 7.01 (m, 1H), 4.68 (m, 1H), 4.60 (m, 1H), 4.52 (m, 1H), 4.34 (s, 2H), 3.39 (m, 2H), 3.19 (m, 2H), 2.99 (m, 1H), 2.32 (m, 2H), 2.20 (m, 2H), 2.03 (m, 3H), 1.93 (s, 6H), 1.83 (m, 2H), 1.68 (m, 3H).

### Example 129

### 4-(((1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexyl)methyl)piperazin-2-one

The title compound was prepared in a similar manner to Example 111 starting from (1S,4S)-4-(5-(3-((1s,4s)-7-oxabicyclo[2.2.1]heptan-1-ylmethoxy)phenyl)-4-amino-7H-pyrrolo[2,3-d]pyrimidin-7-yl)cyclohexanecarbaldehyde (Intermediate AT) and piperazin-2-one. MS m/z 531.2 (M+H⁺) (Method M).

### Example 130

### 5-(3-(7-oxabicyclo[2.2.1]heptan-1-ylmethoxy)phenyl)-7-(1s,4S)-4-(morpholinomethyl)cyclohexyl)-7H-pyrrolo[2,3-d]pyrimidin-4-amine

The title compound was prepared in a similar manner to Example 111 starting from (1S,4S)-4-(5-(3-((1s,4s)-7-oxabicyclo[2.2.1]heptan-1-ylmethoxy)phenyl)-4-amino-7H-pyrrolo[2,3-d]pyrimidin-7-yl)cyclohexanecarbaldehyde (Intermediate AT) and morpholine. MS m/z 518.3 (M+H⁺) (Method M). ¹HNMR (CD₃OD) δ ppm 8.14 (s, 1H), 7.40 (m, 2H), 7.12 (t, J=2.0 Hz, 1H), 7.09 (m, 1H), 7.00 (ddd, J=1.6, 4.0, 9.2 Hz, 1H), 4.61 (m, 2H), 4.34 (s, 2H), 3.72 (t, J=4.6 Hz, 4H), 2.52 (m, 6H), 2.05 (m, 3H), 1.90-1.70 (m, 10H), 1.68 (m, 4H).

### Example 131

### 7-[4-(1,1-Dioxo-1λ6-thiomorpholin-4-yl)-cyclohexyl]-5-[3-(7-oxa-bicyclo[2.2.1]hept-1-ylmethoxy)-phenyl]-7H-pyrrolo[2,3-d]pyrimidin-4-ylamine

To the solution of 5-(3-(7-oxabicyclo[2.2.1]heptan-1-ylmethoxy)phenyl)-7-((1s,4s)-4-aminocyclohexyl)-7H-pyrrolo[2,3-d]pyrimidin-4-amine (Intermediate AW, 12 mg, 0.03 mmol) in DMF (0.5 mL) was added vinylsulfonylethene (3.6 mg, 0.03 mmol). The reaction was heated at 40° C. overnight. The reaction mixture was purified with reverse phase preparative HPLC (Method 8) to afford the title compound. MS m/z 552.3 (M+H⁺) (Method M).

### Example 132

### 5-(3-(7-oxabicyclo[2.2.1]heptan-1-ylmethoxy)phenyl)-7-((1R,4R)-4-((S)-3-fluoropyrrolidin-1-yl)cyclohexyl)-7H-pyrrolo[2,3-d]pyrimidin-4-amine

The title compound was prepared in a similar manner to Example 27 starting from Intermediate AR and (S)-3-fluoropyrrolidine. The trans and cis isomers were separated with prep. TLC, eluting with 10% MeOH in DCM with 0.1 N ammonia. MS m/z 506.3 (M+H⁺) (Method M). ¹HNMR (MeOD-d₄) δ ppm 8.16 (s, 1H), 7.43 (m, 2H), 7.11 (m, 2H), 7.01 (m, 1H), 4.68 (m, 2H), 4.35 (s, 2H), 4.16 (m, 1H), 3.49 (m, 1H), 3.08 (m, 1H), 2.15 (m, 2H), 2.03 (s, 2H), 1.95 (m, 4H), 1.83 (m, 5H), 1.68 (m, 4H).

### Example 133

### N-((1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexyl)acetamide

To a solution 5-(3-(7-oxabicyclo[2.2.1]heptan-1-ylmethoxy)phenyl)-7-((1S,4S)-4-aminocyclohexyl)-7H-pyrrolo[2,3-d]pyrimidin-4-amine (Intermediate AW, 45 mg, 0.1 mmol) in DMF (1.0 mL) was sequentially added acetyl chloride (31 mg, 0.14 mmol) and triethylamine (26 mg, 0.26 mmol). The resulting reaction mixture was stirred at room temperature for 30 min. The crude product was purified by reverse phase preparative HPLC (Method S) to afford the title compound. MS m/z 476.3 (M+H⁺) (Method M).

### Example 134

### 5-(3-(7-oxabicyclo[2.2.1]heptan-1-ylmethoxy)phenyl)-7-((1S,4S)-4-(aminomethyl)cyclohexyl)-7H-pyrrolo[2,3-d]pyrimidin-4-amine

A mixture of 5-(3-(7-oxabicyclo[2.2.1]heptan-1-ylmethoxy)phenyl)-7-((1S,4S)-4-(azidomethyl)cyclohexyl)-7H-pyrrolo[2,3-d]pyrimidin-4-amine (Intermediate AX, 2.1 mmol) and triphenylphosphine (150 mg, 0.57 mmol) in THF (5 mL) and water (0.5 mL) was stirred at 80° C. in a sealed vial for 1 h. The solvent was evaporated and the residue was purified by flash chromatography (SiO₂, MeOH:DCM/10-15%) to give the title compound as an off-white solid. MS m/z 448.2 (M+H⁺) (Method M). ¹HNMR (CD₃OD) δ ppm 8.14 (s, 1H), 7.38 (m, 2H), 7.10 (m, 1H), 7.08 (m, 1H), 7.00 (dd, J=2.4, 8.0 Hz, 1H), 4.65 (m, 1H), 4.59 (t, J=4.4 Hz, 1H), 4.33 (s, 2H), 2.83 (d, J=6.8 Hz, 2H), 2.03 (m, 2H), 1.92-1.72 (m, 11H), 1.7 (m, 4H).

### Example 135

### 7-[4-(3,3-Dimethyl-1,1-dioxo-1λ6-thiomorpholin-4-ylmethyl)-cyclohexyl]-5-[3-(7-oxa-bicyclo[2.2.1]hept-1-ylmethoxy)-phenyl]-7H-pyrrolo[2,3-d]pyrimidin-4-ylamine

The title compound was prepared in a similar manner to Example 111 starting from (1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexanecarbaldehyde (Intermediate AT) and 3,3-dimethyl-thiomorpholine 1,1-dioxide (Intermediate AY). MS m/z 594.3 (M+H⁺) (Method M).

### Example 136

### 5-(3-(7-oxa bicyclo[2.2.1]heptan-1-ylmethoxy)phenyl)-7-((1S,4S)-4-(piperazin-1-ylmethyl)cyclohexyl)-7H-pyrrolo[2,3-d]pyrimidin-4-amine

The title compound was prepared in a similar manner to Example 111 starting from (1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexanecarbaldehyde (Intermediate AT) and piperazine. MS m/z 517.3 (M+H⁺) (Method M). ¹HNMR (CD₃OD) δ ppm 8.14 (s, 1H), 7.40 (m, 2H), 7.12 (m, 1H), 7.09 (m, 1H), 7.10 (ddd, J=0.8, 2.7, 8.4 Hz, 1H), 4.60 (m, 1H), 4.60 (t, J=0.8 Hz, 1H), 4.34 (s, 2H), 2.90 (t, J=4.8 Hz, 4H), 2.51 (m, 6H), 2.03 (m, 3H), 1.94-1.74 (m, 10H), 1.69 (m, 4H).

### Example 137

### 5-(3-(7-oxabicyclo[2.2.1]heptan-1-ylmethoxy)phenyl)-7-((1S,4S)-4-((4-(methylsulfonyl)piperazin-1-yl)methyl)cyclohexyl)-7H-pyrrolo[2,3-d]pyrimidin-4-amine

To a solution of 5-(3-(7-oxabicyclo[2.2.1]heptan-1-ylmethoxy)phenyl)-7-((1S,4S)-4-(piperazin-1-ylmethyl)cyclohexyl)-7H-pyrrolo[2,3-d]pyrimidin-4-amine (Example 136, 36 mg, 0.070 mmol) in DCM (1 mL) at 0° C. was added methanesulfonyl anhydride (13 mg, 0.077 mmol) in DCM (0.5 mL), followed by addition of TFA (30 μL). The solution was stirred at 0° C. for 1 h. The reaction was diluted with DCM (50 mL), washed with water (10 mL), saturated aqueous NaCl (10 mL), dried over Na₂SO₄ and evaporated. The residue was purified by flash chromatography (SiO₂, MeOH:DCM/0-10%) to give the product as a white solid. MS m/z 595.3 (M+H⁺) (Method M). ¹HNMR (CD₃OD) δ ppm 8.14 (s, 1H), 7.40 (m, 2H), 7.12 (m, 1H), 7.09 (m, 1H), 7.00 (dd, J=2.4, 8.4 Hz, 1H), 4.64 (m, 1H), 4.60 (t, J=4.8 Hz, 1H), 4.34 (s, 2H), 3.24 (t, J=4.8 Hz, 4H), 2.84 (s, 3H), 2.59 (t, J=4.6 Hz, 4H), 2.55 (d, J=8.0 Hz, 2H), 2.02 (m, 3H), 1.94-1.74 (m, 10H), 1.68 (m, 4H).

### Example 138

### 7-((1S,4S)-4-(1H-imidazol-1-yl)cyclohexyl)-5-(3-(7-oxabicyclo[2.2.1]heptan-1-ylmethoxy)phenyl)-7H-pyrrolo[2,3-d]pyrimidin-4-amine

To a mixture of (1R,4R)-4-(5-(3-(7-oxabicyclo[2.2.1]heptan-1-ylmethoxy)phenyl)-4-amino-7H-pyrrolo[2,3-d]pyrimidin-7-yl)cyclohexyl methanesulfonate (Step AW.2) (40 mg, 0.078 mmol) and 1H-imidazole (21 mg, 0.31 mmol) in DMF (1 mL), was added sodium hydride (13 mg, 0.34 mmol). The mixture was then sealed and heated at 75° C. overnight. The reaction was quenched by adding a few drops of water. The mixture was purified by reverse phase preparative HPLC (Method S) to afford the title compound. MS m/z 485.3 (M+H⁺) (Method M).

### Example 139

### 8-(5-(3-(7-oxabicyclo[2.2.1]heptan-1-ylmethoxy)phenyl)-4-amino-7H-pyrrolo[2,3-d]pyrimidin-7-yl)-1-oxa-3-azaspiro[4.5]decan-2-one

Under nitrogen, a mixture of 4-(5-(3-(7-oxabicyclo[2.2.1]heptan-1-ylmethoxy)phenyl)-4-amino-7H-pyrrolo[2,3-d]pyrimidin-7-yl)-1-(aminomethyl)cyclohexanol (Intermediate AZ, 57 mg, 0.12 mmol) and CD (20 mg, 0.12 mmol) in THF (3.0 mL) was heated at 70° C. for 3 h, then stirred at room temperature overnight. The reaction mixture was concentrated in vacuo, and purified by reverse phase preparative HPLC (Method S) to afford the title compound. MS m/z 490.3 (M+H⁺) (Method M).

### Example 140

### N-((1R,4R)-4-(5-(3-(7-oxabicyclo[2.2.1]heptan-1-ylmethoxy)phenyl)-4-amino-7H-pyrrolo[2,3-d]pyrimidin-7-yl)cyclohexyl)-4-methylpiperazine-1-carboxamide

The title compound was prepared in a similar manner to Example 133 starting from Intermediate AW and 4-methylpiperazine-1-carbonyl chloride. MS m/z 560.3 (M+H⁺) (Method M). ¹HNMR (MeOD-d₄) δ ppm 8.15 (s, 1H), 7.41 (m, 2H), 7.10 (m, 2H), 7.01 (m, 1H), 4.06 (m, 1H), 4.60 (m, 1H), 4.34 (s, 2H), 4.01 (m, 1H), 3.54 (m, 3H), 2.61 (m, 3H), 2.40 (m, 3H), 2.20 (m, 2H), 2.02 (m, 2H), 1.88 (m, 7H), 1.69 (m, 4H).

### Example 141

### N-((1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexyl)tetrahydro-2H-pyran-4-carboxamide

The title compound was prepared in a similar manner to Example 133 starting from Intermediate AW and tetrahydro-2H-pyran-4-carbonyl chloride. MS m/z 546.3 (M+H⁺) (Method M).

### Example 142

### N-((1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexyl)cyclobutanecarboxamide

The title compound was prepared in a similar manner to Example 133 starting from Intermediate AW and cyclobutanecarbonyl chloride. MS m/z 516.3 (M+H⁺) (Method M).

### Example 143

### 4-((1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexyl)-1-methylpiperazin-2-one

The title compound was prepared in a similar manner to Example 27 starting from Intermediate AR and 1-methylpiperazin-2-one. The trans and cis isomers were separated with prep. TLC, eluting with 10% MeOH in DCM with 0.1 N ammonia. MS m/z 531.3 (M+H⁺) (Method M).

### Example 144

### N-((1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexyl)-3,3,3-trifluoropropanamide

The title compound was prepared in a similar manner to Example 133 starting from Intermediate AW and 3,3,3-trifluoropropanoyl chloride. MS m/z 544.3 (M+H⁺) (Method M).

### Example 145

### 2-((1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexylamino)isonicotinonitrile

To a mixture of 5-(3-(7-oxabicyclo[2.2.1]heptan-1-ylmethoxy)phenyl)-7-((1S,4S)-4-aminocyclohexyl)-7H-pyrrolo[2,3-d]pyrimidin-4-amine (Intermediate AW) and 2-chloroisonicotinonitrile in t-butanol (1 mL) was heated at 130° C. for 2 days. The crude product mixture was purified by reverse phase preparative HPLC (Method S) to afford the title compound. MS m/z 536.3 (M+H⁺) (Method M).

### Example 146

### N-((1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexyl)cyclopropanecarboxamide

The title compound was prepared in a similar manner to Example 133 starting from Intermediate AW and cyclopropanecarbonyl chloride. MS m/z 502.3 (M+H⁺) (Method M). ¹HNMR (MeOD-d₄) δ ppm 8.06 (s, 1H), 7.31 (m, 2H), 7.01 (m, 2H), 6.91 (m, 1H), 4.59 (m, 1H), 4.49 (m, 1H), 4.24 (s, 2H), 4.07 (m, 1H), 2.05 (m, 2H), 1.85 (m, 4H), 1.74 (m, 6H), 1.60 (m, 1H), 1.58 (m, 4H), 0.76 (m, 2H), 0.66 (m, 2H).

### Example 147

### N-((1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexyl)isobutyramide

The title compound was prepared in a similar manner to Example 133 starting from Intermediate AW and isobutyryl chloride. MS m/z 504.3 (M+H⁺) (Method M). ¹HNMR (MeOD-d₄) δ ppm 8.16 (s, 1H), 7.40 (m, 2H), 7.11 (m, 2H), 7.03 (m, 1H), 4.68 (m, 1H), 4.60 (m, 1H), 4.34 (s, 2H), 4.14 (m, 1H), 2.61 (m, 1H), 2.14 (m, 2H), 1.85 (m, 10H), 1.68 (m, 4H), 1.15 (s, 3H), 1.13 (s, 3H).

### Example 148

### 7-((1S,4S)-4-((1H-imidazol-1-yl)methyl)cyclohexyl)-5-(3-(7-oxabicyclo[2.2.1]heptan-1-ylmethoxy)phenyl)-7H-pyrrolo[2,3-d]pyrimidin-4-amine

A mixture of imidazole (30 mg, 0.44 mmol) and sodium hydride (10 mg, 0.25 mmol) in DMF (1 mL) was stirred at room temperature for 10 min. ((1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexyl)methyl methanesulfonate (Step AX.1, 29 mg, 0.055 mmol) was added. After stirring at 60° C. overnight, the reaction was diluted with DCM (50 mL), washed with water (10 mL), saturated aqueous NaCl (10 mL), dried and evaporated. The crude product was purified by flash chromatography (SiO₂, MeOH:DCM/0-10%) to give the title compound as a white solid. MS m/z 499.3 (M+H⁺) (Method M). ¹HNMR (CD₃OD) δ ppm 8.15 (s, 1H), 7.76 (s, 1H), 7.52 (s, 1H), 7.41 (t, J=8.0 Hz, 1H), 7.21 (s, 1H), 7.14 (m, 1H), 7.11 (m, 1H), 7.01 (m, 2H), 4.69 (m, 1H), 4.61 (t, J=4.8 Hz, 1H), 4.35 (s, 2H), 4.26 (d, J=8.0 Hz, 2H), 2.29 (m, 1H), 2.16 (dq, J=2.8, 12.8 Hz, 2H), 1.94-1.76 (m, 8H), 1.70 (m, 6H).

### Example 149

### 4-{4-Amino-5-[3-(7-oxa-bicyclo[2.2.1]hept-1-ylmethoxy)-phenyl]-pyrrolo[2,3-d]pyrimidin-7-yl}-1-(1,1-dioxo-1λ6-thiomorpholin-4-ylmethyl)-cyclohexanol

The title compound was prepared in a similar manner to Example 131 starting from Intermediate AZ and vinylsulfonylethene. MS m/z 582.3 (M+H⁺) (Method M).

### Example 150 and 151

### 5-(3-(7-oxabicyclo[2.2.1]heptan-1-ylmethoxy)phenyl)-7-((1S,4S)-4-(oxazol-5-yl)cyclohexyl)-7H-pyrrolo[2,3-d]pyrimidin-4-amine and 5-(3-(7-oxabicyclo[2.2.1]heptan-1-ylmethoxy)phenyl)-7-((1R,4S)-4-(oxazol-5-yl)cyclohexyl)-7H-pyrrolo[2,3-d]pyrimidin-4-a mine

A mixture of (1S,4S)-4-(5-(3-(7-oxabicyclo[2.2.1]heptan-1-ylmethoxy)phenyl)-4-amino-7H-pyrrolo[2,3-d]pyrimidin-7-yl)cyclohexanecarbaldehyde (Intermediate AT, 45 mg, 0.1 mmol), 2-tosylacetonitrile (23 mg, 0.12 mmol) and sodium tert-butoxide (30 mg, 0.1 mmol) in MeOH was stirred in a sealed vial at 80° C. overnight. The reaction temperature was then increased to 90° C. and stirred for an additional 8 h. After aqueous work up, the crude product was purified by flash chromatography (SiO₂, MeOH:DCM/0-10%) to give the title compounds as pure cis or trans isomers. Isomer 1 (first fraction): MS m/z 486.3 (M+H⁺) (Method M). ¹HNMR (CDCl3) δ ppm 8.35 (s, 1H), 7.88 (s, 1H), 7.37 (t, J=8.0 Hz, 1H), 7.09-7.06 (m, 2H), 7.00-6.96 (m, 3H), 5.24 (br s, 2H), 4.85 (m, 1H), 4.65 (t, J=4.8 Hz, 1H), 4.33 (s, 2H), 2.32 (m, 2H), 2.07 (m, 4H), 1.97-1.82 (m, 7H), 1.65 (m, 4H). Isomer 2 (second fraction): MS m/z 486.3 (M+H⁺) (Method M). ¹HNMR (CDCl3) δ ppm 8.31 (s, 1H), 7.80 (s, 1H), 7.37 (t, J=8.0 Hz, 1H), 7.10-7.06 (m, 3H), 6.68 (ddd, J=0.8, 2.8, 8.4 Hz, 1H), 6.80 (s, 1H), 5.36 (br s, 2H), 4.78 (m, 1H), 4.63 (t, J=5.2 Hz, 1H), 4.31 (s, 2H), 2.82 (m, 1H), 2.27 (m, 4H), 2.00-1.74 (m, 8H), 1.62 (m, 4H).

### Example 152

### 7-(cis-3-methylsuphanylmethyl-cyclobutyl)-5-{3-[(R)-1-(tetrahydro-pyran-2-yl)methoxy]-phenyl}-7H-pyrrolo[2,3-d]pyrimidin-4-ylamine

To a solution of toluene-4-sulphonic acid cis-3-(4-amino-5-{3-[(R)-1-(tetrahydro-pyran-2-yl)methoxy]-phenyl}-pyrrolo[2,3-d]pyrimidin-7-yl)-cyclobutylmethyl ester (Intermediate BA, 0.347 mmol) in dry THF (3 ml) was added sodium methane thiolate (430 mg, 6.14 mmol) and the mixture stirred for 18 hours at room temperature. Water was then added, the mixture extracted 3× with dichloromethane, the organic layers dried over sodium sulphate and evaporated to give the crude product. Purification by reversed phase chromatography (Method R), elution of the product containing fractions through a 300 mg Bond Elut-SCX cartridge, release with 7 Mammonia in methanol (2 ml), after evaporation and trituration with methanol gave the title compound as a white solid. HPLC/MS t_{R} 1.12 min, M+H 439.1 and M−H 437.2 (Method X).

### Example 153

### (cis-3-{4-amino-5-[3-(4-methyl-7-oxa-bicyclo[2.2.1]hept-1-ylmethoxy)-phenyl]-pyrrolo[2,3-d]pyrimidin-7-yl}-cyclobutyl)-methanol

A mixture of [cis-3-(4-amino-5-iodo-pyrrolo[2,3-d]pyrimidin-7-yl)-cyclobutyl]-methanol (Intermediate M, 247 mg, 0.717 mmol), 1-methyl-4-[3-(4,4,5,5-tetramethyl-[1,3,2]dioxaborolan-2-yl)-phenoxymethyl]-7-oxa-bicyclo[2.2.1]heptane (Intermediate BB, 247 mg, 717 mmol), sodium carbonate (152 mg, 1.44 mmol), tetakis(triphenylphosphine)palladium(0) (83 mg, 0.07 mmol), water (0.5 ml) and DMF (1 ml) was purged with argon, then heated for 16 hours at 80° C. in a sealed vessel under an argon atmosphere. After cooling the reaction mixture was partitioned between water and DCM, extracted with DCM, the organic layers dried over sodium sulphate and evaporated. The residue was purified by flash column chromatography, eluting with a hexane/ethyl acetate gradient followed by preparative reversed phase chromatography (Method R). Product containing fractions were eluted through a Bond Elute SCX cartridge, then released upon elution with 7 Mammonia in methanol and evaporated to give the title compound. ¹H NMR (400 MHz, CDCl₃) δ ppm 1.52 (s, 3H), 1.59-1.81 (m, 6H), 1.87-1.99 (m, 2H), 2.41-2.52 (m, 1H), 2.52-2.71 (m, 4H), 3.74 (d, J=4.3 Hz, 2H), 4.26 (s, 2H), 5.11 (quin, J=8.7 Hz, 1H), 5.45 (br, s, 2H), 6.97 (dd, J=8.2, 2.3 Hz, 1H), 7.05 (s, 1H), 7.07 (d, J=2.0 Hz, 1H), 7.12 (s, 1 H), 7.36 (t, J=7.8 Hz, 1H), 8.29 (s, 1H).

### Example 154

### 7-[cis-3-(2-methyl-imidazol-1-ylmethyl)-cyclobutyl]-5-[3-(tetrahydro-pyran-2-ylmethoxy)-phenyl]-7H-pyrrolo[2,3-d]pyrimidin-4-ylamine

Sodium hydride (17.9 mg, 0.448 mmol) was added to a solution of 2-methylimidazole (37.1 mg, 0.448 mmol) in DMF (1.5 ml) at 0° C. After stirring for 30 minutes at room temperature toluene-4-sulfonic acid cis-3-{4-amino-5-[3-(tetrahydro-pyran-2-ylmethoxy)-phenyl]-10 pyrrolo[2,3-d]pyrimidin-7-yl}-cyclobutylmethyl ester (Intermediate AA, 420 mg, 0.299 mmol) was added and the reaction mixture heated at 75° C. for 2 hours. After cooling, water was added and the mixture extracted 2× with ethyl acetate, the organic phases dried over sodium sulphate and evaporated. Purification of the residue by flash column chromatography, eluting with a methanol/dichloromethane gradient, gave teh title compound as a pale green foam. HPLC/MS t_{R} 0.77 min, M+H 473.1 (Method X).

### Example 155

### 7-[cis-3-(1,1-dioxo-1-thiomorpholin-4-ylmethyl)-cyclobutyl]-5-[3-(4-methyl-7-oxa-bicyclo[2.2.1]hept-1-ylmethoxy)-phenyl]-7H-pyrrolo[2,3-d]pyrimidin-4-ylamine

Argon was bubbled through a mixture of 5-bromo-7-[cis-3-(1,1-dioxo-1-thiomorpholin-4-ylmethyl)-cyclobutyl]-7H-pyrrolo[2,3-d]pyrimidin-4-ylamine (Intermediate BC, 100 mg, 0.241 mmol), 1-methyl-4-[3-(4,4,5,5-tetramethyl-[1,3,2]dioxaborolan-2-yl)-phenoxymethyl]-7-oxa-bicyclo[2.2.1]heptane (Intermediate BB, 116 mg, 0.338 mmol), potassium phosphate (106 mg, 0.483 mmol), sodium carbonate (52 mg, 0.483 mmol), DMF (1.9 ml) and water (0.1 ml), tetrakistriphenylphosphinepalladium(0) (22 mg, 0.019 mmol) added, the reaction vessel sealed under argon, and then heated at 100° C. for 75 minutes. The cooled reaction mixture was diluted with ethyl acetate, and the organic layer washed with water, then saturated brine, dried over sodium sulphate and evaporated. Purification of the residue by flash column chromatography, eluting with a methanol/dichloromethane gradient, gave the title compound as a white solid. HPLC/MS t_{R} 1.02 min, M+H 552.6 (Method X).

### Example 156

### 7-[cis-3-(1,1-Dioxo-1-thiomorpholin-4-ylmethyl)-cyclobutyl]-5-{3-[(S)-1-(tetrahydro-furan-2-yl)methoxy]-phenyl}-7H-pyrrolo[2,3-d]pyrimidin-4-ylamine

Argon was bubbled through a mixture of 5-bromo-7-[cis-3-(1,1-dioxo-1-thiomorpholin-4-ylmethyl)-cyclobutyl]-7H-pyrrolo[2,3-d]pyrimidin-4-ylamine (Intermediate BC, 90 mg, 0.217 mmol), 4,4,5,5-tetramethyl-2-{3-[(S)-1-(tetrahydro-furan-2-yl)methoxy]-phenyl}-[1,3,2]dioxaborolane (Intermediate BD, 99 mg, 0.326 mmol), potassium phosphate (95 mg, 0.434 mmol), sodium carbonate (46 mg, 0.434 mmol), DMF (1.7 ml) and water (0.1 ml), tetrakistriphenylphosphinepalladium(0) (20 mg, 0.017 mmol) added, the reaction vessel sealed under argon, and then heated at 100° C. for 1 hour. The cooled reaction mixture was diluted with ethyl acetate, and the organic layer washed with water, then saturated brine, dried over sodium sulphate and evaporated. Purification of the residue by flash column chromatography, eluting with a methanol/dichloromethane gradient, gave the title compound as a white solid. HPLC/MS t_{R} 0.74 min, M+H 512.2 (Method X).

### Example 157

### 7-[cis-3-(2-methyl-imidazol-1-ylmethyl)-cyclobutyl]-5-[3-(4-methyl-7-oxa-bicyclo[2.2.1]hept-1-ylmethoxy)-phenyl]-7H-pyrrolo[2,3-d]pyrimidin-4-ylamine

A 60% dispersion of sodium hydride in mineral oil (10.3 mg, 0.257 mmol) was addded to a solution of 2-methylimidazole (21.1 mg, 257 mmol) in DMF (0.6 ml) at room temperature. After 5 minutes at room temperature a solution of cis-toluene-4-sulphonic acid 3-{4-amino-5-[3-(4-methyl-7-oxa-bicyclo[2.2.1]hept-1-ylmethoxy)-phenyl]-pyrrolo[2,3-d]pyrimidin-7-yl}-cyclobutyl methyl ester (Intermediates BE, 297 mg, 0.214 mmol) in DMF (1 ml) was added. After 5 hours at room temperature the reaction mixture was filtered and directly purified by reversed phase chromatography (Method R). Elution of the product containing fractions through a 300 mg Bond Elut-SCX cartridge, release with 7 Mammonia in methanol (2 ml), after evaporation and trituration with methanol gave the title compound as a pale yellow glass. HPLC/MS t_{R} 0.80 min, M+H 499.2 (Method X).

### Example 158

### 1-(4-[cis-3-(4-amino-5-{3-(S)-1-(tetrahydrofuran-2-yl)methoxy]-phenyl}-pyrrolo[2,3-d]pyrimidin-7-yl)-cyclobutyl]-piperazin-1-yl}-ethanone

Argon was bubbled through a mixture of 1-{4-[cis-3-(4-amino-5-iodo-pyrrolo[2,3-d]pyrimidin-7-yl)-cyclobutyl]-piperazin-1-yl}-ethanone (Intermediate BF, 77 mg, 0.175 mmol), 4,4,5,5-tetramethyl-2-{3-[(S)-1-(tetrahydro-furan-2-yl)methoxy]-phenyl}-[1,3,2]dioxaborolane (Intermediate BD, 86 mg, 0.227 mmol), potassium phosphate (76 mg, 0.350 mmol), sodium carbonate (37 mg, 0.350 mmol), DMF (2.7 ml) and water (0.3 ml) at room temperature for 5 minutes. Tetrakistriphenylphosphinepalladium(0) (10.1 mg, 0.009 mmol) was then added, the reaction vessel sealed under argon and heated at 100° C. for 3 hours. The cooled reaction mixture was partitioned between water and DCM, extracted 2× with DCM, the combined organic layers dried over sodium sulphate and evaporated. Purification of the residue by normal phase chromatography, eluting with a gradient of methanol in DCM, gave the title compound as an off-white solid. HPLC/MS t_{R} 0.69 min, M+H 491.3 (Method X).

### Example 159

### d₂-7-[cis-3-(1,1-dioxo-1-thiomorpholin-4-ylmethyl)-cyclobutyl]-5-[3-(7-oxa-bicyclo[2.2.1]hept-1-ylmethoxy)-phenyl]-7H-pyrrolo[2,3-d]pyrimidin-4-ylamine

The title compound was prepared in a similar manner to Example 155 starting from d₂-1-[3-(4,4,5,5-tetramethyl-[1,3,2]dioxaborolan-2-yl)-phenoxymethyl]-7-oxa-bicyclo[2.2.1]heptane (Intermediate B1). HPLC/MS t_{R} 0.91 min, M+H 540.1 (Method X).

### Example 160

### 7-[cis-3-aminomethyl-cyclobutyl]-5-[3-(7-oxa-bicyclo[2.2.1]hept-1-ylmethoxy)-phenyl]-7H-pyrrolo[2,3-d]pyrimidin-4-ylamine

Argon was bubbled through a mixture of 7-(3-aminomethyl-cyclobutyl)-5-iodo-7H-pyrrolo[2,3-d]pyrimidin-4-ylamine (Intermediate BJ, 1.07 g, 3.12 mmol), 2-(3-((7-oxabicyclo[2.2.1]heptan-1-ylmethoxy)phenyl)-4,4,5,5-tetramethyl-1,3,2-dioxaborolane (Intermediate K, 1.08 g, 3.27 mmol), sodium carbonate (0.66 g, 6.24 mmol), DMF (10 ml) and water (5 ml) at room temperature for 5 minutes. Tetrakistriphenylphosphinepalladium(0) (0.36 g, 0.312 mmol) was then added, the reaction vessel sealed under argon and heated at 80° C. for 24 hours. The cooled reaction mixture was partitioned between water and DCM, extracted 2× with DCM, the combined organic layers dried over sodium sulphate and evaporated. Purification of the residue by normal phase chromatography, eluting with a gradient of methanol in DCM, gave the title compound as a pale-yellow foam. HPLC/MS t_{R} 0.74 min, M+H 420.1 (Method X).

### Example 161

### (3-{4-amino-5-[3-(7-oxa-bicyclo[2.2.1]hept-1-ylmethoxy)-phenyl]-pyrrolo[2,3-d]pyrimidin-7-yl}-cyclobutylmethyl)-carbamic acid methyl ester

Methyl chloroformate (0.013 ml, 0.172 mmol) was added to a solution of 7-[cis-3-aminomethyl-cyclobutyl]-5-[3-(7-oxa-bicyclo[2.2.1]hept-1-ylmethoxy)-phenyl]-7H-pyrrolo[2,3-d]pyrimidin-4-ylamine (Example 160, 60 mg, 0.143 mmol) in triethylamine (0.020 ml, 0.143 mmol) and THF (1 ml) at room temperature. After 50 minutes the reaction mixture was diluted with DCM, washed with water and brine, dried over sodium sulphate and evaporated.

Purification of the residue by normal phase chromatography, eluting with a gradient of methanol in DCM, gave the title compound as a pale-yellow foam. HPLC/MS t_{R} 0.99 min, M+H 478.1 (Method X).

### Example 162

### N-(3-{4-amino-5-[3-(7-oxa-bicyclo[2.2.1]hept-1-ylmethoxy)-phenyl]-pyrrolo[2,3-d]pyrimidin-7-yl}-cyclobutylmethyl)-propionamide

The title compound was prepared in a similar manner to Example 161 using propionyl chloride in place of methyl chloroformate. HPLC/MS t_{R} 0.92 min, M+H 476.2 (Method X).

### Example 163

### 1-(3-{4-amino-5-[3-(7-oxa-bicyclo[2.2.1]hept-1-ylmethoxy)-phenyl]-pyrrolo[2,3-d]pyrimidin-7-yl}-cyclobutyl methyl)-3-ethyl-urea

The title compound was prepared in a similar manner to Example 161 using ethyl isocyanate in place of methyl chloroformate. HPLC/MS t_{R} 0.93 min, M+H 491.3 (Method X).

### Example 164

### N-(3-{4-amino-5-[3-(7-oxa-bicyclo[2.2.1]hept-1-ylmethoxy)-phenyl]-pyrrolo[2,3-d]pyrimidin-7-yl}-cyclobutylmethyl)-isobutyramide

The title compound was prepared in a similar manner to Example 161 using 2-methylpropionyl chloride in place of methyl chloroformate. HPLC/MS t_{R} 0.97 min, M+H 490.2 (Method X).

### Example 165

### d₃-N-(3-{4-amino-5-[3-(7-oxa-bicyclo[2.2.1]hept-1-ylmethoxy)-phenyl]-pyrrolo[2,3-d]pyrimidin-7-yl}-cyclobutylmethyl)-acetamide

The title compound was prepared in a similar manner to Example 161 using d₃-acetyl chloride in place of methyl chloroformate. HPLC/MS t_{R} 0.90 min, M+H 465.2 (Method X).

### Example 166

### d₉-7-[cis-3-(1,1-dioxo-1-thiomorpholin-4-ylmethyl)-cyclobutyl]-5-[3-(7-oxa-bicyclo[2.2.1]hept-1-ylmethoxy)-phenyl]-7H-pyrrolo[2,3-d]pyrimidin-4-ylamine

The title compound was prepared in a similar manner to Example 155 starting from d₉-1-[3-(4,4,5,5-tetramethyl-[1,3,2]dioxaborolan-2-yl)-phenoxymethyl]-7-oxa-bicyclo[2.2.1]heptane (Intermediate BK). HPLC/MS t_{R} 0.91 min, M+H 547.4 (Method X).

### Example 167

### N-(3-{4-amino-5-[3-(7-oxa-bicyclo[2.2.1]hept-1-ylmethoxy)-phenyl]-pyrrolo[2,3-d]pyrimidin-7-yl}-cyclobutylmethyl)-methanesulfonamide

The title compound was prepared in a similar manner to Example 161 using methanesulphonyl chloride in place of methyl chloroformate. HPLC/MS t_{R} 0.96 min, M+H 498.2 (Method X).

### Example 168

### 2-[(3-{4-amino-5-[3-(7-oxa-bicyclo[2.2.1]hept-1-ylmethoxy)-phenyl]-pyrrolo[2,3-d]pyrimidin-7-yl}-cyclobutylmethyl)-amino]-propane-1,3-diol

Sodium triacetoxy borohydride (91 mg, 0.429 mmol) was added potion-wise to a mixture of 7-[cis-3-aminomethyl-cyclobutyl]-5-[3-(7-oxa-bicyclo[2.2.1]hept-1-ylmethoxy)-phenyl]-7H-pyrrolo[2,3-d]pyrimidin-4-ylamine (Example 160, 120 mg, 0.286 mmol), 2,2-dimethyl-1,3-dioxan-5-one (44.7 mg, 0.343 mmol), acetic acid (0.025 ml, 0.429 mmol) and 1,2-dichloroethane (2 ml) at room temperature. After stirring for 5 hours at room tempertature 2N hydrochloric acid was added, the reaction mixture extracted with DCM, dried over sodium sulphate and evaporated to give the title compound as a yellow glass. HPLC/MS t_{R} 0.75 min, M+H 494.2 (Method X).

### Example 169

### (E)-2-{4-amino-5-[3-(7-oxa-bicyclo[2.2.1]hept-1-ylmethoxy)-phenyl]-pyrrolo[2,3-d]pyrimidin-7-yl}-5-oxa-7-aza-spiro[3.4]octan-6-one

Carbonyl diimidazole (13 mg, 0.080 mmol) was added to a solution of 1-aminomethyl-(E)-3-{4-amino-5-[3-(7-oxa-bicyclo[2.2.1]hept-1-ylmethoxy)-phenyl]-pyrrolo[2,3-d]pyrimidin-7-yl}-cyclobutanol (Intermediate BL, 29 mg, 0.067 mmol) in THF (0.5 ml) at room temperature. After 1 hour the reaction was partitioned between water and DCM, the organic layers dried over sodium sulphate and evaporated to give the title compound as a white foam. HPLC/MS t_{R} 1.04 min, M+H 462.1 (Method X).

### Example 170

### 2-[4-(3-{4-amino-5-[3-(7-oxa-bicyclo[2.2.1]hept-1-ylmethoxy)-phenyl]-pyrrolo[2,3-d]pyrimidin-7-yl}-cyclobutyl)-piperazin-1-yl]-ethanol

Sodium triacetoxy borohydride (23.3 mg, 0.110 mmol) was added to a mixture of 3-{4-amino-5-[3-(7-oxa-bicyclo[2]hept-1-ylmethoxy)-phenyl]-pyrrolo[2,3-d]pyrimidin-7-yl}-cyclobutanone (Intermediate BM, 37 mg, 0.073 mmol), N-(2-hydroxyethyl)-piperazine (0.011 ml, 0.088 mmol), acetic acid (0.005 ml, 0.073 mmol) and 1,2-dichloroethane (1 ml) at room temperature. After stirring for 48 hours at room tempertature water was added, the reaction mixture extracted with DCM, dried over sodium sulphate and evaporated. Purification by reversed phase chromatography (Method R), elution of the product containing fractions through a 300 mg Bond Elut-SCX cartridge, release with 7 Mammonia in methanol (2 ml), after evaporation and trituration with methanol gave the title compounds as a white solid. HPLC/MS t_{R} 0.76 min, M+H 519.3 (Method X).

### Example 171

### 7-[cis-3-(4-methanesulfonyl-piperazin-1-yl)-cyclobutyl]-5-[3-(7-oxa-bicyclo[2.2.1]hept-1-ylmethoxy)-phenyl]-7H-pyrrolo[2,3-d]pyrimidin-4-ylamine

The title compound was prepared in a similar manner to Example 170 using 1-methanesulphonyl-piperazine in place of N-(2-hydroxyethyl)-piperazine. The trans isomer was isolated from the cis/trans mixture following flash column chromatography, eluting with a gradient of methanol in DCM, followed by trituration in acetonitrile to give the title compound as an off-white solid. HPLC/MS t_{R} 0.99 min, M+H 553.3 (Method X). ¹H NMR (400 MHz, DMSO) δ ppm 1.55-1.75 (m, 8H), 2.30-2.35 (m, 2H), 2.42-2.45 (m, 4H), 2.57-2.64 (m, 2H), 2.66-2.74 (m, 1H), 2.89 (s, 3H), 3.08-3.14 (m, 4H), 4.32 (s, 2H), 4.49-4.52 (m, 1H), 4.89-4.95 (m, 1H), 6.15 (s, br, 2H), 6.95 (dd, 1H), 7.04 (dd, 1H), 7.11 (dd, 1H), 7.37 (t, 1H), 7.53 (s, 1H), 8.14 (s, 1H).

### Example 172

### 2-[4-(cis-3-{4-amino-5-[3-(7-oxa-bicyclo[2.2.1]hept-1-ylmethoxy)-phenyl]-pyrrolo[2,3-d]pyrimidin-7-yl}-cyclobutyl)-piperazin-1-yl]-ethanol

Normal phase chromatography with the cis/trans mixture of 2-[4-(3-{4-amino-5-[3-(7-oxa-bicyclo[2.2.1]hept-1-ylmethoxy)-phenyl]-pyrrolo[2,3-d]pyrimidin-7-yl}-cyclobutyl)-piperazin-1-yl]-ethanol (Example 170), eluting with a gradient of methanol in DCM, gave the title compound cleanly as the first eluting isomer. HPLC/MS t_{R} 0.78 min, M+H 519.3 (Method X).

### Example 173

### 1-[5-(cis-3-{4-amino-5-[3-(7-oxa-bicyclo[2.2.1]hept-1-ylmethoxy)-phenyl]-pyrrolo[2,3-d]pyrimidin-7-yl}-cyclobutyl)-2,5-diaza-bicyclo[2.2.1]hept-2-yl]-ethanone

Acetic anhydride (0.022 ml, 0.234 mmol)) was added to a mixture of 7-[3-(2,5-diaza-bicyclo[2.2.1]hept-2-yl)-cyclobutyl]-5-[3-(7-oxa-bicyclo[2.2.1]hept-1-ylmethoxy)-phen yl]-7H-pyrrolo[2,3-d]pyrimidin-4-ylannine (Intermediate BO, 120 mg, 0.234 mmol), pyridine (0.019 ml, 0.234 mmol) and THF (2 ml) at room temperature. After stirring for 1 hour the reaction mixture was partitioned between aqueous sodium bicarbonate solution and DCM, extracting 2× with DCM, the combined organic layers washed with brine, dried over sodium sulphate and evaporated. Purification of the residue by normal phase chromatography, eluting with a gradient of methanol in DCM, gave the cis-isomer followed by the trans-isomer. cis-isomer: HPLC/MS t_{R} 0.84 min, M+H 529.3 (Method X).

### Examples 174 and 175

### 2-[5-(cis-3-{4-amino-5-[3-(7-oxa-bicyclo[2.2.1]hept-1-ylmethoxy)-phenyl]-pyrrolo[2,3-d]pyrimidin-7-yl}cyclobutyl)-2,5-diaza-bicyclo[2.2.1]hept-2-yl]-ethanol and 2-[(trans-3-{4-amino-5[3-(7-oxa-bicyclo[2.2.1]hept-1-ylmethoxy)-phenyl]-pyrrolo[2,3-d]pyrimidin-7-yl}-cyclobutyl)-2,5-diaza-bicyclo[2.2.1]hept-2-yl]-ethanol

A mixture of 7-[3-(2,5-diaza-bicyclo[2.2.1]hept-2-yl)-cyclobutyl]-5-[3-(7-oxa-bicyclo[2.2.1]hept-1-ylmethoxy)-phenyl]-7H-pyrrolo[2,3-d]pyrimidin-4-ylamine (Intermediate BO, 110 mg, 0.181 mmol), 2-bromoethanol (34 mg, 0.271 mmol), potassium carbonate (125 mg, 0.904 mmol) in acetonitrile (1.5 ml) was heated at 80° C. for 1 hour. The cooled reaction mixture was partitioned between aqueous sodium bicarbonate solution and DCM, extracting 2× with DCM, the combined organic layers dried over sodium sulphate and evaporated. Purification of the residue by normal phase chromatography, eluting with a gradient of methanol in DCM containing 0.1% concentrated ammonia solution, gave the cis-isomer followed by the trans-isomer.

cis-isomer: HPLC/MS t_{R} 0.76 min, M+H 531.3 (Method X).
trans isomer: HPLC/MS t_{R} 0.78 min, M+H 531.3 (Method X).

### Example 176

### 7-[cis-3-(2-hydroxymethyl-imidazol-1-ylmethyl)-cyclobutyl]-53-(7-oxa-bicyclo[2.2.1]hept-1-ylmethoxy)--phenyl]-7H-pyrrolo[2,3-d]pyrimidin-4-ylamine

7-[cis-3-(2-Carboethoxy-imidazol-1-ylmethyl)-cyclobutyl]-53-(7-oxa-bicyclo[2.2.1]hept-1-ylmethoxy)--phenyl]-7H-pyrrolo[2,3-d]pyrimidin-4-ylamine (example 180, 170 mg, 0.31 mmol) was dissolved in THF/EtOH (1:1, 4 ml) and sodium borohydride (24 mg, 0.62 mmol) and LiCl (27 mg, 0.62 mmol) were added with stirring at ambient temperature. The reaction mixture was then warmed to 55° C. and stirred for 12 h. It was allowed to cool to room temperature and subjected to aqueous workup. The crude product was purified by flash chromatography (SiO₂, DCM/MeOH, gradient elution 0-20% MeOH) to give the title compound as a white solid. M+H 500.2 (Method X). ¹H NMR (400 MHz, MeOH-d₄) δ ppm 1.64-1.73 (m, 4H), 1.78-1.85 (m, 4H), 2.27-2.46 (m, 2H), 2.59-2.75 (m, 4H), 3.34 (s, 2H), 4.27 (d, J=7.0 Hz, 2H), 4.34 (s, 2H), 4.59 (t, J=4.5 Hz, 1H), 4.68 (s, 2H), 5.06-5.19 (m, 1H), 6.91 (s, 1H), 6.96-7.03 (m, 1H), 7.06-7.14 (m, 2H), 7.18 (d, J=1.2 Hz, 1H), 7.38 (d, J=7.8 Hz, 1H), 7.43 (s, 1H), 8.13 (s, 1H).

### Example 177

### 7-[cis-3-(N-methylcarboxy-amino-methyl)-cyclobutyl]-5-3-(7-oxa-bicyclo[2.2.1]hept-1-ylmethoxy)-phenyl]-7H-pyrrolo[2,3-d]pyrimidin-4-ylamine

(3-{4-Amino-5-[3-(7-oxa-bicyclo[2.2.1]hept-1-ylmethoxy)-phenyl]-pyrrolo[2,3-d]pyrimidin-7-yl}-cyclobutyl)-methanol (Example 22; 120 mg, 0.29 mmol) was dissolved in DCM (5 ml) and DIEA (125 μl, 0.71 mmol) added. The mixture was cooled to 0° C. and treated with methylisocyanide (19 mg, 0.34 mmol). The reaction mixture was gradually warmed to rt, stirred for 12 hours and subjected to aqueous workup. The crude material was purified by flash chromatography (SiO₂, DCM/MeOH, gradient 0-10% MeOH) to give the title compound as a white solid. M+H 476.3 (Method X). ¹H NMR (400 MHz, CDCl₃) δ ppm 8.28 (s, 1H), 7.39 (dd, 1H), 7.18 (s, 1H), 7.08 (s, 1H), 7.06 (d, 1H), 7.02 (d, 1H), 6.00 (bs, 2H), 5.10 (quin, 1H), 4.85 (bs, 1H), 4.65 (dd, 1H), 4.41 (bs, 1H), 4.34 (s, 2H), 3.38 (dd, 2H), 2.81 (s, 3H), 2.71-2.67 (m, 2H), 2.45-2.41 (m, 1H). 2.39-2.32 (m, 2H), 1.99-1.92 (m, 2H), 1.89-1.83 (m, 2H), 1.67-1.62 (m, 4H).

### Example 178

### 7-[cis-3-(acetyl-amino-methyl-1-ylmethyl)-cyclobutyl]-5-3-(7-oxa-bicyclo[2.2.1]hept-1-ylmethoxy)-phenyl]-7H-pyrrolo[2,3-d]pyrimidin-4-ylamine

(3-{4-Amino-5-[3-(7-oxa-bicyclo[2.2.1]hept-1-ylmethoxy)-phenyl]-pyrrolo[2,3-d]pyrimidin-7-yl}-cyclobutyl)-methanol (example 22; 120 mg, 0.29 mmol) was dissolved in DCM (5 ml) and DIEA (125 μl, 0.71 mmol) added. The mixture was cooled to −40° C. and treated with acetic anhydride (30 mg, 0.29 mmol). The reaction mixture was gradually warmed to rt within 1 hour and subjected to aqueous workup. The crude material was purified by flash chromatography (SiO₂, DCM/MeOH, gradient 0-10% MeOH) to give the title compound as a white solid. M+H 462.2 (Method X). ¹H NMR (400 MHz, CDCl₃) δ ppm 8.31 (s, 1H), 7.39 (dd, 1H), 7.09 (s, 1H), 7.08 (s, 1H), 7.06 (d, 1H), 7.02 (d, 1H), 6.38 (bs, 1H), 5.97 (bs, 2H), 5.03 (quin, 1H), 4.65 (dd, 1H), 4.34 (s, 2H), 3.50 (s, 2H), 3.45-3.42 (m, 2H), 2.70-2.67 (m, 2H), 2.53-2.46 (m, 3H), 2.09 (5, 3H), 1.93-1.89 (m, 2H), 1.86-1.83 (m, 2H), 1.66-1.63 (m, 4H).

### Example 179

### 7-[cis-3-(4-methyltetrazoyl-1-ylmethyl)-cyclobutyl]-5-3-(7-oxa-bicyclo[2.2.1]hept-1-ylmethoxy)-phenyl]-7H-pyrrolo[2,3-d]pyrimidin-4-ylamine

The title compound was prepared in a similar manner as described for example 154 starting from cis-toluene-4-sulphonic acid 3-{4-amino-5-[3-(4-methyl-7-oxa-bicyclo[2.2.1]hept-1-ylmethoxy)-phenyl]-pyrrolo[2,3-d]pyrimidin-7-yl}-cyclobutylmethyl ester (Intermediate BE) and 4-methyl tetrazole to give the title compound as the less polar regioisomer. M+H 587.2 (Method X). ¹H NMR (400 MHz, CDCl₃) δ ppm 1.64 (d, J=7.0 Hz, 4H), 1.79-2.03 (m, 4H), 2.46 (qd, J=9.4, 2.2 Hz, 2H), 2.55 (s, 3H), 2.71-2.92 (m, 2H), 4.34 (s, 2H), 4.64 (t, J=5.1 Hz, 1H), 4.74 (d, J=6.6 Hz, 2H), 5.18 (t, J=7.8 Hz, 1H), 5.77 (br. s., 2H), 6.95-7.12 (m, 4 H), 7.39 (t, J=8.0 Hz, 1H), 8.28 (br. s., 1H).

### Example 180

### 7-[cis-3-(2-carboethoxy-imidazol-1-ylmethyl)-cyclobutyl]-53-(7-oxa-bicyclo[2.2.1]hept-1-ylmethoxy)-phenyl]-7H-pyrrolo[2,3-d]pyrimidin-4-ylamine

The title compound was prepared in a similar manner as described for Example 154 starting from cis-toluene-4-sulphonic acid 3-{4-amino-5-[3-(4-methyl-7-oxa-bicyclo[2.2.1]hept-1-ylmethoxy)-phenyl]-pyrrolo[2,3-d]pyrimidin-7-yl}-cyclobutylmethyl ester (Intermediate BE) and 2-carbethoxy imidazole. M+H 543.2 (Method X). ¹H NMR (400 MHz, CDCl₃) δ ppm 1.38-1.47 (m, 3H), 1.57-1.69 (m, 4H), 1.78-1.97 (m, 4H), 2.27-2.42 (m, 2H), 2.61-2.75 (m, 3H), 4.27-4.34 (m, 2H), 4.38-4.45 (m, 2H), 4.56-4.66 (m, 3H), 5.07-5.19 (m, 1H), 5.35 (br. s., 2 H), 6.99 (dd, J=8.2, 2.0 Hz, 1H), 7.03-7.15 (m, 4H), 7.19 (s, 1H), 7.38 (t, J=7.8 Hz, 1H), 8.30 (s, 1H).

### Example 181

### 3-[3-(methyl-piperazin-1-yl)-cyclobutyl]-1-[3-(7-oxa-bicyclo[2.2.1]hept-1-ylmethoxy-phenyl]-imidazo[1,5-a]pyrazin-8-ylamine

The title compound was prepared in a similar manner as described for Example 170 starting from 3-{8-amino-1-[3-(7-oxa-bicyclo[2.2.1]hept-1-ylmethoxy)-phenyl]-imidazo[1,5-a]pyrazin-3-yl}-cyclobutanone (Intermediate BQ) and N-methyl piperazine. M+H 489.3 (Method X). ¹H NMR (400 MHz, MeOH-D₄) δ ppm 1.69 (d, J=7.4 Hz, 4H), 1.78-1.90 (m, 4H), 2.36-2.46 (m, 3H), 2.65-2.78 (m, 8H), 3.01 (br. s., 3H), 3.14 (t, J=7.4 Hz, 1H), 3.17-3.24 (m, 1H), 3.66 (dd, J=17.8, 1.8 Hz, 1H), 4.36 (s, 2H), 4.59 (t, J=4.5 Hz, 1H), 7.01 (d, J=5.1 Hz, 1H), 7.13 (dd, J=8.2, 2.3 Hz, 1H), 7.21 (d, J=7.4 Hz, 1H), 7.25 (s, 1H), 7.43-7.49 (m, 2H).

### Example 182

### 7-(trans-4-Amino-cyclohexyl)-5-[3-(7-oxa-bicyclo[2.2.1]hept-1-ylmethoxy)-phenyl]-7H-pyrrolo[2,3-d]pyrimidin-4-ylamine

A mixture of intermediate BV (295 mg, 0.553 mmol) and HCl (4 N in dioxane, 4 mL) was stirred at rt for 15 min, concentrated and diluted with DCM/NaHCO₃^{sat}. The aqueous layer was extracted with DCM. The combined organic extracts were dried (Na₂SO₄), filtered and concentrated. The residue was purified by silica gel column chromatography (DCM/MeOH/NH₃^{aq}, 91.5:7.5:1) to afford 161 mg of the title compound as a white solid: ¹H NMR (400 MHz, d6-DMSO) δ ppm 1.17-1.34 (m, 2H) 1.51-1.91 (m, 14H) 2.64 (t, 1H) 4.29 (s, 2H) 4.44-4.62 (m, 1H) 4.49 (t, 1H) 6.92 (dd, 1H) 7.01 (d, 1H) 7.05 (s, 1H) 7.35 (t, 1H) 7.46 (s, 1H) 8.11 (s, 1H); ES-MS: 434.2 [M+H]⁺; t_{R}=2.77 min (Method C); R_{f}=0.18 (DCM/MeOH/NH₃^{aq}, 91.5:7.5:1).

### Example 183

### 7-(trans-4-Dimethylamino-cyclohexyl)-5-[3-(7-oxa-bicyclo[2.2.1]hept-1-ylmethoxy)-phenyl]-7H-pyrrolo[2,3-d]pyrimidin-4-ylamine

A mixture of the compound prepared in example 182 (110 mg, 0.254 mmol), formaldehyde (30% in H₂O, 38 μL, 0.507 mmol, 2 eq) and sodium triacetoxyborohydride (161 mg, 0.761 mmol, 3 eq) in DCM (2 mL) were stirred for 4 h at rt and diluted with DCM/NaHCO₃^{sat}. The aqueous layer was extracted with DCM. The combined organic extracts were washed with H₂O and brine, dried (Na₂SO₄), filtered and concentrated. The residue was purified by silica gel column chromatography (DCM/MeOH/NH₃^{aq}, 91.5:7.5:1) to afford 92 mg of the title compound as a white solid: ¹H NMR (400 MHz, d6-DMSO) δ ppm 1.35-1.48 (m, 2H) 1.51-1.76 (m, 8H) 1.82-2.06 (m, 2H) 1.82-2.02 (m, 4H) 2.19 (s, 6H) 2.20-2.33 (m, 1H) 4.29 (s, 2H) 4.42-4.63 (m, 2H) 6.93 (dd, 1H) 7.01 (d, 1H) 7.05 (s, 1H) 7.35 (t, 1H) 7.45 (s, 1H) 8.11 (s, 1H); ES-MS: 462.2 [M+H]⁺; t_{R}=2.81 min (Method C); R_{f}=0.20 (DCM/MeOH/NH₃^{aq}, 91.5:7.5:1).

### Example 184

### 1-(cis-3-{4-Amino-5-[3-(7-oxa-bicyclo[2.2.1]hept-1-ylmethoxy)-phenyl]-pyrrolo[2,3-d]pyrimidin-7-yl}-cyclobutylmethyl)-piperidin-4-ol

A mixture of intermediate BW (57 mg, 0.150 mmol), 2-(3((7-oxabicyclo[2.2.1]heptan-1-ylmethoxy)phenyl)-4,4,5,5-tetramethyl-1,3,2-dioxaborolane (Intermediate K, 54.4 mg, 0.165 mmol, 1.1 eq), Pd(PPh₃)₄ (8.7 mg, 7.5 μmol, 0.05 eq), K₃PO₄ (63.6 mg, 0.300 mmol, 2 eq), Na₂CO₃ (31.8 mg, 0.300 mmol, 2 eq) in DMF (2 mL) and H₂O (0.2 mL) was stirred at 100° C. for 30 min under an argon atmosphere, allowed to cool to rt and diluted with ethyl acetate/H₂O. The aqueous layer was extracted with ethyl acetate. The combined organic extracts were washed with H₂O and brine, dried (Na₂SO₄), filtered and concentrated. The residue was purified by silica gel column chromatography (DCM/MeOH, 92.5:7.5) to afford 56 mg of the title compound as a white foam: ¹H NMR (400 MHz, d6-DMSO) δ ppm 1.26-1.45 (m, 2H) 1.52-1.76 (m, 10H) 1.95-2.05 (m, 2H) 2.10-2.20 (m, 2H) 2.21-2.44 (m, 1H) 2.44 (d, 2H) 2.50-2.60 (m, 2H) 2.63-2.71 (m, 2H) 3.34-3.48 (m, 1H) 4.30 (s, 2H) 4.45-4.55 (m, 2H) 5.05 (t, 1H) 6.95 (dd, 1H) 7.04 (d, 1H) 7.08 (s, 1H) 7.36 (t, 1H) 7.62 (s, 1 H) 8.11 (s, 1H); API-ES: 504.3 [M+H]⁺; R_{f}=0.11 (DCM/MeOH, 92.5:7.5).

### Example 185

### 8-(cis-3-{4-Amino-5-[3-(7-oxa-bicyclo[2.2.1]hept-1-ylme thoxy)-phenyl]-pyrrolo[2,3-d]pyrimidin-7-yl}-cyclobutylmethyl)-8-aza-bicyclo[3.2.1]octan-(3-exo)-ol

The title compound was prepared in analogy to the procedure described in Example 184, but using intermediate BX. The title compound: ¹H NMR (400 MHz, d6-DMSO) δ ppm 1.50-2.05 (m, 16H) 2.09-2.27 (m, 3H) 2.30-2.50 (m, 4H) 3.04 (br. s., 2H) 3.78 (br. s., 1H) 4.22 (d, 1H) 4.30 (s, 2H) 4.50 (t, 1H) 4.97-5.14 (m, 1H) 6.94 (dd, 1H) 7.03 (d, 1H) 7.07 (s, 1H) 7.36 (t, 1H) 7.63 (s, 1H) 8.10 (s, 1H); ES-MS: 530.4 [M+H]⁺; R₁=0.13 (DCM/MeOH, 92.5:7.5).

### Example 186

### 1-[cis-4-(3-{4-Amino-5-[3-(7-oxa-bicyclo[2.2.1]hept-1-ylmethoxy)-phenyl]-pyrrolo[2,3-d]pyrimidin-7-yl}-cyclobutyl)-piperazin-1-yl]-ethanone

The title compound was prepared in analogy to the procedure described in Example 184, but using intermediate BY, stirring the reaction mixture for 1 h at 100° C. and quenching it with NaHCO₃^{sat}. The crude material was purified by preparative HPLC (Gilson gx-281. Column: Sunfire C18, 30×100 mm, 5 μm. Flow: 30 mL/min. Gradient: 5% to 100% B in 20 min; A=0.1% TFA in H₂O, B=0.1% TFA in MeCN. Detection: UV). The title compound: ¹H NMR (400 MHz, d6-DMSO) δ ppm 1.52-1.75 (m, 8H) 1.97 (s, 3H) 2.20-2.42 (m, 6H) 2.53-2.70 (m, 3H) 3.36-3.47 (m, 4H) 4.30 (s, 2H) 4.44-4.59 (m, 1H) 4.79-5.01 (m, 1H) 6.12 (br. s, 2H) 6.94 (dd, 1H) 7.04 (d, 1H) 7.10 (br. s, 1H) 7.36 (t, 1H) 7.54 (s, 1H) 8.12 (s, 1H); ES-MS: 517.4 [M+H]⁺.

### Example 187

### 3-{8-amino-1-[3-(7-oxa-bicyclo[2.2.1]hept-1-ylmethoxy)-phenyl]-imidazo[1,5-a]pyrazin-3-yl}-cyclobutyl)-piperidin-4-ol

The title compound was prepared in a similar manner as described for example 170 starting from 3-{8-Amino-1-[3-(7-oxa-bicyclo[2.2.1]hept-1-ylmethoxy)-phenyl]-imidazo[1,5-a]pyrazin-3-yl}-cyclobutanone (Intermediate BQ) and piperidin-4-ol. M+H 490.3 (Method X). ¹H NMR (400 MHz, MeOH-D₄) δ ppm 7.47 (dd, 1H), 7.44 (d, 1H), 7.26 (s, 1H), 7.21 (d, 1H), 7.13 (d, 1H), 7.00 (d, 1H), 4.60 (s, 1H), 4.01-3.96 (m, 1H), 3.69-3.62 (m, 1H), 3.15-3.10 (m, 1H), 2.90 (bs, 2H), 2.75/d, 2H), 2.44 (dd, 2H), 2.34-2.30 (m, 1H), 1.95-1.82 (m, 6H), 1.72-1.68 (m, 4H), 1.62-1.54 (m, 2H).

### Example 188

### 1-[4-(trans-3-{4-Amino-5[3-(7-oxa-bicyclo[2.2.1]hept-1-ylmethoxy)-phenyl]-pyrrolo[2,3-d]pyrimidin-7-yl}-cyclobutyl)-piperazin-1-yl]-ethanone

The title compound was prepared in analogy to the procedure described in Example 184, but using intermediate BZ, 1.2 eq of intermediate K, stirring the reaction mixture for 1 h at 100° C. and quenching it with NaHCO₃^{sat}. The crude material was purified by preparative HPLC (Gilson gx-281. Column: Sunfire C18, 30×100 mm, 5 μM. Flow: 30 mL/min. Gradient: 5% to 100% B in 20 min; A=0.1% TFA in H₂O, B=0.1% TFA in MeCN. Detection: UV). The title compound: ¹H NMR (400 MHz, d6-DMSO) δ ppm 1.50-1.76 (m, 8H) 1.99 (s, 3H) 2.25-2.39 (m, 4H) 2.52-2.65 (m, 4H) 2.97 (br. s, 1H) 3.38-3.55 (m, 4H) 4.30 (s, 2H) 4.50 (t, 1H) 5.11-5.33 (m, 1H) 6.11 (br. s, 2H) 6.95 (dd, 1H) 7.04 (d, 1H) 7.07-7.12 (m, 1H) 7.37 (t, 1H) 7.64 (s, 1H) 8.11 (s, 1H); ES-MS: 517.3 [M+H]⁺.

### Example 189

### (S)-1-(cis-3-{4-Amino-5-[3-(7-oxa-bicyclo[2.2.1]hept-1-ylmethoxy)-phenyl]-pyrrolo[2,3-d]pyrimidin-7-yl}-cyclobutyl)-pyrrolidine-2-carboxylic acid amide

PdCl₂(dppf)-CH₂Cl₂ (10.8 mg, 13 μmol, 0.1 eq) was added to a mixture of intermediate CA (50 mg, 0.132 mmol), 2-(3-((7-oxabicyclo[2.2.1]heptan-1-ylmethoxy)phenyl)-4,4,5,5-tetramethyl-1,3,2-dioxaborolane (Intermediate K, 48 mg, 0.145 mmol, 1.1 eq), a 2 M aqueous solution of Na₂CO₃ (0.3 mL), toluene (2 mL) and EtOH (0.3 mL) at 105° C., under an argon atmosphere. The reaction mixture was stirred for 30 min at 105° C. Further PdCl₂(dppf)-CH₂Cl₂ (15 mg) was added and the mixture was stirred for 1 h at 105° C., allowed to cool to rt, and diluted with EtOAc/H₂O and extracted with EtOAc. The combined organic extracts were washed with H₂O and brine, dried (Na₂SO₄), filtered and concentrated. The reddish residue was purified by silica gel column chromatography (DCM/MeOH/NH₃^{aq}, 94:5:1) to afford 17 mg of the title compound as a white foam: ¹H NMR (600 MHz, d6-DMSO) δ ppm 1.50-1.79 (m, 11H) 1.95-2.09 (m, 1H) 2.40-2.60 (m, 5H) 2.96-3.11 (m, 3H) 4.31 (s, 2H) 4.51 (t, 1H) 4.85-4.95 (m, 1H) 6.10 (br. s., 2H) 6.97 (dd, 1H) 7.01-7.15 (m, 3H) 7.38 (t, 1H) 7.45-7.50 (m, 1H) 7.65 (s, 1H) 8.12 (s, 1H); ES-MS: 503.4 [M+H]⁺; R_{f}=0.10 (DCM/MeOH/NH₃^{aq}, 94:5:1).

### Example 190

### (S)-1-(trans-3-{4-Amino-5-[3-(7-oxa-bicyclo[2.2.1]hept-1-ylmethoxy)-phenyl]-pyrrolo[2,3-d]pyrimidin-7-yl}-cyclobutyl)-pyrrolidine-2-carboxylic acid amide

The title compound was prepared in analogy to the procedure described in Example 189 but using intermediate CB. The compound was further purified by preparative HPLC (Gilson gx-281. Column: Sunfire C18, 30×100 mm, 5 μm. Flow: 30 mL/min. Gradient: 5% to 100% Bin 20 min; A=0.1% TFA in H₂O, B=0.1% TFA in MeCN. Detection: UV). The title compound: ¹H NMR (600 MHz, d6-DMSO) δ ppm 1.57-1.79 (m, 11H) 2.04 (m, 1H) 2.32-2.62 (m, 5 H) 2.98 (dd, 1H) 3.15 (t, 1H) 3.30-3.45 (m, 1H) 4.31 (s, 2H) 4.51 (t, 1H) 5.40 (quin, 1H) 6.15 (br. s, 2H) 6.96 (dd, 1H) 7.05 (d, 1H) 7.10 (s, 1H) 7.16 (d, 1H) 7.38 (t, 1H) 7.47 (d, 1 H) 7.64 (s, 1H) 8.12 (s, 1H); ES-MS: 503.3 [M+H]⁺; R_{f}=0.09 (DCM/MeOH/NH₃^{aq}, 94:5:1).

### Example 191

### 1-(cis-3-{4-Amino-5-[3-(7-oxa-bicyclo[2.2.1]hept-1-ylmethoxy)-phenyl]-pyrrolo[2,3-d]pyrimidin-7-yl}-cyclobutyl)-azetidin-3-ol

A mixture of intermediate BM (100 mg, 0.247 mmol) and 3-hydroxyazetidine hydrochloride (54.2 mg, 0.494 mmol, 2 eq) in DCM (4 mL) was stirred for 2 h at rt and under an argon atmosphere. Sodium triacetoxyborohydride (157 mg, 0.742 mmol, 3 eq) was added dropwise. The resulting mixture was stirred for 16 h at rt and diluted with DCM/NaHCO₃^{sat}. The aqueous layer was extracted with DCM. The combined organic extracts were dried (Na₂SO₄), filtered and concentrated. The residue was purified by silica gel column chromatography (DCM/MeOH/NH₃^{aq}, 91.5:7.5:1) to afford 29 mg of the title compound as a white solid. The title compound: ¹H NMR (400 MHz, d6-DMSO) δ ppm 1.51-1.75 (m, 8H) 2.22-2.34 (m, 2H) 2.40-2.50 (m, 2H) 2.84 (dd, 2H) 2.98 (t, 1H) 3.35-3.45 (m, 2H) 4.08-4.23 (m, 1H) 4.30 (s, 2H) 4.49 (t, 1H) 4.84 (t, 1H) 5.24 (d, 1H) 6.08 (br. s, 2H) 6.94 (dd, 1H) 7.04 (d, 1H) 7.08 (d, 1H) 7.36 (t, 1H) 7.51 (s, 1H) 8.11 (s, 1H); ES-MS: 462.3 [M+H]⁺; R_{f}=0.16 (DCM/MeOH/NH₃^{aq}, 91.5:7.5:1).

### Example 192

### 1-(3-{4-Amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl)-piperidin-4-ol

A mixture of toluene-4-sulfonic acid 3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl ester (Intermediate BL.2, 50 mg, 0.085 mmol), 4-hydroxy-piperidine (171 mg, 1.69 mmol, 20 eq) and triethylamine (236 μL, 1.69 mmol, 20 eq) in DMF (1 mL) was stirred for 15 min at 140° C. under microwave irradiation. The reaction mixture was diluted with ethyl acetate/NaHCO₃^{sat}. The aqueous layer was separated and extracted with ethyl acetate. The combined organic extracts were washed with H₂O and brine, dried (Na₂SO₄), filtered and concentrated. The residue was purified by preparative HPLC (Gilson gx-281. Column: Sunfire C18, 30×100 mm, 5 μm. Flow: 30 mL/min. Gradient: 5% to 100% B in 20 min; A=0.1% TFA in H₂O, B=0.1% TFA in MeCN. Detection: UV) to afford 7 mg of the title compound as a yellow solid: ¹H NMR (400 MHz, d6-DMSO) δ ppm 1.30-1.70 (m, 12H) 2.15-2.25 (m, 2H) 2.33-2.61 (m, 6H) 2.70-2.80 (m, 2H) 3.35-3.45 (m, 1H) 4.31 (s, 2H) 4.45-4.60 (m, 2H) 4.89 (br. s., 1H) 5.30-5.50 (m, 1H) 6.13 (br. s., 2H) 6.96 (dd, 1H) 7.01-7.11 (m, 2H) 7.38 (t, 1H) 7.61 (s, 1H) 8.13 (s, 1H); ES-MS: 520.4.

### Example 193

### Acetic acid N′-(cis-3-{4-amino-5-[3-(7-oxa-bicyclo[2.2.1]hept-1-ylmethoxy)-phenyl]-pyrrolo[2,3-d]pyrimidin-7-yl}-cyclobutyl)-hydrazide

A mixture of intermediate BM (150 mg, 0.371 mmol) and acetic hydrazide (35.7 mg, 0.482 mmol, 1.3 eq) and sodium triacetoxyborohydride (236 mg, 1.11 mmol, 3 eq) in DCM (3 ml) was stirred for 18 h at rt and under an argon atmosphere, diluted with NaHCO₃^{sat} and extracted with DCM. The combined organic extracts were washed with NaHCO₃^{sat}, dried (Na₂SO₄), filtered and concentrated. The residue was purified by silica gel column chromatography (DCM/MeOH/NH₃^{aq}, 98:1:1 then 95:4:1), followed by preparative HPLC (Gilson gx-281. Column: Sunfire C18, 30×100 mm, 5 μm. Flow: 30 mL/min. Gradient: 5% to 100% B in 20 min; A=0.1% TFA in H₂O, B=0.1% TFA in MeCN. Detection: UV) to afford 9 mg of the title compound as a yellow solid: ¹H NMR (400 MHz, d6-DMSO) δ ppm 1.48-1.63 (m, 4H) 1.63-1.75 (m, 4H) 1.79 (s, 3H) 2.21-2.41 (m, 2H) 2.54-2.70 (m, 2H) 3.24-3.28 (m, 1 H) 4.30 (s, 2H) 4.43-4.55 (m, 1H) 4.77-4.94 (m, 1H) 5.05-5.25 (m, 1H) 6.10 (br. s., 2 H) 6.95 (d, 1H) 7.01-7.13 (m, 2H) 7.37 (t, 1H) 7.58 (s, 1H) 8.11 (s, 1H) 9.28 (d, 1H); ES-MS: 463.3 [M+H]⁺.

### Example 194

### 7-{cis-3-[(6-Methyl-pyridin-2-ylamino)-methyl]-cyclobutyl}-5-[3-(7-oxa-bicyclo[2.2.1]hept-1-ylmethoxy)-phenyl]-7H-pyrrolo[2,3-d]pyrimidin-4-ylamine

The title compound was prepared in analogy to the procedure described in Example 184 but using intermediate CC. The title compound: ¹H NMR (400 MHz, d6-DMSO) δ ppm 1.52-1.75 (m, 8H) 2.20-2.45 (m, 6H) 2.50-2.55 (m, 2H) 3.37 (t, 2H) 4.30 (s, 2H) 4.50 (t, 1H) 5.07 (quin, 1H) 6.10 (br. s., 2H) 6.28 (m, 2H) 6.42 (t, 1H) 6.95 (dd, 1H) 7.03 (d, 1H) 7.07 (s, 1H) 7.22 (t, 1H) 7.37 (t, 1H) 7.60 (s, 1H) 8.12 (s, 1H); ES-MS: 511.3 [M+H]⁺; R_{f}=0.18 (DCM/MeOH/NH₃^{aq}, 96.5:2.5:1).

### Example 195

### cis-4-(3-{4-Amino-5-[3-(7-oxa-bicyclo[2.2.1]hept-1-ylmethoxy)-phenyl]-pyrrolo[2,3-d]pyrimidin-7-yl}-cyclobutyl)-1-methyl-piperazin-2-one

The title compound was prepared in analogy to the procedure described in Example 1 but using the intermediate prepared in step 16.1, stirring the reaction mixture for 1 h at 100° C. and quenching it with NaHCO₃^{sat}. The crude material, consisting of a mixture of cis and trans isomers, was first purified by by silica gel column chromatography (DCM/MeOH/NH₃^{aq}, 98:1:1 then 96:3:1). Further purification by preparative HPLC (column: Chiracel OD 20 μm, 186×25 mm; eluent: heptane/EtOH, 1:1, flow: 40 mL/min) afforded pure samples of the cis isomer (title compound) and the trans isomer (example 196), respectively. The title compound (cis-isomer): ¹H NMR (400 MHz, d6-DMSO) δ ppm 1.40-1.82 (m, 8H) 2.09-2.69 (m, 6H) 2.70-2.78 (m, 1H) 2.81 (s, 3H) 3.22-3.29 (m, 4H) 4.30 (s, 2H) 4.41-4.57 (m, 1H) 4.84-5.02 (m, 1H) 6.09 (br. s, 2H) 6.94 (dd, 1H) 7.04 (d, 1H) 7.09 (s, 1H) 7.36 (t, 1H) 7.54 (s, 1H) 8.12 (s, 1H); ES-MS: 503.3 [M+H]⁺.

### Example 196

### 4-(trans-3-{4-Amino-5-[3-(7-oxa-bicyclo[2.2.1]hept-1-ylmethoxy)-phenyl]-pyrrolo[2,3-d]pyrimidin-7-yl}cyclobutyl)-1-methyl-piperazin-2-one

The procedure is described in example 195. The title compound (trans isomer): ¹H NMR (400 MHz, d6-DMSO) δ ppm 1.50-1.75 (m, 8H) 2.53-2.68 (m, 6H) 2.83 (s, 3H) 2.98 (s, 2 H) 3.01-3.09 (m, 1H) 3.25-3.30 (m, 2H) 4.30 (s, 2H) 4.41-4.58 (m, 1H) 5.10-5.39 (m, 1H) 6.11 (br. s, 2H) 6.95 (dd, 1H) 7.04 (d, 1H) 7.08 (s, 1H) 7.37 (t, 1H) 7.64 (s, 1H) 8.11 (s, 1H); ES-MS: 503.3 [M+H]⁺.

### Example 197

### (E)-2-{4-Amino-5-[3-(7-oxa-bicyclo[2.2.1]hept-1-ylmethoxy)-phenyl]-pyrrolo[2,3d]pyrimidin-7-yl}-5,7-dioxa-spiro[3.4]octan-6-one

A mixture of (E)-3-{4-amino-5-[3-(7-oxa-bicyclo[2.2.1]hept-1-ylmethoxy)-phenyl]-pyrrolo[2,3-d]pyrimidin-7-yl}-1-hydroxymethyl-cyclobutanol (Intermediate BN, 40 mg, 0.068 mmol), NEt₃ (0.08 mL), K₂CO₃ (30 mg), and DMF (1 mL) was heated at 75° C. for 1 h. After filtering off the K₂CO₃, and evaporating the DMF, the title compound was isolated from the reaction mixture by means of preparative HPLC (Method R) as white solid (4 mg). HPLC t_{R}=2.55 min (method D), M+H=463.3 (method L). ¹H NMR (600 MHz, DMSO-d₆) δ ppm 8.18 (s, 1H, pyrimidinyl), 7.66 (s, 1H, pyrrolyl), 7.38 (t, 1H, phenyl), 7.08 (s, 1H, phenyl), 7.05/6.96 (d/d, 1H/1H, phenyl), 6.15 (s/b, 2H, NH₂), 5.31 (quintet, 1H, CH-cyclobutyl), 4.65 (s, 2H, CH₂—O), 4.52 (m, 1H, CH-oxabicycloheptanyl), 4.30 (s, 2H, CH₂—O-phenyl), 3.10/2.92 (t/t, 4H, CH₂-cyclobutyl), 1.68/1.52 (m/m, 8H, CH₂-oxabicycloheptanyl).

### Example 198

### (S)-(Z)-1-(3-(4-Amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl)-pyrrolidine-2-carboxylic acid amide

Compound of toluene-4-sulfonic acid (Z)-3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl ester (Step BL.3, 30 mg, 0.051 mmol), D-prolinamide (20.3 mg, 0.254 mmol), K2CO3 (70.9 mg, 0.508 mmol), NEt₃ (0.072 mL, 0.508 mmol), and DMF (1 ml) were stirred at 90° C. for 3 h under Ar. The reaction mixture was concentrated under reduced pressure and partitined between AcOEt (40 mL) and H₂O (40 mL). The aqueous phase was extracted with AcOEt (20 mL, 2×). The combined organic phases were washed with H₂O (10 mL) and brine (10 mL, 2×), dried (Na₂SO₄), and concentrated under reduced pressure to give a white solid (36 mg). The material was purified by column chromatography using a 12 g silica gel column (RediSept (Is-co)) and a Sepacore Control chromatography system from Büchi: DCM (5 min) -->DCM/MeOH (10% NH₃ (25%))=97:3 in 30 min at a flow rate of 30 mL/min) to give the title compound as a white solid (11 mg). HPLC t_{R}=2.195 min (Method D), M+H=533.4 (method L). ¹H NMR (600 MHz, DMSO-d₆) δ ppm 8.13 (s, 1H, pyrimidinyl), 7.64 (s, 1H, pyrrolyl), 7.44/7.06 (d/d, 2H, NH₂-amide), 7.38 (t, 1H, phenyl), 7.09 (s, 1H, phenyl), 7.07/6.96 (d/d, 1H/1H, phenyl), 6.15 (s/b, 2H, NH₂), 5.38 (s, 1H, OH), 4.78 (quintet, 1H, CH-cyclobutyl), 4.50 (s, 1H, CH-oxabicycloheptanyl), 4.30 (s, 2H, CH₂—O-phenyl), 3.25/2.45 (m,/m, 2H, CH₂-proline), 3.03 (m, 1H, CH-proline), 2.75/2.25 (d/d, 2H, CH₂—N), 2.70/2.35 (m/m, 4H, CH₂-cyclobutyl), 1.99/1.71 (m/m, 4H, CH₂-proline), 1.68/1.62 (m/m, 8H, CH₂-oxabicycloheptanyl).

### Example 199

### (Z)-1-(3-{4-Amino-5-[3-(7-oxa-bicyclo[2.2.1]hept-1-ylmethoxy)-phenyl]-pyrrolo[2,3-d]pyrimidin-7-yl}-1-h yd roxy-cyclobutylmethyl)-azetidin-3-ol

The title compound was synthesized by analogy to the preparation of compound of Example 198 starting from toluene-4-sulfonic acid (Z)-3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl ester (Step BL.3) and azetidin-3-ol. White solid. HPLC t_{R}=2.187 min (method D), M+H=492.3 (method L). ¹H NMR (600 MHz, DMSO-d₆) δ ppm 8.13 (s, 1H, pyrimidinyl), 7.64 (s, 1H, pyrrolyl), 7.38 (t, 1H, phenyl), 7.09 (s, 1H, phenyl), 7.07/6.96 (d/d, 1H/1H, phenyl), 6.15 (s/b, 2H, NH₂), 5.25 (s/b, 1H, HO-azetidinyl), 5.10 (s/b, 1H, HO-cyclobutyl), 4.72 (quintet, 1H, CH-cyclobutyl), 4.52 (m, 1H, CH-oxabicycloheptanyl), 4.30 (s, 2H, CH₂—O-phenyl), 4.20 (m, 1H, CH-azetidinyl), 3.66/3.31 (m/m, 4H, CH₂-azetidinyl), 2.70/2.35 (m/m, 4H, CH₂-cyclobutyl), 2.53 (m, 2H, CH₂—N), 1.69/1.54 (m/m, 8H, CH₂-oxabicycloheptanyl).

### Example 200

### (Z)-3-{4-Amino-5-[3-(7-oxa-bicyclo[2.2.1]hept-1-ylmethoxy)-phenyl]-pyrrolo[2,3-d]pyrimidin-7-yl}-1-((R)-2-hydroxymethyl-pyrrolidin-1-ylmethyl)-cyclobutanol

The title compound was synthesized by analogy to the preparation of compound of Example 198 starting from toluene-4-sulfonic acid (Z)-3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl ester (Step BL.3) and. (R)-1-Pyrrolidin-2-yl-methanol. Beige solid. HPLC t_{R}=2.210 min (method D), M+H=520.4 (method L). ¹H NMR (600 MHz, DMSO-d₆) S ppm 8.13 (s, 1H, pyrimidinyl), 7.64 (s, 1H, pyrrolyl), 7.38 (t, 1H, phenyl), 7.09 (s, 1H, phenyl), 7.07/6.96 (d/d, 1H/1H, phenyl), 6.15 (s/b, 2H, NH₂), 5.25 (s/b, 1H, HO-cyclobutyl), 4.72 (quintet, 1H, CH-cyclobutyl), 4.52 (m, 2H, CH-oxabicycloheptanyl/HO—CH₂), 4.30 (s, 2H, CH₂—O-phenyl), 3.91/3.78 (m/m, 2H, HO—CH₂), 3.27/2.35 (m/m, 2H, CH₂-pyrrolidinyl), 2.85/2.60 (m/m, 2H, CH₂—N), 2.70/2.35 (m/m, 4H, CH₂-cyclobutyl), 2.63 (m, 1H, CH-pyrrolidinyl), 1.77/1.50 (m/m, 2H, CH₂-pyrrolidinyl), 1.69/1.54 (m/m, 8H, CH₂-oxabicycloheptanyl), 1.65/1.55 (m/m, 2H, CH₂-pyrrolidinyl).

### Example 201

### (E)-(1-(Aminomethyl-3-{4-amino-5-[3-(7-oxa-bicyclo[2.2.1]hept-1-ylmethoxy)-phenyl]-pyrrolo[2,3-d]pyrimidin-7-yl}-cyclobutanol)

This Example is the same as Intermediate BL.

### Example 202

### (Z)-3-{4-Amino-5-[3-(7-oxa-bicyclo[2.2.1]hept-1-ylmethoxy)-phenyl]-pyrrolo[2,3-d]pyrimidin-7-yl}-1-((S)-2-hydroxymethyl-pyrrolidin-1-ylmethyl)-cyclobutanol

The title compound was synthesized by analogy to the preparation of compound of Example 198 starting from toluene-4-sulfonic acid (Z)-3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl ester (Step BL.3) and (S)-1-Pyrrolidin-2-yl-methanol. Beige solid. HPLC t_{R}=2.210 min (Method D), M+H=520.4 (method L). ¹H NMR (600 MHz, DMSO-d₆) δ ppm 8.13 (s, 1H, pyrimidinyl), 7.64 (s, 1H, pyrrolyl), 7.38 (t, 1H, phenyl), 7.09 (s, 1H, phenyl), 7.07/6.96 (d/d, 1H/1H, phenyl), 6.15 (s/b, 2H, NH₂), 5.25 (s/b, 1H, HO-cyclobutyl), 4.82 (quintet, 1H, CH-cyclobutyl), 4.50 (m, 2H, CH-oxabicycloheptanyl/HO—CH₂), 4.30 (s, 2H, CH₂—O-phenyl), 3.41/3.30 (m/m, 2H, CH₂—OH), 3.27/2.35 (m/m, 2H, CH₂-pyrrolidinyl), 2.85/2.60 (m/m, 2H, CH₂—N), 2.70/2.35 (m/m, 4H, CH₂-cyclobutyl), 2.63 (m, 1H, CH-pyrrolidinyl), 1.77/1.50 (m/m, 2H, CH₂-pyrrolidinyl), 1.69/1.54 (m/m, 8H, CH₂-oxabicycloheptanyl), 1.65/1.55 (m/m, 2H, CH₂-pyrrolidinyl).

### Example 203

### (S)-(E)-1-(3-{4-Amino-5-[3-(7-oxa-bicyclo[2.2.1]hept-1-ylmethoxyphenyl]pyrrolo[2,3-d]pyrimidin-7-yl}-1-hydroxy-cyclobutylmethyl)-pyrrolidin-3-ol

Toluene-4-sulfonic acid (E)-3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl ester (Intermediate BL.2, 11 mg, 0.019 mmol), (S)-3-hydroxypyrrolidine (45 mg, 0.517 mmol), NEt₃ (0.05 mL) dissolved in DMF (1 mL) were heated in a microwave ofen at 140° C. for 15 min under Ar. After evaporation of the solvent under vacuum, the reaction mixture was purified by preparative TLC using a 20×20 cm silica gel plate (Merck) with the solvent system MeOH/25% NH3/DCM (15:2:83) yielding the title compound as white solid (1 mg). HPLC t_{R}=2.18 min (method D), M+H=506.3 (Method L). ¹H NMR (600 MHz, DMSO-d₆) δ ppm 8.13 (s, 1H, pyrimidinyl), 7.63 (s, 1H, pyrrolyl), 7.37 (t, 1H, phenyl), 7.09 (s, 1H, phenyl), 7.07/6.96 (d/d, 1H/1H, phenyl), 6.15 (s/b, 2H, NH₂), 4.80 (s/b, 1H, HO-cyclobutyl), 4.82 (quintet, 1H, CH-cyclobutyl), 4.67 (2/b, 1H, HO-pyrrolidinyl), 5.42 (m, 1H, CH-oxabicycloheptanyl), 4.30 (s, 2H, CH₂—O-phenyl),4.19 (m, 1H, CH-pyrrolinyl), 3.27/2.35 (m/m, 2H, CH₂-pyrrolidinyl), 2.85/2.60 (m/m, 2H, CH₂—N), 2.75-2.35 (m/m, 4H, CH₂-pyrrolidinyl), 2.60/2.41 (m/m, 4H, CH₂-cyclobutyl), 1.69/1.54 (m/m, 8H, CH₂-oxabicycloheptanyl).

### Example 204

### (R)-(E)-1-(3-{4-Amino-5-[3-(7-oxa-bicyclo[2.2.1]hept-1-ylmethoxy)-phenyl]-pyrrolo[2,3-cl]pyrimidin-7-yl}-1-hydroxy-cyclobutylmethyl)-pyrrolidin-3-ol

Toluene-4-sulfonic acid (E)-3-{4-amino-5-[3-(7-oxa-bicyclo[2.2.1]hept-1-ylmethoxy)-phenyl]-pyrrolo[2,3-cl]pyrimidin-7-yl}-1-hydroxy-cyclobutylmethyl ester (Intermediate BL.2, 20 mg, 0.034 mmol), (R)-3-hydroxypyrrolidine (45 mg, 0.517 mmol), NEt₃ (0.05 mL) dissolved in DMF (1 mL) were heated in a microwave ofen at 100° C. for 30 min under Ar. After evaporation of the solvent under vacuum, conc. Bicarbonate solution (4 mL) was added. The reaction mixture. extraction was extracted with AcOEt (2 mL, 3×). The combined organic phases were dried (MgSO₄) and concentrated under reduced pressure. The title compound was isolated by means of preparative TLC using a 20×20 cm silica gel plate (Merck) with the solvent system MeOH/25% NH₃/DCM (15:2:83) yielding the title compound as white solid (4 mg). HPLC t_{R}=2.19 min (Method D), M+H=506.3 (method L). ¹H NMR (600 MHz, DMSO-d₆) δ ppm 8.14 (s, 1H, pyrimidinyl), 7.63 (s, 1H, pyrrolyl), 7.37 (t, 1H, phenyl), 7.09 (s, 1H, phenyl), 7.07/6.96 (d/d, 1H/1H, phenyl), 6.15 (s/b, 2H, NH₂), 5.42 (quintet, 1H, CH-cyclobutyl), 4.80 (s/b, 1H, HO-cyclobutyl),), 4.71 (s/b, 1H, HO-pyrrolidinyl), 5.42 (m, 1H, CH-oxabicycloheptanyl), 4.30 (s, 2H, CH₂—O-phenyl),4.22 (m, 1H, CH-pyrrolinyl), 2.74/2.43 (m/m, 2H, CH₂-pyrrolidinyl), 2.70/2.40 (m/m, 2H, CH₂-pyrrolidinyl),2.66 (m, 2H, CH₂—N), 1.90/1.51 (m, 2H, CH₂-pyrrolidinyl), 2.57/2.36 (m/m, 4H, CH₂-cyclobutyl), 1.69/1.54 (m/m, 8H, CH₂-oxabicycloheptanyl).

### Example 205

### (E)-3-{4-Amino-5-[3-(7-oxa-bicyclo[2.2.1]hept-1-ylmethoxy)-phenyl]-pyrrolo[2,3-d]pyrimidin-7-yl}-1-(1,1-dioxo-1lambda*6*-thiomorpholin-4-ylmethyl)-cyclobutanol

To 1-aminomethyl-(E)-3-{4-amino-5-[3-(7-oxa-bicyclo[2.2.1]hept-1-ylmethoxy)-phenyl]-pyrrolo[2,3-d]pyrimidin-7-yl}-cyclobutanol (Intermediate BL, 20 mg, 0.046 mmol) and divinylsulfone (5.9 mg, 0.050 mmol) dissolved in DMF (0.9 mL) a tace of silica gel was added and the reaction mixture was stirred at RT for 19 h. After evaporation of the solvent the title compound was isolated by means of preparative TLC using a 20×20 cm silica gel plate (Merck) with the solvent system MeOH/25% NH₃/DCM (15:2:83) yielding the title compound as white solid (4 mg). HPLC t_{R}=2.252 min (Method D), M+H=554.3 (method L). ¹H NMR (600 MHz, DMSO-d₆) δ ppm 8.14 (s, 1H, pyrimidinyl), 7.63 (s, 1H, pyrrolyl), 7.37 (t, 1H, phenyl), 7.09 (s, 1H, phenyl), 7.07/6.96 (d/d, 1H/1H, phenyl), 6.15 (s/b, 2H, NH₂), 5.39 (quintet, 1H, CH-cyclobutyl), 5.15 (s/b, 1H, HO-cyclobutyl),), 5.42 (m, 1H, CH-oxabicycloheptanyl), 4.30 (s, 2H, CH₂—O-phenyl), 3.08 (s/b, 8H, CH₂-thiomorpholinedioxide), 2.74 (s, 2H, CH₂—N), 2.57/2.36 (m/m, 4H, CH₂-cyclobutyl), 1.69/1.54 (m/m, 8H, CH₂-oxabicycloheptanyl).

### Example 206

### (R)-(Z)-1-(3-(4-Amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl)-pyrrolidin-3-ol

The title compound was synthesized by analogy to the preparation of compound of Example 198 starting from toluene-4-sulfonic acid (Z)-3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl ester (Intermediate BL.3) and R-pyrrolidin-3-ol. White solid. HPLC t_{R}=2.188 min (method D), M+H=506.3 (method L). ¹H NMR (600 MHz, DMSO-d₆) δ ppm 8.14 (s, 1H, pyrimidinyl), 7.63 (s, 1H, pyrrolyl), 7.37 (t, 1H, phenyl), 7.09 (s, 1H, phenyl), 7.07/6.96 (d/d, 1H/1H, phenyl), 6.15 (s/b, 2H, NH₂), 4.79 (quintet, 1H, CH-cyclobutyl), 4.70 (s/b, 1H, HO-cyclobutyl),), 4.71 (s/b, 1H, HO-pyrrolidinyl), 4.50 (m, 1H, CH-oxabicycloheptanyl), 4.30 (s, 2H, CH₂—O-phenyl),4.22 (m, 1H, CH-pyrrolinyl), 2.80/2.43 (m/m, 2H, CH₂-pyrrolidinyl), 2.70/2.40 (m/m, 2H, CH₂-pyrrolidinyl),2.66 (m, 2H, CH₂—N), 2.57/2.36 (m/m, 4H, CH₂-cyclobutyl), 1.90/1.51 (m, 2H, CH₂-pyrrolidinyl), 1.69/1.54 (m/m, 8H, CH₂-oxabicycloheptanyl).

### Example 207

### (S)-(Z)-1-(3-{4-Amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl)-pyrrolidin-3-ol

The title compound was synthesized by analogy to the preparation of compound of Example 198 starting from toluene-4-sulfonic acid (Z)-3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl ester (Intermediate BL.3) and S-pyrrolidin-3-ol. Beige solid. HPLC t_{R}=2.187 min (Method D), M+H=506.3 (method L). ¹H NMR (600 MHz, DMSO-d₆) δ ppm 8.14 (s, 1H, pyrimidinyl), 7.63 (5, 1H, pyrrolyl), 7.37 (t, 1H, phenyl), 7.09 (s, 1H, phenyl), 7.07/6.96 (d/d, 1H/1H, phenyl), 6.15 (s/b, 2H, NH₂), 5.15 (s/b, 1H, HO-cyclobutyl), 4.79 (quintet, 1H, CH-cyclobutyl), 4.70. (s/b, 1H, HO-pyrrolidinyl), 4.51 (m, 1H, CH-oxabicycloheptanyl), 4.30 (s, 2H, CH₂—O-phenyl),4.19 (m, 1H, CH-pyrrolinyl), 2.80/2.43 (m/m, 2H, CH₂-pyrrolidinyl), 2.70/2.40 (m/m, 2H, CH₂-pyrrolidinyl),2.66 (m, 2H, CH₂—N), 2.57/2.36 (m/m, 4H, CH₂-cyclobutyl), 1.90/1.51 (m, 2H, CH₂-pyrrolidinyl), 1.69/1.54 (m/m, 8H, CH₂-oxabicycloheptanyl).

### Example 208

### (Z)-1-Aminomethyl-3-{4-amino-5-[3-(7-oxa-bicyclo[2.2.1]hept-1-ylmethoxy)-phenyl]-pyrrolo[2,3-d]pyrimidin-7-yl}-cyclobutanol

The title compound was synthesized by analogy to the preparation of compound of Example 201 starting from toluene-4-sulfonic acid (Z)-3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylnnethyl ester (Intermediate BL.3) via the corresponding azide (Z)-3-{4-Amino-5-[3-(7-oxa-bicyclo[2.2.1]hept-1-ylmethoxy)-phenyl]-pyrrolo[2,3-d]pyrimidin-7-yl}-1-azidomethyl-cyclobutanol (Intermediate CD) White solid. HPLC/MS t_{R}=0.66 min, M+H=436.3 (method X). ¹H NMR (600 MHz, DMSO-d₆) S ppm 8.14 (s, 1H, pyrimidinyl), 7.60 (s, 1H, pyrrolyl), 7.37 (t, 1H, phenyl), 7.08 (s, 1H, phenyl), 7.05/6.96 (d/d, 1H/1H, phenyl), 6.15 (s/b, 2H, NH₂), 5.24 (s, 1H, OH), 4.79 (quintet, 1H, CH-cyclobutyl), 4.51 (s, 1H, CH-oxabicycloheptanyl), 4.30 (s, 2H, CH₂—O), 2.70/2.43 (m/m, 4H, CH₂-cyclobutyl), 2.65 (s, 2H, CH₂—N), 1.70/1.55 (m/m, 8H, CH₂-oxabicycloheptanyl).

### Example 209

### (E)-3-{4-Amino-5-[3-(7-oxa-bicyclo[2.2.1]hept-1-ylmethoxy)-phenyl]-pyrrolo[2,3-d]pyrimidin-7-yl}-1-((R)-2-hydroxymethyl-pyrrolidin-1-ylmethyl)-cyclobutanol

The title compound was synthesiued by analogy to the preparation of compound of Example 202 starting from toluene-4-sulfonic acid (E)-3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl ester (Intermediate BL.2) and (R)-1-pyrrolidin-2-ylmethanlol. White solid. HPLC t_{R}=2.221 min (method D), M+H=520.3 (method L). ¹H NMR (600 MHz, a trace of TFA added, DMSO-d₆) ppm 8.14 (s, 1H, pyrimidinyi), 8.44 (s/b, 2H, NH₂), 7.98 (s, 1H, pyrrolyl), 7.42 (t, 1H, phenyl), 7.12 (s, 1H, phenyl), 7.08/7.04 (d/d, 1H/1H, phenyl), 5.65 (quintet, 1H, CH-cyclobutyl), 4.50 (m, 1H, CH-oxabicycloheptany11HO—CH₂), 4.83 (s/b, 1H, HO-cyclobutyl),), 4.30 (s, 2H, CH₂—O-phenyl), 3.73/3.39 (mlm, 2H, HO—CH₂-pyrrolidinyl), 3.70 (m, 2H, CH₂—N), 3.65/3.21 (m/m, 2H, CH₂-pyrrolidinyl), 3.51 (m, 1H, CH-pyrrolinyl), 2.12-1.81 (m, 4H, CH₂-pyrrolidinyl), 2.57/2.36 (mlm, 4H, CH₂-cyclobutyl), 1.69/1.54 (m/m, 8H, CH₂-oxabicycloheptanyl).

### Example 210

### 4-(cis-3-{4-Amino-5-[3-(7-oxa-bicyclo[2.2.1]hept-1-ylmethoxy)-phenyl]-pyrrolo[2,3-d]pyrimidin-7-yl}-cyclobutylmethyl)-1-methyl-piperazin-2-one

Methylpiperazinone hydrochloride (70 mg, 0.465 mmol) was partitioned between ether (50 mL) and conc. KOH solution (5 mL). After drying the ether phase (Na₂SO₄) and evaporating the solvent, toluene-4-sulfonic acid 3-{4-amino-5-[3-(7-oxa-bicyclo[2.2.1]hept-1-ylmethoxy)-phenyl]-pyrrolo[2,3-d]pyrimidin-7-yl}-cyclobutylmethyl ester (Intermediate CF, 71 mg, 0.124 mmol), DMA (0.4 mL) and NEt₃ (0.15 mL) were added and the reaction mixture was heated at 90° C. for 40 min in a sealed tube. After evaporation of the solvent, half concentrated NaHCO₃ (5 mL) and the extracted with AcOEt (4 mL). After drying (MgSO₄) the organic phase and evaporation the solvent, the title compound was isolated by means of preparative TLC (20×20 cm TLC-plate, MeOH/NH₃/CH₂Cl₂=10:1:89): colorless solid (8.8 mg). HPLC t_{R}=2.207 min (Method D), M+H=517.2 (method L). NMR (600 MHz, DMSO-d₆) δ ppm 8.14 (s, 1H, pyrimidinyl), 7.66 (s, 1H, pyrrolyl), 7.37 (t, 1H, phenyl), 7.09 (s, 1H, phenyl), 7.06/6.96 (d/d, 1H/1H, phenyl), 6.15 (s/b, 2H, NH₂), 5.13 (quintet, 1H, CH-cyclobutyl), 4.51 (m, 1H, CH-oxabicycloheptanyl), 4.30 (s, 2H, CH₂—O), 3.27 (m, 2H, CH₂-piperazinone), 2.99 (s, 2H, 2.77 CH₂-piperazinone), (s, 3H, CH₃—N), 2.64 (m, 2H, CH₂-piperazinone), 2.55/2.24 (m/m, 4H, CH₂-cyclobutyl), 2.44 (d, 2H, CH₂—N), 2.30 (m, 1H, CH-cyclobutyl), 1.70/1.55 (m/m, 8H, CH₂-oxabicycloheptanyl).

### Example 211

### (E)-3-{4-Amino-5-[3-(7-oxa-bicyclo[2.2.1]hept-1-ylmethoxy)-phenyl]-pyrrolo[2,3-d]pyrimidin-7-yl}-1-((S)-2-hydroxymethyl-pyrrolidin-1-ylmethyl)-cyclobutano

The title compound was synthesized by analogy to the preparation of compound of Example 202 starting from toluene-4-sulfonic acid (E)-3-(4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl ester (Intermediate BL.2) and (S)-1-pyrrolidin-2-yl methanol. White solid. HPLC t_{R}=2.219 min (method D), M+H=520.3 (Method L). ¹H NMR (600 MHz, DMSO-d₆) δ ppm 8.13 (s, 1H, pyrimidinyl), 7.62 (s, 1H, pyrrolyl), 7.38 (t, 1H, phenyl), 7.07 (s, 1H, phenyl), 7.04/6.95 (d/d, 1H/1H, phenyl), 6.15 (s/b, 2H, NH₂), 5.85 (quintet, 1H, CH-cyclobutyl), 4.90 (s/b, 1H, HO-cyclobutyl), 4.50 (m, 2H, CH-oxabicycloheptanyl), 4.42 (s/b, HO—CH₂), 4.30 (s, 2H, CH₂—O-phenyl), 3.34 (m/m, 2H, CH₂—N), 3.30 (m, 2H, CH₂—OH), 3.20 (m, 1H, CH-pyrrolidinyl),2.85/2.50 (m/m, 2H, CH₂-pyrrolidinyl), 2.55/2.41 (m/m, 4H, CH₂-cyclobutyl), 1.69/1.54 (m/m, 8H, CH₂-oxabicycloheptanyl), 1.65-1.55 (m, 4H, CH₂-pyrrolidinyl).

### Example 212

### (R)-(E)-1-(3-{4-Amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl)-pyrrolidine-2-carboxylic acid amide

The title compound was synthesized by analogy to the preparation of compound of Example 198 starting from toluene-4-sulfonic acid (E)-3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl ester (Step BL.2) and D-prolinamide. White solid. HPLC t_{R}=2.192 min (Method D), M+H=533.3 (method Q. ¹H NMR (600 MHz, DMSO-d₆) δ ppm 8.14 (s, 1H, pyrimidinyl), 7.64 (s, 1H, pyrrolyl), 7.54/7.06 (d/d, 2H, NH₂-amide), 7.38 (t, 1H, phenyl), 7.09 (s, 1H, phenyl), 7.07/6.96 (d/d, 1H/1H, phenyl), 6.15 (s/b, 2H, NH₂), 5.45 (quintet, 1H, CH-cyclobutyl), 5.23 (s, 1H, OH), 4.50 (s, 1H, CH-oxabicycloheptanyl), 4.30 (s, 2H, CH₂—O-phenyl), 3.21/2.46 (m,/m, 2H, CH₂-proline), 2.97 (m, 1H, CH-proline), 2.65/2.20 (d/d, 2H, CH₂—N), 2.70/2.35 (mlm, 4H, CH₂-cyclobutyl), 2.00 (m, 2H, CH₂-proline), 21.70 (m, 2H, CH₂-proline), 1.68/1.62 (m/m, 8H, CH₂-oxabicycloheptanyl).

### Example 213

### (R)-1-(3-{4-Amino-5-[3-(7-oxa-bicyclo[2.2.1]hept-1-ylmethoxy)-phenyl]-pyrrolo[2,3-d]pyrimidin-7-yl}-cyclobutylmethyl)-pyrrolidin-3-ol

The title compound was synthesized by analogy to the preparation of compound of Example 198 starting from toluene-4-sulfonic acid 3-{4-amino-5-[3-(7-oxa-bicyclo[2.2.1]hept-1-ylmethoxy)-phenyl]-pyrrolo[2,3-d]pyrimidin-7-yl}-cyclobutylmethyl ester (Intermediate CF) and (R)-3-hydroxypyrrolidie. Beige solid. HPLC t_{R}=2.222 min (Method D), HPLCIMS t_{R}=0.69 min, M+H=490.3 (method NMR (600 MHz, DMSO-d₆) δ ppm 8.13 (s, 1H, pyrimidinyl), 7.64 (s, 1H, pyrrolyl), 7.37 (t, 1H, phenyl), 7.09 (s, 1H, phenyl), 7.07/6.96 (d/d, 1H/1H, phenyl), 6.15 (s/b, 2H, NH₂), 4.80 (s/b, 1H,Hβ-cyclobutyl), 5.10 (quintet, 1H, CH-cyclobutyl), 4.70 (s/b, 1H, HO-pyrrolidinyl), 4.51 (m, 1H, CH-oxabicycloheptanyl), 4.30 (s, 2H, CH₂-β-phenyl),4.18 (m, 1H, CH-pyrrolinyl), 2.59 (m/m, 2H, CH₂—N), 2.57-2.39 (m/m, 2H, CH₂-pyrrolidinyl), 2.55/2.20 (mlm, 4H, CH₂-cyclobutyl), 2.46/1.50 (m/m, 2H, CH₂-pyrrolidinyl), 2.25 (m, 1H, CH-pyrrolidinyl), 1.69/1.54 (m/m, 8H, CH₂-oxabicycloheptanyl).

### Example 214

### (R)-1-(cis-3-{4-Amino-5-[3-(7-oxa-bicyclo[2.2.1]hept-1-ylmethoxy)-phenyl]-pyrrolo[2,3-d]pyrimidin-7-yl}-cyclobutylmethyl)-pyrrolidin-3-ol

The title compound was synthesized by analogy to the preparation of compound of Example 198 starting from toluene-4-sulfonic acid 3-{4-amino-5-[3-(7-oxa-bicyclo[2.2.1]hept-1-ylmethoxy)-phenyl]-pyrrolo[2,3-d]pyrimidin-7-yl}-cyclobutylmethyl ester (Intermediate CF) and (S)-3-hydroxypyrrolidie. Beige solid. HPLC t_{R}=2.224 min (Method D), HPLCIMS t_{R}=0.70 min, M+H=490.3 (method X). ¹H NMR (600 MHz, DMSO-d₆) δ ppm 8.13 (s, 1H, pyrimidinyl), 7.64 (s, 1H, pyrrolyl), 7.37 (t, 1H, phenyl), 7.09 (s, 1H, phenyl), 7.07/6.96 (d/d, 1H/1H, phenyl), 6.15 (s/b, 2H, NH₂), 4.80 (s/b, 1H,Hβ-cyclobutyl), 5.10 (quintet, 1H, CH-cyclobutyl), 4.70 (s/b, 1H, HO-pyrrolidinyl), 4.51 (m, 1H, CH-oxabicycloheptanyl), 4.30 (s, 2H, CH₂—O-phenyl),4.18 (m, 1H, CH-pyrrolinyl), 2.59 (m/m, 2H, CH₂—N), 2.57-2.39 (m/nn, 2H, CH₂-pyrrolidinyl), 2.55/2.20 (m/m, 4H, CH₂-cyclobutyl), 2.46/1.50 (m/m, 2H, CH₂-pyrrolidinyl), 2.25 (m, 1H, CH-pyrrolidinyl), 1.69/1.54 (m/m, 8H, CH₂-oxabicycloheptanyl).

### Example 215

### 7-[(R)-1-(1,1-dioxo-hexahydro-thiopyran-4-yl)-pyrrolidin-3-yl]-5-[3-(7-oxa-bicyclo[2.2.1]hept-1-ylmethoxy)-phenyl]-7H-pyrrolo[2,3-d]pyrimidin-4-ylamine

To the stirred mixture of (R)-5-[3-(7-oxa-bicyclo[2.2.1]hept-1-ylmethoxy)-phenyl]-7-pyrrolidin-3-yl-7H-pyrrolo[2,3-d]pyrimidin-4-ylamine (Intermediate CG, 41 mg, 0.10 mmol), 1,1-dioxo-tetrahydro-thiopyran-4-one (CAS 89365-50-4) (16 mg, 0.105 mmol, 1.05 eq) and 1,2-dichloroethane (1 mL) was added sodium triacetoxyborohydride (33 mg, 16.20 mmol, 1.4 eq) and acetic acid (0.0573 mL, 0.10 mmol, 1 eq) and stirred for 21 h at rt. The reaction mixture was quenched by addition of Na₂CO₃ 1 M and extracted with EtOAc. The combined organic layers were washed with water and brine, dried (Na₂SO₄), filtered and concentrated. The residue was purified by silica gel column chromatography (DCM/MeOH/NH₃^{aq}, 200:20:1) to afford 31 mg of the title compound as yellow foam: ES-MS: M+H=538.2; NMR (600 MHz, DMSO-d₆) δ ppm 8.14 (s, 1H), 7.51 (s, 1H), 7.36 (t, 1H), 7.07 (s, 1H), 7.04 (d, 1H), 6.94 (d, 1H), 6.22 (s/b, 2H), 5.39 (m, 1H), 4.51 (t, 1H), 4.32 (s, 2H), 3.31 (m, 2H), 3.12 (m, 2H), 3.00 (m, 2H), 2.79 (m, 2H), 2.46 (m, 2H), 2.07 (m, 5H), 1.69 (m, 4H), 1.57 (m, 4H).

### Example 216

### 7-[(R)-1-(1-Methyl-piperidin-4-yl)-pyrrolidin-3-yl]-5-[3-(7-oxa-bicycl o[2.2.1]hept-1-ylmethoxy)-phenyl]-7H-pyrrolo[2,3-d]pyrimidin-4-ylannine

The title compound was prepared in a similar manner to Example 215 starting from (R)-5-[3-(7-oxa-bicyclo[2.2.1]hept-1-ylmethoxy)-phenyl]-7-pyrrolidin-3-yl-7H-pyrrolo[2,3-d]pyrimidin-4-ylamine (example 215.1) and 1-methyl-piperidin-4-one (CAS1445-73-4): ES-MS: M+H=503.2; ¹H NMR (600 MHz, DMSO-d₆) δ ppm 8.13 (s, 1H), 7.49 (s, 1H), 7.37 (t, 1H), 7.07 (s, 1H), 7.04 (d, 1H), 6.96 (d, 1H), 6.15 (s/b, 2H), 5.30 (m, 1H), 4.51 (t, 1H), 4.31 (s, 2H), 3.01 (m, 1H), 2.83 (m, 2H), 2.65 (m, 2H), 2.52 (m, 1H), 2.38 (m, 2H), 2.13 (s, 3H), 2.06 (m, 1H), 1.95 (m, 3H), 1.35-1.85 (m, 11H).

### Example 217

### (E)-3-{4-Amino-5-[3-(7-oxa-bicyclo[2.2.1]hept-1-ylmethoxy)-phenyl]-pyrrolo[2,3-d]pyrimidin-7-yl}-1-(1-methyl-1H-tetrazol-5-ylmethyl)-cyclobutanol

To the stirred solution of (E)-3-{4-amino-5-[3-(7-oxa-bicyclo[2.2.1]hept-1-ylmethoxy)-phenyl]-pyrrolo[2,3-d]pyrimidin-7-yl}-1-(1H-tetrazol-5-ylmethyl)-cyclobutanol (Intermediate CH, 118 mg, 0.181 mmol) and THF (10 ml) was added an excess of a diazomethane (freshly prepared—in diethyl ether) at 0° C. After 0.5 h methanol (5 ml) was added and the reaction mixture was concentrated. HPLC-MS: Rₜ=0.85 (M+H=503.3) (3-{4-amino-5-[3-(7-oxa-bicyclo[2.2.1]hept-1-ylmethoxy)-phenyl]-pyrrolo[2,3-d]pyrimidin-7-yl}-1-(2-methyl-2H-tetrazol-5-ylmethyl)-cyclobutanol and Rₜ=0.89 (M+H=503.4) (title compound). The residue was purified by reverse phase preparative HPLC (Method R) to afford 18 mg (second eluting peak) of the title compound as a white foam. HPLC-MS: M+H=503.4 (Rₜ=0.89) (methode X); ¹H NMR (600 MHz, DMSO-d₆) δ ppm 8.11 (s, 1H), 7.56 (s, 1H), 7.38 (t, 1H), 7.08 (s, 1H), 7.05 (d, 1H), 6.97 (d, 1H), 6.15 (s/b, 2H), 5.40 (m, 1H), 5.31 (s, 1H), 4.51 (t, 1H), 4.32 (s, 2H), 4.30 (s, 3H), 3.23 (m, 2H), 2.78 (m, 2H), 2.44 (m, 2H), 1.69 (m, 4H), 1.58 (m, 4H).

### Example 218

### (E)-(3-{4-Amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl)-carbamic acid methyl ester

To the stirred solution of 1-aminomethyl-(E)-3-{4-amino-5-[3-(7-oxa-bicyclo[2.2.1]hept-1-ylmethoxy)-phenyly]-pyrrolo[2,3-d]pyrimidin-7-yl}-cyclobutanol (Intermediate BL; 44 mg, 0.10 mmol) and pyridine (1 mL) was added methyl chloroformate (0.0094 mL, 0.12 mmol, 1.2 eq) at 0° C. The reaction mixture was stirred for an additional 15 min at 0° C. and then partitioned between water and EtOAc. The combined organic layers were washed with water, dried

(Na₂SO₄), filtered and concentrated. The residue was purified by silica gel column chromatography (DCM/MeOH/NH₃^{aq}, 200:20:1) to afford 86 mg of the title compound as a beige foam: HPLC-MS: M+H=494.3 (Rₜ=0.87) (Method X); TLC; R_{f}=0.24 (DCM/MeOH/NH₃^{aq}, 200:20:1).

### Example 219

### (E)-3-(4-amino-5-[3-(7-oxa-bicyclo[2.2.1]hept-1-ylmethoxy)-phenyl]-pyrrolo[2,3-d]pyrimidin-7-yl}-1-methyl-cyclobutanol

To the stirred solution of 3-{4-amino-5-[3-(7-oxa-bicyclo[2.2.1]hept-1-ylmethoxy)-phenyl]-pyrrolo[2,3-d]pyrimidin-7-yl}-cyclobutanone (Intermediate BM; 71 mg, 0.149 mmol) and THF (2 mL) was slowly added methylmagnesium bromide (3 M in diethyl ether) (0.298 mL, 0.418 mmol, 2.8 eq) at −78° C. The reaction mixture was stirred for 1 h at 0° C. and then partitioned between ammonium chloride 1 Mand EtOAc. The combined organic layers were washed with brine, dried (Na₂SO₄), filtered and concentrated. The residue was purified by reverse phase preparative HPLC (Methode R) to afford 18 mg of the title compound as a yellow foam: HPLC-MS: M+H=421.3 (Rₜ=0.89) (Method X); TLC; R_{f}=0.33 (DCM/MeOH/NH₃^{aq}, 200:20:1). ¹H NMR (600 MHz, DMSO-d₆) δ ppm 8.41 (s, 1H), 7.92 (s, 1H), 7.42 (t, 1H), 7.14 (s, 1H), 7.09 (d, 1H), 7.05 (d, 1H), 4.91 (m, 1H), 4.52 (t, 1H), 4.32 (s, 2H), 3.54 (bs, 3H(OH, NH₂ and H₂O)) 2.58 (m, 4H), 1.70 (m, 4H), 1.58 (m, 4H), 1.36 (s, 3H).

### Example 220

### (E)-2-{4-amino-5-[3-(7-oxa-bicyclo[2.2.1]hept-1-ylmethoxy)-phenyl]-pyrrolo[2,3-d]pyrimidin-7-yl}-5-oxa-8-aza-spiro[3.5]nonan-7-one

To the stirred solution of (E)-N-(3-{4-amino-5-[3-(7-oxa-bicyclo[2.2.1]hept-1-ylmethoxy)-phenyl]-pyrrolo[2,3-d]pyrimidin-7-yl}-1-hydroxy-cyclobutylmethyl)-2-chloro-acetamide (Intermediate CI, 50 mg, 0.098 mmol) and benzene (5 mL) was added KOtBu (56.5 mg, 0.488 mmol, 5.0 eq) in portions over 1 h at rt. The reaction mixture was stirred for an additional 1.5 h at it and then partitioned between ice-water and EtOAc. The combined organic layers were washed with brine, dried (Na₂SO₄), filtered and concentrated. The residue was purified by silica gel column chromatography (DCM/MeOH/NH₃^{aq}, 200:20:1) to afford 16 mg of the title compound as a white foam: HPLC-MS: M+H=476.3 (Rₜ=0.83) (Method X); TLC; R_{f}=0.32 (DCM/MeOH/NH₃^{aq}, 200:20:1). ¹H NMR (600 MHz, DMSO-d₆) δ ppm 8.14 (s, 1H), 8.05 (s, 1H), 7.73 (s, 1H), 7.39 (t, 1H), 7.09 (s, 1H), 7.06 (d, 1H), 6.98 (d, 1H), 6.15 (bs, 2H), 5.33 (m, 1H), 4.51 (t, 1H), 4.31 (s, 2H), 4.13 (s, 2H), 3.37 (m, 2H), 2.72 (m, 2H), 2.62 (m, 2H), 1.69 (m, 4H), 1.58 (m, 4H).

### Example 221

### cis-3-{4-amino-5-[3-(7-oxa-bicyclo[2.2.1]hept-1-ylmethoxy)-phenyl]-pyrrolo[2,3-d]pyrimidin-7-yl}cyclobutanol

To the stirred solution of 3-{4-amino-5-[3-(7-oxa-bicyclo[2.2.1]hept-1-ylmethoxy)-phenyl]-pyrrolo[2,3-d]pyrimidin-7-yl}-cyclobutanone (Intermediate BM; 60 mg, 0.127 mmol) and MeOH (1.27 mL) was added sodium borohydride (10 mg, 0.253 mmol, 2.0 eq) at 0° C. The reaction mixture was stirred for 0.5 h at 0° C. and then partitioned between ammonium chloride 1 Mand EtOAc. The reaction mixture was concentrated and residue was purified by silica gel column chromatography (DCM/MeOH/NH₃^{aq}, 200:20:1) to afford 30 mg of the title compound as white foam: HPLC-MS: M+H=407.3 (Rₜ=0.84) (Method X); TLC; R_{f}=0.40 (DCM/MeOH/NH₃^{aq}, 200:20:1). ¹H NMR (600 MHz, DMSO-d₆) δ ppm 8.12 (s, 1H), 7.58 (s, 1H), 7.38 (t, 1H), 7.10 (s, 1H), 7.06 (d, 1H), 6.97 (d, 1H), 6.15 (bs, 2H), 5.26 (s, 1H), 4.73 (m, 1H), 4.51 (t, 1H), 4.32 (s, 2H), 4.02 (m, 1H), 2.77 (m, 2H), 2.36 (m, 2H), 1.69 (m, 4H), 1.58 (m, 4H).

### Example 222

### trans-3-{4-amino-5-[3-(7-oxa-bicyclo[2.2.1]hept-1-ylmethoxy)-phenyl]-pyrrolo[2,3-d]pyrimidin-7-yl}-cyclobutanol

To the stirred solution of (Z)-3-{4-amino-5-[3-(7-oxa-bicyclo[2.2.1]hept-1-ylmethoxy)-phenyl]-pyrrolo[2,3-d]pyrimidin-7-yl}-cyclobutanol (Example 221; 61 mg, 0.149 mmol) and THF (1.5 mL) were added subsequently at rt, benzoic acid (36.7 mg, 0.297 mmol, 2.0 eq), triphenyl-phosphine (79 mg, 0.297 mmol, 2.0 eq) and diisopropyl azodicarboxylate (0.061 mL, 0.297 mmol, 2.0 eq). The reaction mixture was stirred for 45 min at rt and then partitioned between brine and EtOAc. The combined organic layers were dried (Na₂SO₄), filtered and concentrated. The residue was dissolved in MeOH (1.5 mL) and THF (0.3 mL) and potassium cabonat (104 mg, 0.743 mmol) were added. The mixture was stirred for 16 h at rt and then partitioned between brine and EtOAc. The combined organic layers were dried (Na₂SO₄), filtered and concentrated. The residue was purified by silica gel column chromatography (DCM/MeOH/NH₃^{aq}, 200:20:1) to afford 30 mg of the title compound as white foam: HPLC-MS: M+H=407.2 (R=0.85) (Method X); TLC; R_{f}=0.40 (DCM/MeOH/NH₃^{aq}, 200:20:1). ¹H NMR (600 MHz, DMSO-d₆) δ ppm 8.13 (s, 1H), 7.61 (s, 1H), 7.38 (t, 1H), 7.10 (s, 1H), 7.06 (d, 1H), 6.96 (d, 1H), 6.15 (bs, 2H), 5.39 (m, 1H), 5.23 (s, 1H), 4.51 (t, 1H), 4.46 (m, 1H), 4.32 (s, 2H), 2.71 (m, 2H), 2.38 (m, 2H), 1.70 (m, 4H), 1.58 (m, 4H).

### Example 223

### N-(cis-3-{4-amino-5-[3-(7-oxa-bicyclo[2.2.1]hept-1-ylmethoxy)-phenyl]-pyrrolo[2,3-d]pyrimidin-7-yl}-cyclobutyl)-acetamide

To the stirred solution of 3-{4-amino-5-[3-(7-oxa-bicyclo[2.2.1]hept-1-ylmethoxy)-phenyl]-pyrrolo[2,3-d]pyrimidin-7-yl}-cyclobutanol (Intermediate CJ, 35 mg, 0.082 mmol) and pyridine (0.82 mL) was added acetyl chloride (0.0071 mL, 0.098 mmol, 1.2 eq) at 0° C. The reaction mixture was stirred for an additional 30 min at 0° C. and then partitioned between water and EtOAc. The combined organic layers were washed with water, dried (Na₂SO₄), filtered and concentrated. The residue was purified by silica gel column chromatography (DCM/MeOH/NH₃^{aq}, 200:20:1) to afford 26 mg of the title compound as white foam: HPLC-MS: M+H=448.3 (Rₜ=0.83) (Method X); TLC; R_{f}=0.12 (DCM/MeOH/NH₃^{aq}, 200:20:1). ¹H NMR (600 MHz, DMSO-d₆) δ ppm 8.17 (d, 1H), 8.14 (s, 1H), 7.58 (s, 1H), 7.39 (t, 1H), 7.10 (s, 1H), 7.06 (d, 1H), 6.98 (d, 1H), 6.17 (bs, 2H), 4.95 (m, 1H), 4.52 (t, 1H), 4.32 (s, 2H), 4.14 (m, 1H), 2.80 (m, 2H), 2.44 (m, 2H), 1.81 (s, 3H), 1.69 (m, 4H), 1.58 (m, 4H).

### Example 224

### 7-[1-(1,1-dioxo-thietan-3-yl)-azetidin-3-yl]-5-[3-(7-oxa-bicyclo[2.2.1]hept-1-ylmethoxy)-phenyl]-7H-pyrrolo[2,3-d]pyrimidin-4-ylamine

To the stirred solution of 7-azetidin-3-yl-5-[3-(7-oxa-bicyclo[2.2.1]hept-1-ylmethoxy)-phenyl]-7H-pyrrolo[2,3-d]pyrimidin-4-ylamine (Intermediate CK, 78.4 mg, 0.20 mmol) and MeOH (1.0 mL) was added subsequently triethylamine (0.056 mL, 0.40 mmol, 2 eq) and 3-bromothietane 1,1-dioxide 70% (68.8 mg, 0.260 mmol, 1.3 eq). The reaction mixture was stirred for 16 h at rt and then partitioned between water and EtOAc. The combined organic layers were washed with water and brine, dried (Na₂SO₄), filtered and concentrated. The residue was purified by silica gel column chromatography (DCM/MeOH/NH₃^{aq}, 200:20:1) to afford 65 mg of the title compound as white foam: HPLC-MS: M+H=496.2 (Rₜ=0.87) (Method X); TLC; R_{f}=0.50 (DCM/MeOH/NH₃^{aq}, 200:20:1). ¹H NMR (600 MHz, DMSO-d₆) δ ppm 8.15 (s, 1H), 7.71 (s, 1H), 7.39 (t, 1H), 7.10 (s, 1H), 7.07 (d, 1H), 6.99 (d, 1H), 6.15 (bs, 2H), 5.28 (m, 1H), 4.51 (t, 1H), 4.32 (s, 2H), 4.30 (m, 2H), 4.04 (m, 2H), 3.79 (t, 2H), 3.65 (m, 1H), 3.58 (t, 2H), 1.70 (m, 4H), 1.58 (m, 4H).

### Example 225

### 7-[(R)-1-(1,1-dioxo-thietan-3-yl)-pyrrolidin-3-yl]-5-[3-(7-oxa-bicyclo[2.2.1]hept-1-ylmethoxy)-phenyl]-7H-pyrrolo[2,3-d]pyrimidin-4-ylamine

The title compound was prepared in a similar manner to Example 224 starting from (R)-5-[3-(7-oxa-bicyclo[2.2.1]hept-1-ylmethoxy)-phenyl]-7-pyrrolidin-3-yl-7H-pyrrolo[2,3-d]pyrimidin-4-ylamine (example 215.1) and 3-bromo-thietane 1,1-dioxide (CAS 59463-72-8): HPLC-MS: M+H=510.3(Rₜ=0.91) (methode X); TLC; R_{f}=0.56 (DCM/MeOH/NH₃^{aq}, 200:20:1).

### Examples 226 and 227

### 5-[3-(7-Oxa-bicyclo[2.2.1]hept-1-ylmethoxy)-phenyl]-7-{cis-3-[(15,45)-1-(2-thia-5-aza-bicyclo[2.2.1]hept-5-yl)methyl]-cyclobutyl}-7H-pyrrolo[2,3-d]pyrimidin-4-ylamine and 5-[3-(7-Oxa-bicyclo[2.2.1]hept-1-ylmethoxy)-phenyl]-7-{(trans-3-[(1S,4S)-1-(2-thia-5-aza-bicyclo[2.2.1]hept-5-yl)methyl]-cyclobutyl}-7H-pyrrolo[2,3-d]pyrimidin-4-ylamine

5-lodo-7-{3-[(1S,4S)-1-(2-thia-5-aza-bicyclo[2.2.1]hept-5-yl)methyl]-cyclobutyl}-7H pyrrolo[2,3-d]pyrimidin-4-ylamine (Intermediate CL, 100 mg, 0.215 mmol) and 1-methyl-4-[3-(4,4,5,5-tetramethyl-[1,3,2]dioxaborolan-2-yl)-phenoxymethyl]-7-oxa-bicyclo[2.2.1]heptane (Intermediate BB; 85 mg, 0.258 mmol, 1.2 eq) were dissolved in DMF (2.5 mL). Water (0.10 mL), K₃PO₄ (91 mg, 0.431 mmol, 2 eq), Na₂CO₃ (45.6 mg, 0.431 mmol, 2 eq) and tetrakis (triphenylphosphine) palladium (24.9 mg, 0.022 mmol, 0.1 eq) were added and the reaction mixture purged with argon. It was then heated to 100° C. and stirred at this temperature for 2 h. The reaction mixture was then partitioned between brine and EtOAc. The combined organic layers were washed with water and brine, dried (Na₂SO₄), filtered and concentrated. The residue was purified by silica gel column chromatography (DCM/MeOH/NH₃^{aq}, 200:20:1) to afford: 20 mg of the title compound (Example 227) and 54 mg of the title compound (Example 226) as yellow foams:

Example 226 (second eluting fraction): HPLC-MS: M+H=518.3 (Rₜ=0.71) (Method X); TLC; R_{f}=0.36 (DCM/MeOH/NH₃^{aq}, 200:20:1).
Example 227 (first eluting fraction): HPLC-MS: M+H=518.2 (Rₜ=0.71) (Method X); TLC; R_{f}=0.40 (DCM/MeOH/NH₃^{aq}, 200:20:1).

### Examples 228 and 229

### (endo)-5-[3-(7-oxa-bicyclo[2.2.1]hept-1-ylmethoxy)-phenyl]-7-[3-((1S,2S,4S)-2-oxo-2-thia-5-aza-bicyclo[2.2.1]hept-5-ylmethyl)-cyclobutyl]-7H-pyrrolo[2,3-d]pyrimidin-4-ylamine and (exo)-5-[3-(7-oxa-bicyclo[2.2.1]hept-1-ylmethoxy)-phenyl]-7-[3-((1S,2R,4S)-2-oxo-2-thia-5-aza-bicyclo[2.2.1]hept-5-ylmethyl)-cyclobutyl]-7H-pyrrolo[2,3-d]pyrimidin-4-ylamine

To the stirred solution of 5-[3-(7-oxa-bicyclo[2.2.1]hept-1-ylmethoxy)-phenyl]-7-(cis-3-[(1S,4S)-1-(2-thia-5-aza-bicyclo[2.2.1]hept-5-yl)methyl]-cyclobutyl}-7H-pyrrolo[2,3-d]pyrimidin-4-ylamine (Example 226, 51 mg, 0.10 mmol) and DCM (5 mL) was added 3-chloroperoxybenzoic acid (33 mg, 0.15 mmol, 1.5 eq) in portions over 1 h at 0° C. The reactaion mixture was stirred an additional 0.5 h at 0° C. and then partitioned between NaHCO₃ 1 Mand DCM. The combined organic layers were washed with brine, dried (Na₂SO₄), filtered and concentrated. The residue was purified by silica gel column chromatography (DCM/MeOH/NH₃^{aq}, 200:20:1 and DCM/MeOH/NH₃^{aq}, 40:10:1) to afford: 14 mg of the title compound (Example 228) and 15 mg of the title compound (Example 229) as yellow foams:

Example 228 (first eluting fraction): HPLC-MS: M+H=534.3 (Rₜ=0.64) (Method X); TLC; R_{f}=0.29 (DCM/MeOH/NH₃^{aq}, 200:20:1).
Example 229 (second eluting fraction): HPLC-MS: M+H=534.1 (Rₜ=0.94) (Method X); TLC; R_{f}=0.07 (DCM/MeOH/NH₃^{aq}, 200:20:1).

### Example 230

### 7-[cis-3-(1,1-dioxo-thiomorpholin-4-yl)-cyclobutyl]-5-[3-(7-oxa-bicyclo[2.2.1]hept-1-ylmethoxy)-phenyl]-7H-pyrrolo[2,3-d]pyrimidin-4-ylamine

To the stirred solution of 3-{4-amino-5-[3-(7-oxa-bicyclo[2.2.1]hept-1-ylmethoxy)-phenyl]-pyrrolo[2,3-d]pyrimidin-7-yl}-cyclobutanol (Intermediate CJ, 86 mg, 0.20 mmol) and DMF (2.0 mL) was added subsequently divinylsulfone (0.022 mL, 0.22 mmol, 1.1 eq) and SiO2 (1 mg) at rt. The reaction mixture was stirred 21 h at rt and then partitioned between water and EtOAc. The combined organic layers were washed with water and brine, dried (Na₂SO₄), filtered and concentrated. The residue was purified by silica gel column chromatography (DCM/MeOH/NH₃^{aq}, 200:20:1) to afford 80 mg of the title compound as white crystals: HPLC-MS: M+H=524.4 (Rₜ=0.81) (Method X); TLC; R_{f}=0.52 (DCM/MeOH/NH₃^{aq}, 200:20:1). ¹H NMR (600 MHz, DMSO-d₆) δ ppm 8.13 (s, 1H), 7.54 (s, 1H), 7.38 (t, 1H), 7.10 (s, 1H), 7.06 (d, 1H), 6.96 (d, 1H), 6.17 (bs, 2H), 4.88 (m, 1H), 4.51 (t, 1H), 4.32 (s, 2H), 3.11 (s, 4H), 2.96 (m, 1H), 2.85 (s, 4H), 2.63 (m, 2H), 2.36 (m, 2H), 1.70 (m, 4H), 1.58 (m, 4H).

### Example 231

### 5-[cis-3-(7-oxa-bicyclo[2.2.1]hept-1-ylmethoxy)-phenyl]-7-[3-(1-oxo-thiomorpholin-4-ylmethyl)-cyclobutyl]-7H-pyrrolo[2,3-d]pyrimidin-4-ylamine

The title compound was prepared in a similar manner to Example 226 starting from 5-iodo-7-[cis-3-(1-oxo-thiomorpholin-4-ylmethyl)-cyclobutyl]-7H-pyrrolo[2,3-d]pyrimidin-4-ylamine (Intermediate CM) and 1-methyl-4-[3-(4,4,5,5-tetramethyl-[1,3,2]dioxaborolan-2-yl)-phenoxymethyl]-7-oxa-bicyclo[2.2.1]heptane (Intermediate BB): HPLC-MS: M+H=522.4 (Rₜ=0.63) (methode X); ¹H NMR (600 MHz, DMSO-d₆) δ ppm 8.15 (bs, 1H), 7.66 (s, 1H), 7.38 (t, 1H), 7.09 (s, 1H), 7.05 (d, 1H), 6.97 (d, 1H), 6.15 (s/b, 2H), 5.09 (m, 1H), 4.51 (t, 1H), 4.31 (s, 2H), 2.87 (m, 4H), 2.50-2.72 (m, 8H), 2.35 (m, 1H), 2.19 (m, 2H), 1.70 (m, 4H), 1.58 (m, 4H).

### Example 232

### 5-[3-(7-Oxa-bicyclo[2.2.1]hept-1-ylmethoxy)-phenyl]-7-[cis-3-(1-oxo-thiomorpholin-4-yl)-cyclobutyl]-7H-pyrrolo[2,3-d]pyrimidin-4-ylamine

To the stirred solution of cis-3-{4-amino-5-[3-(7-oxa-bicyclo[2.2.1]hept-1-ylmethoxy)-phenyl]-pyrrolo[2,3-d]pyrimidin-7-yl}-cyclobutanol (Intermediate CJ, 132 mg, 0.307 mmol) and acetonitrile (3.07 mL) was added divinylsulfoxide (0.032 mL, 0.338 mmol, 1.1 eq).The reaction mixture was stirred 4 days at 80° C. (every day additional divinylsulfoxide (0.032 mL, 0.338 mmol, 1.1 eq was added). The reaction mixture was partitioned between water and EtOAc. The combined organic layers were washed with brine, dried (Na₂SO₄), filtered and concentrated. The residue was purified by silica gel column chromatography (DCM/MeOH/NH₃^{aq}, 200:20:1) to afford 58 mg of the title compound as orange foam: HPLC-MS: M+H=508.3 (Rₜ, =0.67) (Method X); TLC; R_{f}=0.35 (DCM/MeOH/NH₃^{aq}, 200:20:1). ¹H NMR (600 MHz, DMSO-d₆) δ ppm 8.14 (s, 1H), 7.55 (s, 1H), 7.38 (t, 1H), 7.11 (s, 1H), 7.06 (d, 1H), 6.96 (d, 1H), 6.17 (bs, 2H), 4.91 (m, 1H), 4.51 (t, 1H), 4.32 (s, 2H), 2.86 (m, 2H), 2.78 (m, 5H), 2.64 (m, 4H), 2.33 (m, 2H), 1.70 (m, 4H), 1.58 (m, 4H).

### Example 233

### 7-[cis-3-(1-oxo-thiomorpholin-4-ylmethyl)-cyclobutyl]-5-{3-[(S)-1-(tetrahydrofuran-2-yl)methoxy]-phenyl}-7H-pyrrolo[2,3-d]pyrimidin-4-ylamine

The title compound was prepared in a similar manner to Example 226 starting from 5-iodo-7-[cis-3-(1-oxo-thiomorpholin-4-ylmethyl)-cyclobutyl]-7H-pyrrolo[2,3-d]pyrimidin-4-ylamine and 4,4,5,5-tetramethyl-2-{3-[(S)-1-(tetrahydro-furan-2-yl)methoxy]-phenyl}-[1,3,2]dioxaborolane (Intermediate BD): HPLC-MS: M+H=496.3 (Rₜ=0.57) (Method X); TLC; R_{f}=0.22 (DCM/MeOH/NH₃^{aq}, 200:20:1). ¹H NMR (600 MHz, DMSO-d₆) δ ppm 8.13 (s, 1H), 7.66 (s, 1H), 7.37 (t, 1H), 7.05 (d, 1H), 7.04 (s, 1H), 6.92 (d, 1H), 6.15 (bs, 2H), 5.10 (m, 1H), 4.18 (m, 1H), 4.00 (m, 2H), 3.78 (m, 1H), 3.69 (m, 1H), 2.87 (m, 4H), 2.50-2.65 (m, 8H), 2.35 (m, 1H), 2.19 (m, 2H), 2.10 (m, 1H), 1.88 (m, 1H), 1.84 (m, 1H), 1.69 (m, 1H).

### Example 234

### 7-[cis-3-(1-oxo-thiomorpholin-4-yl)-cyclobutyl]-5-{3-[(S)-1-(tetrahydro-furan-2-yl)methoxy]-phenyl}-7H-pyrrolo[2,3-d]pyrimidin-4-ylamine

The title compound was prepared in a similar manner to Example 226 starting from 5-iodo-7-[cis-3-(1-oxo-thiomorpholin-4-yl)-cyclobutyl]-7H-pyrrolo[2,3-d]pyrimidin-4-ylamine (Intermediate CN) and 4,4,5,5-tetramethyl-2-{3-[(S)-1-(tetrahydro-furan-2-yl)methoxy]-phenyl}-[1,3,2]dioxaborolane (Intermediate BD): HPLC-MS: M41=482.3 (R=0.60) (methode X); TLC; R_{f}=0.37 (DCM/MeOH/NH₃^{aq}, 200:20:1). ¹H NMR (600 MHz, DMSO-d₆) δ ppm 8.16 (bs, 1H), 7.54 (s, 1H), 7.37 (t, 1H), 7.05 (s, 1H), 7.04 (d, 1H), 6.91 (d, 1H), 6.15 (bs, 2H), 4.90 (m, 1H), 4.17 (m, 1H), 4.01 (m, 2H), 3.78 (m, 1H), 3.69 (m, 1H), 2.86-2.60 (m, 11H), 2.32 (m, 2H), 2.10 (m, 1H), 1.88 (m, 1H), 1.84 (m, 1H), 1.69 (m, 1H).

### Example 235

### 3-{4-Amino-5-[3-(7-oxa-bicyclo[2.2.1]hept-1-ylmethoxy)-phenyl]-pyrrolo[2,3-d]pyrimidin-7-yl}-1-dimethylaminomethyl-cyclobutanol

To a stirred solution of (E)-(1-(Aminomethyl-3-{4-amino-5-[3-(7-oxa-bicyclo[2.2.1]hept-1-ylmethoxy)-phenyl]-pyrrolo[2,3-d]pyrimidin-7-yl}-cyclobutanol) (Example 201) (20 mg) in MeOH (0.23 ml) was added formaldehyde (3.7 mg) and decaborane (5.6 mg). The reaction mixture was stirred at ambient temperature for 30 min and the reaction mixture was directly purified by preparative HPLC (method R) to give the title compound as a yellow solid (10 mg). HPLC/MS t_{R} 0.68 min, M+H 464.4 (Method X). ¹H NMR (400 MHz, DMSO-d₆) δ ppm 1.56 (m, 4H), 1.67 (m, 4H), 2.22 (s, 6H), 2.28-2.46 (m, 2H), 2.46-2.60 (m, 2H), 4.30 (s, 2H), 4.49 (bs, 1H), 4.86 (bs, 1H), 5.31-5.48 (m, 1H), 6.10 (s, 2H), 6.95 (d, 1H), 6.99-7.15 (m, 2H), 7.36 (t, 1H), 7.51-7-64 (m, 1H), 8.12 (s, 1H).

### Example 236

### 2-{4-Amino-5-[3-(7-oxa-bicyclo[2.2.1]hept-1-ylmethoxy)-phenyl]-pyrrolo[2,3d]pyrimidin-7-yl}-7-methyl-5-oxa-7-aza-spiro[3.4]octan-6-one

Potassium tert-butoxide (5.8 mg) was added to a cooled (0° C.) solution of Example 169: (E)-2-{4-amino-5-[3-(7-oxa-bicyclo[2.2.1]hept-1-ylmethoxy)-phenyl]-pyrrolo[2,3-d]pyrimidin-7-yl}-5-oxa-7-aza-spiro[3.4]octan-6-one (20 mg) in THF (1 ml) under nitrogen and the reaction was stirred at this temperature for 1 h. Iodomethane (9 mg) was introduced and the mixture stirred for 3 hours at 0° C. and 16 hours at ambient temperature. Water was added and extracted twice with EtOAc. The organics were combined, washed with brine, dried over sodium sulfate and the solvent removed. The crude product was purified by preparative HPLC to give the title compound as a white solid (5 mg). HPLC/MS t_{R} 0.93 min, M+H 476.3 (Method X). ¹H NMR (400 MHz, DMSO-dₑ) δ ppm 1.48-1.62 (m, 4H), 1.68 (m, 4H), 2.74 (s, 3H), 2.77-2.90 (m, 2H), 2.90-3.04 (m, 2H), 3.66 (s, 2H), 4.30 (s, 2H), 4.50 (t, 1H), 5.31 (t, 1H), 6.91-6.99 (m, 1H), 7.00-7.10 (m, 2H), 7.37 (t, 1H), 7-64 (s, 1H), 8.13 (s, 1H).

### Example 237

### 2-{4-Amino-5-[3-(7-oxa-bicyclo[2.2.1]hept-1-ylmethoxy)-phenyl]-pyrrolo[2,3d]pyrimidin-7-yl}-7-ethyl-5-oxa-7-aza-spiro[3.4]octan-6-one

The title compound was prepared in a similar manner to Example 236 starting from Example 169: (E)-2-{4-amino-5-[3-(7-oxa-bicyclo[2.2.1]hept-1-ylmethoxy)-phenyl]-pyrrolo[2,3-d]pyrimidin-7-yl}-5-oxa-7-aza-spiro[3.4]octan-6-one (29 mg) and iodoethane (15 mg) to give the title compound as a white solid (4 mg). HPLC/MS t_{R} 1.05 min, M+H 490.3 (Method X). ¹H NMR (400 MHz, CDCl3) δ ppm 1.19 (t, 3H), 1.56-1.71 (m, 4H), 1.71-1.99 (m, 4H), 3.03-3.27 (m, 4H), 3.35 (q, 2H), 3.73 (s, 2H), 4.31 (s, 2H), 4.63 (t, 1H), 5.19 (t, 1H), 5.62 (bs, 2H), 6.82-7.12 (m, 4H), 7.37 (t, 1H), 8.27 (s, 1H).

### Example 238

### 2-(4-Amino-5-{3-[(S)-1-(tetrahydro-furan-2-yl)methoxy]-phenyl}-pyrrolo[2,3-d]pyrimidin-7-yl)-5-oxa-7-aza-spiro[3.4]octan-6-one

The title compound was prepared in a similar manner to Example 226 starting from 2-(4-Amino-5-iodo-pyrrolo[2,3-d]pyrimidin-7-yl)-5-oxa-7-aza-spiro[3.4]octan-6-one (Intermediate CO) and 4,4,5,5-tetramethyl-2-{3-[(S)-1-(tetrahydrofuran-2-yl)methoxy]-phenyl}-[1,3,2]dioxaborolane (Intermediate BD). HPLC/MS t_{R} 0.70 min, M+H 436.3 (Method X). ¹H NMR (400 MHz, DMSO-d₅) δ ppm 1.68 (m, 1H), 1.74-1.92 (m, 2H), 1.92-2.09 (m, 1H), 2.77-2.89 (m, 2H), 2.89-3.00 (m, 2H), 3.59 (s, 2H), 3.61-3.72 (m, 1H), 3.72-3.83 (m, 1H), 3.90-4.04 (m, 2H), 4.15 (m, 1H), 5.30 (t, 1H), 6.91 (m, 1H), 6.98-7.07 (m, 2H), 7.36 (t, 1H), 7.54 (s, 1H) 7.62 (s, 1H) 8.13 (5, 1H).

Cellular IGF-1R and InsR assays

Compound-mediated inhibition of IGF1R and INSR phosphorylation in Hek293 cells trans-duced with the corresponding receptors were assessed in a capture ELISA format using the MSD (Meso Scale Discovery) platform. Briefly, 30,000 cells washed and diluted in starvation medium (DMEM high glucose supplemented with 0.1% BSA) were seeded in 90 μL per well into 96-well plates pre-coated with poly-D-lysine (0.1 mg/mL in PBS/O). After 24 h incubation at 37° C. and 5% CO₂, dose-response effects were determined with 3-fold serial compound dilutions, starting at 10 μM. The final vehicle concentration was 0.1% DMSO in all wells. Following pre-incubation with compounds for 1 h, receptor phosphorylation was triggered by a 10 min exposure to 1.0 ng/μL IGF for Hek293-IGF1 R cells, and 5.0 ng/μL insulin for Hek293-InsR cells. Cell lysis was achieved by addition of 80 μL MSD lysis buffer per aspirated well, incubation on ice for 20 min, and a freeze-thaw cycle. Target phosphorylation was then assessed by transferring volumes corresponding to approx. 6 μg Hek293-IGF1R or 0.6 μg Hek293-InsR lysates to MSD assay plates pre-coated with total-IGF1 R or total-InsR Abs, respectively. After incubation for 2 h at rt, wells were exposed for 1 hr to a rabbit monoclonal antibody (CST #3024, 1:1000) detecting pIGF1R(Tyr1135/1136) as well as pINSR(Tyr1150/1151). Immune complexes were detected by a SULFO-Tag™-coupled anti-rabbit IgG antibody in the presence of 150 μL MSD read-buffer. Light emission at 620 nm triggered by application of electric current was recorded on a MSD Sectorlmager 6000. Acquired raw data (mean Ru-ECL units) were processed in an Excel analysis template. The plate blank (MSD lysis buffer) was subtracted from all data points. The effect of a particular test compound concentration on receptor phosphorylation was expressed relative to the window defined by ligand-stimulated vs unstimulated control cells (set as 100%). IC₅₀ values [nM] were determined using 4-parametric curve-fitting (XLfit software, V4.3.2).

Test results obtained using the above describe method are summarized in the table below.

| Ex. | General Formula | 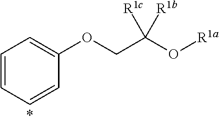 | 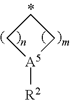 | IGF-1R IC₅₀ (nM) |
|---|---|---|---|---|
| 1 | I-1 | 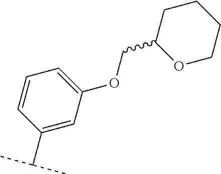 | 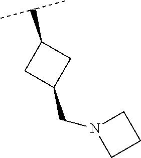 | 119-139 |
| 2 | I-1 | 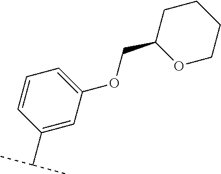 | 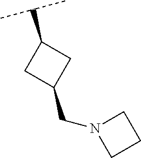 | 51-99 |
| 3 | I-1 | 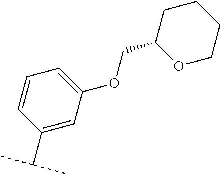 | 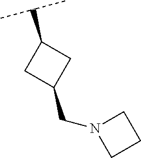 | 105-141 |
| 4 | I-1 | 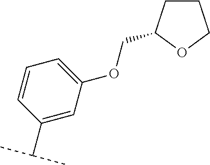 | 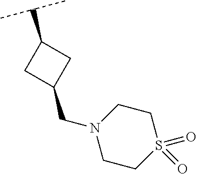 | 36 |
| 5 | I-1 | 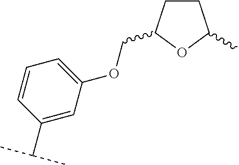 | 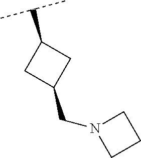 | 51-58 |
| 6 | I-1 | 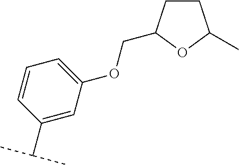 | 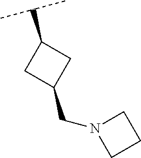 | 30 |
| 7 | I-1 | 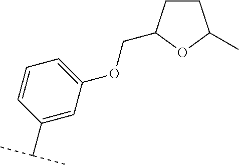 | 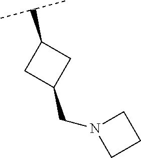 | 95-117 |
| 8 | I-1 | 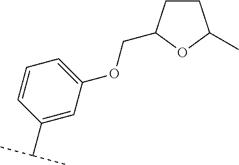 | 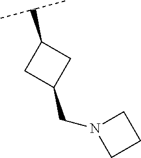 | |
| 9 | I-1 | 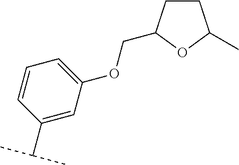 | 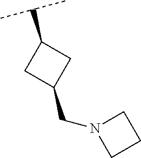 | |
| 10 | I-1 | 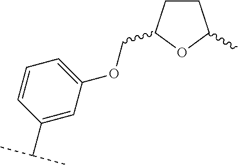 | 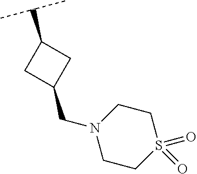 | |
| 11 | I-1 | 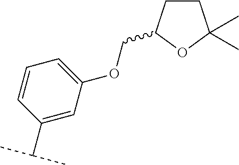 | 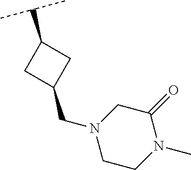 | |
| 12 | I-1 | 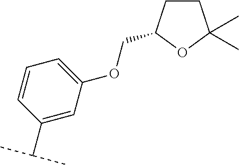 | 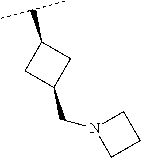 | |
| 13 | I-1 | 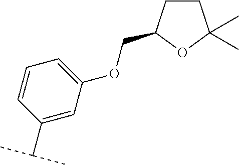 | 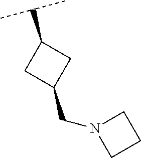 | |
| 14 | I-1 | 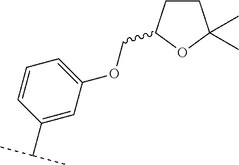 | 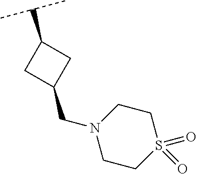 | 17 |
| 15 | I-1 | 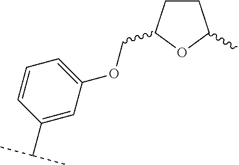 | 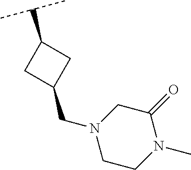 | |
| 16 | I-1 | 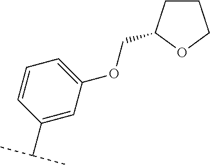 | 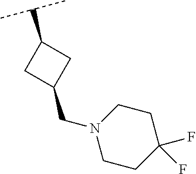 | 90-219 |
| 17 | I-1 | 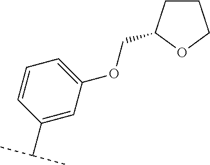 | 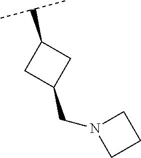 | |
| 18 | I-1 | 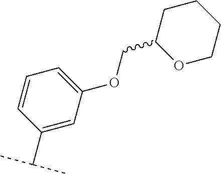 | 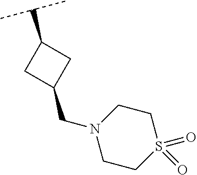 | |
| 19 | I-1 | 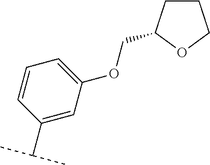 | 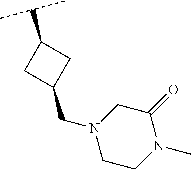 | |
| 20 | I-1 | 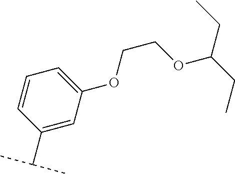 | 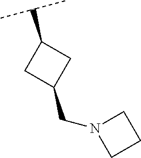 | |
| 21 | I-1 | 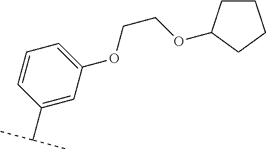 | 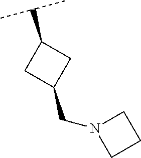 | |
| 22 | I-1 | 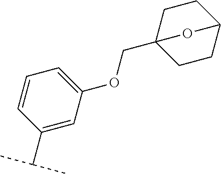 | 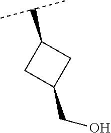 | 128 |
| 23 | I-1 | 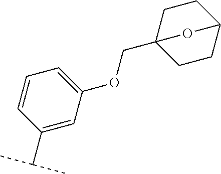 | 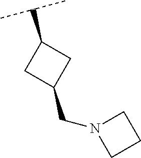 | 45-60 |
| 24 | I-1 | 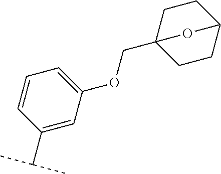 | 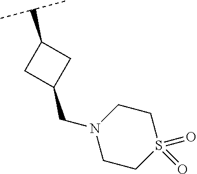 | 3.9-4 |
| 25 | I-1 | 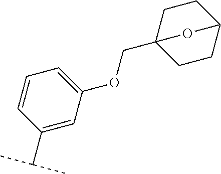 | 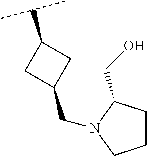 | <4-14 |
| 26 | I-1 | 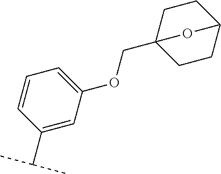 | 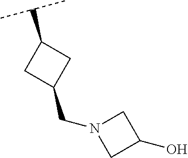 | 38 |
| 27 | I-1 | 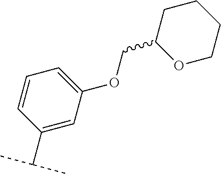 | 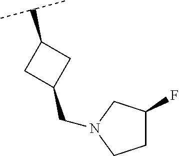 | 345 |
| 28 | I-1 | 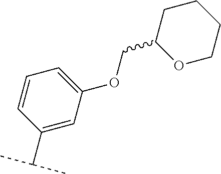 | 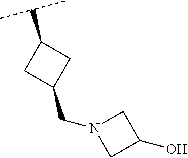 | 294 |
| 29 | I-1 | 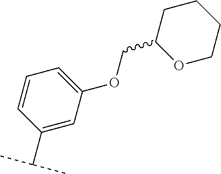 | 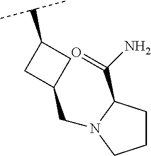 | 149-152 |
| 30 | I-2 | 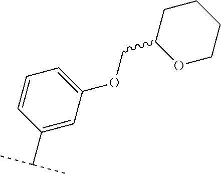 | 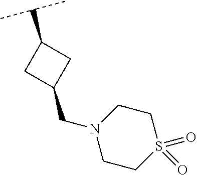 | 254 |
| 31 | I-1 | 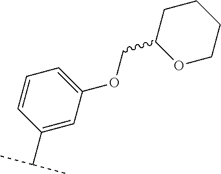 | 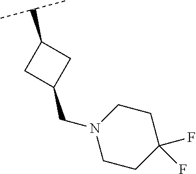 | 408 |
| 32 | I-1 | 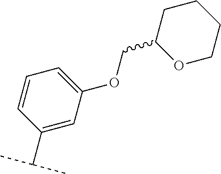 | 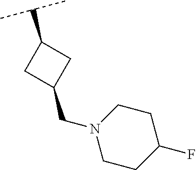 | 309 |
| 33 | I-1 | 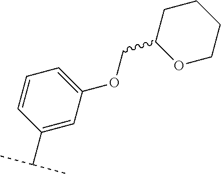 | 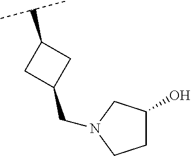 | 235 |
| 34 | I-1 | 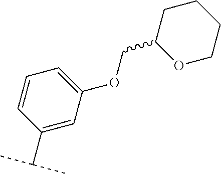 | 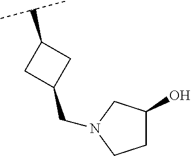 | 333 |
| 35 | I-1 | 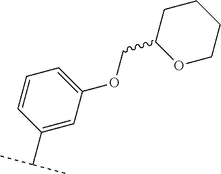 | 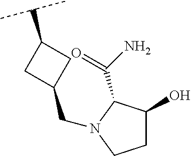 | 41-103 |
| 36 | I-1 | 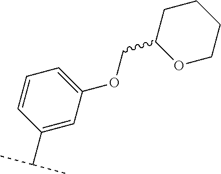 | 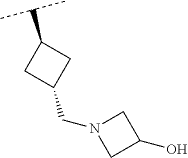 | 372 |
| 37 | I-1 | 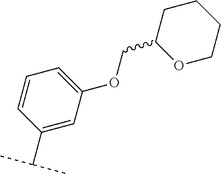 | 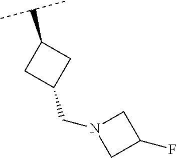 | 497 |
| 38 | I-1 | 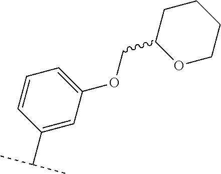 | 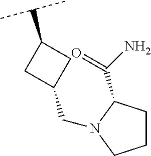 | 276 |
| 39 | I-1 | 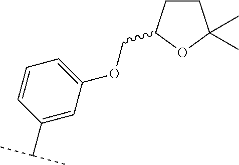 | 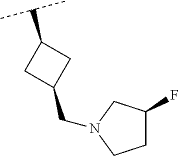 | 224 |
| 40 | I-1 | 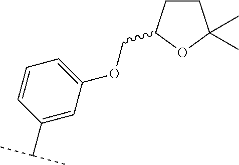 | 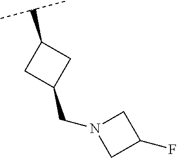 | 39-52 |
| 41 | I-1 | 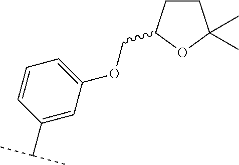 | 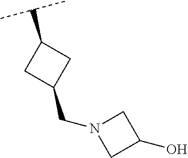 | 41 |
| 42 | I-1 | 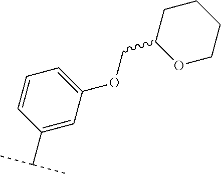 | 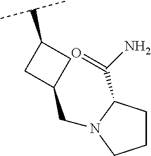 | 26-39 |
| 43 | I-1 | 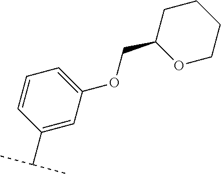 | 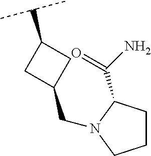 | <4-22 |
| 44 | I-1 | 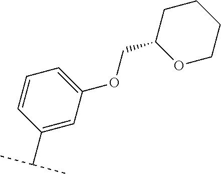 | 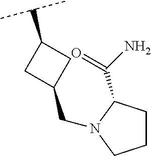 | 7-15 |
| 45 | I-1 | 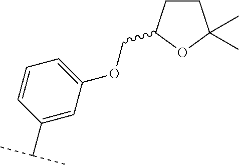 | 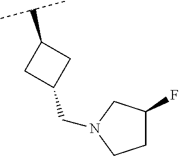 | 35 |
| 46 | I-1 | 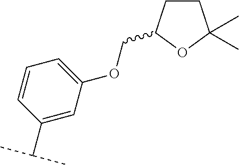 | 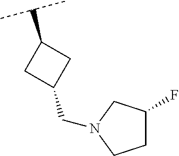 | 15 |
| 47 | I-1 | 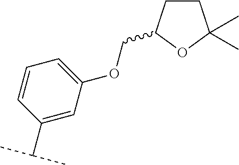 | 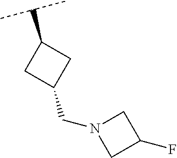 | 34 |
| 48 | I-1 | 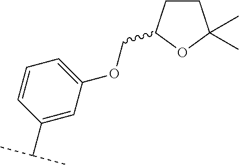 | 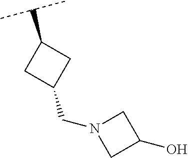 | 26 |
| 49 | I-1 | 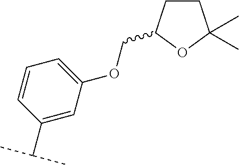 | 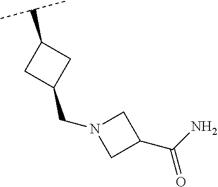 | 192-241 |
| 50 | I-1 | 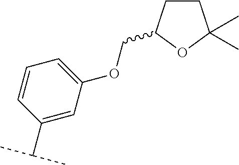 | 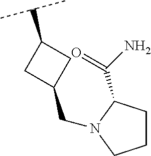 | 8 |
| 51 | I-1 | 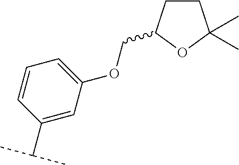 | 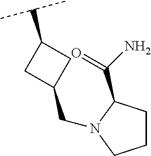 | 53 |
| 52 | I-1 | 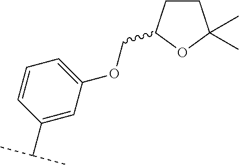 | 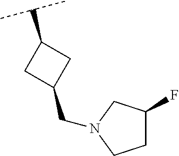 | 37 |
| 53 | I-1 | 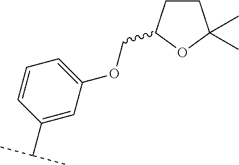 | 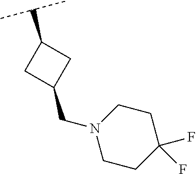 | 32-37 |
| 54 | I-1 | 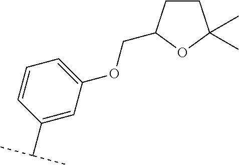 | 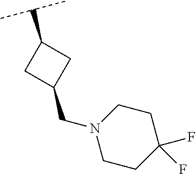 | 45-120 |
| 55 | I-1 | 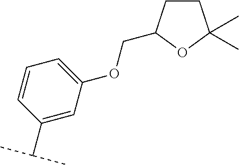 | 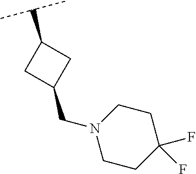 | 22-38 |
| 56 | I-1 | 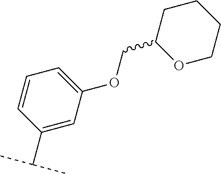 | 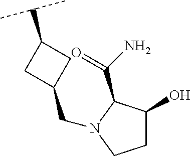 | 97 |
| 57 | I-1 | 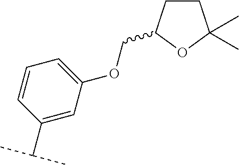 | 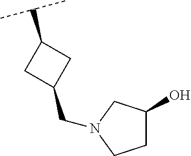 | 15 |
| 58 | I-1 | 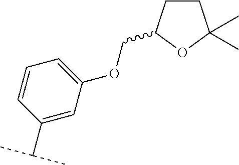 | 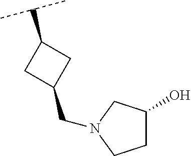 | 50 |
| 59 | I-1 | 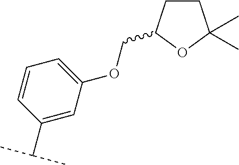 | 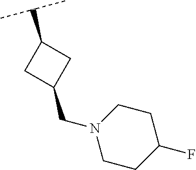 | 18 |
| 60 | I-1 | 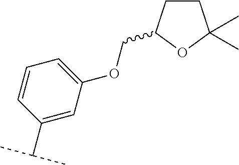 | 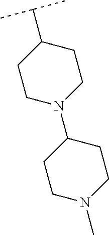 | 339 |
| 61 | I-1 | 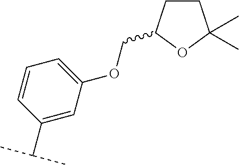 | 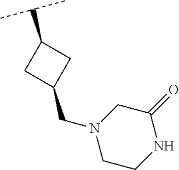 | 212 |
| 62 | I-1 | 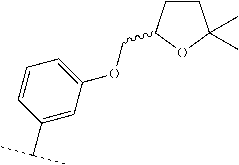 | 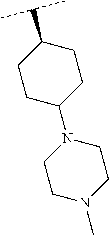 | 314 |
| 63 | I-1 | 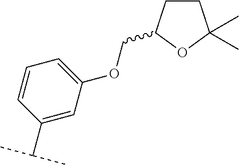 | 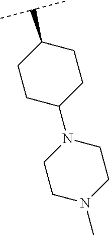 | 61 |
| 64 | I-1 | 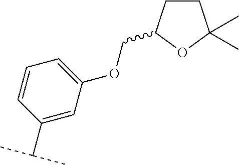 | 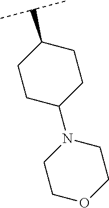 | 367 |
| 65 | I-1 | 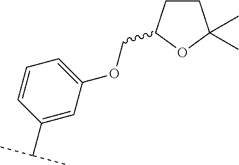 | 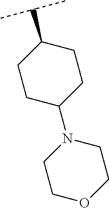 | 128 |
| 66 | I-1 | 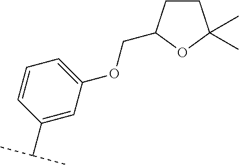 | 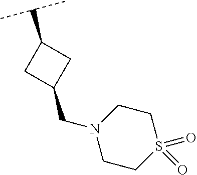 | <4 |
| 67 | I-1 | 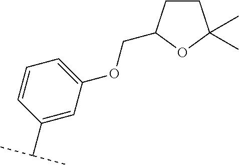 | 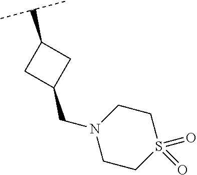 | 15 |
| 68 | I-1 | 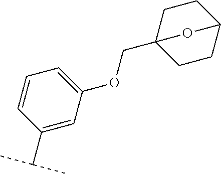 | 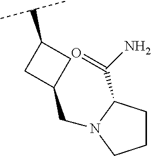 | 12 |
| 69 | I-1 | 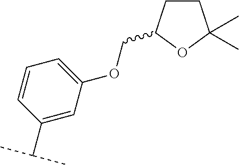 | 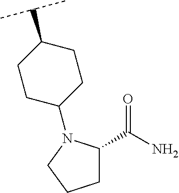 | 388 |
| 70 | I-1 | 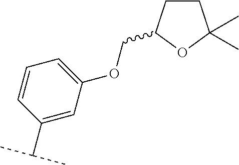 | 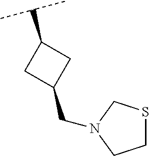 | 59 |
| 71 | I-1 | 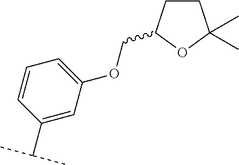 | 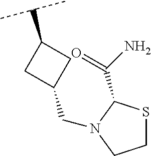 | <4 |
| 72 | I-1 | 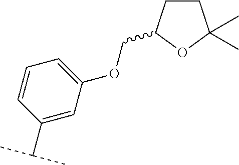 | 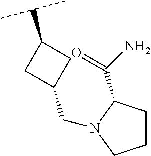 | 57 |
| 73 | I-1 | 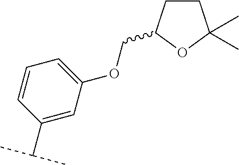 | 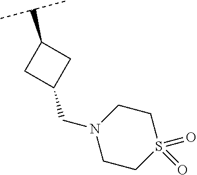 | 20 |
| 74 | I-1 | 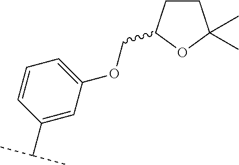 | 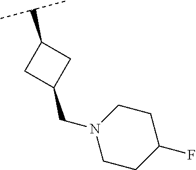 | 52 |
| 75 | I-1 | 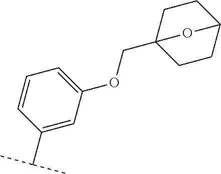 | 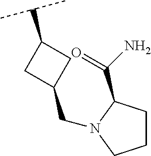 | 8 |
| 76 | I-1 | 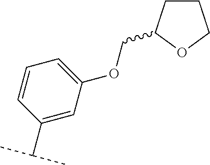 | 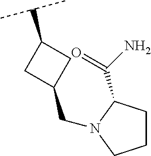 | 39 |
| 77 | I-1 | 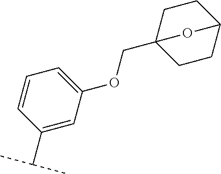 | 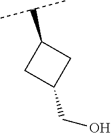 | |
| 78 | I-1 | 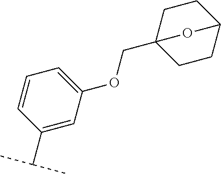 | 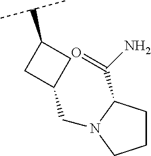 | 5 |
| 79 | I-1 | 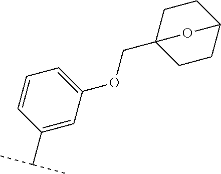 | 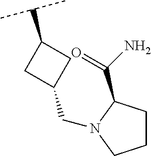 | 7 |
| 80 | I-1 | 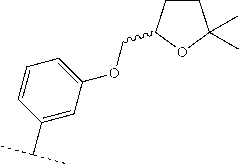 | 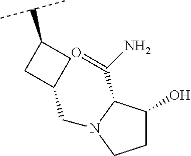 | 13 |
| 81 | I-1 | 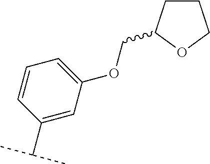 | 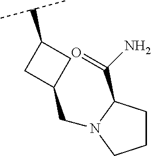 | 157 |
| 82 | I-1 | 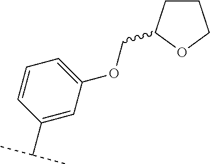 | 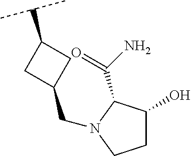 | 205 |
| 83 | I-1 | 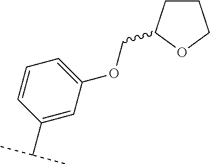 | 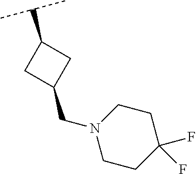 | 372 |
| 84 | I-1 | 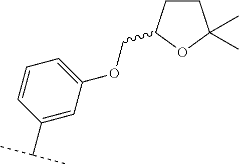 | 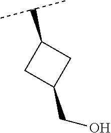 | 382 |
| 85 | I-1 | 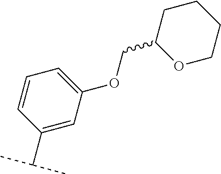 | 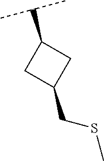 | 557 |
| 86 | I-1 | 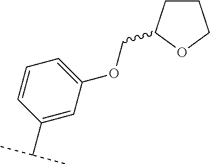 | 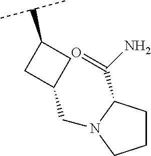 | 47 |
| 87 | I-1 | 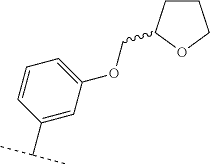 | 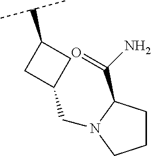 | 116 |
| 88 | I-1 | 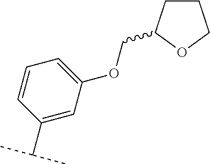 | 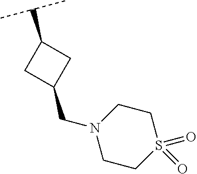 | 234 |
| 89 | I-1 | 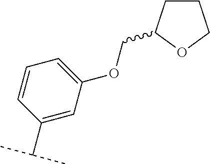 | 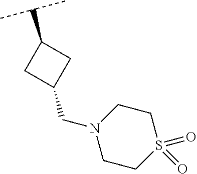 | 316 |
| 90 | I-1 | 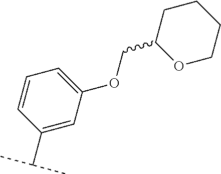 | 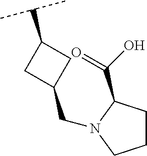 | 538 |
| 91 | I-1 | 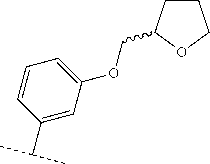 | 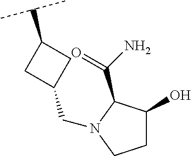 | 108 |
| 92 | I-1 | 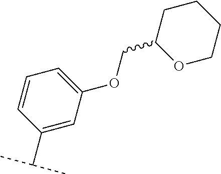 | 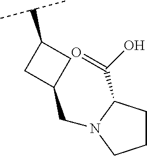 | 559 |
| 93 | I-1 | 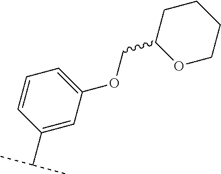 | 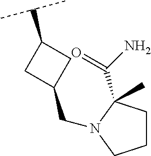 | 26 |
| 94 | I-1 | 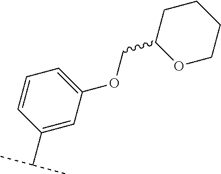 | 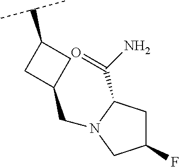 | 85 |
| 95 | I-1 | 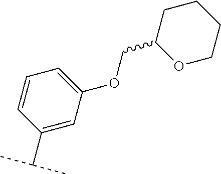 | 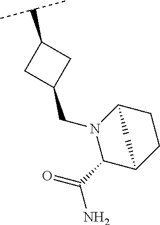 | 219 |
| 96 | I-1 | 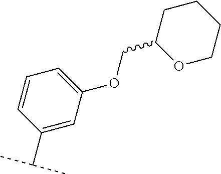 | 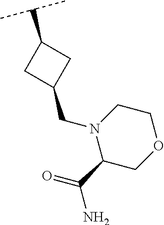 | 59 |
| 97 | I-1 | 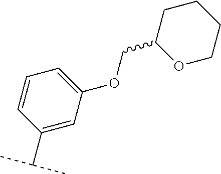 | 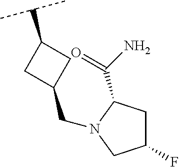 | 105 |
| 98 | I-1 | 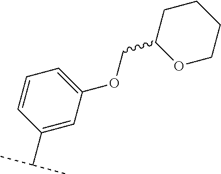 | 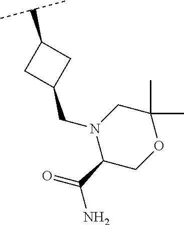 | 28 |
| 99 | I-1 | 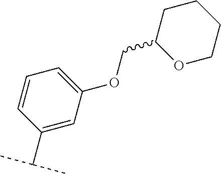 | 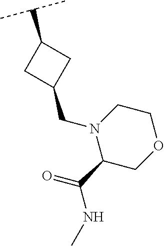 | 536 |
| 100 | I-1 | 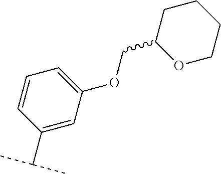 | 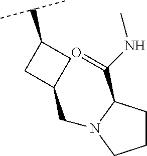 | 444 |
| 101 | I-1 | 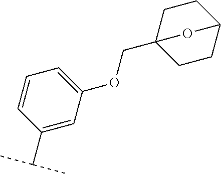 | 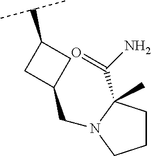 | 8 |
| 102 | I-1 | 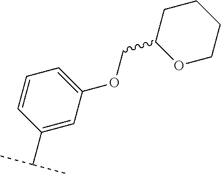 | 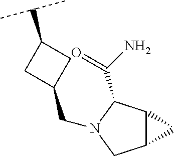 | 43 |
| 103 | I-1 | 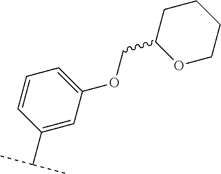 | 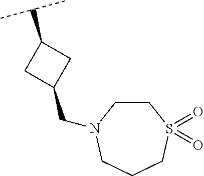 | 293 |
| 104 | I-1 | 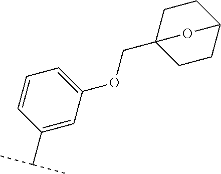 | 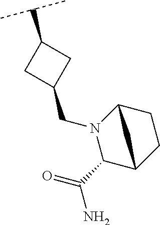 | 6 |
| 105 | I-1 | 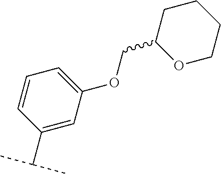 | 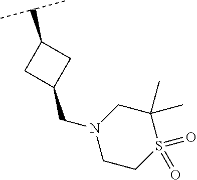 | 409 |
| 106 | I-1 | 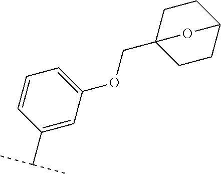 | 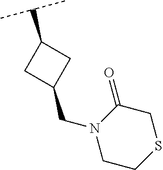 | 61 |
| 107 | I-1 | 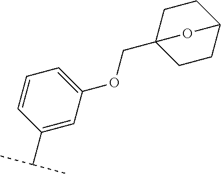 | 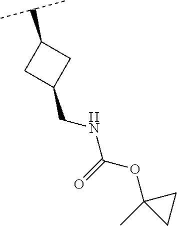 | 21 |
| 108 | I-1 | 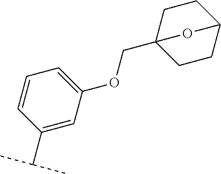 | 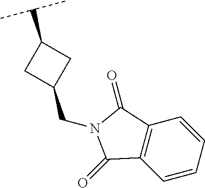 | 327 |
| 109 | I-1 | 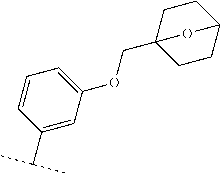 | 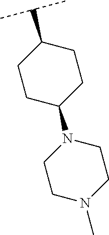 | 101 |
| 110 | I-1 | 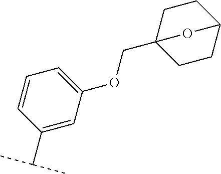 | 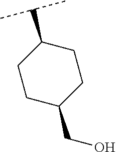 | 590 |
| 111 | I-1 | 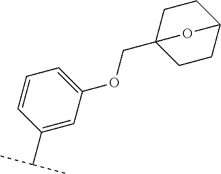 | 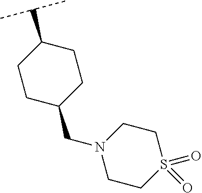 | 97 |
| 112 | I-1 | 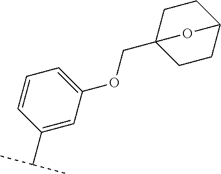 | 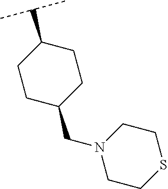 | 36 |
| 113 | I-1 | 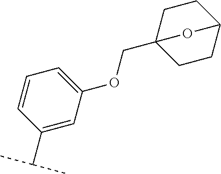 | 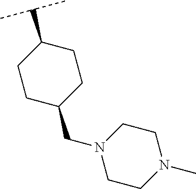 | 178 |
| 114 | I-1 | 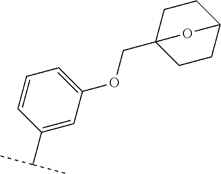 | 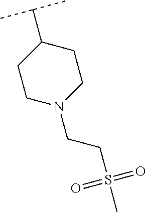 | 299 |
| 115 | I-1 | 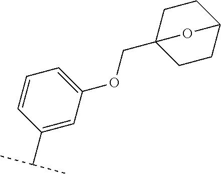 | 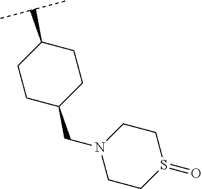 | 287 |
| 116 | I-1 | 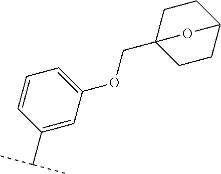 | 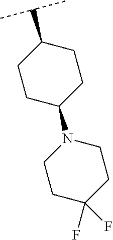 | 224 |
| 117 | I-1 | 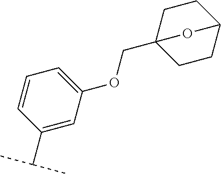 | 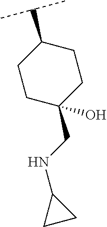 | 160 |
| 118 | I-1 | 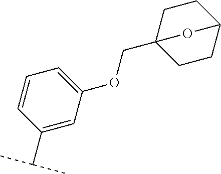 | 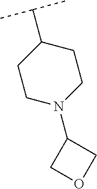 | 397 |
| 119 | I-1 | 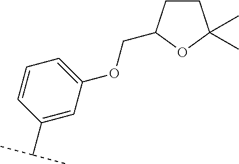 | 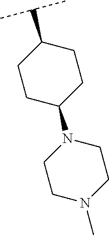 | 36 |
| 120 | I-1 | 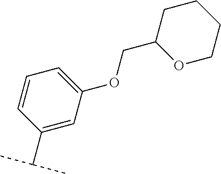 | 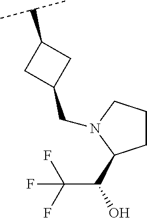 | 195 |
| 121 | I-1 | 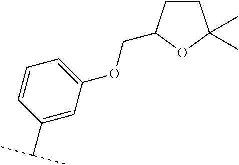 | 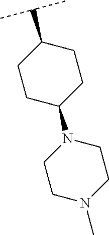 | 149 |
| 122 | I-2 | 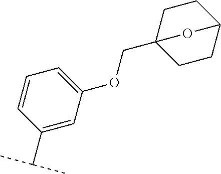 | 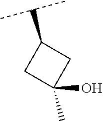 | 485 |
| 123 | I-1 | 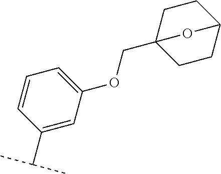 | 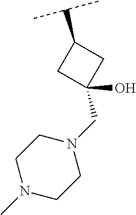 | 69 |
| 124 | I-1 | 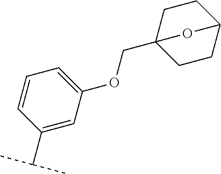 | 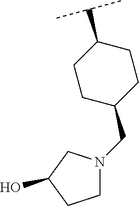 | 25 |
| 125 | I-1 | 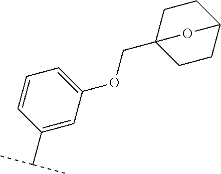 | 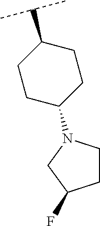 | 419 |
| 126 | I-1 | 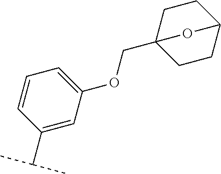 | 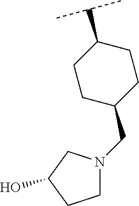 | 50 |
| 127 | I-1 | 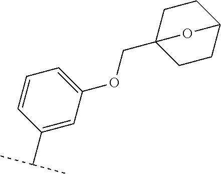 | 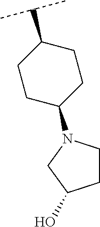 | 374 |
| 128 | I-1 | 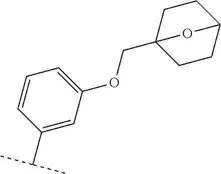 | 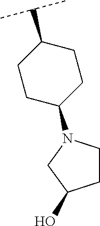 | 150 |
| 129 | I-1 | 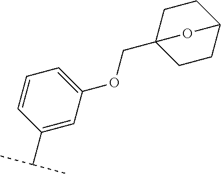 | 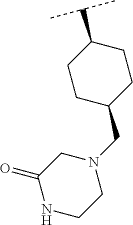 | 320 |
| 130 | I-1 | 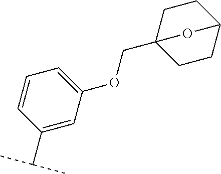 | 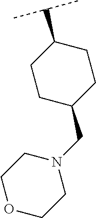 | 45 |
| 131 | I-1 | 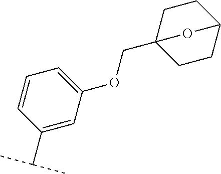 | 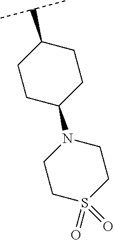 | 108 |
| 132 | I-1 | 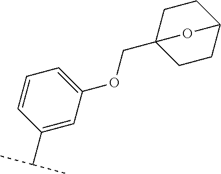 | 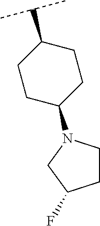 | 114 |
| 133 | I-1 | 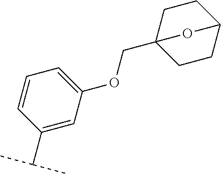 | 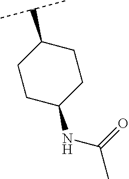 | 238 |
| 134 | I-1 | 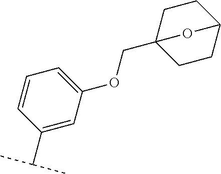 | 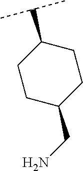 | 227 |
| 135 | I-1 | 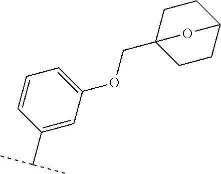 | 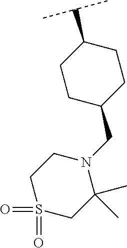 | 456 |
| 136 | I-1 | 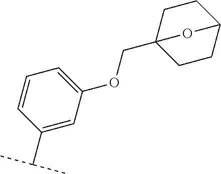 | 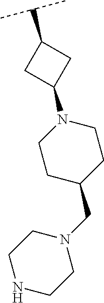 | 597 |
| 137 | I-1 | 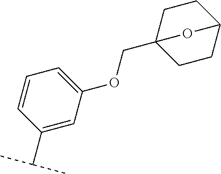 | 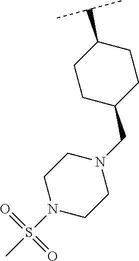 | 164 |
| 138 | I-1 | 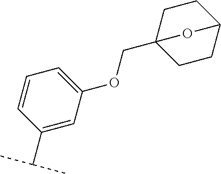 | 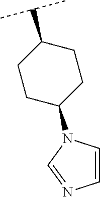 | 241 |
| 139 | I-1 | 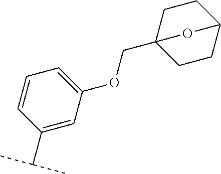 | 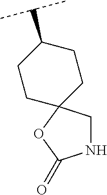 | 543 |
| 140 | I-1 | 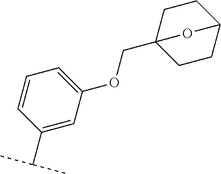 | 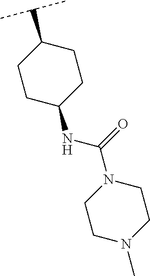 | 473 |
| 141 | I-1 | 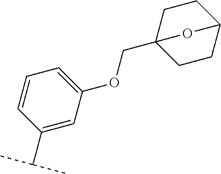 | 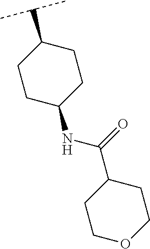 | 21 |
| 142 | I-1 | 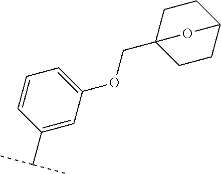 | 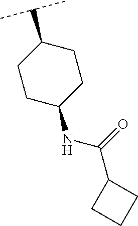 | 54 |
| 143 | I-1 | 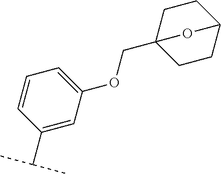 | 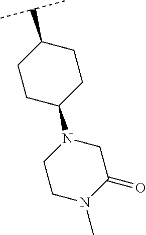 | 319-534 |
| 144 | I-1 | 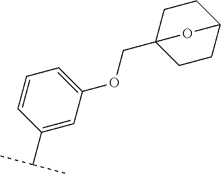 | 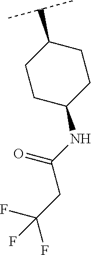 | 593 |
| 145 | I-1 | 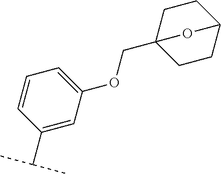 | 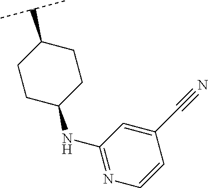 | 229 |
| 146 | I-1 | 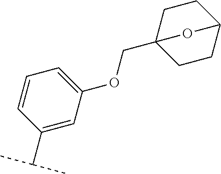 | 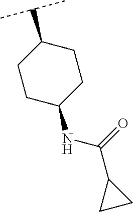 | 174 |
| 147 | I-1 | 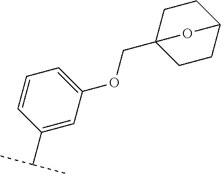 | 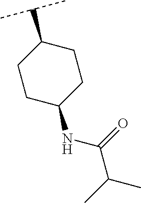 | 231 |
| 148 | I-1 | 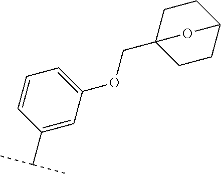 | 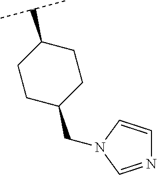 | 247 |
| 149 | I-1 | 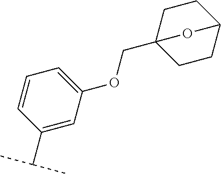 | 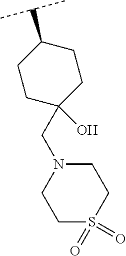 | 464 |
| 150 | I-1 | 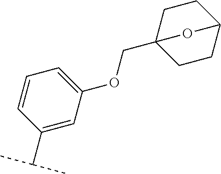 | 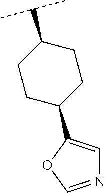 | 335 |
| 151 | I-1 | 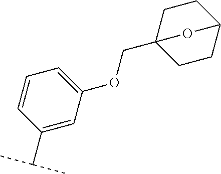 | 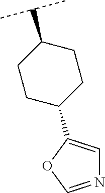 | 271 |
| 152 | I-1 | 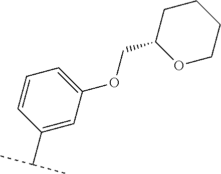 | 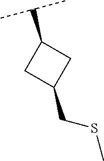 | 281 |
| 153 | I-1 | 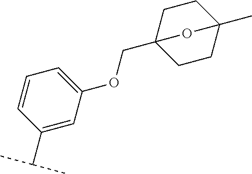 | 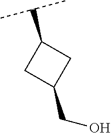 | 183 |
| 154 | I-1 | 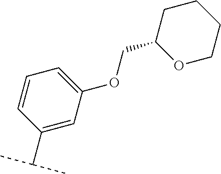 | 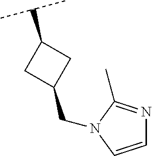 | 71 |
| 155 | I-1 | 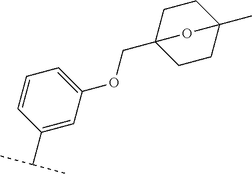 | 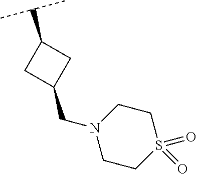 | 24-54 |
| 156 | I-1 | 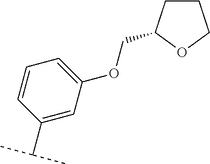 | 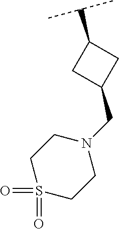 | 166 |
| 157 | I-1 | 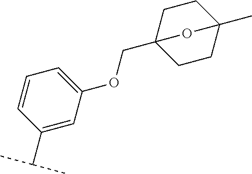 | 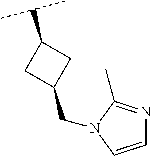 | 106-448 |
| 158 | I-1 | 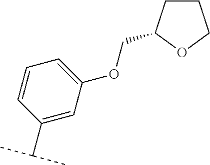 | 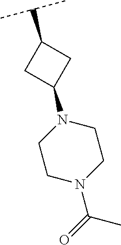 | |
| 159 | I-1 | 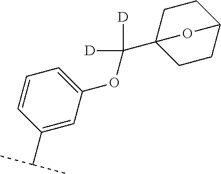 | 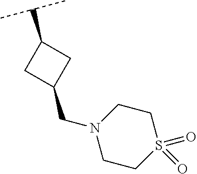 | 7-14 |
| 160 | I-1 | 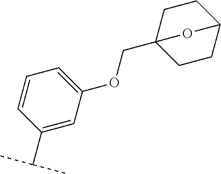 | 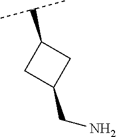 | 24 |
| 161 | I-1 | 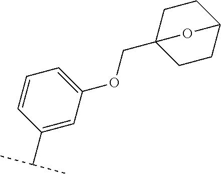 | 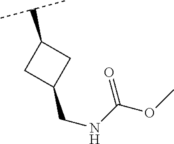 | 16 |
| 162 | I-1 | 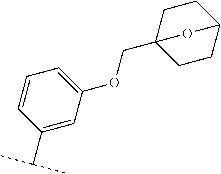 | 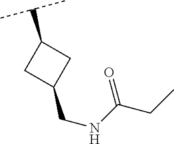 | 101 |
| 163 | I-1 | 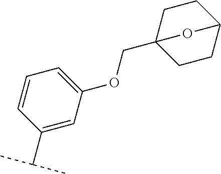 | 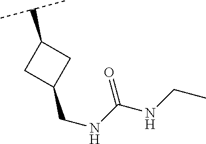 | 153 |
| 164 | I-1 | 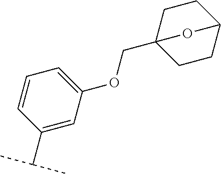 | 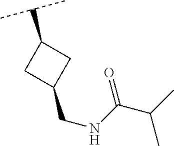 | 143 |
| 165 | I-1 | 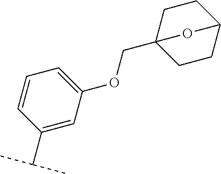 | 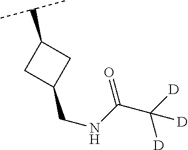 | 52 |
| 166 | I-1 | 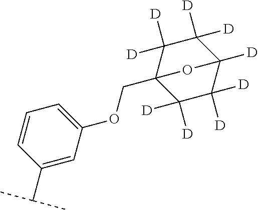 | 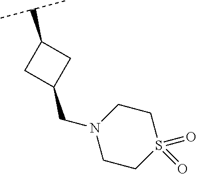 | 16 |
| 167 | I-1 | 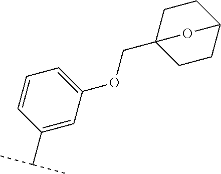 | 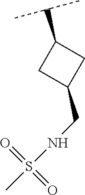 | 167 |
| 168 | I-1 | 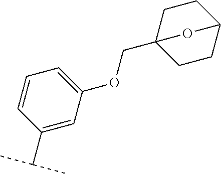 | 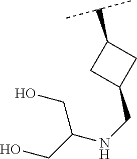 | 109 |
| 169 | I-1 | 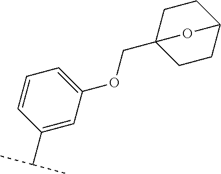 | 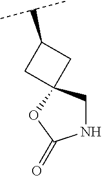 | 66 |
| 170 | I-1 | 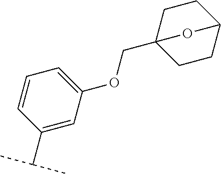 | 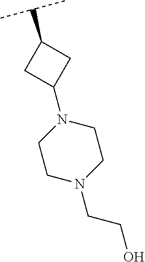 | 44 |
| 171 | I-1 | 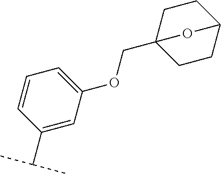 | 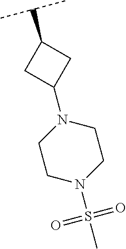 | 33 |
| 172 | I-1 | 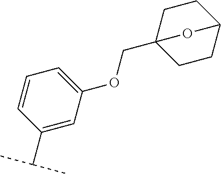 | 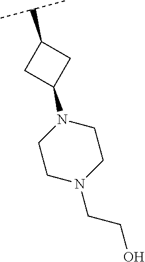 | 21 |
| 173 | I-1 | 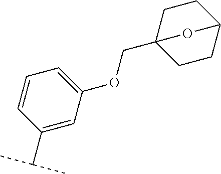 | 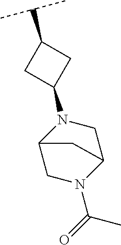 | 182 |
| 174 | I-1 | 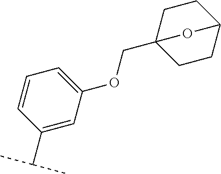 | 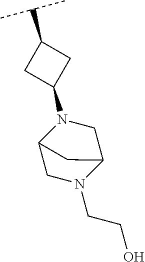 | 303 |
| 175 | I-1 | 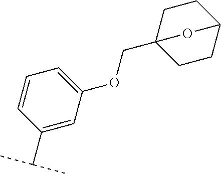 | 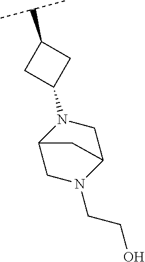 | 436 |
| 176 | I-1 | 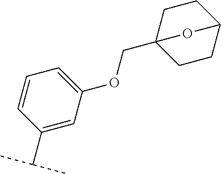 | 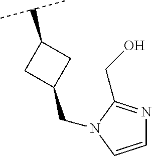 | 149 |
| 177 | I-1 | 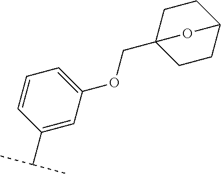 | 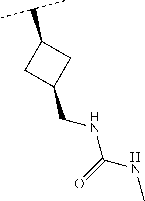 | 138 |
| 178 | I-1 | 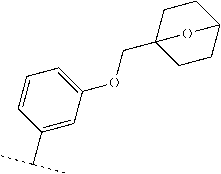 | 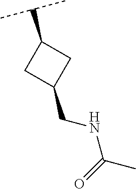 | 47-112 |
| 179 | I-1 | 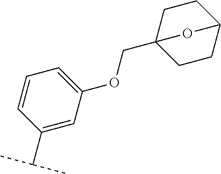 | 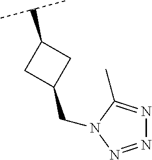 | 345 |
| 180 | I-1 | 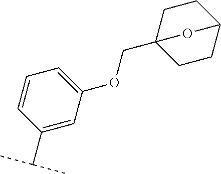 | 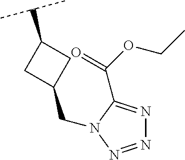 | 57 |
| 181 | I-2 | 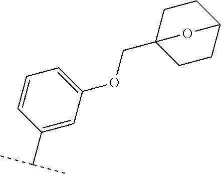 | 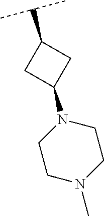 | 47 |
| 182 | I-1 | 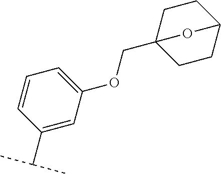 | 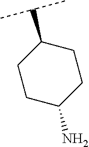 | 211 |
| 183 | I-1 | 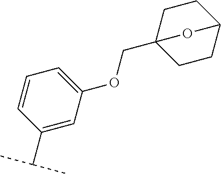 | 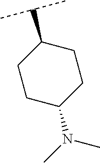 | 88 |
| 184 | I-1 | 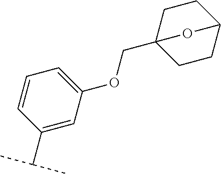 | 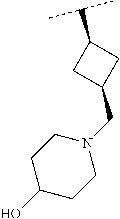 | 9 |
| 185 | I-1 | 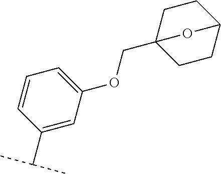 | 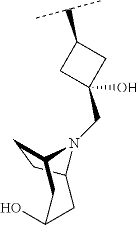 | 6 |
| 186 | I-1 | 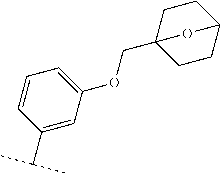 | 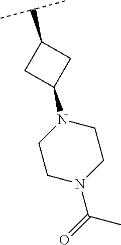 | 10-116 |
| 187 | I-2 | 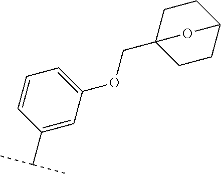 | 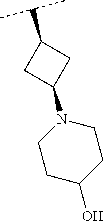 | 128 |
| 188 | I-1 | 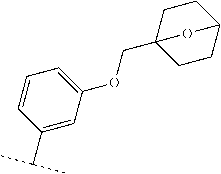 | 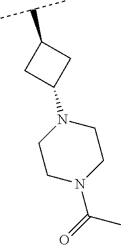 | 153 |
| 189 | I-1 | 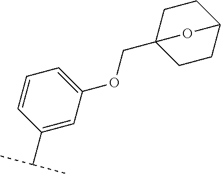 | 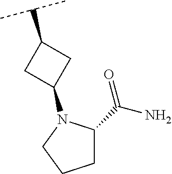 | 132 |
| 190 | I-1 | 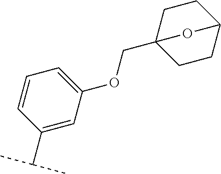 | 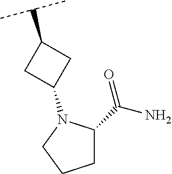 | 435 |
| 191 | I-1 | 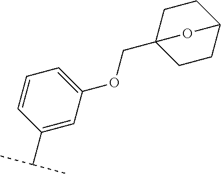 | 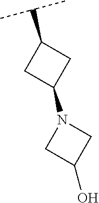 | 138 |
| 192 | I-1 | 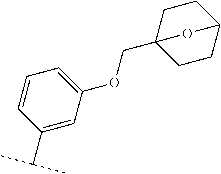 | 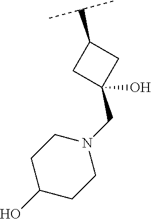 | 63 |
| 193 | I-1 | 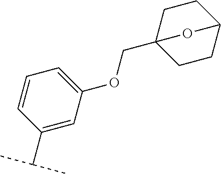 | 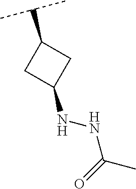 | 112 |
| 194 | I-1 | 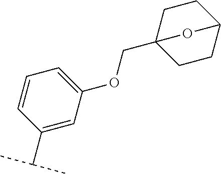 | 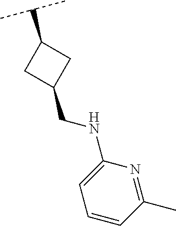 | 83 |
| 195 | I-1 | 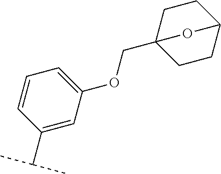 | 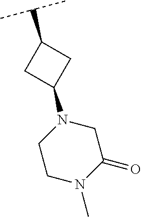 | 385 |
| 196 | I-1 | 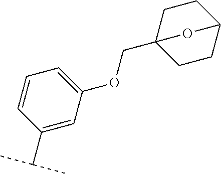 | 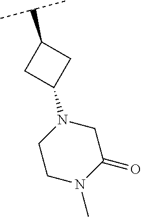 | 192 |
| 197 | I-1 | 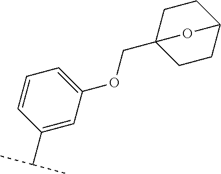 | 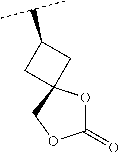 | 99 |
| 198 | I-1 | 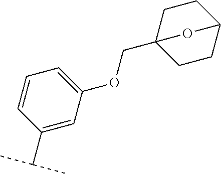 | 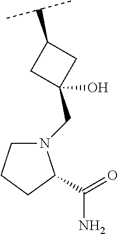 | 33 |
| 199 | I-1 | 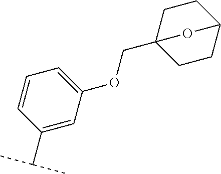 | 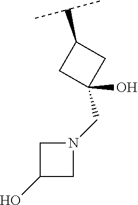 | 201 |
| 200 | I-1 | 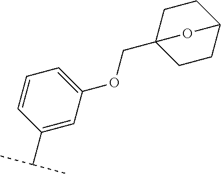 | 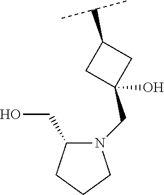 | 263 |
| 201 | I-1 | 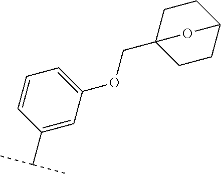 | 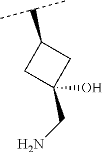 | 478 |
| 202 | I-1 | 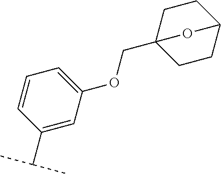 | 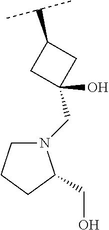 | 390 |
| 203 | I-1 | 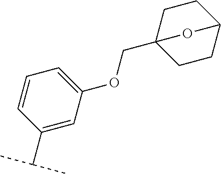 | 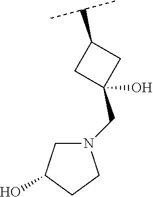 | 501 |
| 204 | I-1 | 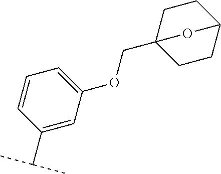 | 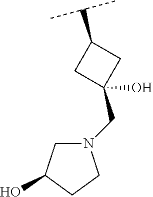 | 53 |
| 205 | I-1 | 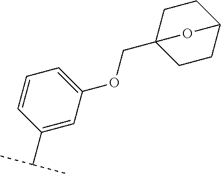 | 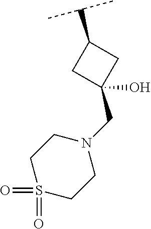 | 14 |
| 206 | I-1 | 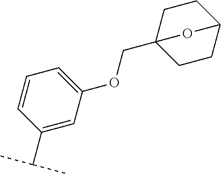 | 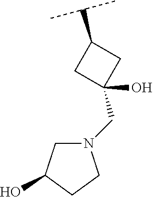 | 288 |
| 207 | I-1 | 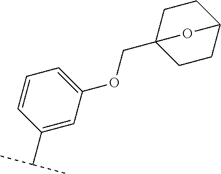 | 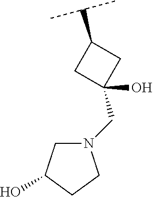 | 327 |
| 208 | I-1 | 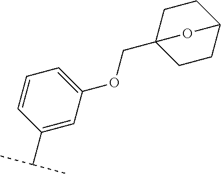 | 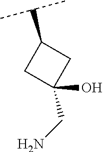 | 175 |
| 209 | I-1 | 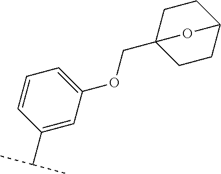 | 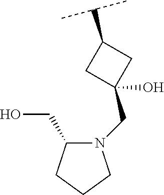 | 60 |
| 210 | I-1 | 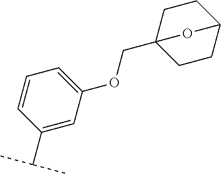 | 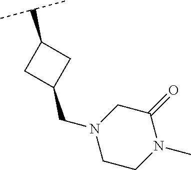 | 115-194 |
| 211 | I-1 | 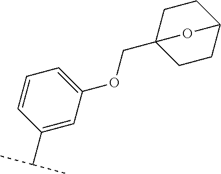 | 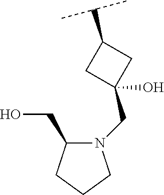 | 224 |
| 212 | I-1 | 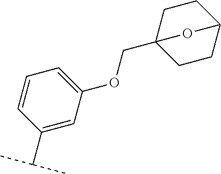 | 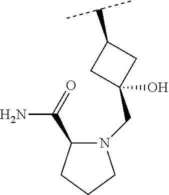 | 312 |
| 213 | I-1 | 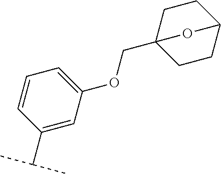 | 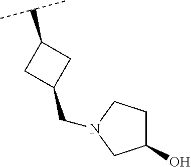 | 15 |
| 214 | I-1 | 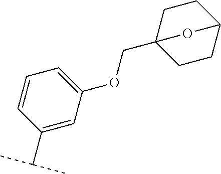 | 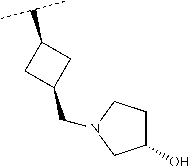 | 18 |
| 215 | I-1 | 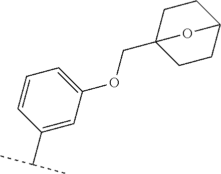 | 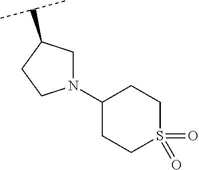 | 332 |
| 216 | I-1 | 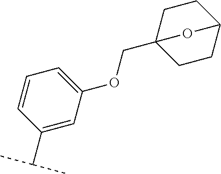 | 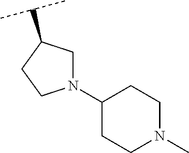 | 80 |
| 217 | I-1 | 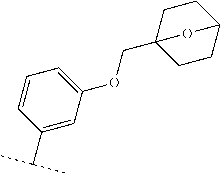 | 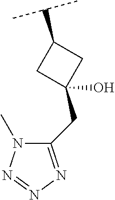 | 598 |
| 218 | I-1 | 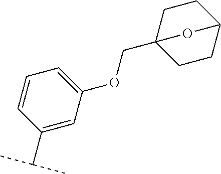 | 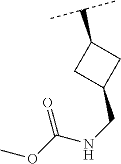 | 404 |
| 219 | I-1 | 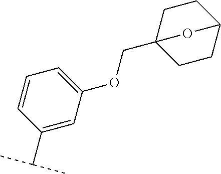 | 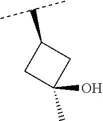 | 579 |
| 220 | I-1 | 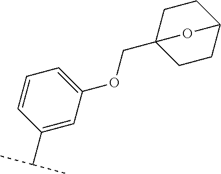 | 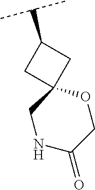 | 364 |
| 221 | I-1 | 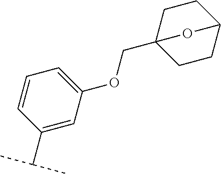 | 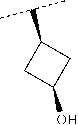 | 411 |
| 222 | I-1 | 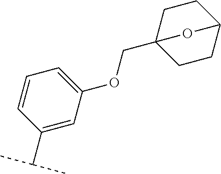 | 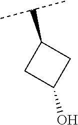 | 352 |
| 223 | I-1 | 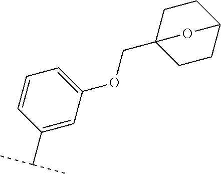 | 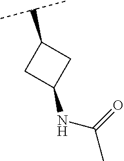 | 308 |
| 224 | I-1 | 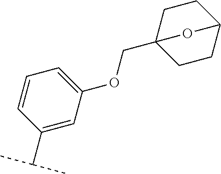 | 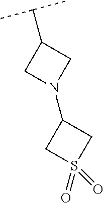 | 518 |
| 225 | I-1 | 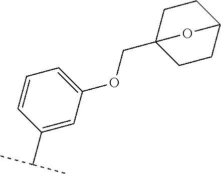 | 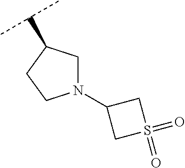 | 319 |
| 226 | I-1 | 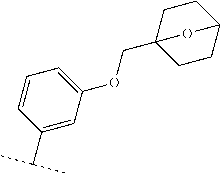 | 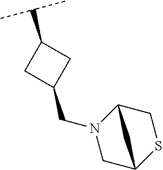 | |
| 227 | I-1 | 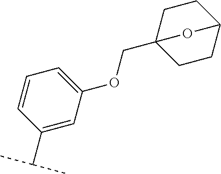 | 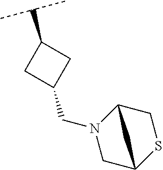 | 74 |
| 228 | I-1 | 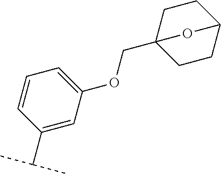 | 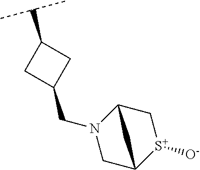 | 10-15 |
| 229 | I-1 | 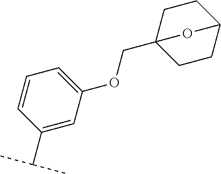 | 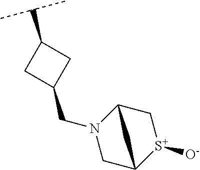 | 633 |
| 230 | I-1 | 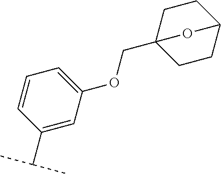 | 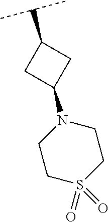 | 17 |
| 231 | I-1 | 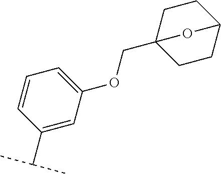 | 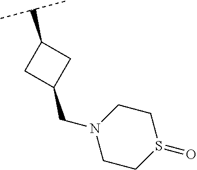 | 64 |
| 232 | I-1 | 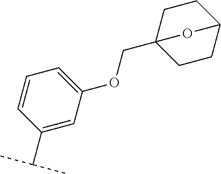 | 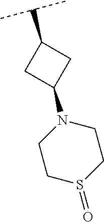 | 35-38 |
| 233 | I-1 | 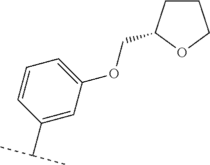 | 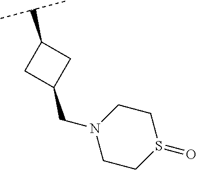 | 232 |
| 234 | I-1 | 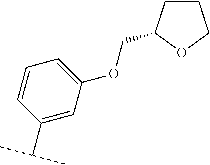 | 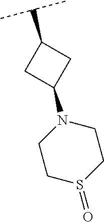 | 309 |
| 235 | I-1 | 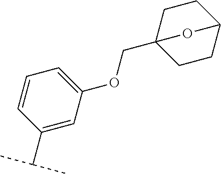 | 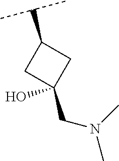 | 49 |
| 236 | I-1 | 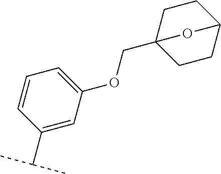 | 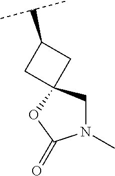 | |
| 237 | I-1 | 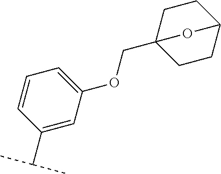 | 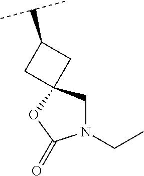 | |
| 238 | I-1 | 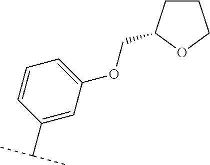 | 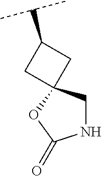 | 366 |

### Metabolic Stability

The compounds of the invention show improved efficacy and tolerability when compared to known IGF-1R inhibitors. Metabolic factors are anticipated to contribute to the observed improvements in efficacy and tolerability.

Effects of Metabolism: Known compounds have been shown to produce desirable effects in in-vivo models through the inhibition of IGF-1 receptor activity, they have been found to undergo extensive metabolism at the methylene group of the benzyl ether leading to cleavage to the corresponding phenol metabolite X:

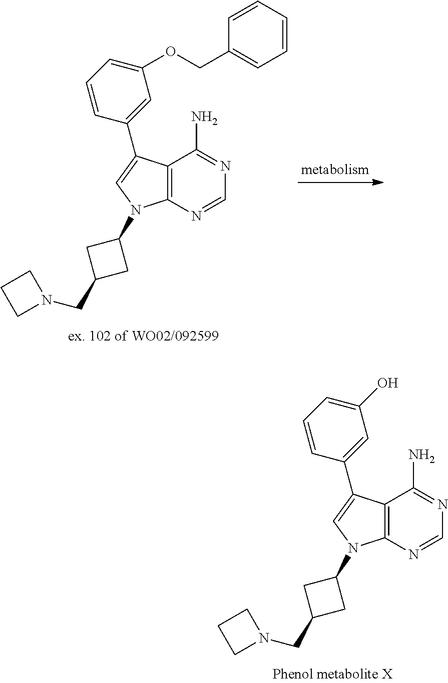

This not only limits the pharmacokinetic profile of such derivatives, but also generates phenolic metabolites, such as the specific example X, which shows multiple potent kinase activities, as shown in the following tests. In this tests, the parent compound, ex.102 of WO021092599 is compared with the corresponding phenol metabolite.

| | IC₅₀ (nM) | |
|---|---|---|
| Kinase | WO02/092599, ex.102 | Phenol metabolite X |
| IGF-1R | 82 | 420 |
| InsR | 230 | 3800 |
| C-Abl | >10000 | 47 |
| c-src | >10000 | 4 |
| EphB4 | >10000 | 9 |
| RET | 4400 | 8 |

Comparison of prior art compounds to inventive compounds: In a further series of tests, ex. 102 of WO021092599, is compared with Example 2 and Example 3 of the present invention.

The inventive compounds showed increased metabolic stability. In vitro metabolic identification studies with, Example 2 and Example 3 with human and rat liver microsomes are summarized in the table below in which the compounds were incubated for 1 hour with rat or human liver microsome preparations.

| | | Relative peak area after incubation | | |
|---|---|---|---|---|
| Metabolite | Species | WO02/092599, ex. 102 | Example 2 | Example 3 |
| Parent | rat | 49.2% | 69.3% | 75.8% |
| Phenol | rat | 11.4% | not detected | not detected |
| metabolite X | | | | |
| Parent | human | 71.5% | 90.3% | 81.7% |
| Phenol | human | 5.6% | not detected | not detected |
| metabolite X | | | | |

Greater metabolic stability being observed for Examples 2 and 3 relative to ex.102 of WO02/092559, with no evidence of formation of the phenolic metabolite X for the inventive compounds. Similarly, in vivo pharmacokinetic studies in the rat show greater metabolic stability and no detectable levels of the phenol metabolites for the inventive compounds compared to ex.102 of WO02/092559.

### Met Id Iv Vitro Incubation Method

The metabolism of unlabelled compound was examined following in vitro incubations with liver microsomes from rat and human. The samples were analyzed by capillary-HPLC/MS⁽ⁿ⁾ and screened for potential metabolites of the compound.

| In vitro | |
|---|---|
| Microsomal | Protein: 0.3 mg/ml; |
| incubation | Alamethicin: 1.25 μM; |
| | UDPGA: 2.4 mM |
| | GSH: 0, 1.5, 10 mM; |
| | Parent: 5 μM, NADPH-Reg. system |
| Species: | Rat, human |
| Incubation time | 1 h |
| Analytical method | Capillary-HPLC/MS(-MS) |
| MS | MS: LTQ XL (MS/MS) |
| | ITFS: 200-1500 amu |
| | ITMS2-5: 20-35% rel. coll. energy and |
| | wideband activation, |
| | Isolation width: 1.5 amu |
| HPLC column/ | Column switching: Reprosil ODS3 |
| temp. | C18, 5 μM, 0.4 × 11 mm |
| | Column: Reprosil ODS3 |
| | C18, 3 μM, 0.3 × 150 mm |
| | Analytical column thermostated at +40° C. |
| Mobile phase | A (H₂O/ACN, 95/5 + |
| | 0.1% CH₃COO NH₄ ⁺ 0.02% TFA) |
| | B (H₂O/ACN/MeOH, 5/47.5/47.5 + |
| | 0.1% CH₃COO NH₄ ⁺ 0.02% TFA) |
| | D₂O experiment performed, H/D exchange |
| | confirmed for all metabolites |
| Gradient | 5-95% B, 2-30 min at 4.5 μL/min |
| | (Pump: Chorus220) |

### Pharmaceutical Formualtions

### Tablets

Tablets comprising 50 mg of active ingredient, for example, one of the compounds of formula I described in Examples 1 to 238, and having the following composition are prepared in customary manner:

Composition:

| | | |
|---|---|---|
| active ingredient | 50 | mg |
| wheat starch | 150 | mg |
| lactose | 125 | mg |
| colloidal silicic acid | 12.5 | mg |
| talc | 22.5 | mg |
| magnesium stearate | 2.5 | mg |
| Total: | 362.5 | mg |

Preparation: The active ingredient is mixed with a portion of the wheat starch, with the lactose and the colloidal silicic acid and the mixture is forced through a sieve. A further portion of the wheat starch is made into a paste, on a water bath, with five times the amount of water and the powder mixture is kneaded with the paste until a slightly plastic mass is obtained. The plastic mass is pressed through a sieve of about 3 mm mesh size and dried, and the resulting dry granules are again forced through a sieve. Then the remainder of the wheat starch, the talc and the magnesium stearate are mixed in and the mixture is compressed to form tablets weighing 145 mg and having a breaking notch.

### Soft Capsules

5000 soft gelatin capsules comprising each 50 mg of active ingredient, for example one, of the compounds of formula I described in Examples 1 to 238, are prepared in customary manner:

Composition:

| | | |
|---|---|---|
| active ingredient | 250 | g |
| Lauroglykol | 2 | litres |

Preparation: The pulverized active ingredient is suspended in Lauroglykol® (propylene glycol laurate, Gattefosse S.A., Saint Priest, France) and ground in a wet pulverizer to a particle size of approx. 1 to 3 μm. 0.419 g portions of the mixture are then dispensed into soft gelatin capsules using a capsule-filling machine.

What is claimed is:

1. A compound according to formula I, 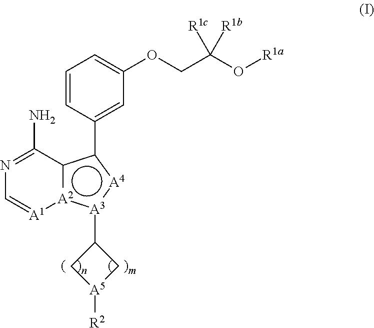
or a salt thereof, wherein
A¹ represents N, A² represents C, A³ represents N, and A⁴ represents CH; or
A¹ represents CH, A² represents N, A³ represents C, and A⁴ represents N;
R^{1a} and R^{1b} together with the atoms to which they are attached form a 3-12 membered monocyclic or bicyclic, saturated or partly saturated, heterocyclyl having 1-3 oxygen atoms, 0-3 nitrogen atoms, and 0-2 sulfur atoms; said heterocyclyl being optionally substituted with one to three substituents each independently selected from the group consisting of C₁₋₇alkyl; C₁₋₇alkoxy; halo; cyano; hydroxy; oxo; nitro; amino; C₁₋₇alkylamino; and di(C₁₋₇ alkyl)amino; and
R^{1c} represents hydrogen or C₁₋₇alkyl; or
R^{1a} and R^{1b} and R^{1c} together with the atoms to which they are attached form a 6-12 membered bicyclic, saturated or partly saturated, heterocyclyl, having 1-3 oxygen atoms, 0-3 nitrogen atoms, and 0-2 sulfur atoms; said heterocyclyl being optionally substituted with one to three substituents each independently selected from the group consisting of C₁₋₇alkyl; C₁₋₇alkoxy; halo; cyano; hydroxy; oxo; nitro; amino; C₁₋₇alkylamino; and di(C₁₋₇ alkyl)amino; or
R^{1a} represents branched C₃₋₈ alkyl or C₃₋₁₀cycloalkyl;
R^{1b} represents hydrogen or C₁₋₇ alkyl; and
R^{1c} represents hydrogen or C₁₋₇ alkyl;
m represents 1 or 2;
n represents 1 or 2;
A⁵-R² represents N—R², NC(H)R²R³, CR²R³, or CR³—CH₂—R²;
R³ represents hydrogen, C₁₋₇alkyl, or hydroxy; and
R² represents a 3-12 membered monocyclic or bicyclic, saturated or partly saturated, heterocyclyl having 1-3 nitrogen atoms, 0-3 oxygen atoms, and 0-3 sulfur atoms; said heterocyclyl being optionally substituted with one to four substituents each independently selected from the group consisting of halo; cyano; oxo; hydroxy; carboxy; amino; nitro; SO₂R⁴; COR⁵; C₁₋₇alkyl; C₁₋₇alkyl halo optionally substituted with one hydroxy; C₁₋₇alkoxy; hydroxy-C₁₋₇alkyl; piperazinyl C₁₋₃alkyl; aminocarbonyl; C₁₋₇alkylaminocarbonyl; and di(C₁₋₇alkyl)aminocarbonyl; or
R² represents OH; SH; C₁₋₇alkoxy; C₁₋₇alkylthio; C₁₋₇ alkyl optionally substituted with one SO₂R⁴ or NHR⁴ group; NHR⁵; NHC(O)R⁵; NHC(O)NHR⁵; NHC(O)OR⁵; SO₂R⁴; NHSO₂R⁵; NHNHC(O)R⁴; imidazolyl optionally substituted with one methyl, CH₂OH or C(O)OR⁷; tetrazolyl optionaly substituted with one methyl; or oxazoly; or
R² and R³ together with 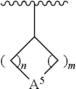
the moiety form 5,7-dioxa-spiro[3.4]octanyl, 5-oxa-7-aza-spiro[3.4]octanyl, or 5-oxa-8-aza-spiro[3.5]nonanyl, each of which is optionally substituted with one to three substituents each independently selected from the group consisting of: halo; cyano; oxo; hydroxy; amino; nitro; C₁₋₇ alkyl; C₁₋₇alkoxy; hydroxy-C₁₋₇alkyl; aminocarbonyl; C₁₋₇alkylaminocarbonyl; and di(C₁₋₇alkyl)aminocarbonyl;
R⁴ represents hydrogen or C₁₋₇ alkyl; and
R⁵ represents hydrogen; C₁₋₇ alkyl; hydroxy-C₁₋₇alkyl; C₁₋₇alkyl; halo; C₃₋₇ cycloalkyl optionally substituted with one or two C₁₋₃ alkyl groups; piperazinly optionally substituted with one C₁₋₃ alkyl; tetrahydropyranyl; pyridinyl optionally substituted with one methyl or cyano.

2. The compound according to claim 1, depicted by the formula I-1

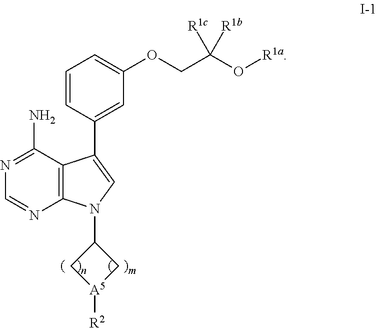

3. The compound according to claim 1, wherein
R^{1a} and R^{1b} together with the atoms to which they are attached form a 3-12 membered monocyclic, saturated heterocyclyl, containing 1-3 oxygen atoms,
said heterocyclyl being optionally substituted, with one to three substituents each independently selected from the group consisting of C₁₋₇alkyl; C₁₋₇alkoxy; halo; cyano; hydroxy; oxo; nitro; amino; C₁₋₇alkylamino; and di(C₁₋₇alkyl)amino; and
R^{1c} represents hydrogen; or
R^{1a}R^{1b} and R^{1c} together with the atoms to which they are attached form a bicyclic heterocyclyl, said heterocyclyl being selected from the following moieties: 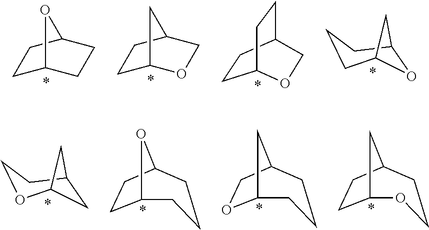
said heterocyclyl being bound to the molecule via the marked carbon atom, and
said heterocyclyl being optionally substituted with one to three substituents each independently selected from the group consisting of C₁₋₇alkyl; C₁₋₇alkoxy halo; cyano; hydroxy; oxo; nitro; amino; C₁₋₇alkylamino; and di(C₁₋₇alkyl)amino; or
R^{1a} represents branched C₃₋₈alkyl or C₃₋₁₀cycloalkyl,
R^{1b} represents hydrogen and
R^{1c} represents hydrogen.

4. The compound according to claim 3, wherein
R^{1a} and R^{1b} together with the atoms to which they are attached form tetrahydrofuryl or tetrahydro-2H-pyranyl optionally substituted with one to three substituents each independently selected from the group consisting of C₁₋₇alkyl; C₁₋₇alkoxy; halo; cyano; hydroxy; oxo; nitro; amino; C₁₋₇alkylamino; and di(C₁₋₇alkyl)amino; and
R^{1c} represents hydrogen; or R^{1a}, R^{1b} and R^{1c} together with the atoms to which they are attached form 8-oxabicyclo[3.2.1]octanyl, 7-oxabicyclo[2.2.1]heptanyl, or d₉-7-oxabicyclo[2.2.1]heptanyl optionally substituted with one to three substituents each independently selected from the group consisting of C₁₋₇alkyl; C₁₋₇alkoxy; halo; cyano; hydroxy; oxo; nitro; amino; C₁₋₇alkylamino; and di(C₁₋₇alkyl)amino; or
R^{1a} represents branched C₃₋₈alkyl or C₃₋₁₀cycloalkyl,
R^{1b} represents hydrogen; and
R^{1c} represents hydrogen.

5. The compound according to claim 1, wherein A⁵-R² represents CHR² or CH—CH₂—R².

6. The compound according to claim 1, wherein m represents 1.

7. The compound according to claim 1, wherein m and n represent 2.

8. The compound according to claim 1, wherein m and n represent 1.

9. The compound according to claim 5, wherein R² is selected from the group consisting of azetidinyl, pyrrolidinyl, piperidinyl, azepanyl, piperazinyl, tetrahydropyranyl, morpholinyl and thiomorpholinyl, each optionally substituted with one to four substituents each independently selected from the group consisting of halo; cyano; oxo; hydroxy;
carboxy; amino; nitro; SO₂R⁴; COR⁵; C₁₋₇alkyl; C₁₋₇alkyl halo optionally substituted with one hydroxy; C₁₋₇alkoxy; hydroxy-C₁₋₇alkyl; piperazinyl C₁₋₃alkyl; aminocarbonyl; C₁₋₇alkylaminocarbonyl; and di(C₁₋₇alkyl)aminocarbonyl.

10. The compound according to claim 9, wherein R² is selected from the group consisting of azetidinyl, pyrrolidinyl, piperidinyl, azepanyl, piperazinyl, tetrahydropyranyl, morpholinyl and thiomorpholinyl, each optionally substituted with one or two substituents each independently selected from the group consisting of C₁₋₃alkyl; fluoro; hydroxy; oxo; carboxy; C₁₋₃alkoxy carbonyl; C₁₋₃alkyl halo optionally substituted with one hydroxy; hydroxy-C₁₋₃alkyl; piperazinyl C₁₋₃alkyl; aminocarbonyl; C₁₋₃alkylaminocarbonyl; methoxycarbonyl; methylsulfonyl; and methylcarboxy.

11. The compound according to claim 10, wherein R² is selected from the group consisting of the following moieties: 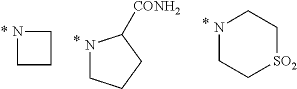
in which the marked atom is bound to A⁵.

12. The compound according to claim 1, wherein A⁵-R² represents CR²R³.

13. The compound according to claim 1, of formula I-7 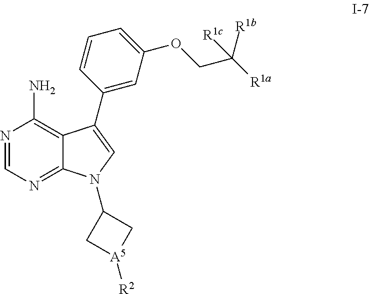
wherein
R^{1a} and R^{1b} together with the atoms to which they are attached form a tetrahydrofuranyl ring optionally substituted with one to three substituents each independently selected from the group consisting of C₁₋₇alkyl; C₁₋₇alkoxy; halo; cyano; hydroxy; oxo; nitro; amino; C₁₋₇alkylamino; and di(C₁₋₇alkyl)amino; and
R^{1c} represents hydrogen or C₁₋₇alkyl; or
R^{1a} and R^{1b} and R^{1c} together with the atoms to which they are attached form 7-oxabicyclo[2.2.1]heptanyl or d₉-7-oxabicyclo[2.2.1]heptanyl either being optionally substituted with one to three substituents each independently selected from the group consisting of C₁₋₇alkyl; C₁₋₇alkoxy; halo; cyano; hydroxy; oxo; nitro; amino C₁₋₇alkylamino, and di(C₁₋₇alkyl)amino;
A⁵-R² represents CR²R³ or CR³—CH₂—R²;
R³ represents hydrogen, C₁₋₇alkyl, or hydroxy; and
R² represents piperazinyl, thiomorpholinyl, or 2-thia-5-aza-bicyclo[2.2.1]heptanyl optionally substituted with one to four substituents each independently selected from the group consisting of halo; cyano; oxo; hydroxy; carboxy; amino; nitro; SO₂R⁴;
COR⁵; C₁₋₇alkyl; C₁₋₇alkyl halo optionally substituted with one hydroxy; C₁₋₇alkoxy;
hydroxy-C₁₋₇alkyl; piperazinly C₁₋₃ alkyl; aminocarbonyl; C₁₋₇alkylaminocarbonyl; and
di(C₁₋₇alkyl)aminocarbonyl; or
R² represents OH; or
R² and R³ together with the 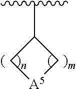
moiety form 5-oxa-7-aza-spiro[3.4]octanyl optionally substituted with one to three substituents each independently selected from the group consisting of: halo; cyano; oxo; hydroxy; amino; nitro; C₁₋₇alkyl; C₁₋₇alkoxy; hydroxy-C₁₋₇alkyl; aminocarbonyl; C₁₋₇alkylaminocarbonyl; and di(C₁₋₇alkyl)aminocarbonyl;
R⁴ represents hydrogen or C₁₋₇ alkyl; and
R⁵ represents hydrogen; C₁₋₇alkyl; hydroxy-C₁₋₇alkyl; C₁₋₇ alkyl halo; C₃₋₇ cycloalkyl optionally substituted with one or two C₁₋₃ alkyl groups; piperazinly optionally substituted with one C₁₋₃ alkyl; tetrahydropyranyl; or pyridinyl optionally substituted with one methyl or cyano.

14. The compound according to claim 1 which is:
cis-7-{3-[(1,1-dioxidothiomorpholin-4-yl)methyl]cyclobutyl}-5-{3-[(2S)-tetrahydrofuran-2-ylmethoxy]phenyl}-7H-pyrrolo[2,3-d]pyrimidin-4-amine;
7-[3-(1,1-dioxo-1-thiomorpholin-4-ylmethyl)-cyclobutyl]-5-[3-(7-oxa-bicyclo[2.2.1]hept-1-ylmethoxy)-phenyl]-7H-pyrrolo[2,3-d]pyrimidin-4-ylamine;
d₂-7-[cis-3-(1,1-dioxo-1-thiomorpholin-4-ylmethyl)-cyclobutyl]-5-[3-(7-oxa-bicyclo[2.2.1]hept-1-ylmethoxy)-phenyl]-7H-pyrrolo[2,3-d]pyrimidin-4-ylamine;
d₉-7-[cis-3-(1,1-dioxo-1-thiomorpholin-4-ylmethyl)-cyclobutyl]-5-[3-(7-oxa-bicyclo[2.2.1]hept-1-ylmethoxy)-phenyl]-7H-pyrrolo[2,3-d]pyrimidin-4-ylamine; or
7-[cis-3-(1,1-dioxo-thiomorpholin-4-yl)-cyclobutyl]-5-[3-(7-oxa-bicyclo[2.2.1]hept-1-ylmethoxy)-phenyl]-7H-pyrrolo[2,3-d]pyrimidin-4-ylamine; or a salt thereof.

15. The compound according to claim 1 which is:
(R)-1-(cis-3-{4-amino-5-[3-(7-oxa-bicyclo[2.2.1]hept-1-ylmethoxy)-phenyl]-pyrrolo[2,3-d]pyrimidin-7-yl}-cyclobutylmethyl)-pyrrolidine-2-carboxylic acid amide;
(S)-1-(trans-3-{4-amino-5-[3-(7-oxa-bicyclo[2.2.1]hept-1-ylmethoxy)-phenyl]-pyrrolo[2,3-d]pyrimidin-7-yl}-cyclobutylmethyl)-pyrrolidine-2-carboxylic acid amide; or
(R)-1-(trans-3-{4-amino-5-[3-(7-oxa-bicyclo[2.2.1]hept-1-ylmethoxy)-phenyl]-pyrrolo[2,3-d]pyrimidin-7-yl}-cyclobutylmethyl)-pyrrolidine-2-carboxylic acid amide; or a salt thereof.

16. The compound according to claim 1 which is:
1-{4-[cis-3-(4-amino-5-{3-(S)-1-(tetrahydrofuran-2-yl)methoxy]-phenyl}-pyrrolo[2,3-d]pyrimidin-7-yl)-cyclobutyl]-piperazin-1-yl}-ethanone;
7-[cis-3-(4-methanesulfonyl-piperazin-1-yl)-cyclobutyl]-5-[3-(7-oxa-bicyclo[2.2.1]hept-1-ylmethoxy)-phenyl]-7H-pyrrolo[2,3-d]pyrimidin-4-ylamine;
3-[3-(methyl-piperazin-1-yl)-cyclobutyl]-1-[3-(7-oxa-bicyclo[2.2.1]hept-1-ylmethoxy-phenyl]-imidazo[1,5-a]pyrazin-8-ylamine; or
1-[cis-4-(3-{4-Amino-5-[3-(7-oxa-bicyclo[2.2.1]hept-1-ylmethoxy)-phenyl]-pyrrolo[2,3-d]pyrimidin-7-yl}-cyclobutyl)-piperazin-1-yl]-ethanone; or a salt thereof.

17. The compound according to claim 1 which is:
(3-{4-amino-5-[3-(7-oxa-bicyclo[2.2.1]hept-1-ylmethoxy)-phenyl]-pyrrolo[2,3-d]pyrimidin-7-yl}-cyclobutylmethyl)-carbamic acid methyl ester, or a salt thereof.

18. The compound according to claim 1 which is:
(endo)-5-[3-(7-oxa-bicyclo[2.2.1]hept-1-ylmethoxy)-phenyl]-7-[3-((1S,2S,4S)-2-oxo-2-thia-5-aza-bicyclo[2.2.1]hept-5-ylmethyl)-cyclobutyl]-7H-pyrrolo[2,3-d]pyrimidin-4-ylamine;
5-[cis-3-(7-oxa-bicyclo[2.2.1]hept-1-ylmethoxy)-phenyl]-7-[3-(1-oxo-thiomorpholin-4-ylmethyl)-cyclobutyl]-7H-pyrrolo[2,3-d]pyrimidin-4-ylamine; or
5-[3-(7-Oxa-bicyclo[2.2.1]hept-1-ylmethoxy)-phenyl]-7-[cis-3-(1-oxo-thiomorpholin-4-yl)-cyclobutyl]-7H-pyrrolo[2,3-d]pyrimidin-4-ylamine; or a salt thereof.

19. A pharmaceutical composition comprising a therapeutically effective amount of a compound of formula I according to claim 1, and one or more pharmaceutically acceptable carriers.

20. A pharmaceutical combination comprising a therapeutically effective amount of a compound of formula I according to claim 1, and an antiproliferative agent.

21-25. (canceled)

26. A method of modulating IGF-1R activity in a subject in need thereof, comprising the step of administering to the subject a therapeutically effective amount of a compound of formula I according to claim 1.

27. A method for the treatment of an IGF-1R mediated disorder or disease comprising the step of administering to a subject a therapeutically effective amount of a compound of formula I according to claim 1.

28. The method of claim 27 wherein the disorder or disease is selected from the group consisting of multiple myeloma, neuroblastoma, synovial, hepatocellular, Ewing's Sarcoma, adrenocotical carcinoma (ACC) or a solid tumor selected from osteosarcoma, melanoma, tumor of breast, renal, prostate, colorectal, thyroid, ovarian, pancreatic, lung, uterine and gastrointestinal tumor.

29. The method of claim 27 wherein the disorder or disease is selected from acute lung injury and pulmonary fibrosis.
